(19) United States
(12) Patent Application Publication
(10) Pub. No.: US 2024/0252506 A1
(43) Pub. Date: Aug. 1, 2024

(54) COMPOUNDS FOR TREATMENT OF BACTERIAL INFECTION

(71) Applicant: Hoffmann-La Roche Inc., Little Falls, NJ (US)

(72) Inventors: Daniele ANDREOTTI, Verona (IT); John G. CUMMING, Therwil (CH); Bruno FASSARI, Verona (IT); Giacomo FOSSATI, Verona (IT); Elena SERRA, Verona (IT); Silvia PAOLETTA, Verona (IT)

(73) Assignee: Hoffmann-La Roche Inc., Little Falls, NJ (US)

(21) Appl. No.: 18/609,853

(22) Filed: Mar. 19, 2024

Related U.S. Application Data
(63) Continuation of application No. PCT/EP2022/075881, filed on Sep. 19, 2022.

(30) Foreign Application Priority Data
Sep. 20, 2021 (EP) 21197659.2

(51) Int. Cl. A61K 31/5383; A61K 9/20; A61K 9/48; A61P 31/04; C07D 498/04; C07D 519/00

(52) U.S. Cl. CPC A61K 31/5383; A61K 9/2013; A61K 9/2018; A61K 9/2054; A61K 9/485; A61K 9/4858; A61K 9/4866; A61P 31/04; C07D 498/04; C07D 519/00

(57) ABSTRACT

The invention provides novel compounds having the general formula (I)

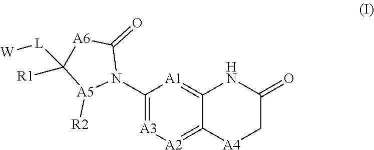

wherein R¹, R², A¹, A², A³, A⁴, A⁵, A⁶, L and W are as described herein, compositions including the compounds and methods of using the compounds.

### CROSS REFERENCE TO RELATED APPLICATIONS

This application is a continuation of International Application No. PCT/EP2022/075881, filed Sep. 19, 2022, which claims priority to European Patent Application No. 21197659.2 filed Sep. 20, 2021 both of which are incorporated herein by reference in their entirety.

### BACKGROUND OF THE INVENTION

Bacterial infections pose a continuing medical problem because anti-bacterial drugs eventually engender resistance in the bacteria on which they are used. Bacterial resistance against virtually all current antibiotic drugs is increasing. Many forms of antibiotic resistance can even cross international boundaries and spread with remarkable speed. Thus novel classes of antibacterial compounds are urgently needed.

### SUMMARY OF THE INVENTION

The invention provides compounds which exhibit broad spectrum antibacterial activity, their manufacture, pharmaceutical compositions comprising them and their use as medicaments for the treatment of microbial infections, in particular for the treatment of diseases and infections caused by Pseudomonas aeruginosa, Klebsiella pneumoniae, Escherichia coli, Acinetobacter baumannii and Staphylococcus aureus, more particularly caused by Gram negative bacterias, furthermore particularly to Escherichia coli.

The invention provides novel compounds of formula (I)

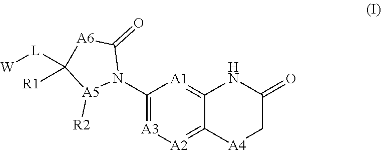

wherein
A¹ is —N—;
A² is selected from
   i) —N—, and
   ii) —CH—;
A³ is —CH—;
A⁴ is selected from
   i) —O—, and
   ii) —S—;
A⁵ is —CH—;
A⁶ is —O—;
W is selected from ring-systems:

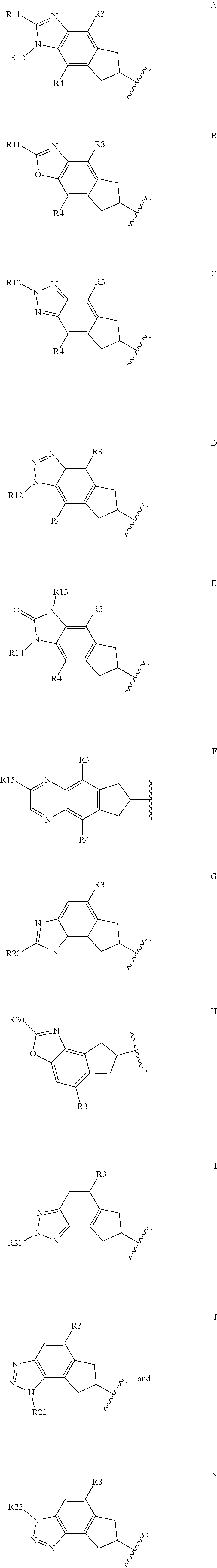

R² is H;
R³ and R⁴ are independently selected from
   i) H,
   ii) C₁₋₆-alkyl,
   iii) halogen,
   iv) OH, and
   v) cyano;
L is -L₁-NR¹⁰-L₂-;
L₁ is (CH₂)ₓ, wherein x represents an integer of 1 to 6;
L₂ is (CH₂)_{y} wherein y represents an integer of 1 to 5;
R¹ is H and R¹⁰ is selected from
   i) H, and
   ii) C₁₋₆-alkyl;
or R¹, R¹⁰ and L₂ and the atoms to which they are bonded form a 4-6 membered heterocycle ring comprising a single N heteroatom,
or R¹ and L₂ and the atoms to which they are bonded form a 3-6 membered cycloalkyl ring;
R¹¹ is selected from
   i) NH₂,
   ii) C₁₋₆-alkyl,
   iii) heterocycloalkyl,
   iv) heterocycloalkylalkyl,
   v) substituted cycloalkyl,
   vi) substituted heterocycloalkyl,
   vii) substituted heterocycloalkylalkyl,
   viii) aminoalkyl,
   ix) alkylaminoalkyl,
   x) (dialkylamino)alkyl,
   xi) alkylaminoacetamido,
   xii) aminohydroxyalkyl,
   xiii) hydroxyalkyl,
   xiv) hydroxy(alkylamino)alkyl,
   xv) —(CH₂)ₙNR^{a}R^{b}, and
   xvi) —CH₂—O—(CH₂)ₙNR^{a}R^{b},
   xvii) (dialkylamino)hydroxyalkyl, and
   xviii) H
   wherein n is an integer that is either 1 or 2,
   wherein substituted cycloalkyl, substituted heterocycloalkyl and substituted heterocycloalkyalkyl are substituted with 1-to-2 substituents independently selected from alkyl, alkoxy, amino, alkylamino, dialkylamino, OH, oxo and dioxo,
   wherein R^{a} is selected from

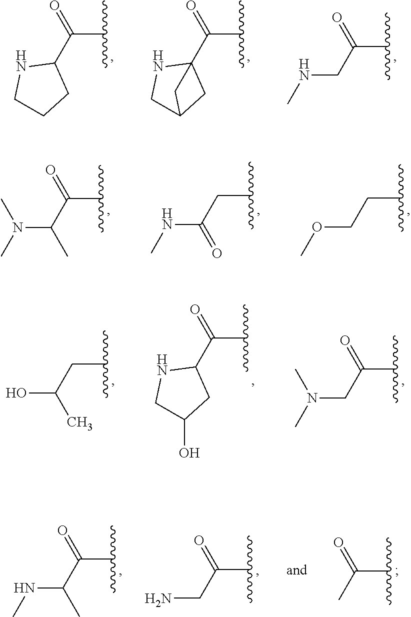

and R^{b} is selected from H or C₁₋₆-alkyl;
R¹² is selected from
   i) H,
   ii) C₁₋₆-alkyl,
   iii) aminoalkyl,
   iv) (alkylamino)acetamidoalkyl, and
   v) —(CH₂)₂NR^{c}R^{d},
   vi) Cycloalkylalkyl substituted by amino,
   vii) Cyclopropylaminoalkyl,
   viii) Heterocycloalkyl,
   ix) Alkylaminoalkyl, and
   x) (dialkylamino)alkyl
   wherein R^{c} is selected from

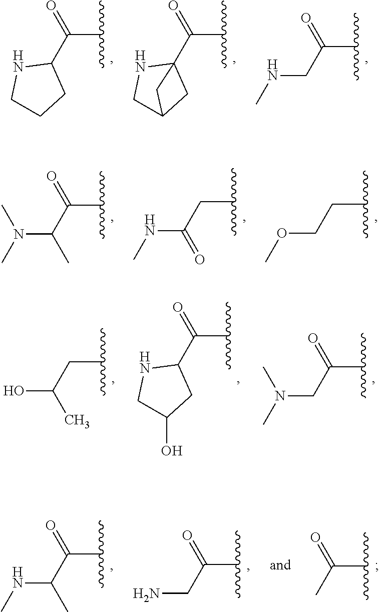

and R^{d} is selected from H or C₁₋₆-alkyl;
or R¹¹ and R¹² together form a 6-membered heterocycle ring comprising 1 or 2 N heteroatoms;
R¹³ is selected from
   i) H, and
   ii) aminoalkyl;
R¹⁴ is aminoalkyl;
R¹⁵ is selected from
   i) NH₂, and
   ii) (alkylamino)alkylamino;
R²⁰ is alkylaminoalkyl;
R²¹ is aminoalkyl, alkylaminoalkyl or (dialkylamino)alkyl;
R²² is aminoalkyl;
or pharmaceutically acceptable salts thereof.

Objects of the present invention are compounds of formula (I), their manufacture, medicaments based on a compound in accordance with the invention and their production as well as the use of compounds of formula (I), for the treatment or prophylaxis of bacterial infection, in particular for the treatment of diseases and infections caused by Escherichia coli.

### DETAILED DESCRIPTION OF THE INVENTION

The term “C₁₋₆-alkyl” denotes a monovalent linear or branched saturated hydrocarbon group of 1 to 6 carbon atoms. Examples of C₁₋₆-alkyl include methyl, ethyl, propyl, isopropyl, n-butyl, iso-butyl, sec-butyl, tert-butyl and pentyl. Particular C₁₋₆-alkyl groups are methyl, ethyl and n-butyl. More particular example is methyl.

The term “C₁₋₆-alkoxy” denotes a group of the formula —O—R′, wherein R′ is a C₁₋₆-alkyl group.

Examples of C₁₋₆-alkoxy groups include methoxy, ethoxy, n-propoxy, isopropoxy, n-butoxy, isobutoxy and tert-butoxy. Particular example is methoxy.

The term “C₃₋₈-cycloalkyl” or “cycloalkyl” denotes a monovalent saturated monocyclic or bicyclic hydrocarbon group of 3 to 8 ring carbon atoms. Bicyclic means a ring system consisting of two saturated carbocycles having one or two carbon atoms in common. Examples of monocyclic C₃-s-cycloalkyl are cyclopropyl, cyclobutanyl, cyclopentyl, cyclohexyl or cycloheptyl. Particular monocyclic cycloalkyl groups are C₃₋₆ cycloalkyl, such as cyclopropyl, cyclobutanyl, cyclopentyl and cyclohexyl. More particular monocyclic cycloalkyl group is cyclobutyl.

The term “cyano” denotes a —CN group.

The term “halogen” denotes fluoro, chloro, bromo or iodo. Particular halogens are chloro and fluoro. More particular example is fluoro.

The term “heterocycloalkyl” or “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon which is optionally substituted with oxo. Bicyclic means consisting of two cycles having one or two ring atoms in common. The heterocycloalkyl is preferably a monovalent saturated or partly unsaturated monocyclic ring system of 4 to 6 ring atoms, comprising 1 or 2 ring heteroatoms selected from N, O and S (4- to 6-membered heterocycloalkyl). Examples for monocyclic saturated heterocycloalkyl are 4,5-dihydro-oxazolyl, oxetanyl, azetidinyl, pyrrolidinyl, 2-oxo-pyrrolidin-4-yl, 3-oxo-morpholin-6-yl, tetrahydrofuranyl, tetrahydrothiophenyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are oxabicyclo[2.2.1]heptanyl, oxaspiro[3.3]heptanyl, 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl, or dihydropyranyl. Heterocyclyl is preferably azetidinyl, piperidyl, pyrrolidinyl, tetrahydrofuranyl, morpholinyl, azabicyclo[2.1.1]hexan-1-yl, or thiazinanyl.

The term “heterocycloalkylalkyl” denotes an alkyl group wherein at least one of the hydrogen atoms of the alkyl group has been replaced a heterocycloalkyl group. Examples are tetrahydrofuranylalkyl, pyrrolidinylalkyl, piperidylalkyl, morpholinylalkyl and thiazinanylalkyl. Particular examples are azetidinylmethyl, morpholinylmethyl and thiazinanylmethyl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxymethyl-ethyl, hydroxypropyl, hydroxymethylpropyl and dihydroxypropyl. Particular examples are hydroxypropyl and hydroxymethylethyl.

The term “amino” denotes a —NH₂ group.

The term “alkylamino” denotes an amino group wherein one of the hydrogen atoms of the amino group has been replaced by an alkyl group. Example is methylamino.

The term “dialkylamino” or (dialkyl)amino denotes an amino group wherein two of the hydrogen atoms of the amino group have been replaced by two alkyl groups. Example is dimethylamino.

The term “aminoalkyl” denotes an alkyl group wherein at least one of the hydrogen atoms of the alkyl group has been replaced by an amino group. Examples of aminoalkyl include aminomethyl, aminoethyl and aminopropyl.

The term “alkylaminoalkyl” denotes an aminoalkyl group wherein one of the hydrogen atoms of the amino group has been replaced by an alkyl group. Examples of alkylaminoalkyl groups include methylaminomethyl and methylaminoethyl.

The term “(dialkylamino)alkyl” denotes an alkyl group wherein at least one of the hydrogen atoms of the alkyl group has been replaced by a dialkylamino group. Examples of (dialkylamino)alkyl include (dimethylamino)methyl and (dimethylamino)ethyl.

The term “(alkylamino)alkylamino” denotes an alkylamino group wherein one of the hydrogen atoms of the alkylamino group have been replaced by an alkylamino group. Example is (methylamino)ethylamino.

The term “hydroxy(alkylamino)alkyl” denotes an “alkylaminoalkyl” group wherein one of the hydrogen atoms of the alkylaminoalkyl group is replaced by a hydroxyl group. Examples of hydroxy(alkylamino)alkyl include hydroxy(methylamino)ethyl, and hydroxy(methylamino)propyl. Specifically, 2-hydroxy-1-(methylamino)ethyl and 3-hydroxy-2-(methylamino)propyl.

The term “aminohydroxyalkyl” denotes a hydroxyalkyl group wherein one of the hydrogen atoms of the hydroxyalkyl group is replaced by an amino group. Examples of aminohydroxyalkyl include aminohydroxypropyl (specifically, 1-amino-2-hydroxy-propyl), and aminohydroxyethyl.

The term “(dialkylamino)hydroxyalkyl” denotes a hydroxyalkyl group wherein one of the hydrogen atoms of the hydroxyalkyl group is replaced by a dialkylamino group. An example of (dialkylamino)hydroxyalkyl) is (dimethylamino)-3-hydroxypropyl.

The term “acetamido” denotes a group of the formula —NH—C(═O)CH₃.

The term “alkylaminoacetamido” denotes an acetamido group wherein one of the hydrogens on the methyl portion of the acetamido group is replaced by an alkylamino group. Examples of aminoacetamido include methylaminoacetamido, specifically (—NH—C(═O)—CH₂NHCH₃).

The term “alkylaminoacetamidoalkyl” or “(alkylamino)acetamidoalkyl” denotes an alkyl group wherein at least one of the hydrogen atoms of the alkyl group has been replaced by an alkylaminoacetamido group. Example of “alkylaminoacetamidoalkyl” is 2-[[2-(methylamino)acetyl]amino]ethyl, specifically (—CH₂CH₂NHC(═O)CH₂NHCH₃).

The term “oxo” denotes a divalent oxygen atom ═O.

The term “4- to 6-membered ring” denotes a monocyclic saturated or unsaturated ring system of 4 to 6 ring atoms. “4- to 6-membered ring” can comprise 1, 2, or 3 ring heteroatoms selected from N, O and S, the remaining ring atoms being carbon which is optionally substituted with oxo. Example for 4- to 6-membered ring are cyclohexane, cyclopentane, cyclobutane, azetidine, pyrrolidine, and piperidine. Particular 4- to 6-membered ring is cyclohexane.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine, lactic acid and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are the hydrochloride salts, methanesulfonic acid salts, lactic acid salts and citric acid salts.

The compounds of the present invention may absorb moisture, have absorbed water or form hydrates when left in the air. Such hydrates are also included in the salts of the present invention.

Further, the compounds of the present invention may absorb certain other solvents to form solvates. Such solvates are also encompassed in the present invention as salts of the compounds of the formula (I).

The term “protecting group” (PG) denotes a group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups. Particular protecting groups are the tert-butoxycarbonyl (Boc), benzyloxycarbonyl (Cbz), fluorenylmethoxycarbonyl (Fmoc) and benzyl (Bn) groups. Further particular protecting groups are the tert-butoxycarbonyl (Boc) and the fluorenylmethoxycarbonyl (Fmoc) groups. More particular protecting group is the tert-butoxycarbonyl (Boc) group.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention are compounds according to formula (I) as described herein and pharmaceutically acceptable salts thereof, in particular compounds according to formula (I) as described herein and pharmaceutically acceptable salts thereof, more particularly compounds according to formula (I) as described herein.

An embodiment of the present invention provides compounds according to formula (I) as described herein, wherein A⁴ is —O—.

An embodiment of the present invention provides compounds according to formula (I) as described herein, wherein A² is —N—.

An embodiment of the present invention provides compounds according to formula (I) as described herein, wherein R³ and R⁴ are independently selected from
i) H, and
ii) halogen.

An embodiment of the present invention provides compounds according to formula (I) as described herein, wherein W is selected from ring systems A, B, C, D, G, H and I.

A particular embodiment of the present invention provides compounds according to formula (I) as described herein, wherein W is selected from ring systems A, B, C, D and I.

An embodiment of the present invention provides compounds according to formula (I) as described herein, wherein R¹¹ is selected from
i) NH₂,
ii) C₁₋₆-alkyl,
iii) a 5- to 6-membered heterocycloalkyl comprising 1 N atom, 1 O atom, or both 1 N atom and 1 O atom,
iv) A 6-membered heterocycloalkylalkyl comprising 1 N atom and 1 O atom,
v) a 5-to-6-membered substituted heterocycloalkyl comprising 1 N atom, 1 O atom, or both 1 N atom and 1 O atom,
vi) a 6-membered substituted heterocycloalkylalkyl comprising 1 N atom and 1 S atom;
vii) aminoalkyl,
viii) alkylaminoalkyl,
ix) (dialkylamino)alkyl,
x) alkylaminoacetamido,
xi) aminohydroxyalkyl,
xii) hydroxyalkyl, and
xiii) hydroxy(alkylamino)alkyl,
xiv) H,
xv) (dialkylamino)hydroxyalkyl, and
xvi) —(CH₂)ₙNR^{a}R^{b}
   wherein n is an integer that is either 1 or 2,
   wherein R^{a} is selected from

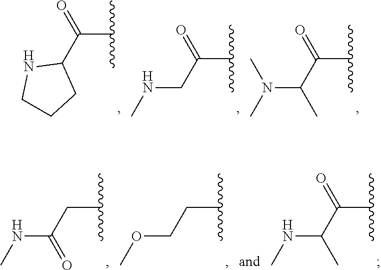

and R^{b} is H;
wherein substituted heterocycloalkyl and substituted heterocycloalkyalkyl are substituted with 1-to-2 substituents independently selected from alkyl, alkoxy, amino, alkylamino, OH, oxo and dioxo.

A particular embodiment of the present invention provides compounds according to formula (I) as described herein, wherein R¹¹ is selected from
i) a 4- to 6-membered heterocycloalkyl comprising 1 N atom, 1 O atom, or both 1 N atom and 1 O atom,
ii) a 5-to-6-membered substituted heterocycloalkyl comprising 1 N atom, 1 O atom, or both 1 N atom and 1 O atom,
iii) aminoalkyl,
iv) alkylaminoalkyl,
v) (dialkylamino)alkyl,
vi) alkylaminoacetamido,
vii) aminohydroxyalkyl, and
viii) hydroxy(alkylamino)alkyl,
wherein substituted heterocycloalkyl is substituted with alkoxy, amino, or OH.

A most particular embodiment of the present invention provides compounds according to formula (I) as described herein, wherein R¹¹ is selected from
i) a 5-membered substituted heterocycloalkyl comprising 1 N atom,
ii) a 4-membered heterocycloalkyl cinorusubg 1 N atom,
iii) aminoalkyl,
iv) alkylaminoalkyl,
v) (dialkylamino)alkyl, and
vi) aminohydroxyalkyl
wherein substituted heterocycloalkyl is substituted with OH.

An embodiment of the present invention provides compounds according to formula (I) as described herein, wherein R¹² is selected from
i) H,
ii) C₁₋₆-alkyl,
iii) aminoalkyl,
iv) a 4-membered heterocycloalkyl comprising 1 N atom,
v) alkylaminoalkyl, and
vi) (dialkylamino)alkyl.

A particular embodiment of the present invention provides compounds according to formula (I) as described herein, wherein R¹² is selected H, alkylaminoalkyl, aminoalkyl, or a 4-membered heterocycloalkyl ring comprising 1 N atom.

A preferred embodiment of the present invention provides compounds according to formula (I) as described herein, wherein R¹² is H.

An embodiment of the present invention provides compounds according to formula (I) as described herein, wherein R¹¹ and R¹² together form a 6-membered heterocycle ring comprising 2 N heteroatoms.

An embodiment of the present invention provides compounds according to formula (I) as described herein, wherein R¹ and R¹⁰ are H,
or R¹, R¹⁰ and L₂ and the atoms to which they are bonded form the piperidine ring as below

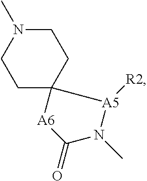

or R¹ and L₂ and the atoms to which they are bonded form the 4-membered cycloalkyl ring as below

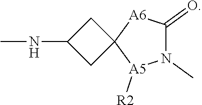

An embodiment of the present invention provides compounds according to formula (I) as described herein, wherein R¹, R¹⁰ and L₂ and the atoms to which they are bonded form the piperidine ring as below,

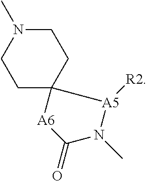

An embodiment of the present invention are compounds according to formula (I) as described herein, wherein
A¹ is —N—;
A² is selected from
   i) —N—, and
   ii) —CH—;
A³ is —CH—;
A⁴ is —O—;
A⁵ is —CH—;
A⁶ is —O—;
W is selected from ring-systems:

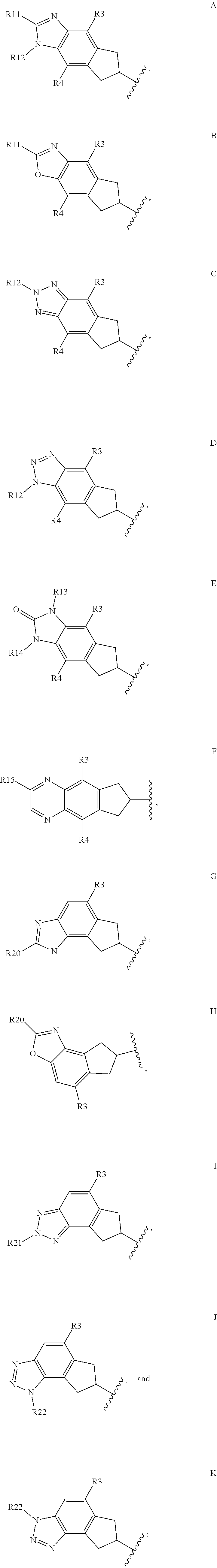

R² is H;
R³ and R⁴ are independently selected from H and halogen;
L is -L₁-NR¹⁰-L₂-;
L₁ is (CH₂)ₓ, wherein x represents an integer of 1 to 6;
L₂ is (CH₂)_{y} wherein y represents an integer of 1 to 5;
R¹ is H and R¹⁰ is selected from
   i) H, and
   ii) C₁₋₆-alkyl;
or R¹, R¹⁰ and L₂ and the atoms to which they are bonded form a 4-6 membered heterocycle ring comprising a single N heteroatom,
or R¹ and L₂ and the atoms to which they are bonded form a 3-6 membered cycloalkyl ring;
R¹¹ is selected from
   i) NH₂,
   ii) C₁₋₆-alkyl,
   iii) a 5- to 6-membered heterocycloalkyl comprising 1 N atom, 1 O atom, or both 1 N atom and 1 O atom,
   iv) A 6-membered heterocycloalkylalkyl comprising 1 N atom and 1 O atom,
   v) a 5-to-6-membered substituted heterocycloalkyl comprising 1 N atom, 1 O atom, or both 1 N atom and 1 O atom,
   vi) a 6-membered substituted heterocycloalkylalkyl comprising 1 N atom and 1 S atom;
   vii) aminoalkyl,
   viii) alkylaminoalkyl,
   ix) (dialkylamino)alkyl,
   x) alkylaminoacetamido,
   xi) aminohydroxyalkyl,
   xii) hydroxyalkyl, and
   xiii) hydroxy(alkylamino)alkyl,
   xiv) H,
   xv) (dialkylamino)hydroxyalkyl, and
   xvi) —(CH₂)ₙNR^{a}R^{b}
      wherein n is an integer that is either 1 or 2,
      wherein R^{a} is selected from

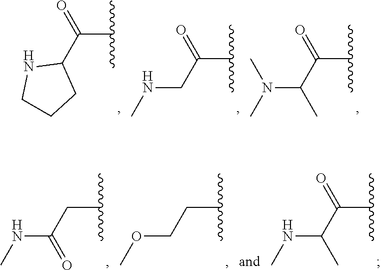

and R^{b} is H;
wherein substituted heterocycloalkyl and substituted heterocycloalkyalkyl are substituted with 1-to-2 substituents independently selected from alkyl, alkoxy, amino, alkylamino, OH, oxo and dioxo.
R¹² is selected from
   i) H,
   ii) C₁₋₆-alkyl,
   iii) aminoalkyl,
   iv) (alkylamino)acetamidoalkyl,
   v) —(CH₂)₂NR^{c}R^{d},
   vi) cycloalkylalkyl substituted by amino,
   vii) cyclopropylaminoalkyl,
   viii) heterocycloalkyl,
   ix) alkylaminoalkyl, and
   x) (dialkylamino)alkyl
   wherein R^{c} is selected from

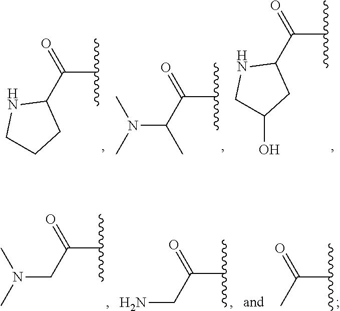

and R^{d} is selected from H or C₁₋₆-alkyl;
or R¹¹ and R¹² together form a 6-membered heterocycle ring comprising 1 or 2 N heteroatoms;
R¹³ is selected from H and aminoalkyl;
R¹⁴ is aminoalkyl;
R¹⁵ is selected from NH-2, and (alkylamino)alkylamino;
R²⁰ is alkylaminoalkyl;
R²¹ is aminoalkyl or alkylaminoalkyl;
R²² is aminoalkyl;
or pharmaceutically acceptable salts thereof.

A preferred embodiment of the present invention are compounds according to formula (I) as described herein, wherein
A¹ is —N—;
A² is selected from
   i) —N—, and
   ii) —CH—;
A³ is —CH—;
A⁴ is —O—;
A⁵ is —CH—;
A⁶ is —O—;
W is selected from ring-systems A, B, C, D, G, H and I.
R² is H;
R³ and R⁴ are independently selected from
   i) H, and
   ii) halogen;
L is -L₁-NR¹⁰-L₂-;
L₁ is (CH₂)ₓ, wherein x is 1;
R¹, R¹⁰ and L₂ and the atoms to which they are bonded form the piperidine ring as below,

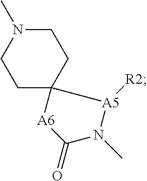

R¹¹ is selected from
   i) a 4- to 6-membered heterocycloalkyl comprising 1 N atom, 1 O atom, or both 1 N atom and 1 O atom,
   ii) a 5-to-6-membered substituted heterocycloalkyl comprising 1 N atom, 1 O atom, or both 1 N atom and 1 O atom,
   iii) aminoalkyl,
   iv) alkylaminoalkyl,
   v) (dialkylamino)alkyl,
   vi) alkylaminoacetamido,
   vii) aminohydroxyalkyl, and
   viii) hydroxy(alkylamino)alkyl,
   wherein substituted heterocycloalkyl is substituted with alkoxy, amino, or OH.
R¹² is selected from
   i) H,
   ii) C₁₋₆-alkyl,
   iii) aminoalkyl,
   iv) a 4-membered heterocycloalkyl comprising 1 N atom,
   v) aminoalkyl,
   vi) alkylaminoalkyl, and
   vii) (dialkylamino)alkyl
R²⁰ is alkylaminoalkyl;
R²¹ is alkylaminoalkyl or amimoalkyl;
or pharmaceutically acceptable salts thereof.

A preferred embodiment of the present invention are compounds according to formula (I) as described herein, wherein
A¹ is —N—;
A² is —N—;
A³ is —CH—;
A⁴ is —O—;
A⁵ is —CH—;
A⁶ is —O—;
W is selected from ring-systems A, B, C, D and I.
R² is H;
R³ and R⁴ are independently selected from
   i) H, and
   ii) fluoro;
L is -L₁-NR¹⁰-L₂-;
L₁ is (CH₂)ₓ, wherein x is 1;
R¹, R¹⁰ and L₂ and the atoms to which they are bonded form the piperidine ring as below,

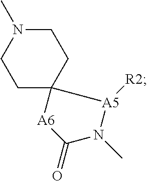

R¹¹ is selected from
   i) a 5-membered substituted heterocycloalkyl comprising 1 N atom substituted with OH;
   ii) a 4-membered heterocycloalkyl comprising 1 N atom,
   iii) aminoalkyl,
   iv) alkylaminoalkyl,
   v) (dialkylamino)alkyl, and
   vi) aminohydroxyalkyl
R¹² is selected from H, alkylaminoalkyl, aminoalkyl, or a 4-membered heterocycloalkyl ring comprising 1 N atom;
or pharmaceutically acceptable salts thereof.

An embodiment of the invention are compounds according to formula (I) as described herein, wherein
A¹ is —N—;
A² is selected from
   i) —N—, and
   ii) —CH—;
A³ is —CH—;
A⁴ is selected from
   i) —O—, and
   ii) —S—;
A⁵ is —CH—;
A⁶ is —O—;
W is selected from ring-systems:

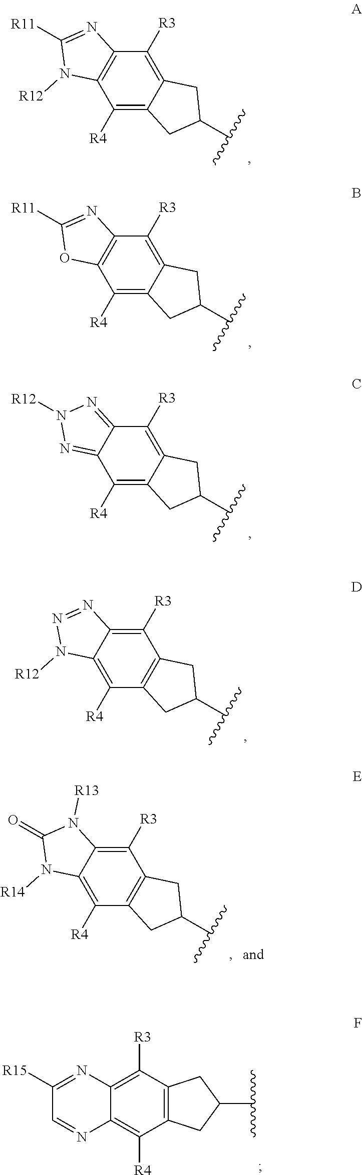

R² is H;
R³ and R⁴ are independently selected from
   i) H,
   ii) C₁₋₆-alkyl,
   iii) halogen,
   iv) OH, and
   v) cyano;
L is -L₁-NR¹⁰-L₂-;
L₁ is (CH₂)ₓ, wherein x represents an integer of 1 to 6;
L₂ is (CH₂)_{y} wherein y represents an integer of 1 to 5;
R¹ is H and R¹⁰ is selected from
   i) H, and
   ii) C₁₋₆-alkyl;
or R¹, R¹⁰ and L₂ and the atoms to which they are bonded form a 4-6 membered heterocycle ring comprising a single N heteroatom,
or R¹ and L₂ and the atoms to which they are bonded form a 3-6 membered cycloalkyl ring;
R¹¹ is selected from
   i) NH₂,
   ii) C₁₋₆-alkyl,
   iii) heterocycloalkyl,
   iv) heterocycloalkylalkyl,
   v) substituted cycloalkyl,
   vi) substituted heterocycloalkyl,
   vii) substituted heterocycloalkylalkyl,
   viii) aminoalkyl,
   ix) alkylaminoalkyl,
   x) (dialkylamino)alkyl,
   xi) alkylaminoacetamido,
   xii) aminohydroxyalkyl,
   xiii) hydroxyalkyl,
   xiv) hydroxy(alkylamino)alkyl,
   xv) —(CH₂)ₙNR^{a}R^{b},
   xvi) —CH₂—O—(CH₂)ₙNR^{a}R^{b},
   wherein n is an integer that is either 1 or 2,
   wherein substituted cycloalkyl, substituted heterocycloalkyl and substituted heterocycloalkyalkyl are substituted with 1-to-2 substituents independently selected from alkyl, alkoxy, amino, alkylamino, dialkylamino, OH, oxo and dioxo,
   wherein R^{a} is selected from

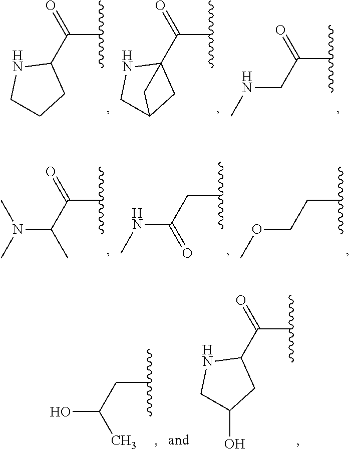

and R^{b} is selected from H or C₁₋₆-alkyl;
R¹² is selected from
   i) H,
   ii) C₁₋₆-alkyl, and
   iii) aminoalkyl,
   iv) (alkylamino)acetamidoalkyl, and
   v) —(CH₂)₂NR^{c}R^{d},
   wherein R^{c} is selected from

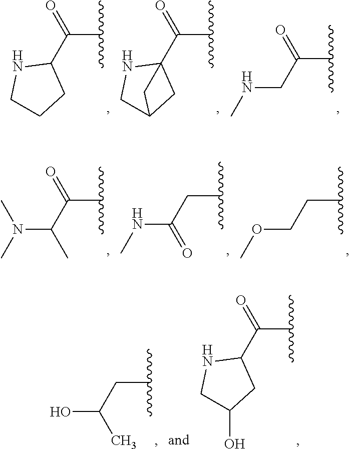

and R^{d} is selected from H or C₁₋₆-alkyl;
or R¹¹ and R¹² together form a 6-membered heterocycle ring comprising 1 or 2 N heteroatoms;
R¹³ is selected from
   i) H, and
   ii) aminoalkyl;
R¹⁴ is aminoalkyl;
R¹⁵ is selected from
   i) NH₂, and
   ii) (alkylamino)alkylamino;
or pharmaceutically acceptable salts thereof.

Another embodiment of the present invention are compounds according to formula (I) as described herein, wherein
A¹ is —N—;
A² is selected from
   i) —N—, and
   ii) —CH—;
A³ is —CH—;
A⁴ is —O—;
A⁵ is —CH—;
A⁶ is —O—;
W is selected from ring-systems

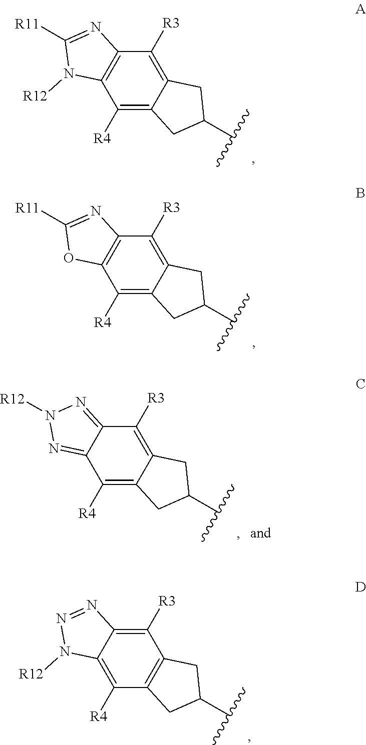

R² is H;
R³ and R⁴ are independently selected from
   i) H, and
   ii) halogen;
L is -L₁-NR¹⁰-L₂-;
L₁ is (CH₂)ₓ, wherein x is 1;
R¹, R¹⁰ and L₂ and the atoms to which they are bonded form the piperidine ring as below,

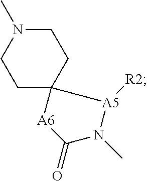

R¹¹ is selected from
   i) a 5- to 6-membered heterocycloalkyl comprising 1 N atom, 1 O atom, or both 1 N atom and 1 O atom,
   ii) a 5-to-6-membered substituted heterocycloalkyl comprising 1 N atom, 1 O atom, or both 1 N atom and 1 O atom,
   iii) aminoalkyl,
   iv) alkylaminoalkyl,
   v) (dialkylamino)alkyl,
   vi) alkylaminoacetamido,
   vii) aminohydroxyalkyl,
   viii) hydroxy(alkylamino)alkyl,
   wherein substituted heterocycloalkyl is substituted with alkoxy, amino, or OH.
R¹² is selected from
   i) H,
   ii) C₁₋₆-alkyl, and
   iii) aminoalkyl;
or pharmaceutically acceptable salts thereof.

Also a particular embodiment of the present invention provides compounds according to formula (I) as described herein, wherein
A¹ is —N—;
A² is —N—;
A³ is —CH—;
A⁴ is —O—;
A⁵ is —CH—;
A⁶ is —O—;
W is selected from ring-systems

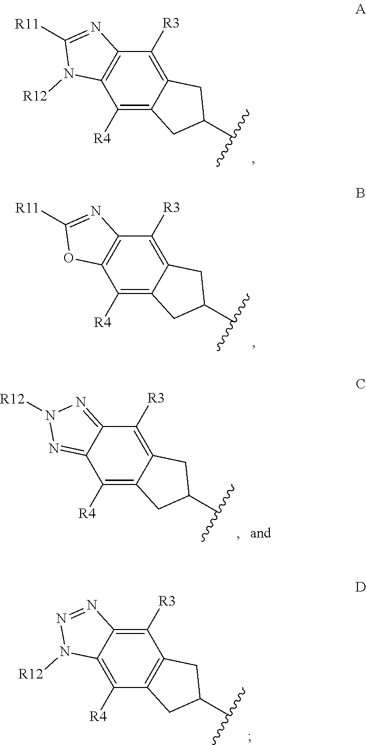

R² is H;
R³ and R⁴ are independently selected from
   i) H, and
   ii) fluoro;
L is -L₁-NR¹⁰-L₂-;
L₁ is (CH₂)ₓ, wherein x is 1;
R¹, R¹⁰ and L₂ and the atoms to which they are bonded form the piperidine ring as below,

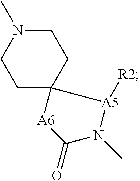

R¹¹ is selected from
   i) a 5-membered substituted heterocycloalkyl comprising 1 N atom,
   ii) aminoalkyl,
   iii) alkylaminoalkyl, and
   iv) (dialkylamino)alkyl,
   wherein substituted heterocycloalkyl is substituted with OH.
R¹² is selected from
   i) H, and
   ii) aminoethyl;
or pharmaceutically acceptable salts thereof.

An embodiment of the present invention provides compounds according to formula (I) as described herein, wherein A⁴ is —O—.

An embodiment of the present invention provides compounds according to formula (I) as described herein, wherein A² is —N—.

An embodiment of the present invention provides compounds according to formula (I) as described herein, wherein R³ and R⁴ are independently selected from
i) H, and
ii) halogen.

An embodiment of the present invention provides compounds according to formula (I) as described herein, wherein W is selected from ring systems

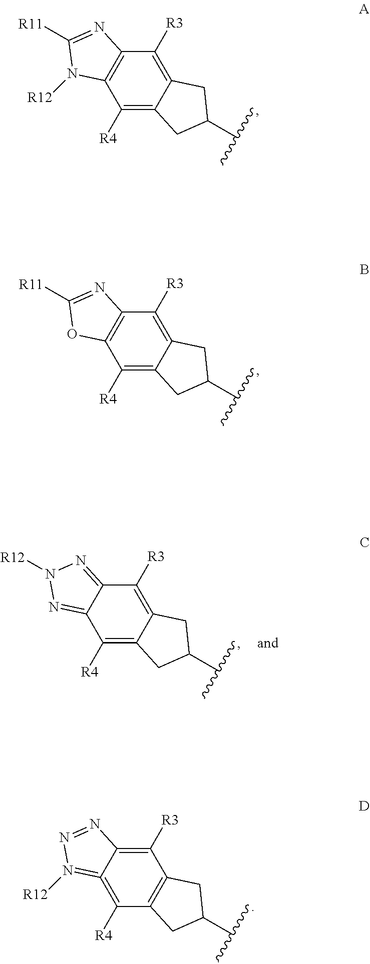

An embodiment of the present invention provides compounds according to formula (I) as described herein, wherein R¹¹ is selected from
i) NH₂,
ii) C₁₋₆-alkyl,
iii) a 5- to 6-membered heterocycloalkyl comprising 1 N atom, 1 O atom, or both 1 N atom and 1 O atom,
iv) A 6-membered heterocycloalkylalkyl comprising 1 N atom and 1 O atom,
v) a 5-to-6-membered substituted heterocycloalkyl comprising 1 N atom, 1 O atom, or both 1 N atom and 1 O atom,
vi) a 6-membered substituted heterocycloalkylalkyl comprising 1 N atom and 1 S atom;
vii) aminoalkyl,
viii) alkylaminoalkyl,
ix) (dialkylamino)alkyl,
x) alkylaminoacetamido,
xi) aminohydroxyalkyl,
xii) hydroxyalkyl, and
xiii) hydroxy(alkylamino)alkyl,
   wherein substituted heterocycloalkyl and substituted heterocycloalkyalkyl are substituted with 1-to-2 substituents independently selected from alkyl, alkoxy, amino, alkylamino, OH, oxo and dioxo.

A particular embodiment of the present invention provides compounds according to formula (I) as described herein, wherein R¹¹ is selected from
i) a 5- to 6-membered heterocycloalkyl comprising 1 N atom, 1 O atom, or both 1 N atom and 1 O atom,
ii) a 5-to-6-membered substituted heterocycloalkyl comprising 1 N atom, 1 O atom, or both 1 N atom and 1 O atom,
iii) aminoalkyl,
iv) alkylaminoalkyl,
v) (dialkylamino)alkyl,
vi) alkylaminoacetamido,
vii) aminohydroxyalkyl, and
viii) hydroxy(alkylamino)alkyl,
wherein substituted heterocycloalkyl is substituted with alkoxy, amino, or OH.

A particular embodiment of the present invention provides compounds according to formula (I) as described herein, wherein R¹¹ is selected from
i) a 5-membered substituted heterocycloalkyl comprising 1 N atom,
ii) aminoalkyl,
iii) alkylaminoalkyl,
iv) (dialkylamino)alkyl,
wherein substituted heterocycloalkyl is substituted with OH.

An embodiment of the present invention provides compounds according to formula (I) as described herein, wherein R¹² is selected from
i) H,
ii) C₁₋₆-alkyl, and
iii) aminoalkyl.

A particular embodiment of the present invention provides compounds according to formula (I) as described herein, wherein R¹² is selected from
i) H, and
ii) aminoethyl.

An embodiment of the present invention provides compounds according to formula (I) as described herein, wherein R¹¹ and R¹² together form a 6-membered heterocycle ring comprising 2 N heteroatoms.

An embodiment of the present invention provides compounds according to formula (I) as described herein, wherein R¹ and R¹⁰ are H,
or R¹, R¹⁰ and L₂ and the atoms to which they are bonded form the piperidine ring as below

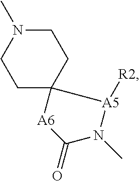

or R¹ and L₂ and the atoms to which they are bonded form the 4-membered cycloalkyl ring as below

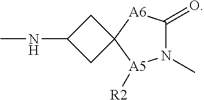

A particular embodiment of the present invention provides compounds according to formula (I) as described herein, wherein R¹, R¹⁰ and L₂ and the atoms to which they are bonded form the piperidine ring as below,

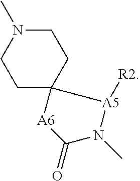

Processes for the manufacture of compounds of formula (I) as described herein are also an object of the invention.

The preparation of compounds of formula (I) of the present invention may be carried out in sequential or convergent synthetic routes. Syntheses of the invention are shown in the following general schemes. The skills required for carrying out the reactions and purifications of the resulting products are known to those skilled in the art. The substituents and indices used in the following description of the processes have the significance given herein before unless indicated to the contrary.

In more detail, the compounds of formula (I) can be manufactured by the methods given below, by the methods given in the examples or by analogous methods. Appropriate reaction conditions for the individual reaction steps are known to a person skilled in the art. The reaction sequence is not limited to the ones displayed in schemes 1-10, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The present compounds of formula (I) and their pharmaceutically acceptable salts can be prepared by a process described below (Scheme 1).

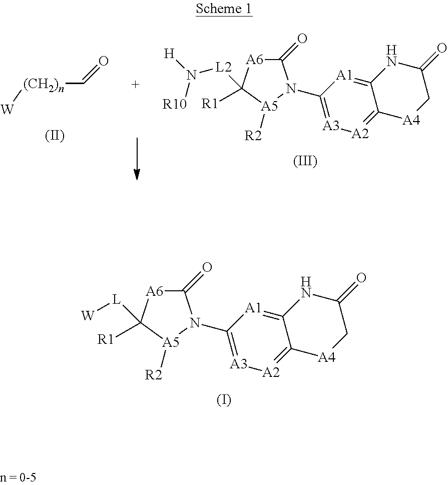

Reductive amination of aldehydes of formula (II) with amines of formula (III) affords compounds of formula (I). Typical conditions include sodium triacetoxyborohydride or sodium cyanoborohydride, optionally in the presence of additives such as N,N-diisopropylethylamine or triethlyamine or acetic acid or powdered molecular sieves in a solvent such as methanol, THF or 1,2-dichloroethane at a temperature such as room temperature.

Alternatively, compounds of formula (I) may be prepared as illustrated in scheme 2.

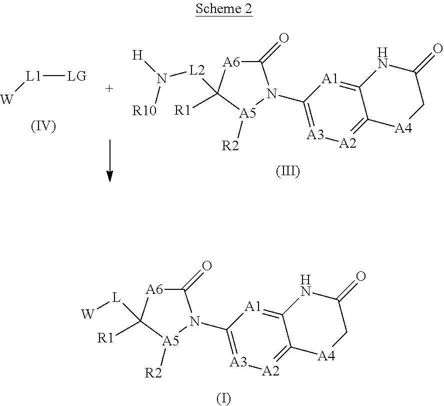

Reaction of intermediates of formula (IV) wherein LG is a leaving group such as iodo, bromo, mesyloxy or tosyloxy with amines of formula (III) affords compounds of formula (I). Typical conditions include a base such as potassium carbonate or caesium carbonate in a solvent such as MeCN, DMF, DMA or NMP at an elevated temperature such as 80° C.

Intermediates of formula (III) may be prepared as illustrated in scheme 3.

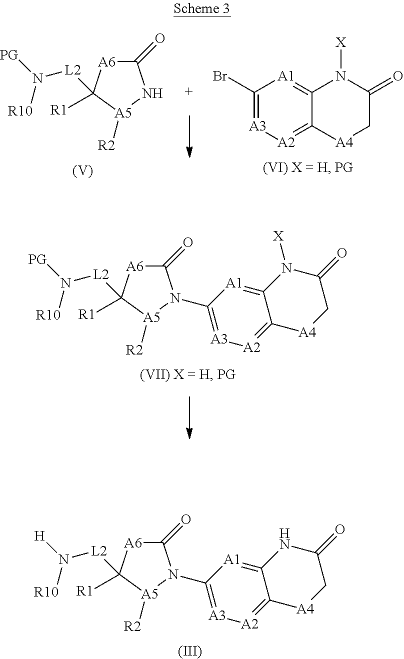

Coupling of compounds of formula (V), wherein PG is a suitable amine protecting group, with heteroaryl bromides of formula (VI), optionally protected with a protecting group (PG), affords intermediates of formula (VII) optionally protected. Typical conditions include the use of a catalytic quantity of copper (I) iodide and trans-N,N′-dimethylcyclohexane-1,2-diamine or of tris(dibenzylideneacetone)dipalladium (0) and 9,9-dimethyl-4,5-bis(diphenylphosphino)xanthene in the presence of a base such as potassium carbonate in a solvent such as 1,4-dioxane at a temperature such as 100° C. Typical protecting groups in protected amines (V) include BOC and benzyloxycarbonyl. Typical optional protecting groups in heteroaryl bromides of formula (VI) include 2-trimethylsilylethoxymethyl. Deprotection of intermediates of formula (VII) affords intermediates of formula (III). Typical conditions for the removal of BOC and 2-trimethylsilylethoxymethyl protecting groups include an acid such as hydrochloric acid in a solvent such as methanol or ethyl acetate, or trifluroacetic acid in a solvent such as DCM at a temperature such as 25° C. Typical conditions for the removal of benzyloxycarbonyl protecting group include hydrogenation under an atmosphere of hydrogen in the presence of a catalyst such as palladium on carbon in a solvent such as ethanol or THF at a temperature such as room temperature.

Alternatively, intermediates of formula (VII) may be prepared as illustrated in scheme 4.

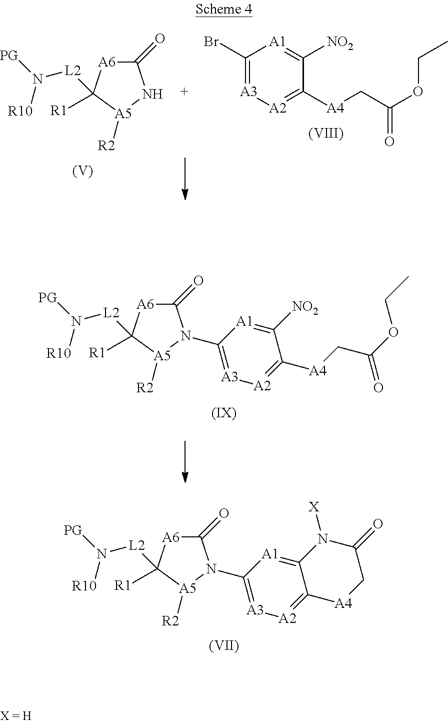

Coupling of protected amine compounds of formula (V) with heteroaryl bromides of formula (VIII) affords intermediates of formula (IX). Typical conditions include the use of a catalytic quantity of copper (I) iodide and trans-N,N′-dimethylcyclohexane-1,2-diamine or of tris(dibenzylideneacetone)dipalladium (0) and 9,9-dimethyl-4,5-bis(diphenylphosphino)xanthene in the presence of a base such as potassium carbonate in a solvent such as 1,4-dioxane at a temperature such as 100° C. Reduction of intermediates (IX) affords intermediates of formula (VII). Typical conditions include iron in acetic acid at a temperature such as 60° C.

Intermediates of formula (II) may be prepared as illustrated in scheme 5.

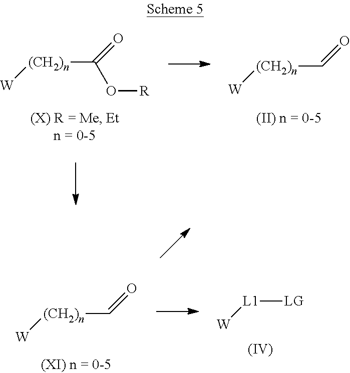

Reduction of esters of formula (X) affords alcohols of formula (XI). Typical conditions include a reducing agent such as lithium aluminium hydride in a solvent such as THF at a temperature such as between −78° C. and room temperature, or sodium borohydride in the presence of calcium chloride in a solvent such as THF and ethanol or methanol at a temperature such as 0° C. Oxidation of alcohols of formula (XI) affords aldehydes of formula (II). Typical conditions include Dess-Martin periodiane in a solvent such as DCM at a temperature such as 0 to 25° C., or sulfur trioxide pyridine complex in the presence of triethylamine and dry dimethylsulfoxide in a solvent such as DCM at a temperature such as 0 to 25° C.

Alternatively, reduction of esters of formula (X) affords aldehydes of formula (II). Typical conditions include a reducing agent such as lithium aluminium hydride or diisobutylaluminium hydride in a solvent such as THF at a reduced temperature such as between −78° C. and −20° C.

Intermediates of formula (IV), wherein LG is a leaving group such as iodo, bromo, mesyloxy or tosyloxy, may be prepared as illustrated in scheme 5. Activation of alcohols of formula (XI) affords intermediates of formula (IV). Typical conditions include p-toluenesulfonyl chloride or methanesulfonyl chloride, N,N-diisopropylethylamine or triethylamine and DMAP in a solvent such as DCM at a temperature such as between 0° C. and room temperature, optionally followed by reaction of the resulting mesyloxy or tosyloxy derivative with a halide salt such as sodium iodide or potassium bromide in a solvent such as MeCN at a temperature such as 60° C.

Intermediates of formula (X), wherein W is ring-system A, may be prepared as illustrated in scheme 6. In the following schemes, R₃/R4 represents either R³ or R⁴ and R4/R3 represents either R⁴ or R³ such that each ring-system has an R³ and an R⁴ substituent.

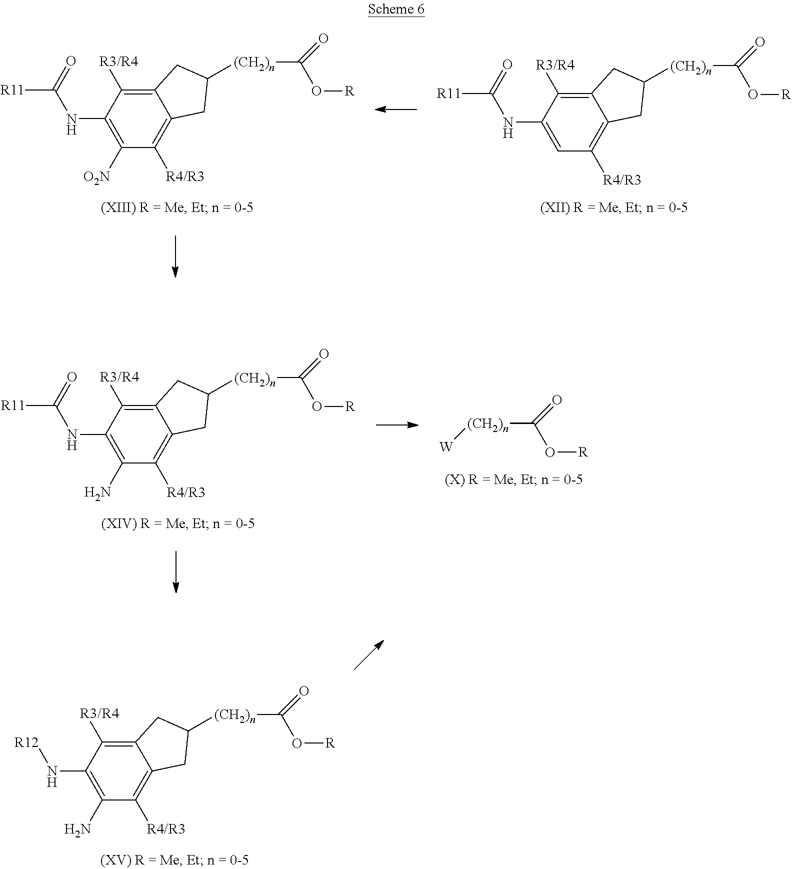

Nitration of intermediates of formula (XII) affords intermediates of formula (XIII). Typical conditions include a mixture of nitric acid and sulfuric acid at a reduced temperature such as between -10 and 0° C. Reduction of intermediates of formula (XIII) affords intermediates of formula (XIV). Typical conditions include hydrogenation under an atmosphere of hydrogen in the presence of a catalyst such as palladium on carbon in a solvent such as ethanol at a temperature such as room temperature. Cyclisation of intermediates of formula (XIV) affords intermediates of formula (X), wherein W is ring-system A and R¹² is H. Typical conditions include acetic acid at a temperature such as 60° C. Reaction of intermediates of formula (X), wherein W is ring-system A and R¹² is H, with alkylating agents of formula R¹²-LG affords intermediates of formula (X), wherein W is ring-system A. Typical conditions include an alkylating agent such as dimethyl carbonate (optionally used as solvent) in the presence of a base such as DBU at a temperature such as 90° C.

Alternatively, reduction of intermediates of formula (XIV) affords intermediates of formula (XV). Typical conditions include borane-THF complex in a solvent such as THF at a temperature such as between 0 and 25° C. Intermediates of formula (XV) may be converted into intermediates of formula (X), wherein W is ring-system A, using a two step procedure. Typical conditions for the first step include reaction with a carboxylic acid of formula R¹¹COOH in the presence of a coupling agent such as TCFH, T3P or HATU in the presence of a base such as N,N-diisopropylethylamine, DMAP or 1-methylimidazole in a solvent such as DCM, DMF or MeCN at a temperature such as room temperature. Alternatively, typical conditions for the first step include reaction with an acid chloride of formula R¹¹COCl in the presence of a base such as triethylamine or N,N-diisopropylethylamine in a solvent such as DCM, DMA or THF at a temperature such as between −10° C. and 25° C. Typical conditions for the second step include acetic acid at a temperature such as 60° C.

Intermediates of formula (X), wherein W is ring-system C and D, may be prepared as illustrated in scheme 7.

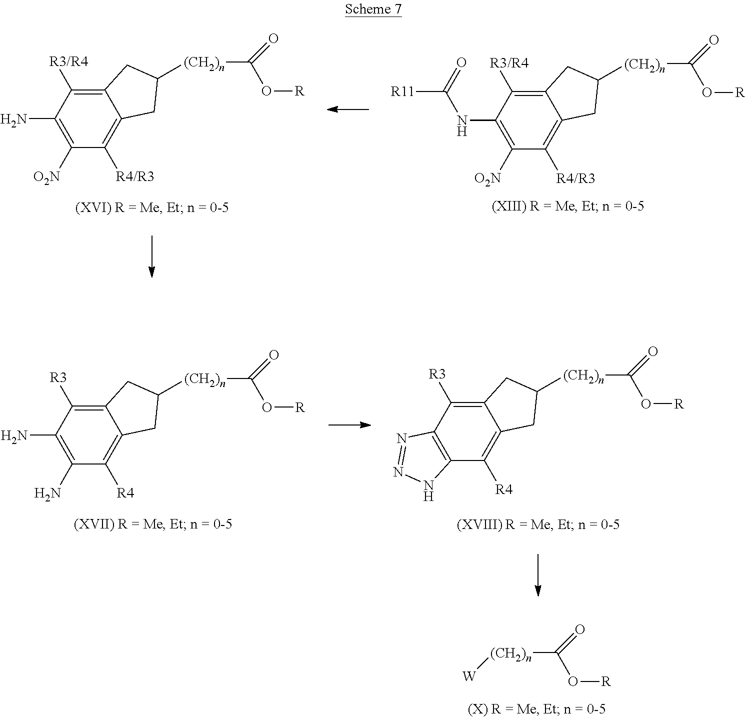

Hydrolysis of amides of formula (XIII) affords amines of formula (XVI). Typical conditions include an acid such as sulfuric acid in a solvent such as ethanol at a temperature such as 78° C. Reduction of amines of formula (XVI) affords diamines of formula (XVII). Typical conditions include hydrogenation under an atmosphere of hydrogen in the presence of a catalyst such as palladium on carbon in a solvent such as ethanol at a temperature such as room temperature. Diamines of formula (XVII) may be converted into intermediates of formula (XVIII). Typical conditions include reaction with sodium nitrite in aqueous hydrochloric acid at a temperature such as 0° C. Alkylation of intermediates of formula (XVIII) affords intermediates of formula (X), wherein W is ring-system C and D. Typical conditions include reaction with an alkyl halide in the presence of a base such as potassium carbonate in a solvent such as MeCN at a temperature such as 80° C.

Intermediates of formula (X), wherein W is ring-system B, may be prepared as illustrated in scheme 8.

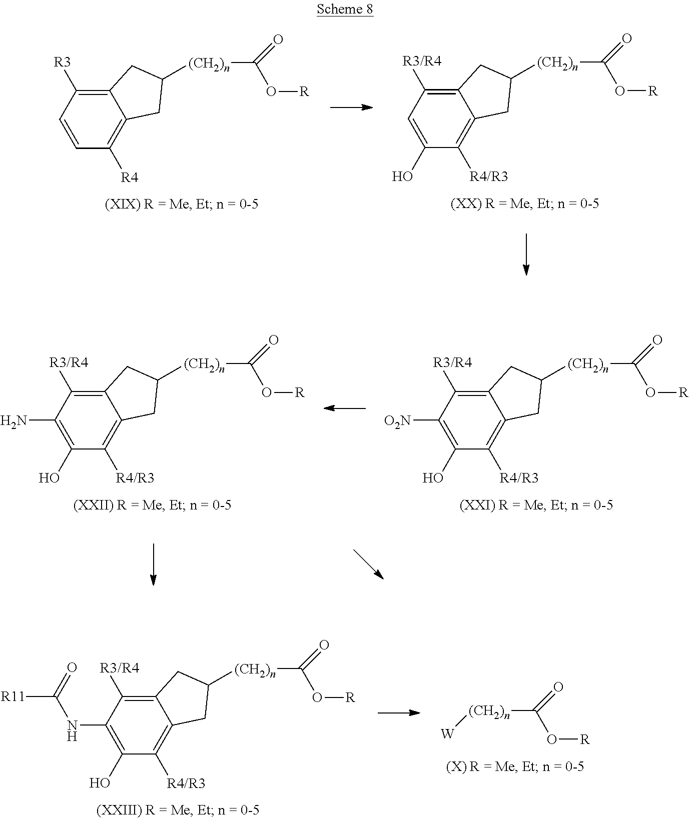

Esters of formula (XIX) may be converted into phenols of formula (XX) using a two step procedure. Typical conditions for the first step include 4,4,5,5-tetramethyl-1,3,2-dioxaborolane and 3,4,7,8-tetramethyl-1,10-phenanthroline in the presence of a catalyst such as (1,5-cyclooctadiene)(methoxy)iridium(I) dimer at a temperature such as 65° C. Typical conditions for the second step include sodium perborate monohydrate in water at a temperature such as between 0 and 25° C. Nitration of phenols of formula (XX) affords nitro compounds of formula (XXI). Typical conditions include nitric acid in a solvent such as DCM at a reduced temperature such as 0° C. Reduction of nitro compounds of formula (XXI) affords amines of formula (XXII). Typical conditions include hydrogenation under an atmosphere of hydrogen in the presence of a catalyst such as palladium on carbon in a solvent such as ethanol at a temperature such as room temperature. Reaction of amines of formula (XXII) with carboxylic acids of formula R¹¹COOH affords amides of formula (XXIII). Typical conditions include a coupling agent such as TCFH, T3P or HATU in the presence of a base such as N,N-diisopropylethylamine, DMAP or 1-methylimidazole in a solvent such as DCM, DMF or MeCN at a temperature such as room temperature. Cyclisation of intermediates of formula (XXIII) affords intermediates of formula (X), wherein W is ring-system B. Typical conditions include triphenylphosphine and diisopropyl azodicarboxylate in the presence of 4 Å molecular sieves in a solvent such as THF at a temperature such as between 0 and 25° C.

Alternatively, reaction of amines of formula (XXII) with an imidate of formula R¹¹C(═NH)OCH₂CH₃ or R¹¹C(═NH)OCH₃ affords intermediates of formula (X), wherein W is ring-system B. Typical conditions include reaction with the hydrochloride salt of the imidate in a solvent such as DCM at a temperature such as room temperature.

Intermediates of formula (X), wherein W is ring-system E, may be prepared as illustrated in scheme 9.

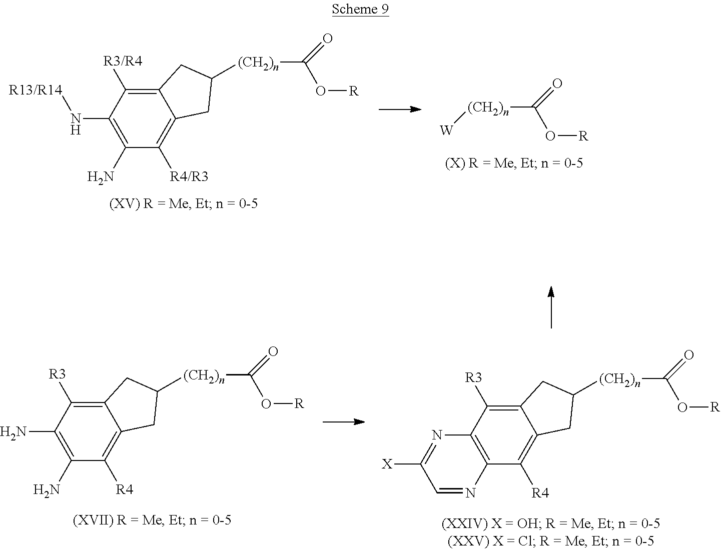

Mono-N-alkylated diamines of formula (XV) may be converted into intermediates of formula (X), wherein W is ring-system E. Typical conditions include N,N′-carbonyldiimidazole in a solvent such as DCM at a temperature such as room temperature.

Intermediates of formula (X), wherein W is ring-system F, may be prepared as illustrated in scheme 9. Diamines of formula (XVII) may be converted into intermediates of formula (XXIV). Typical conditions include ethyl glyoxalate in a solvent such as MeCN at a temperature such as room temperature. Intermediates of formula (XXIV) may be converted into intermediates of formula (XXV). Typical conditions include phosphorus oxychloride at a temperature such as between room temperature and 100° C. Intermediates of formula (XXV) may be converted into intermediates of formula (X), wherein W is ring-system F. Typical conditions include reaction with an amine of formula R¹⁵H in the presence of a base such as N,N-diisopropylethylamine in a solvent such as THF at a temperature such as between 60 and 120° C.

Intermediates of formula (XII), wherein n=0, may be prepared as illustrated in scheme 10.

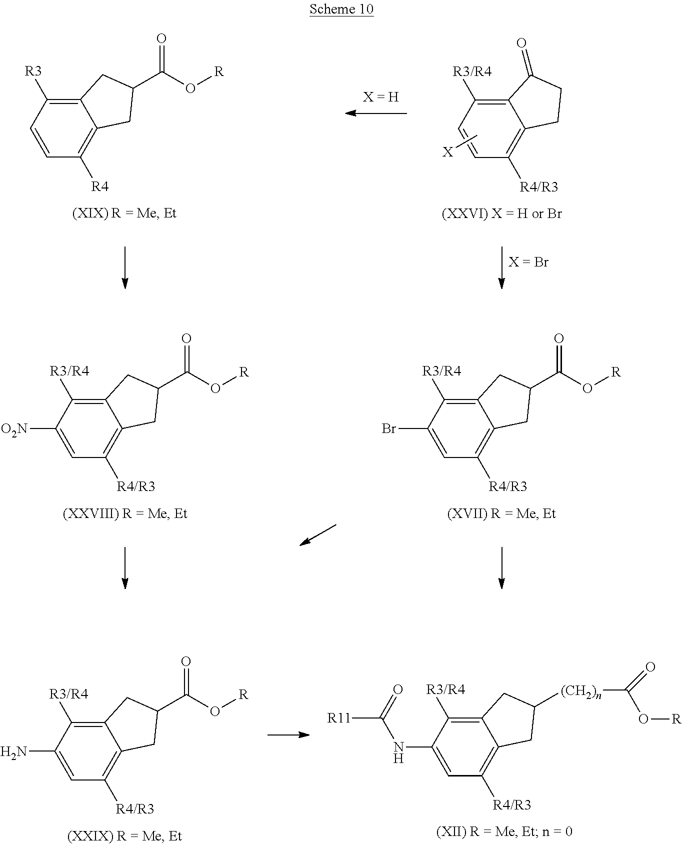

Indan-1ones of formula (XXVI) may be converted into esters of formula (XVII) or esters of formula (XIX) using a two step procedure. Typical conditions for the first step include reaction with dimethyl or diethyl carbonate in the presence of a base such as sodium hydride in a solvent such as toluene or THF at a temperature such as between 60 and 120° C. Typical conditions for the second step include hydrogenation in the presence of an acid such as HCl or sulfuric acid using a catalyst such as palladium on carbon in a solvent such as ethanol at a temperature such as room temperature. Bromides of formula (XVII) may be converted into intermediates of formula (XII). Typical conditions include reaction with an amide of formula R¹¹CONH₂ in the presence of a palladium catalyst such as tris(dibenzylideneacetone)dipalladium (0) and a phosphine ligand such as xantphos or XPhos and a base such as cesium carbonate in a solvent such as 1,4-dioxane at a temperature such as between 90 and 100° C. Alternatively, reaction under similar conditions with tert-butyl carbamate in place of an amide of formula R¹¹CONH₂ affords an intermediate which may be readily converted into amines of formula (XXIX) using an acid such as HCl or TFA in a solvent such as EtOH, EtOAc or DCM at a temperature such as room temperature.

Nitration of intermediates of formula (XIX) affords intermediates of formula (XXVIII). Typical conditions include nitric acid in a solvent such as DCM at a reduced temperature such as 0° C. Reduction of nitro compounds of formula (XXVIII) affords amines of formula (XXIX). Typical conditions include hydrogenation under an atmosphere of hydrogen in the presence of a catalyst such as palladium on carbon in a solvent such as ethanol at a temperature such as room temperature. Reaction of amines of formula (XXIX) with carboxylic acids of formula R¹¹COOH affords amides of formula (XXII). Typical conditions include a coupling agent such as TCFH, T3P or HATU in the presence of a base such as N,N-diisopropylethylamine, DMAP or 1-methylimidazole in a solvent such as DCM, DMF or MeCN at a temperature such as room temperature.

Alternatively, reaction of amines of formula (XXIX) with acid chlorides of formula R¹¹COCl affords amides of formula (XII). Typical conditions include the presence of a base such as triethylamine or N,N-diisopropylethylamine in a solvent such as DCM, DMA or THF at a temperature such as between −10° C. and 25° C.

Particular examples of compounds of formula (I) as described herein are selected from
6-[5-[2-[(4,8-difluoro-2-methyl-3,5,6,7-tetrahydrocyclopenta[f]benzimidazol-6-yl)methylamino]ethyl]-2-oxo-oxazolidin-3-yl]-4H-pyrazino[2,3-b][1,4]oxazin-3-one;
6-[5-[2-[[2-[(dimethylamino)methyl]-4,8-difluoro-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;
6-[8-[[2-[(dimethylamino)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(1-hydroxy-1-methyl-ethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1,1-dioxo-1,4-thiazinan-4-yl)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(4-methylmorpholin-2-yl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(morpholinomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3R)-morpholin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3R)-morpholin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-(methylaminomethyl)-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-5-oxopyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-5-oxopyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[(10,16-difluoro-2,5,8-triazatetracyclo[7.7.0.02,7.011,15]hexadeca-1(9),7,10,15-tetraen-13-yl)methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-[(1R)-1-(methylamino)ethyl]-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-2-[(2S,4R)-4-(methylamino)pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-4-hydroxy-2-piperidyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-4-hydroxy-2-piperidyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[2-[(dimethylamino)methyl]-4,8-difluoro-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[2-(1-amino-1-methyl-ethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S,4R)-4-aminotetrahydrofuran-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[(2-amino-4,8-difluoro-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-(methylaminomethyl)-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-azabicyclo[2.1.1]hexan-1-yl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
N-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-2-(methylamino)acetamide;
6-[8-[[2-[(1S,2S)-1-amino-2-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-methoxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[5-[2-[[4,8-difluoro-2-[(2S,4R)-4-hydroxypyrrolidin-2-yl]-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;
6-[5-[2-[[4,8-difluoro-2-[(2S,4R)-4-hydroxypyrrolidin-2-yl]-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;2,2,2-trifluoroacetic acid;
6-[8-[[8-fluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[[4,8-difluoro-2-[(2S,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-[(2R,4R)-4-hydroxypyrrolidin-2-yl]-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S,4R)-4-hydroxypyrrolidin-2-yl]-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-3-hydroxy-2-(methylamino)propyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxy-1-methyl-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(methylamino)acetamide;
   or pharmaceutically acceptable salts thereof.
      Other particular examples of compounds of formula (I) as described herein are selected from
6-[8-[[2-[2-(dimethylamino)ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-(dimethylamino)-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(dimethylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-[2-(dimethylamino)ethyl]-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[2-(methylamino)ethyl]-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2-methoxyethylamino)methyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
2-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]methylamino]-N-methyl-acetamide;
6-[8-[[1-(2-aminoethyl)-8-fluoro-2-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-2-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[1-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-5-fluoro-7,8-dihydro-6H-cyclopenta[e]benzotriazol-7-yl]methyl]-2-keto-1-oxa-3,8-diazaspiro[4.5]decan-3-yl]-4H-pyrazino[2,3-b][1,4]oxazin-3-one;
6-[8-[[3-(2-aminoethyl)-5-fluoro-7,8-dihydro-6H-cyclopenta[e]benzotriazol-7-yl]methyl]-2-keto-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(1R)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-(methylaminomethyl)-1,6,7,8-tetrahydrocyclopenta[e]benzimidazol-7-yl]methyl]-2-oxo-1-oxa-3,8-diazaspiro[4.5]decan-3-yl]-4H-pyrazino[2,3-b][1,4]oxazin-3-one;
6-[8-[[4-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[8-[[5-fluoro-2-(methylaminomethyl)-7,8-dihydro-6H-cyclopenta[e][1,3]benzoxazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(3-hydroxyazetidin-1-yl)methyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(aminomethyl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(azetidin-3-yl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
formic acid;6-[2-oxo-8-[[3-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[3-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[2-oxo-8-[[4,8-difluoro-1-[2-(methylamino)ethyl]-6,7-dihydro-5H-cyclopenta[f]benzimidazol-6-yl]methyl]-1-oxa-3,8-diazaspiro[4.5]decan-3-yl]-4H-pyrazino[2,3-b][1,4]oxazin-3-one;
6-[8-[[1-[(2S)-2-aminopropyl]-4,8-difluoro-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[1-[(2S)-2-aminopropyl]-4,8-difluoro-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;hydrochloric acid;
6-[8-[[1-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[8-[[2-[(3R,4R)-4-aminotetrahydrofuran-3-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(1S)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)propanamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(dimethylamino)propanamide;
(2R)-2-(dimethylamino)-N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]propanamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(dimethylamino)-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-N-methyl-acetamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]pyrrolidine-2-carboxamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(methylamino)acetamide;
N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]-2-(methylamino)acetamide;2,2,2-trifluoroacetic acid;
N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]-2-(methylamino)acetamide;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]pyrrolidine-2-carboxamide;2,2,2-trifluoroacetic acid;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]pyrrolidine-2-carboxamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]pyrrolidine-2-carboxamide;
(2R,4S)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-4-hydroxy-pyrrolidine-2-carboxamide;
(2R,4S)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-4-hydroxy-pyrrolidine-2-carboxamide;
2-amino-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;formic acid;
2-amino-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;
2-amino-N-methyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)acetamide;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-2-(methylamino)propanamide;
6-[2-oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride;
   or pharmaceutically acceptable salts thereof.
      Further particular examples of formula (I) as described herein are selected from
6-[8-[[2-[(dimethylamino)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(1-amino-1-methyl-ethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S,4R)-4-aminotetrahydrofuran-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-(methylaminomethyl)-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-azabicyclo[2.1.1]hexan-1-yl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
N-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-2-(methylamino)acetamide;
6-[8-[[2-[(1S,2S)-1-amino-2-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-methoxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
   or pharmaceutically acceptable salts thereof.
      Other, further particular examples of formula (I) as described herein are selected from
6-[8-[[3-[2-(dimethylamino)ethyl]-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[1-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-(methylaminomethyl)-1,6,7,8-tetrahydrocyclopenta[e]benzimidazol-7-yl]methyl]-2-oxo-1-oxa-3,8-diazaspiro[4.5]decan-3-yl]-4H-pyrazino[2,3-b][1,4]oxazin-3-one;
6-[8-[[4-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[8-[[5-fluoro-2-(methylaminomethyl)-7,8-dihydro-6H-cyclopenta[e][1,3]benzoxazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(aminomethyl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(azetidin-3-yl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[2-oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride;
   or pharmaceutically acceptable salts thereof.
      More particular examples of formula (I) as described herein are selected from
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
   or pharmaceutically acceptable salts thereof.
      Other particular examples of formula (I) as described herein are selected from
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
   or pharmaceutically acceptable salts thereof.

Also an object of the present invention is a compound according to formula (I) as described herein for use as a therapeutically active substance.

Likewise an object of the present invention is a pharmaceutical composition comprising a compound according to formula (I) as described herein and a therapeutically inert carrier.

As described above, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

The compounds of formula (I) and their pharmaceutically acceptable salts exhibit activity as antibiotics, particularly as antibiotics against Pseudomonas aeruginosa, Klebsiella pneumoniae, Escherichia coli, Acinetobacter baumannii or Staphylococcus aureus, more particularly against Gram negative bacterias, furthermore particularly against Escherichia coli.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to pharmaceutical compositions comprising compounds of formula (I) as defined above or their pharmaceutically acceptable salts as defined above and one or more pharmaceutically acceptable excipients.

A particular embodiment of the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to compounds of formula (I) or their pharmaceutically acceptable salts as defined above for use as therapeutically active substances, especially for use as therapeutically active substances for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to compounds of formula (I) or their pharmaceutically acceptable salts as defined above for the use in the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to a method for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli, which method comprises administering compounds of formula (I) or their pharmaceutically acceptable salts as defined above to a subject.

A particular embodiment of the present invention relates to the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli. Such medicaments comprise compounds of formula (I) or their pharmaceutically acceptable salts as defined above.

Also an embodiment of the present invention are compounds of formula (I) as described herein, when manufactured according to any one of the described processes.

### Assay Procedures

### Minimal Inhibitory Concentration (MIC) Determination:

The MIC of antibacterial compounds against multiple species and strains was determined using the broth microdilution method according to M07-A10 Clinical and Laboratory Standards Institute (CLSI) guidelines [1], using the appropriate broth medium. In short, the compound plates were prepared by dispensing 100 μL of each stock solution in the first well of a 96 well microtiter plate (MTP) at a concentration 100-fold higher than the final concentration desired in broth. Eleven serial 2-fold dilutions of the highest concentration were made in DMSO for new compounds and in water (or appropriate solution) for reference compounds [2]. 1 μL of each well was transferred in a new MTP, which served as the test plate by subsequent inoculation. This bacterial suspension was prepared from strains that were first sub-cultured on agar plates and incubated for 18-24 hours as appropriate. Following incubation, the inoculum was prepared from isolated colonies and adjusted to 0.5 McFarland turbidity standard (1 to 2×108 Colony Forming Units (CFU)/mL) in 0.9% saline. The bacterial suspension was then diluted 1:200 in appropriate sterile medium and within 15 minutes 100 μL/well were dispensed. Negative controls (lack of bacterial cells) and growth control wells (lack of compound) were included in all plates. MTPs were incubated for 18-24 hours at 35±2° C. in ambient air. The MIC value of each compound, expressed as μg/mL, was determined as the lowest concentration required for complete growth inhibition (no visible growth).
[1] CLSI (2015) Methods for dilution antimicrobial susceptibility tests for bacteria that grow aerobically; Approved Standard, Tenth Edition. CLSI Document M7-A10 (ISBN 1-56238-987-4). Wayne, PA: Clinical and Laboratory Standards Institute, 19087, USA.
[2] CLSI (2017) Performance Standards for Antimicrobial Susceptibility Testing; Twenty-Fifth Informational Supplement. CLSI Document M100-S27, CLSI, Wayne, Pennsylvania 19087, USA.

Table 1 provides the Minimal Inhibitory Concentrations (MIC) in micrograms per millilitre of the compounds of present invention obtained against the strain Escherichia coli ATCC 25922.

Particular compounds of the present invention exhibit an MIC (Escherichia coli ATCC 25922)≤4 μg/mL.

More particular compounds of the present invention exhibit an MIC (Escherichia coli ATCC 25922)≤1 μg/mL.

Most particular compounds of the present invention exhibit an MIC (Escherichia coli ATCC 25922)≤0.25 μg/mL.

TABLE 1

| | Escherichia coli |
|---|---|
| | growth inhibition |
| Example | MIC (μg/mL) |
| 1 | 0.5 |
| 2 | 0.5 |
| 3 | 0.25 |
| 4 | 0.5 |
| 5 | 2 |
| 6 | 1 |
| 7 | 0.125 |
| 8 | 1 |
| 9 | 0.125 |
| 10 | 0.125 |
| 11 | 0.125 |
| 12 | 0.125 |
| 13 | 0.25 |
| 14 | 0.25 |
| 15 | 0.25 |
| 16 | 0.25 |
| 17 | 0.125 |
| 18 | 0.5 |
| 19 | 0.5 |
| 20 | 0.25 |
| 21 | 1 |
| 22 | 0.25 |
| 23 | 0.5 |
| 24 | 0.125 |
| 25 | ≤0.06 |
| 26 | 0.5 |
| 27 | 0.25 |
| 28 | 0.25 |
| 29 | 0.5 |
| 30 | 0.125 |
| 31 | 0.25 |
| 32 | 0.25 |
| 33 | 0.25 |
| 34 | 0.25 |
| 35 | 0.125 |
| 36 | 0.5 |
| 37 | 0.25 |
| 38 | 0.125 |
| 39 | 0.5 |
| 40 | 0.5 |
| 41 | ≤0.06 |
| 42 | ≤0.06 |
| 43 | 2 |
| 44 | 0.25 |
| 45 | 0.125 |
| 46 | 0.25 |
| 47 | 2 |
| 48 | 0.25 |
| 49 | 0.25 |
| 50 | 0.25 |
| 51 | 0.5 |
| 52 | 0.5 |
| 53 | 4 |
| 54 | 0.125 |
| 55 | 0.25 |
| 56 | 0.25 |
| 57 | 0.5 |
| 58 | 1 |
| 59 | 0.125 |
| 60 | 0.125 |
| 61 | 0.5 |
| 62 | 0.25 |
| 63 | 0.25 |
| 64 | 0.25 |
| 65 | 0.125 |
| 66 | 0.25 |
| 67 | 0.25 |
| 68 | 0.125 |
| 69 | 0.25 |
| 70 | 0.25 |
| 71 | 1 |
| 72 | 2 |
| 73 | 2 |
| 74 | 0.06 |
| 75 | ≤0.06 |
| 76 | 0.125 |
| 77 | 0.25 |
| 78 | 0.25 |
| 79 | 0.125 |
| 80 | 0.125 |
| 81 | ≤0.06 |
| 82 | 0.125 |
| 83 | ≤0.06 |
| 84 | 0.25 |
| 85 | 0.25 |
| 86 | ≤0.06 |
| 87 | 0.125 |
| 88 | ≤0.06 |
| 89 | 0.125 |
| 90 | 0.125 |
| 91 | 0.25 |
| 92 | 0.125 |
| 93 | 0.125 |
| 94 | 0.25 |
| 95 | 0.25 |
| 96 | 0.125 |
| 97 | 0.125 |
| 98 | 0.5 |
| 99 | 1 |
| 100 | 0.125 |
| 101 | 0.25 |
| 102 | 0.125 |
| 103 | ≤0.06 |
| 104 | 0.125 |
| 105 | 0.25 |
| 106 | 0.125 |
| 107 | 0.125 |
| 108 | 0.25 |
| 109 | 0.5 |
| 110 | 1 |
| 111 | 0.5 |
| 112 | 0.5 |
| 113 | 0.125 |
| 114 | 0.25 |
| 115 | 0.5 |
| 116 | 0.25 |
| 117 | 0.25 |
| 118 | 0.125 |
| 119 | 0.125 |
| 120 | 0.25 |
| 121 | 0.125 |
| 122 | 0.5 |
| 123 | 0.125 |
| 124 | 0.5 |
| 125 | 0.25 |
| 126 | 0.25 |
| 127 | 0.25 |
| 128 | 0.25 |
| 129 | 0.25 |
| 130 | 0.25 |
| 131 | 0.125 |
| 132 | 0.25 |
| 133 | 1 |
| 134 | 0.125 |
| 135 | ≤0.06 |
| 136 | 1 |
| 137 | 0.5 |
| 138 | 1 |
| 139 | 1 |
| 140 | 0.25 |
| 141 | 0.5 |
| 142 | 0.5 |
| 143 | 0.25 |
| 144 | 0.25 |
| 145 | 0.5 |
| 146 | 0.5 |
| 147 | 0.5 |
| 148 | 1 |
| 149 | 1 |
| 150 | 1 |
| 151 | 0.5 |
| 152 | 2 |
| 153 | 2 |
| 154 | 2 |
| 155 | 1 |
| 156 | 8 |
| 157 | 4 |
| 158 | 0.25 |
| 159 | 0.25 |
| 160 | 0.5 |
| 161 | 0.25 |
| 162 | 1 |
| 163 | 0.5 |
| 164 | 2 |
| 165 | 0.5 |
| 166 | 1 |
| 167 | 1 |
| 168 | 0.5 |
| 169 | 1 |
| 170 | 0.25 |
| 171 | ≤0.06 |

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays), rectally (e.g. in the form of suppositories) or topical ocularly (e.g. in the form of solutions, ointments, gels or water soluble polymeric inserts). However, the administration can also be effected parenterally, such as intramuscularly, intravenously, or intraocularly (e.g. in the form of sterile injection solutions).

The compounds of formula (I) and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

### Preparation of Pharmaceutical Compositions Comprising Compounds of the Invention

Tablets of the following composition are manufactured in the usual manner:

mg/tablet

| Ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 1161 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

Capsules of the following composition are manufactured:

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

A Compound of formula I lactose and corn starch are firstly mixed in a mixer and then in a comminuting machine. The mixture is returned to the mixer; the talc is added thereto and mixed thoapproximatively. The mixture is filled by machine into suitable capsules, e.g. hard gelatin capsules.

Injection solutions of the following composition are manufactured:

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

The invention is illustrated hereinafter by Examples, which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

### Abbreviations

MeOH: methanol, AcOH: acetic acid, prep-HPLC: preparative reversed phase high-performance liquid chromatography, MS: mass spectrum, M: molecular mass, ESP: electrospray ionisation (positive charge detection), ESN: electrospray ionisation (negative charge detection), DCM: dichloromethane, EtOAc: ethyl acetate, DMF: N,N-dimethylformamide, RT, r.t. or rt: room temperature, DIPEA: N,N-diisopropylethylamine, i-PrOH: 2-propanol, h: hours, THF: tetrahydrofuran, xantphos: 4,5-bis(diphenylphosphino)-9,9-dimethylxanthene, TFA: trifluoroacetic acid, dioxane: 1,4-dioxane, sat.: saturated, quant.: quantitative, EL: electron ionization, dppf: 1,1′-ferrocenediyl-bis(diphenylphosphine), HATU: 1-[bis(dimethylamino)methylene]-1H-1,2,3-triazolo[4,5-b]pyridinium 3-oxide hexafluorophosphate, RM: reaction mixture, t-BuOH: 2-methylpropan-2-ol, prep-TLC: preparative thin layer chromatography, SFC: supercritical fluid chromatography, ether: diethyl ether, BOC: tert-butyloxycarbonyl, Pd/C: palladium on carbon, TMSCl: trimethylsilyl chloride, MeCN: acetonitrile, NMR: nuclear magnetic resonance spectroscopy, s: singlet, d (NMR): doublet, t: triplet, q: quartet, m: multiplet, dd: doublet of doublets, dt: doublet of triplets, br: broad, J: coupling constant, 6, chemical shift in parts per million, PE: petroleum ether, NMP: N-methyl-2-pyrrolidone, DMA: N,N-dimethylacetamide, Dess-Martin periodinane or DMP: 1,1,1-triacetoxy-1,1-dihydro-1,2-benziodoxol-3(1H)-one, LDA: lithium diisopropylamide, MTBE: methyl tert-butyl ether, MOMCl: methylchloromethyl ether, DMAP: 4-dimethylaminopyridine, tBu: tert-butyl, LiHMDS: lithium bis(trimethylsilyl)amide, TsCl: tosyl chloride, HPLC: preparative reversed phase high-performance liquid chromatography, TBSOTf: tert-butyldimethylsilyl trifluoromethanesulfonate, DMSO: dimethyl sulfoxide, d (time): days, TEMPO: (2,2,6,6-tetramethyl-piperidin-1-yl)oxyl, TBSCl: tert-butyldimethylsilyl chloride, dba: dibenzylideneacetone, t-BuXphos: 2-di-tert-butylphosphino-2′,4′,6′-triisopropylbiphenyl, TBAF: tetra-n-butylammonium fluoride, t-BuXPhos-Pd-G3: [(2-di-tert-butylphosphino-2′,4′,6′-triisopropyl-1,1′-biphenyl)-2-(2′-amino-1,1′-biphenyl)] palladium(II) methanesulfonate, TCFH: chloro-N,N,N′,N′-tetramethylformamidinium hexafluorophosphate, T3P: 2,4,6-tripropyl-1,3,5,2,4,6-trioxatriphosphorinane-2,4,6-trioxide, PyBOP: benzotriazole-1-yl-oxy-tris-pyrrolidino-phosphonium hexafluorophosphate.

### Example 1

### 6-[5-[2-[(4,8-Difluoro-2-methyl-3,5,6,7-tetrahydrocyclopenta[f]benzimidazol-6-yl)methylamino]ethyl]-2-oxo-oxazolidin-3-yl]-4H-pyrazino[2,3-b][1,4]oxazin-3-one

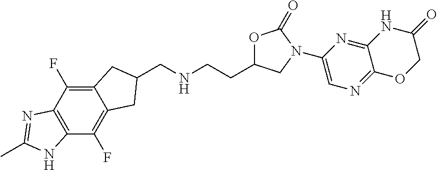 [SMILES: Cc1nc2c(F)c3c(c(F)c2[nH]1)CC(CNCCC1CN(c2cnc4c(n2)NC(=O)CO4)C(=O)O1)C3]

A solution of 4,8-difluoro-2-methyl-3,5,6,7-tetrahydrocyclopenta[f]benzimidazole-6-carbaldehyde (Intermediate 8, 30.7 mg, 0.130 mmol, 1 eq), 6-[5-(2-aminoethyl)-2-oxo-oxazolidin-3-yl]-4H-pyrazino[2,3-b][1,4]oxazin-3-one; 2,2,2-trifluoroacetic acid (Intermediate 1, 51.1 mg, 0.130 mmol, 1 eq) and DIPEA (34.33 uL, 0.200 mmol, 1.5 eq) in DMA (0.657 mL) and THF (0.657 mL) was stirred at 25° C. for 15 min. Acetic acid (18.79 uL, 0.330 mmol, 2.5 eq) was added followed by sodium triacetoxyborohydride (41.77 mg, 0.200 mmol, 1.5 eq). The mixture was stirred overnight at 25° C. The mixture was diluted with saturated aqueous NaHCO₃ and extracted with EtOAc (×3). The organic phase was dried over Na₂SO₄ and concentrated in vacuo. The mixture was purified by silica cartridge chromatography (DCM/MeOH 9:1, then gradient to 100% DCM/MeOH/NH₄OH 10:1:0.1) to afford the title compound (15.5 mg, 0.030 mmol, 22.4% yield) as a white solid. MS (ESP) m/z=500.2 [M+H]⁺.

The following examples were prepared by analogy to Example 1.

| Ex. | Name, Structure, MS | Starting Materials |
|---|---|---|
| 2 | 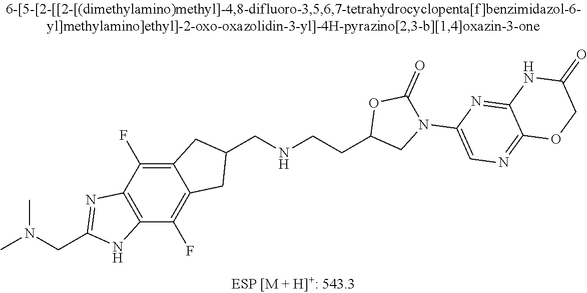 [SMILES: CN(C)Cc1nc2c(F)c3c(c(F)c2[nH]1)CC(CNCCC1CN(c2cnc4c(n2)NC(=O)CO4)C(=O)O1)C3] | Intermediate 1 and Intermediate 9 |
| 3 | 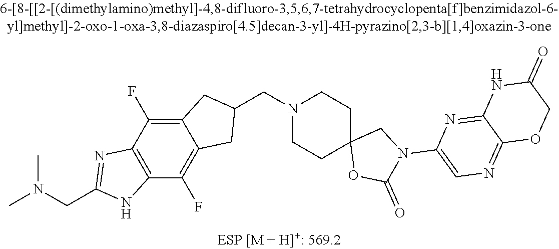 [SMILES: CN(C)Cc1nc2c(F)c3c(c(F)c2[nH]1)CC(CN1CCC2(CC1)CN(c1cnc4c(n1)NC(=O)CO4)C(=O)O2)C3] | Intermediate 2 and Intermediate 9 |
| 4 | 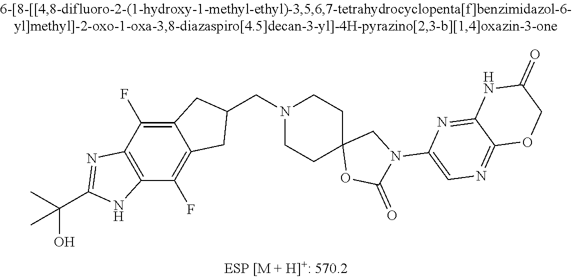 [SMILES: CC(C)(O)c1nc2c(F)c3c(c(F)c2[nH]1)CC(CN1CCC2(CC1)CN(c1cnc4c(n1)NC(=O)CO4)C(=O)O2)C3] | Intermediate 2 and Intermediate 23 |
| 5 | 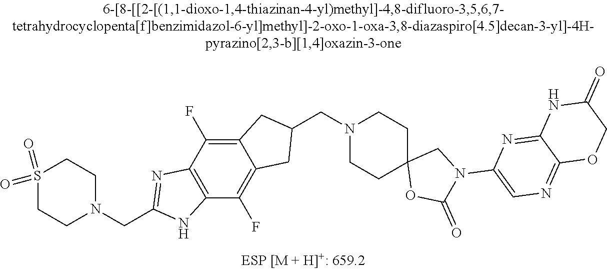 [SMILES: O=C1COc2ncc(N3CC4(CCN(CC5Cc6c(c(F)c7[nH]c(CN8CCS(=O)(=O)CC8)nc7c6F)C5)CC4)OC3=O)nc2N1] | Intermediate 2 and Intermediate 10 |
| 6 | 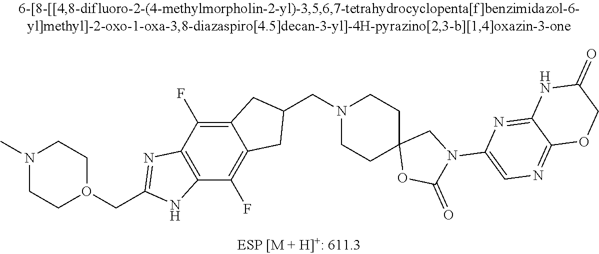 | Intermediate 2 and Intermediate 24 |
| 7 | 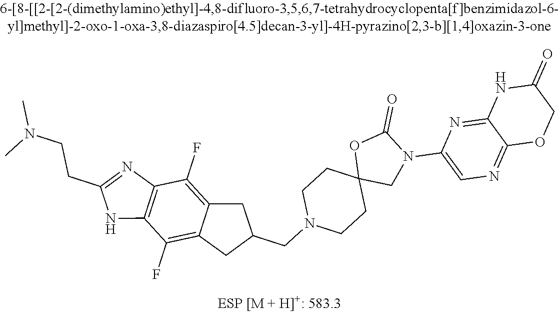 [SMILES: CN(C)CCc1nc2c(F)c3c(c(F)c2[nH]1)CC(CN1CCC2(CC1)CN(c1cnc4c(n1)NC(=O)CO4)C(=O)O2)C3] | Intermediate 2 and Intermediate 25 |
| 8 | 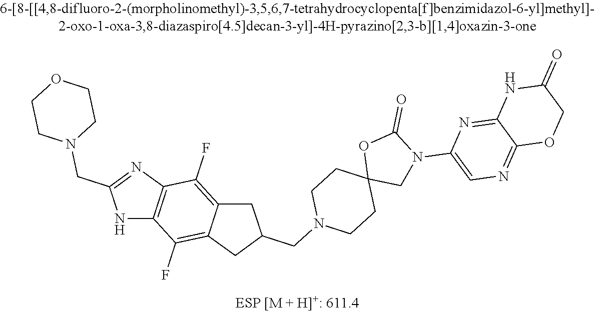 [SMILES: O=C1COc2ncc(N3CC4(CCN(CC5Cc6c(c(F)c7[nH]c(CN8CCOCC8)nc7c6F)C5)CC4)OC3=O)nc2N1] | Intermediate 2 and Intermediate 22 |

### Example 9

### 6-[8-[[4,8-Difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

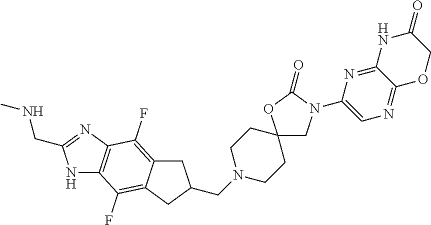 [SMILES: CNCc1nc2c(F)c3c(c(F)c2[nH]1)CC(CN1CCC2(CC1)CN(c1cnc4c(n1)NC(=O)CO4)C(=O)O2)C3]

### Step 1: tert-butyl N-[[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]methyl]-N-methyl-carbamate

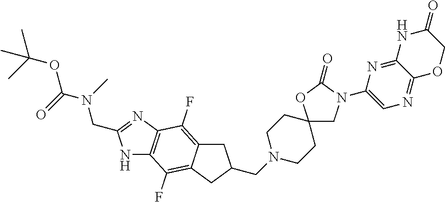 [SMILES: CN(Cc1nc2c(F)c3c(c(F)c2[nH]1)CC(CN1CCC2(CC1)CN(c1cnc4c(n1)NC(=O)CO4)C(=O)O2)C3)C(=O)OC(C)(C)C]

A solution of 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2) (3.28 g, 7.81 mmol, 1.05 eq), tert-butyl N-[(4,8-difluoro-6-formyl-3,5,6,7-tetrahydrocyclopenta[f]benzimidazol-2-yl)methyl]-N-methyl-carbamate (Intermediate 11) (2.72 g, 7.44 mmol, 1 eq) and DIPEA (1.94 mL, 11.16 mmol, 1.5 eq) in DMA (26.15 mL) and THF (48.24 mL) was stirred at 25° C. for 15 min. Acetic acid (1.06 mL, 18.6 mmol, 2.5 eq) was added followed by sodium triacetoxyborohydride (2.36 g, 11.16 mmol, 1.5 eq). The mixture was stirred for 0.5 h at 25° C. The mixture was diluted with saturated aqueous NaHCO₃ and extracted with EtOAc (xl). The organic phase was washed with brine (xl), dried over Na₂SO₄ and concentrated in vacuo. The mixture was purified by silica cartridge chromatography [DCM/(DCM/MeOH/NH₄OH 10:1:0.1) from 8:2 to 2:8) to give the title compound (2070 mg, 3.16 mmol, 42.5% yield). MS (ESP) m/z=655.3 [M+H]⁺.

### Step 2: 6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

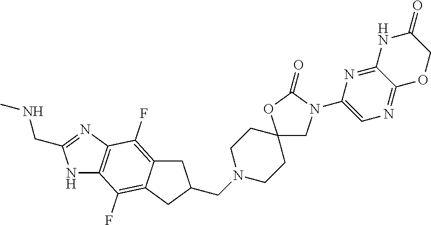 [SMILES: CNCc1nc2c(F)c3c(c(F)c2[nH]1)CC(CN1CCC2(CC1)CN(c1cnc4c(n1)NC(=O)CO4)C(=O)O2)C3]

To a stirred suspension of tert-butyl N-[[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]methyl]-N-methyl-carbamate (2070.0 mg, 3.16 mmol, 1 eq) in DCM (32.08 mL), trifluoroacetic acid (7.31 mL, 94.86 mmol, 30 eq) was added and the mixture was stirred for 16 h at RT. The reaction was concentrated in vacuo and co-evaporated in the presence of MeCN. The resulting mixture was purified by silica cartridge chromatography [DCM/(DCM/MeOH/NH₄OH 5:1:0.1) from 70:30 to 20:80] to afford the title compound (1700 mg, 3.07 mmol, 96.95% yield). MS (ESP) m/z=555.2 [M+H]⁺.

The following examples were prepared by analogy to Example 9.

| Ex. | Name, Structure, MS | Starting Materials |
|---|---|---|
| 10 | 6-[8-[[2-(aminomethyl)-4,8-difluoro-3,5,6,7- | Intermediate 2 and |
| | tetrahydrocyclopenta[f]benzimidazol-6-yl]methyl]-2-oxo-1-oxa-3,8- | Intermediate 12 |
| | diazaspiro[4.5]decan-3-yl]-4H-pyrazino[2,3-b][1,4]oxazin-3-one; | |
| | 2,2,2-trifluoroacetic acid | |
| | 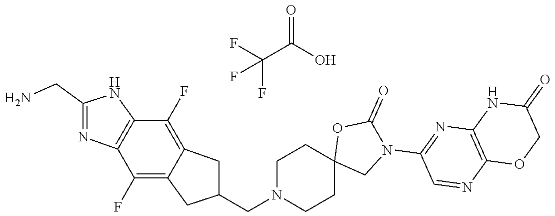 [SMILES: NCc1nc2c(F)c3c(c(F)c2[nH]1)CC(CN1CCC2(CC1)CN(c1cnc4c(n1)NC(=O)CO4)C(=O)O2)C3 ; O=C(O)C(F)(F)F] | |
| | ESP [M + H]⁺: 541.2 | |
| 11 | 6-[8-[[4,8-difluoro-2-[(2S)-pyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 13 |
| | diazaspiro[4.5]decan-3-yl]-4H-pyrazino[2,3-b][1,4]oxazin-3-one; | |
| | 2,2,2-trifluoroacetic acid | |
| | 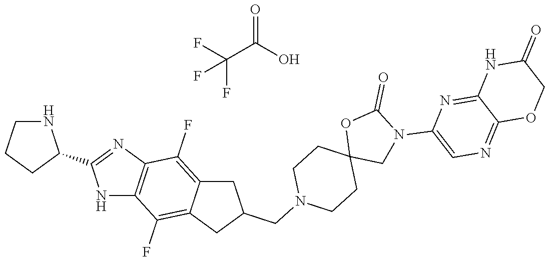 [SMILES: O=C1COc2ncc(N3CC4(CCN(CC5Cc6c(c(F)c7[nH]c([C@@H]8CCCN8)nc7c6F)C5)CC4)OC3=O)nc2N1 ; O=C(O)C(F)(F)F] | |
| | ESP [M + H]⁺: 581.3 | |
| 12 | 6-[8-[[8-fluoro-2-(methylaminomethyl)-1,5,6,7- | Intermediate 2 and |
| | tetrahydrocyclopenta[f]benzimidazol-6-yl]methyl]-2-oxo-1-oxa-3,8- | Intermediate 14 |
| | diazaspiro[4.5]decan-3-yl]-4H-pyrazino[2,3-b][1,4]oxazin-3-one; | |
| | 2,2,2-trifluoroacetic acid | |
| | 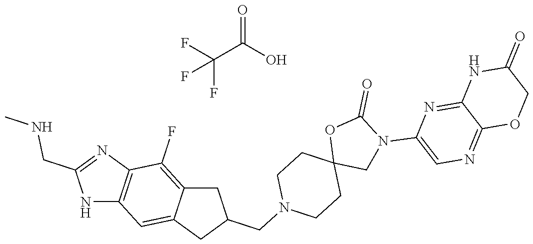 [SMILES: CNCc1nc2c(F)c3c(cc2[nH]1)CC(CN1CCC2(CC1)CN(c1cnc4c(n1)NC(=O)CO4)C(=O)O2)C3 ; O=C(O)C(F)(F)F] | |
| | ESP [M + H]⁺: 537.2 | |
| 13 | 6-[8-[[4,8-difluoro-2-[2-(methylamino)ethyl]-3,5,6,7- | Intermediate 2 and |
| | tetrahydrocyclopenta[f]benzimidazol-6-yl]methyl]-2-oxo-1-oxa-3,8- | Intermediate 15 |
| | diazaspiro[4.5]decan-3-yl]-4H-pyrazino[2,3-b][1,4]oxazin-3-one; | |
| | 2,2,2-trifluoroacetic acid | |
| | 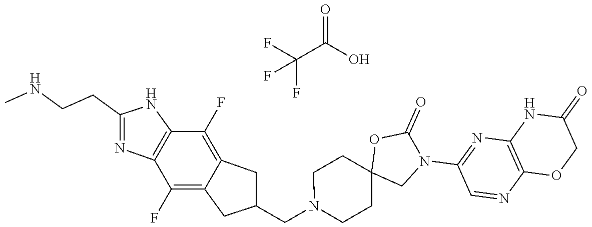 [SMILES: CNCCc1nc2c(F)c3c(c(F)c2[nH]1)CC(CN1CCC2(CC1)CN(c1cnc4c(n1)NC(=O)CO4)C(=O)O2)C3 ; O=C(O)C(F)(F)F] | |
| | ESP [M + H]⁺: 569.3 | |
| 14 | 6-[8-[[4,8-difluoro-2-[(3S)-pyrrolidin-3-yl]-3,5,6,7- | Intermediate 2 and |
| | tetrahydrocyclopenta[f]benzimidazol-6-yl]methyl]-2-oxo-1-oxa-3,8- | Intermediate 16 |
| | diazaspiro[4.5]decan-3-yl]-4H-pyrazino[2,3-b][1,4]oxazin-3- | |
| | one; 2,2,2-trifluoroacetic acid | |
| | 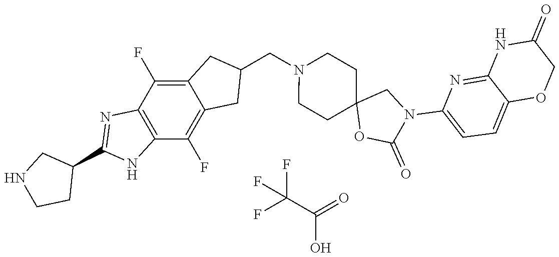 [SMILES: O=C1COc2ccc(N3CC4(CCN(CC5Cc6c(c(F)c7[nH]c([C@H]8CCNC8)nc7c6F)C5)CC4)OC3=O)nc2N1 ; O=C(O)C(F)(F)F] | |
| | ESP [M + H]⁺: 581.4 | |
| 15 | 6-[8-[[4,8-difluoro-2-[(3S)-pyrrolidin-3-yl]-3,5,6,7- | Intermediate 3 and |
| | tetrahydrocyclopenta[f]benzimidazol-6-yl]methyl]-2-oxo-1-oxa-3,8- | Intermediate 16 |
| | diazaspiro[4.5]decan-3-yl]-4H-pyrido[3,2-b][1,4]oxazin-3-one; 2,2,2- | |
| | trifluoroacetic acid | |
| | 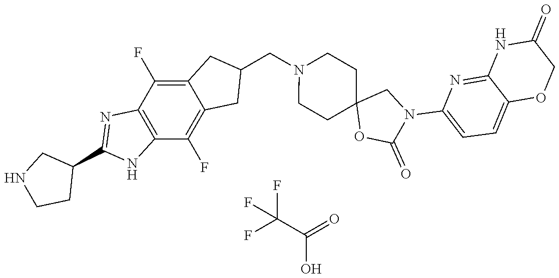 [SMILES: O=C1COc2ccc(N3CC4(CCN(CC5Cc6c(c(F)c7[nH]c([C@H]8CCNC8)nc7c6F)C5)CC4)OC3=O)nc2N1 ; O=C(O)C(F)(F)F] | |
| | ESP [M + H]⁺: 580.4 | |
| 16 | 6-[8-[(4,8-difluoro-2-morpholin-2-yl-3,5,6,7- | Intermediate 2 and |
| | tetrahydrocyclopenta[f]benzimidazol-6-yl)methyl]-2-oxo-1-oxa-3,8- | Intermediate 17 |
| | diazaspiro[4.5]decan-3-yl]-4H-pyrazino[2,3-b][1,4]oxazin-3- | |
| | one; 2,2,2-trifluoroacetic acid | |
| | 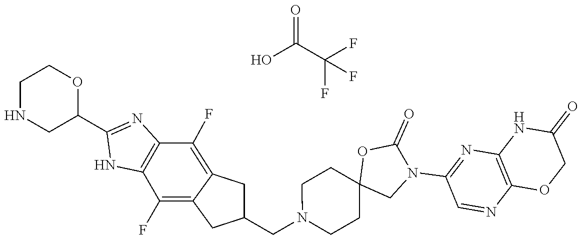 [SMILES: O=C1COc2ncc(N3CC4(CCN(CC5Cc6c(c(F)c7[nH]c(C8CNCCO8)nc7c6F)C5)CC4)OC3=O)nc2N1 ; O=C(O)C(F)(F)F] | |
| | ESP [M + H]⁺: 597.2 | |
| 17 | 6-[8-[[4,8-difluoro-2-[(1R)-1-(methylamino)ethyl]-3,5,6,7- | Intermediate 2 and |
| | tetrahydrocyclopenta[f]benzimidazol-6-yl]methyl]-2-oxo-1-oxa-3,8- | Intermediate 26 |
| | diazaspiro[4.5]decan-3-yl]-4H-pyrazino[2,3-b][1,4]oxazin-3- | |
| | one; 2,2,2-trifluoroacetic acid | |
| | 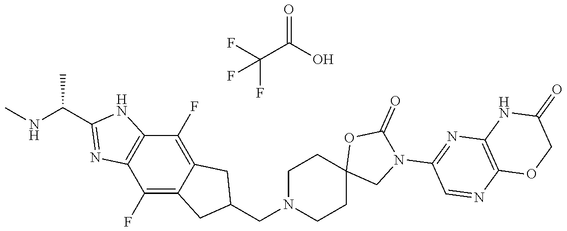 [SMILES: CN[C@H](C)c1nc2c(F)c3c(c(F)c2[nH]1)CC(CN1CCC2(CC1)CN(c1cnc4c(n1)NC(=O)CO4)C(=O)O2)C3 ; O=C(O)C(F)(F)F] | |
| | ESP [M + H]⁺: 569.3 | |
| 18 | 6-[8-[[4,8-difluoro-2-[(3R)-morpholin-3-yl]-3,5,6,7- | Intermediate 2 and |
| | tetrahydrocyclopenta[f]benzimidazol-6-yl]methyl]-2-oxo-1-oxa-3,8- | Intermediate 27 |
| | diazaspiro[4.5]decan-3-yl]-4H-pyrazino[2,3-b][1,4]oxazin-3- | |
| | one; 2,2,2-trifluoroacetic acid | |
| | 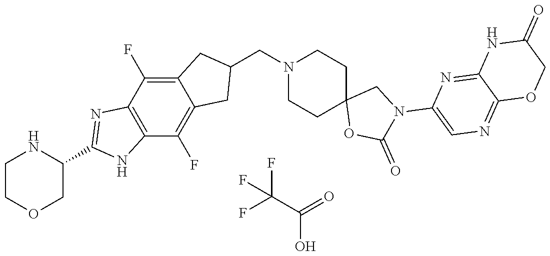 [SMILES: O=C1COc2ncc(N3CC4(CCN(CC5Cc6c(c(F)c7[nH]c([C@@H]8COCCN8)nc7c6F)C5)CC4)OC3=O)nc2N1 ; O=C(O)C(F)(F)F] | |
| | ESP [M + H]⁺: 597.3 | |
| 19 | 6-[2-[[4,8-difluoro-2-(methylaminomethyl)-3,5,6,7- | Intermediate 4 and |
| | tetrahydrocyclopenta[f]benzimidazol-6-yl]methylamino]-6-oxo-5- | Intermediate 11 |
| | oxa-7-azaspiro[3.4]octan-7-yl]-4H-pyrazino[2,3-b][1,4]oxazin-3-one | |
| | 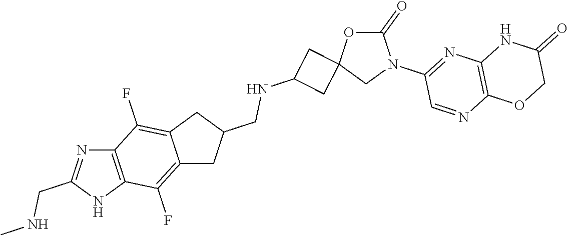 [SMILES: CNCc1nc2c(F)c3c(c(F)c2[nH]1)CC(CNC1CC2(C1)CN(c1cnc4c(n1)NC(=O)CO4)C(=O)O2)C3] | |
| | ESP [M + H]⁺: 541.3 | |
| 20 | 6-[8-[[4,8-difluoro-1-methyl-2-[2-(methylamino)ethyl]-6,7-dihydro- | Intermediate 2 and |
| | 5H-cyclopenta[f]benzimidazol-6-yl]methyl]-2-oxo-1-oxa-3,8- | Intermediate 28 |
| | diazaspiro[4.5]decan-3-yl]-4H-pyrazino[2,3-b][1,4]oxain-3- | |
| | one; 2,2,2-trifluoroacetic acid | |
| | 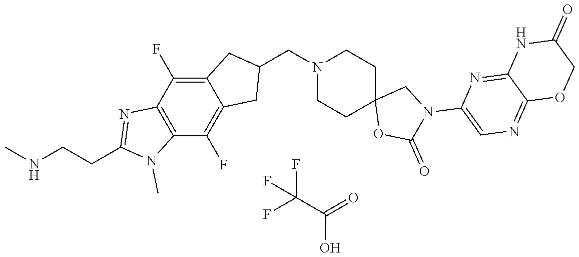 | |
| | ESP [M + H]⁺: 583.4 | |
| 21 | 6-[8-[[4,8-difluoro-2-[(2S)-5-oxopyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 18 |
| | diazaspiro[4.5]decan-3-yl]-4H-pyrazino[2,3-b][1,4]oxazin-3- | |
| | one; 2,2,2-trifluoroacetic acid | |
| | 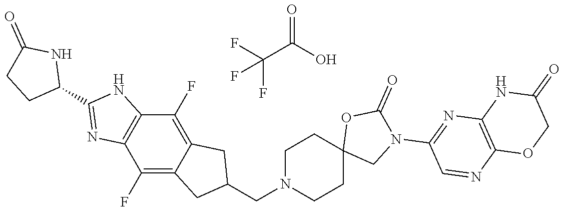 | |
| | ESP [M + H]⁺: 595.3 | |
| 22 | 6-[8-[[8-fluoro-2-(methylaminomethyl)-6,7-dihydro-5H- | Intermediate 2 and |
| | cyclopenta[f][1,3]benzoxazol-6-yl]methyl]-2-oxo-1-oxa-3,8- | Intermediate 29 |
| | diazaspiro[4.5]decan-3-yl]-4H-pyrazino[2,3-b][1,4]oxazin-3- | |
| | one; 2,2,2-trifluoroacetic acid | |
| | 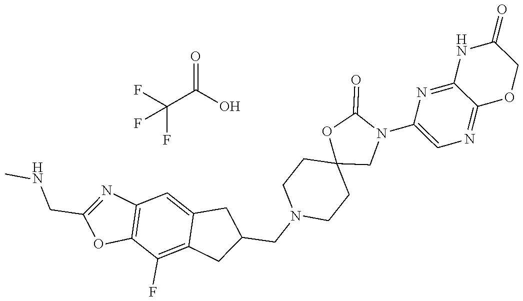 [SMILES: CNCc1nc2cc3c(c(F)c2o1)CC(CN1CCC2(CC1)CN(c1cnc4c(n1)NC(=O)CO4)C(=O)O2)C3 ; O=C(O)C(F)(F)F] | |
| | ESP [M + H]⁺: 538.3 | |
| 23 | 6-[8-[(10,16-difluoro-2,5,8- | Intermediate 2 and |
| | triazatetracyclo[7.7.0.02,7.011,15]hexadeca-1(9),7,10,15-tetraen-13- | Intermediate 30 |
| | yl)methyl]-2-oxo-1-oxa-3,8-diazaspiro[4.5]decan-3-yl]-4H- | |
| | pyrazino[2,3-b][1,4]oxazin-3-one | |
| | 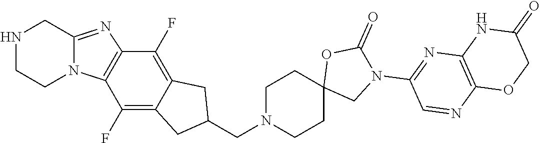 [SMILES: O=C1COc2ncc(N3CC4(CCN(CC5Cc6c(c(F)c7c(nc8n7CCNC8)c6F)C5)CC4)OC3=O)nc2N1] | |
| | ESP [M + H]⁺; 567.3 | |
| 24 | 6-[8-[[4,8-difluoro-2-[(2R)-pyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 19 |
| | diazaspiro[4.5]decan-3-yl]-4H-pyrazino[2,3-b][1,4]oxazin-3-one | |
| | 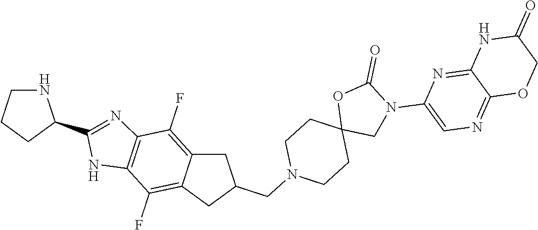 [SMILES: O=C1COc2ncc(N3CC4(CCN(CC5Cc6c(c(F)c7[nH]c([C@H]8CCCN8)nc7c6F)C5)CC4)OC3=O)nc2N1] | |
| | ESP [M + H]⁺: 581.3 | |
| 25 | 6-[8-[[2-[(1R)-1-aminoethyl]-4,8-difluoro-3,5,6,7- | Intermediate 2 and |
| | tetrahydrocyclopenta[f]benzimidazol-6-yl]methyl]-2-oxo-1-oxa-3,8- | Intermediate 20 |
| | diazaspiro[4.5]decan-3-yl]-4H-pyrazino[2,3-b][1,4]oxazin-3- | |
| | one; 2,2,2-trifluoroacetic acid | |
| | 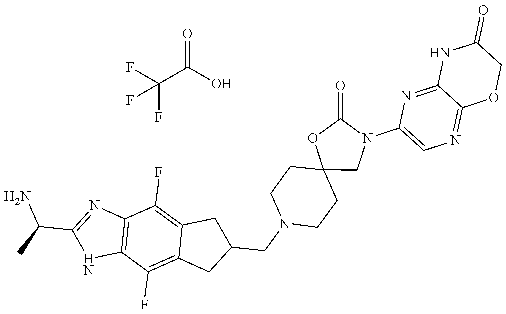 [SMILES: C[C@@H](N)c1nc2c(F)c3c(c(F)c2[nH]1)CC(CN1CCC2(CC1)CN(c1cnc4c(n1)NC(=O)CO4)C(=O)O2)C3 ; O=C(O)C(F)(F)F] | |
| | ESP [M + H]⁺: 555.3 | |
| 26 | 6-[2-[[4,8-difluoro-2-[(1R)-1-(methylamino)ethyl]-3,5,6,7- | Intermediate 4 and |
| | tetrahydrocyclopenta[f]benzimidazol-6-yl]methylamino]-6-oxo-5- | Intermediate 26 |
| | oxa-7-azaspiro[3.4]octan-7-yl]-4H-pyrazino[2,3-b][1,4]oxazin-3-one | |
| | 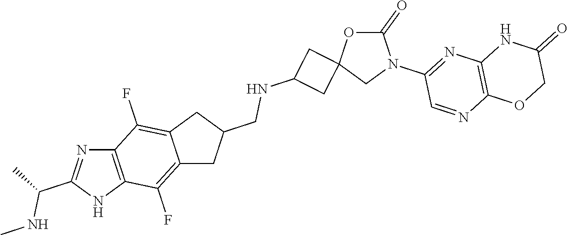 [SMILES: CN[C@H](C)c1nc2c(F)c3c(c(F)c2[nH]1)CC(CNC1CC2(C1)CN(c1cnc4c(n1)NC(=O)CO4)C(=O)O2)C3] | |
| | ESP [M + H]⁺: 555.2 | |
| 27 | 6-[8-[[4,8-difluoro-2-[(2S,4R)-4-(methylamino)pyrrolidin-2-yl]- | Intermediate 2 and |
| | 3,5,6,7-tetrahydrocyclopenta[f]benzimidazol-6-yl]methyl]-2-oxo-1- | Intermediate 31 |
| | oxa-3,8-diazaspiro[4.5]decan-3-yl]-4H-pyrazino[2,3-b][1,4]oxazin-3- | |
| | one | |
| | 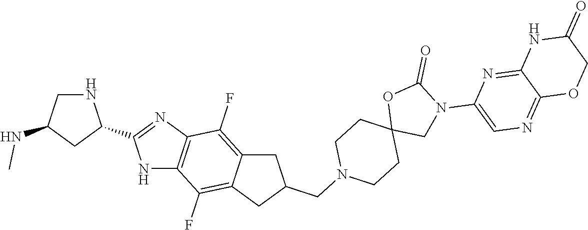 [SMILES: CN[C@H]1CN[C@H](c2nc3c(F)c4c(c(F)c3[nH]2)CC(CN2CCC3(CC2)CN(c2cnc5c(n2)NC(=O)CO5)C(=O)O3)C4)C1] | |
| | ESP [M + H]⁺: 610.3 | |
| 28 | 6-[8-[[4,8-difluoro-2-[(2S)-4-hydroxy-2-piperidyl]-3,5,6,7- | Intermediate 2 and |
| | tetrahydrocyclopenta[f]benzimidazol-6-yl]methyl]-2-oxo-1-oxa-3,8 | Intermediate 21 |
| | diazaspiro[4.5]decan-3-yl]-4H-pyrazino[2,3-b][1,4]oxazin-3- | |
| | one; 2,2,2-trifluoroacetic acid | |
| | 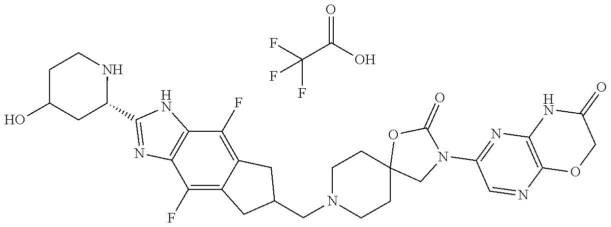 | |
| | ESP [M + H]⁺: 611.3 | |
| 29 | 6-[2-[[2-[(dimethylamino)methyl]-4,8-difluoro-3,5,6,7- tetrahydrocyclopenta[f]benzimidazol-6-yl]methylamino]-6-oxo-5- oxa-7-azaspiro[3.4]octan-7-yl]-4H-pyrazino[2,3-b][1,4]oxazin-3-one 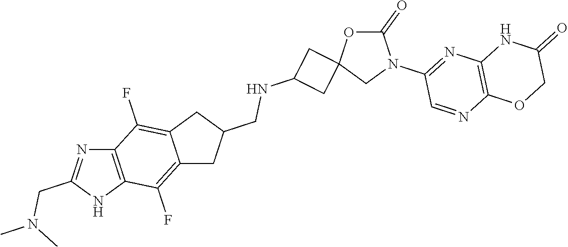 [SMILES: CN(C)Cc1nc2c(F)c3c(c(F)c2[nH]1)CC(CNC1CC2(C1)CN(c1cnc4c(n1)NC(=O)CO4)C(=O)O2)C3] | 6-(2-amino-6-oxo-5- oxa-7- azaspiro[3.4]octan-7- yl)-4-(2- trimethylsilylethoxy- methyl)pyrazino[2,3- b][1,4]oxazin-3-one (Intermediate 4, Step 6) and Intermediate 9 |
| | ESP [M + H ]⁺: 555.3 | |
| 30 | 6-[8-[[2-(1-amino-1-methyl-ethyl)-4,8-difluoro-3,5,6,7- | Intermediate 2 and |
| | tetrahydrocyclopenta[f]benzimidazol-6-yl]methyl]-2-oxo-1-oxa-3,8- | Intermediate 46 |
| | diazaspiro[4.5]decan-3-yl]-4H-pyrazino[2,3-b][1,4]oxazin-3-one | |
| | 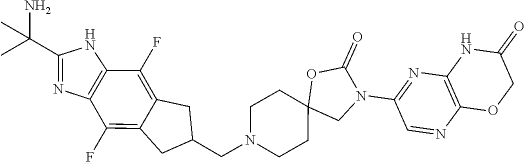 [SMILES: CC(C)(N)c1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 569.3 | |
| 31 | 6-[8-[[4,8-difluoro-2-[(3S,4R)-4-aminotetrahydrofuran-3-yl]-3,5,6,7- | Intermediate 2 and |
| | tetrahydrocyclopenta[f]benzimidazol-6-yl]methyl]-2-oxo-1-oxa-3,8- | Intermediate 41 |
| | diazaspiro[4.5]decan-3-yl]-4H-pyrazino[2,3-b][1,4]oxazin-3-one | |
| | 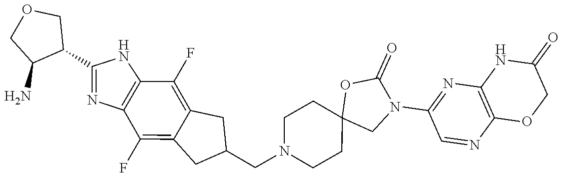 [SMILES: N[C@H]1COC[C@@H]1c1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 597.3 | |
| 32 | 6-[8-[(2-amino-4,8-difluoro-3,5,6,7- | Intermediate 2 and |
| | tetrahydrocyclopenta[f]benzimidazol-6-yl)methyl]-2-oxo-1-oxa-3,8- | Intermediate 42 |
| | diazaspiro[4.5]decan-3-yl]-4H-pyrazino[2,3-b][1,4]oxazin-3-one | |
| | 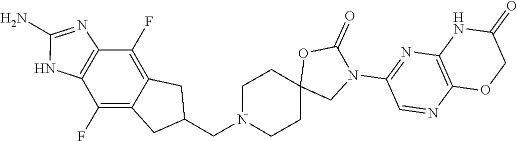 [SMILES: Nc1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 527.2 | |
| 33 | 6-[8-[[4,8-difluoro-1-methyl-2-(methylaminomethyl)-6,7-dihydro- | Intermediate 2 and |
| | 5H-cyclopenta[f]benzimidazol-6-yl]methyl]-2-oxo-1-oxa-3,8- | Intermediate 43 |
| | diazaspiro[4.5]decan-3-yl]-4H-pyrazino[2,3-b][1,4]oxazin-3-one | |
| | 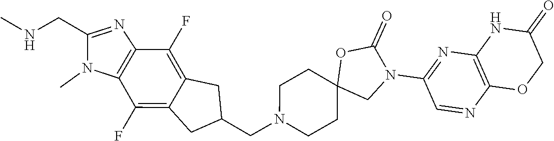 [SMILES: CNCc1nc2c(F)c3c(c(F)c2n1C)CC(CN1CCC2(CC1)CN(c1cnc4c(n1)NC(=O)CO4)C(=O)O2)C3] | |
| | ESP [M + H]⁺: 569.4 | |
| 34 | 6-[8-[[2-(2-azabicyclo[2.1.1]hexan-1-yl)-4,8-difluoro-3,5,6,7- | Intermediate 2 and |
| | tetrahydrocyclopenta[f]benzimidazol-6-yl]methyl]-2-oxo-1-oxa-3,8- | Intermediate 44 |
| | diazaspiro[4.5]decan-3-yl]-4H-pyrazino[2,3-b][1,4]oxazin-3-one | |
| | 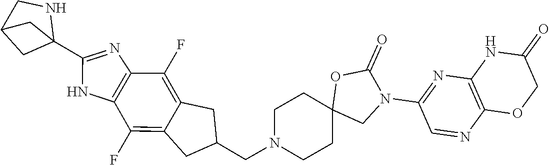 [SMILES: O=C1COc2ncc(N3CC4(CCN(CC5Cc6c(c(F)c7[nH]c(C89CC(CN8)C9)nc7c6F)C5)CC4)OC3=O)nc2N1] | |
| | ESP [M + H]⁺: 593.3 | |
| 35 | N-[4,8-difluoro-6-[[2-oxo-3-(3-oxo-4H-pyrazino[2,3-b][1,4]oxazin- | Intermediate 2 and |
| | 6-yl)-1-oxa-3,8-diazaspiro[4.5]decan-8-yl]methyl]-3,5,6,7- | Intermediate 45 |
| | tetrahydrocyclopenta[f]benzimidazol-2-yl]-2- | |
| | (methylamino)acetamide | |
| | 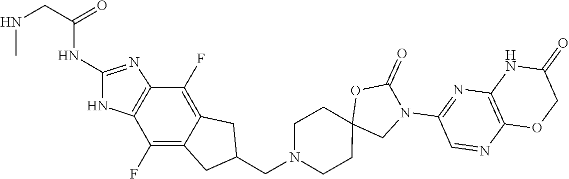 [SMILES: CNCC(=O)Nc1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 598.2 | |
| 36 | 6-[8-[[2-[(1S,2S)-1-amino-2-hydroxy-propyl]-4,8-difluoro-3,5,6,7- | Intermediate 2 and |
| | tetrahydrocyclopenta[f]benzimidazol-6-yl]methyl]-2-oxo-1-oxa-3,8- | Intermediate 37 |
| | diazaspiro[4.5]decan-3-yl]-4H-pyrazino[2,3-b][1,4]oxazin-3-one | |
| | 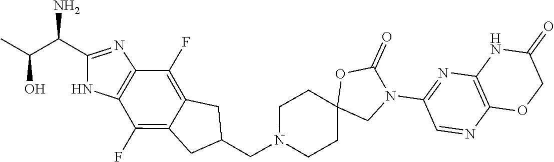 [SMILES: C[C@H](O)[C@@H](N)c1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 585.3 | |
| 37 | 6-[8-[[4,8-difluoro-2-[(2R,4S)-4-methoxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 57 |
| | diazaspiro[4.5]decan-3-yl]-4H-pyrazino[2,3-b][1,4]oxazin-3-one | |
| | 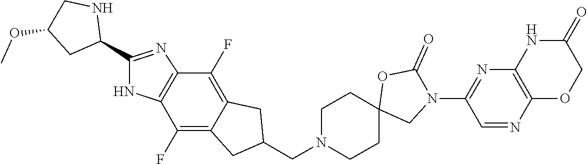 [SMILES: CO[C@@H]1CN[C@@H](c2nc3c(F)c4c(c(F)c3[nH]2)CC(CN2CCC3(CC2)CN(c2cnc5c(n2)NC(=O)CO5)C(=O)O3)C4)C1] | |
| | ESP [M + H]⁺: 611.5 | |
| 38 | 6-[8-[[4,8-difluoro-2-[(1S)-1-(methylamino)ethyl]-3,5,6,7- | Intermediate 2 and |
| | tetrahydrocyclopenta[f]benzimidazol-6-yl]methyl]-2-oxo-1-oxa-3,8- | Intermediate 47 |
| | diazaspiro[4.5]decan-3-yl]-4H-pyrazino[2,3-b][1,4]oxazin-3-one | |
| | 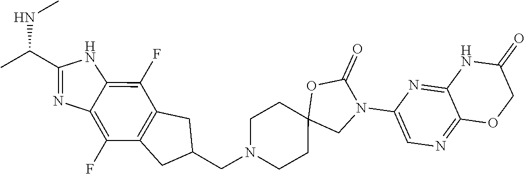 [SMILES: CN[C@@H](C)c1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 569.4 | |
| 39 | 6-[8-[(3-amino-5,9-difluoro-7,8-dihydro-6H- | Intermediate 2 and |
| | cyclopenta[g]quinoxalin-7-yl)methyl]-2-oxo-1-oxa-3,8- | Intermediate 49 |
| | diazaspiro[4.5]decan-3-yl]-4H-pyrazino[2,3-b][1,4]oxazin-3-one | |
| | 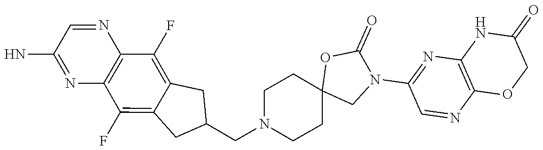 [SMILES: Nc1cnc2c(F)c3c(c(F)c2n1)CC(CN1CCC2(CC1)CN(c1cnc4c(n1)NC(=O)CO4)C(=O)O2)C3] | |
| | ESP [M + H]⁺: 539.3 | |
| 40 | 6-[8-[[5,9-difluoro-3-[2-(methylamino)ethylamino]-7,8-dihydro-6H- | Intermediate 2 and |
| | cyclopenta[g]quinoxalin-7-yl]methyl]-2-oxo-1-oxa-3,8- | Intermediate 50 |
| | diazaspiro[4.5]decan-3-yl]-4H-pyrazino[2,3-b][1,4]oxazin-3-one | |
| | 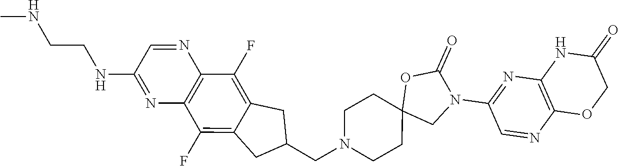 [SMILES: CNCCNc1cnc2c(F)c3c(c(F)c2n1)CC(CN1CCC2(CC1)CN(c1cnc4c(n1)NC(=O)CO4)C(=O)O2)C3] | |
| | ESP [M + H]⁺: 596.3 | |
| 41 | 6-[8-[[2-(2-aminoethyl)-4,8-difluoro-6,7-dihydro-5H- | Intermediate 2 and |
| | cyclopenta[f]benzotriazol-6-yl]methyl]-2-oxo-1-oxa-3,8- | Intermediate 51 |
| | diazaspiro[4.5]decan-3-yl]-4H-pyrazino[2,3-b][1,4]oxazin-3-one | |
| | 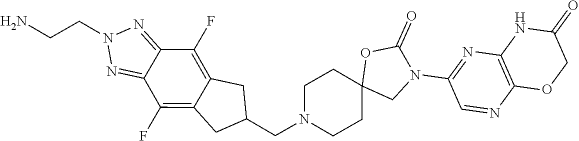 [SMILES: NCCn1nc2c(F)c3c(c(F)c2n1)CC(CN1CCC2(CC1)CN(c1cnc4c(n1)NC(=O)CO4)C(=O)O2)C3] | |
| | ESP [M + H]⁺: 556.3 | |
| 42 | 6-[8-[[1-(2-aminoethyl)-4,8-difluoro-6,7-dihydro-5H- | Intermediate 2 and |
| | cyclopenta[f]benzotriazol-6-yl]methyl]-2-oxo-1-oxa-3,8- | Intermediate 52 |
| | diazaspiro[4.5]decan-3-yl]-4H-pyrazino[2,3-b][1,4]oxazin-3-one | |
| | 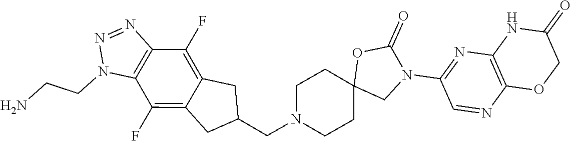 [SMILES: NCCn1nnc2c(F)c3c(c(F)c21)CC(CN1CCC2(CC1)CN(c1cnc4c(n1)NC(=O)CO4)C(=O)O2)C3] | |
| | ESP [M + H]⁺: 556.3 | |
| 43 | 6-[8-[[1,3-bis(2-aminoethyl)-4,8-difluoro-2-oxo-6,7-dihydro-5H- | Intermediate 2 and |
| | cyclopenta[f]benzimidazol-6-yl]methyl]-2-oxo-1-oxa-3,8- | Intermediate 53 |
| | diazaspiro[4.5]decan-3-yl]-4H-pyrazino[2,3-b][1,4]oxazin-3- | |
| | one; 2,2,2-trifluoroacetic acid | |
| | 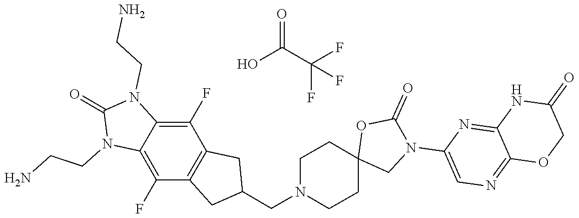 [SMILES: NCCn1c(=O)n(CCN)c2c(F)c3c(c(F)c21)CC(CN1CCC2(CC1)CN(c1cnc4c(n1)NC(=O)CO4)C(=O)O2)C3 ; O=C(O)C(F)(F)F] | |
| | ESP [M + H]⁺: 614.3 | |
| 44 | 6-[8-[[1-(2-aminoethyl)-4,8-difluoro-2-oxo-3,5,6,7- | Intermediate 2 and |
| | tetrahydrocyclopenta[f]benzimidazol-6-yl]methyl]-2-oxo-1-oxa-3,8- | Intermediate 54 |
| | diazaspiro[4.5]decan-3-yl]-4H-pyrazino[2,3-b][1,4]oxazin-3-one | |
| | 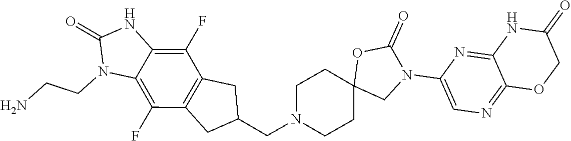 [SMILES: NCCn1c(=O)[nH]c2c(F)c3c(c(F)c21)CC(CN1CCC2(CC1)CN(c1cnc4c(n1)NC(=O)CO4)C(=O)O2)C3] | |
| | ESP [M + H]⁺: 571.3 | |
| 45 | 6-[8-[[2-[(1R)-1-aminoethyl]-8-fluoro-1,5,6,7- | Intermediate 2 and |
| | tetrahydrocyclopenta[f]benzimidazol-6-yl]methyl]-2-oxo-1-oxa-3,8- | Intermediate 55 |
| | diazaspiro[4.5]decan-3-yl]-4H-pyrazino[2,3-b][1,4]oxazin-3-one | |
| | 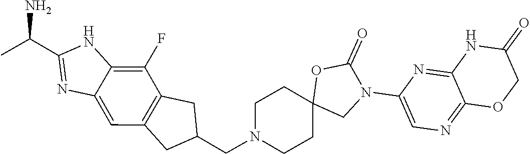 [SMILES: C[C@@H](N)c1nc2cc3c(c(F)c2[nH]1)CC(CN1CCC2(CC1)CN(c1cnc4c(n1)NC(=O)CO4)C(=O)O2)C3] | |
| | ESP [M + H]⁺: 537.3 | |

### Example 46

### 6-[8-[[4,8-Difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid

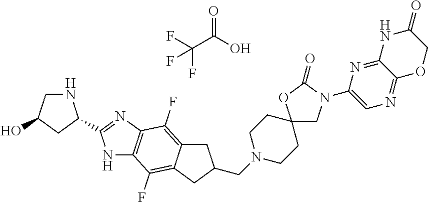

### Step 1: tert-butyl (2S,4R)-4-[tert-butyl(dimethyl)silyl]oxy-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]pyrrolidine-1-carboxylate

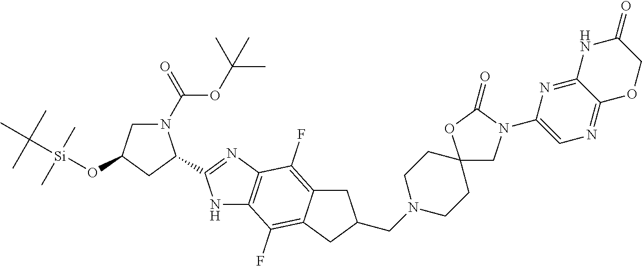 [SMILES: CC(C)(C)OC(=O)N1C[C@H](O[Si](C)(C)C(C)(C)C)C[C@H]1c1nc2c(F)c3c(c(F)c2[nH]1)CC(CN1CCC2(CC1)CN(c1cnc4c(n1)NC(=O)CO4)C(=O)O2)C3]

A solution of crude tert-butyl (2S,4R)-4-[tert-butyl(dimethyl)silyl]oxy-2-(4,8-difluoro-6-formyl-3,5,6,7-tetrahydrocyclopenta[f]benzimidazol-2-yl)pyrrolidine-1-carboxylate (Intermediate 32) (250.0 mg, 0.480 mmol, 1 eq), 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2) (221.04 mg, 0.530 mmol, 1.1 eq) and DIPEA (125.21 uL, 0.720 mmol, 1.5 eq) in DMA (2.4 mL) and THF (2.4 mL) was stirred at 25° C. for 15 min. Acetic acid (68.52 uL, 1.2 mmol, 2.5 eq) was added followed by sodium triacetoxyborohydride (152.35 mg, 0.720 mmol, 1.5 eq). The mixture was stirred for 1 h at 25° C. The mixture was diluted with saturated aqueous NaHCO₃ and extracted with EtOAc (×1). The organic phase was dried over Na₂SO₄ and concentrated in vacuo. The mixture was purified by silica cartridge chromatography (50 g, DCM/MeOH from 99.5:0.5 to 95:5) to afford the title compound (237 mg, 0.290 mmol, 60.98% yield) as a light yellow solid. MS (ESP) m/z=811.3 [M+H]⁺.

### Step 2: tert-butyl (2S,4R)-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-4-hydroxy-pyrrolidine-1-carboxylate

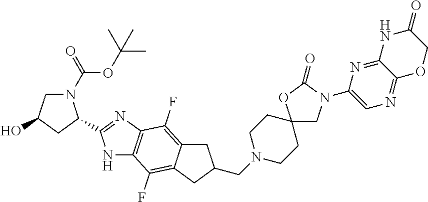 [SMILES: CC(C)(C)OC(=O)N1C[C@H](O)C[C@H]1c1nc2c(F)c3c(c(F)c2[nH]1)CC(CN1CCC2(CC1)CN(c1cnc4c(n1)NC(=O)CO4)C(=O)O2)C3]

To a stirred solution of tert-butyl (2S,4R)-4-[tert-butyl(dimethyl)silyl]oxy-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]pyrrolidine-1-carboxylate (237.0 mg, 0.290 mmol, 1 eq) in dry THF (2.92 mL), tetrabutylammonium fluoride 1M in THF (584.49 uL, 0.580 mmol, 2 eq) was added and the mixture was stirred for 16 h at RT. The reaction was diluted with saturated aqueous NaHCO₃ and extracted with EtOAc (×2). The organic phase was filtered over a phase separator and concentrated in vacuo. The mixture was purified by silica cartridge chromatography (25 g, [DCM/(DCM/MeOH/NH₄OH 10:1:0.1) from 95:5 to 0:100) to afford the title compound (144 mg, 0.210 mmol, 70.72% yield), as a white solid. MS (ESP) m/z=697.2 [M+H]⁺.

### Step 3: 6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid

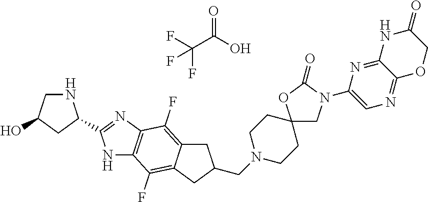

To a stirred suspension of tert-butyl (2S,4R)-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-4-hydroxy-pyrrolidine-1-carboxylate (100.0 mg, 0.140 mmol, 1 eq) in DCM (2 mL), trifluoroacetic acid (221.16 uL, 2.87 mmol, 20 eq) was added and the mixture was stirred for 2 h at RT. The reaction was concentrated in vacuo. The resulting mixture was triturated in diethyl ether and filtered. The solid was suspended in H₂O/MeCN and lyophilised to afford the title compound (110 mg, 0.130 mmol, 88.29% yield), as a white solid. MS (ESP) m/z=597.2 [M+H]⁺.

The following examples were prepared by analogy to Example 46.

| Ex. | Name, Structure, MS | Starting Materials |
|---|---|---|
| 47 | 6-[5-[2-[[4,8-difluoro-2-[(2S,4R)-4-hydroxypyrrolidin-2-yl]-3,5,6,7- | Intermediate 1 and |
| | tetrahydrocyclopenta[f]benzimidazol-6-yl]methylamino]ethyl]-2- | Intermediate 32 |
| | oxo-oxazolidin-3-yl]-4H-pyrazino[2,3-b][1,4]oxazin-3-one; 2,2,2- | |
| | trifluoroacetic acid | |
| | 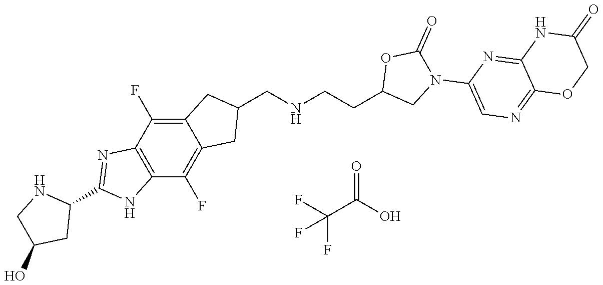 [SMILES: O=C1COc2ncc(N3CC(CCNCC4Cc5c(c(F)c6[nH]c([C@@H]7C[C@@H](O)CN7)nc6c5F)C4)OC3=O)nc2N1 ; O=C(O)C(F)(F)F] | |
| | ESP [M + H]⁺: 571.2 | |
| 48 | 6-[8-[[8-fluoro-2-[(2S,4R)-4-hydroxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 33 |
| | diazaspiro[4.5]decan-3-yl]-4H-pyrazino[2,3-b][1,4]oxazin-3- | |
| | one; 2,2,2-trifluoroacetic acid | |
| | 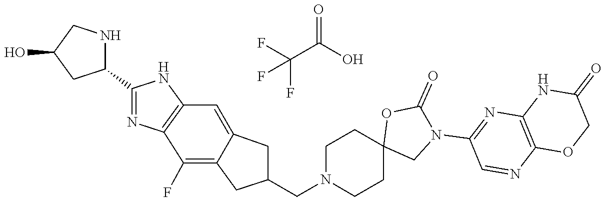 [SMILES: O=C1COc2ncc(N3CC4(CCN(CC5Cc6cc7[nH]c([C@@H]8C[C@@H](O)CN8)nc7c(F)c6C5)CC4)OC3=O)nc2N1 ; O=C(O)C(F)(F)F] | |
| | ESP [M + H]⁺: 579.2 | |
| 49 | 6-[8-[[4,8-difluoro-2-[(2R,4S)-4-hydroxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 34 |
| | diazaspiro[4.5]decan-3-yl]-4H-pyrazino[2,3-b][1,4]oxazin-3- | |
| | one; 2,2,2-trifluoroacetic acid | |
| | 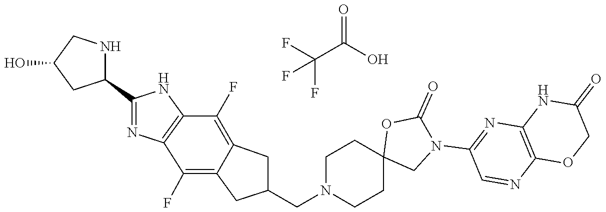 [SMILES: O=C1COc2ncc(N3CC4(CCN(CC5Cc6c(c(F)c7[nH]c([C@H]8C[C@H](O)CN8)nc7c6F)C5)CC4)OC3=O)nc2N1 ; O=C(O)C(F)(F)F] | |
| | ESP [M + H]⁺: 597.3 | |
| 50 | 6-[8-[[4,8-difluoro-2-[(2R,4R)-4-hydroxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 35 |
| | diazaspiro[4.5]decan-3-yl]-4H-pyrazino[2,3-b][1,4]oxazin-3- | |
| | one; 2,2,2-trifluoroacetic acid | |
| | 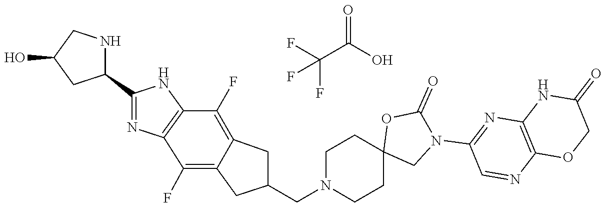 [SMILES: O=C1COc2ncc(N3CC4(CCN(CC5Cc6c(c(F)c7[nH]c([C@H]8C[C@@H](O)CN8)nc7c6F)C5)CC4)OC3=O)nc2N1 ; O=C(O)C(F)(F)F] | |
| | ESP [M + H]⁺: 597.2 | |
| 51 | 6-[8-[[4,8-difluoro-2-[(2R,4R)-4-hydroxypyrrolidin-2-yl]-3,5,6,7- | Intermediate 3 and |
| | tetrahydrocyclopenta[f]benzimidazol-6-yl]methyl]-2-oxo-1-oxa-3,8- | Intermediate 35 |
| | diazaspiro[4.5]decan-3-yl]-4H-pyrido[3,2-b][1,4]oxazin-3-one; 2,2,2- | |
| | trifluoroacetic acid | |
| | 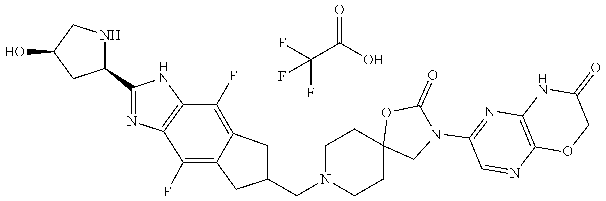 [SMILES: O=C1COc2ncc(N3CC4(CCN(CC5Cc6c(c(F)c7[nH]c([C@H]8C[C@@H](O)CN8)nc7c6F)C5)CC4)OC3=O)nc2N1 ; O=C(O)C(F)(F)F] | |
| | ESP [M + H]⁺: 596.3 | |
| 52 | 6-[8-[[4,8-difluoro-2-[(2S,4S)-4-hydroxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 36 |
| | diazaspiro[4.5]decan-3-yl]-4H-pyrazino[2,3-b][1,4]oxazin-3- | |
| | one; 2,2,2-trifluoroacetic acid | |
| | 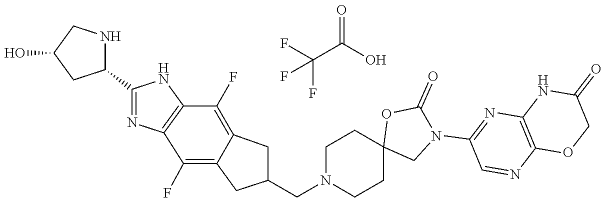 [SMILES: O=C1COc2ncc(N3CC4(CCN(CC5Cc6c(c(F)c7[nH]c([C@@H]8C[C@H](O)CN8)nc7c6F)C5)CC4)OC3=O)nc2N1 ; O=C(O)C(F)(F)F] | |
| | ESP [M + H]⁺: 597.2 | |
| 53 | 6-[2-[[4,8-difluoro-2-[(2R,4R)-4-hydroxypyrrolidin-2-yl]-3,5,6,7- | Intermediate 4 and |
| | tetrahydrocyclopenta[f]benzimidazol-6-yl]methylamino]-6-oxo-5- | Intermediate 35 |
| | oxa-7-azaspiro[3.4]octan-7-yl]-4H-pyrazino[2,3-b][1,4]oxazin-3-one | |
| | 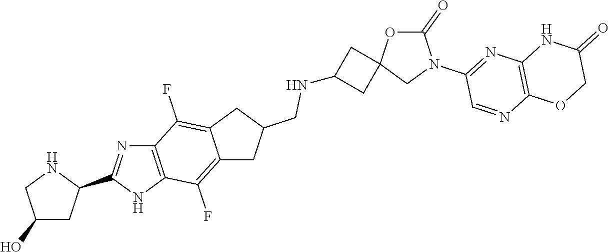 [SMILES: O=C1COc2ncc(N3CC4(CC(NCC5Cc6c(c(F)c7[nH]c([C@H]8C[C@@H](O)CN8)nc7c6F)C5)C4)OC3=O)nc2N1] | |
| | ESP [M + H]⁺: 583.2 | |
| 54 | 6-[8-[[4,8-difluoro-2-[(2R,3S)-3-hydroxypyrrolidin-2-yl]-3,5,6,7- | Intermediate 2 and |
| | tetrahydrocyclopenta[f]benzimidazol-6-yl]methyl]-2-oxo-1-oxa-3,8- | Intermediate 38 |
| | diazaspiro[4.5]decan-3-yl]-4H-pyrazino[2,3-b][1,4]oxazin-3- | |
| | one; 2,2,2-trifluoroacetic acid | |
| | 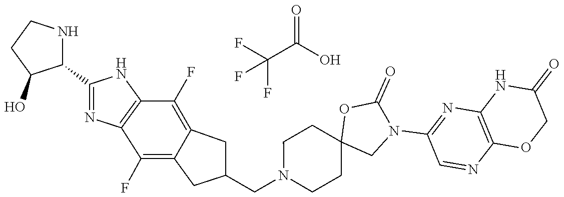 [SMILES: O=C1COc2ncc(N3CC4(CCN(CC5Cc6c(c(F)c7[nH]c([C@H]8NCC[C@@H]8O)nc7c6F)C5)CC4)OC3=O)nc2N1 ; O=C(O)C(F)(F)F] | |
| | ESP [M + H]⁺: 597.3 | |
| 55 | 6-[8-[[4,8-difluoro-2-[(2S,4R)-4-hydroxypyrrolidin-2-yl]-1-methyl- | Intermediate 2 and |
| | 6,7-dihydro-5H-cyclopenta[f]benzimidazol-6-yl]methyl]-2-oxo-1- | Intermediate 39 |
| | oxa-3,8-diazaspiro[4.5]decan-3-yl]-4H-pyrazino[2,3-b][1,4]oxazin-3- | |
| | one; 2,2,2-trifluoroacetic acid | |
| | 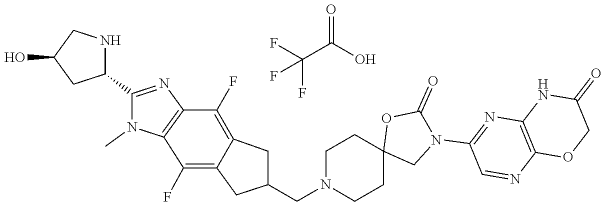 | |
| | ESP [M + H ]⁺: 611.5 | |
| 56 | 6-[8-[[4,8-difluoro-2-[(1S)-2-hydroxy-1-(methylamino)ethyl]-3,5,6,7- | Intermediate 2 and |
| | tetrahydrocyclopenta[f]benzimidaozl-6-yl]methyl]-2-oxo-1-oxa-3,8- | Intermediate 40 |
| | diazaspiro[4.5]decan-3-yl]-4H-pyrazino[2,3-b][1,4]oxazin-3- | |
| | one; 2,2,2-trifluoroacetic acid | |
| | 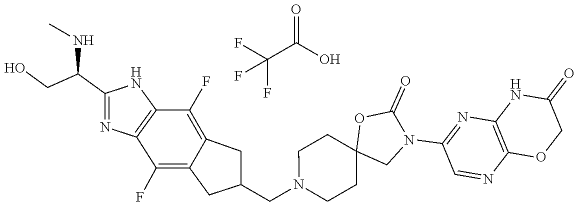 | |
| | ESP [M + H]⁺: 585.2 | |
| 57 | 6-[8-[[4,8-difluoro-2-[(2S)-3-hydroxy-2-(methylamino)propyl]- | Intermediate 2 and |
| | 3,5,6,7-tetrahydrocyclopenta[f]benzimidazol-6-yl]methyl]-2-oxo-1- | Intermediate 48 |
| | oxa-3,8-diazaspiro[4.5]decan-3-yl]-4H-pyrazino[2,3-b][1,4]oxazin-3- | |
| | one | |
| | 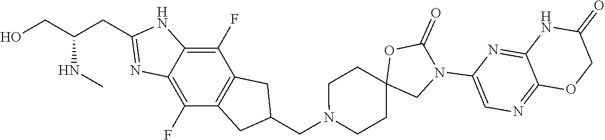 [SMILES: CN[C@H](CO)Cc1nc2c(F)c3c(c(F)c2[nH]1)CC(CN1CCC2(CC1)CN(c1cnc4c(n1)NC(=O)CO4)C(=O)O2)C3] | |
| | ESP [M + H]⁺: 599.3 | |

### Example 58

### 6-[8-[[4,8-Difluoro-2-[(2S,4R)-4-hydroxy-1-methyl-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

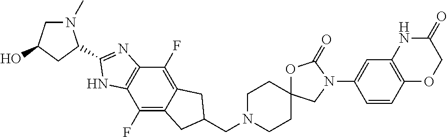

### Step 1: 6-[8-[[2-[(2S,4R)-4-[tert-butyl(dimethyl)silyl]oxy-1-methyl-pyrrolidin-2-yl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

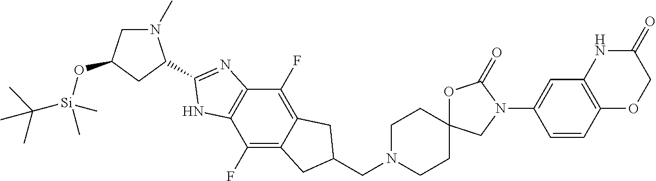

To a suspension of 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2, 67.28 mg, 0.160 mmol, 1 eq) and 2-[(2S,4R)-4-[tert-butyl(dimethyl)silyl]oxy-1-methyl-pyrrolidin-2-yl]-4,8-difluoro-3,5,6,7-tetrahydrocyclopenta[f]benzimidazole-6-carbaldehyde (Intermediate 56, 69.89 mg, 0.13 mmol, 1 eq) in DMA (0.401 mL) and THF (1.2 mL) at r.t., DIPEA (0.07 mL, 0.400 mmol, 2.5 eq) was added. The reaction mixture was stirred at r.t. After 30 min., acetic acid (0.04 mL, 0.720 mmol, 4.5 eq) and sodium triacetoxyborohydride (85.01 mg, 0.400 mmol, 2.5 eq) were added and the mixture stirred at r.t. for 16 h. The reaction mixture was diluted with EtOAc and NaHCO₃ (sat. aq.). The phases were separated and the organic layer was washed with NaHCO₃ (sat. aq.) (×2) and brine (×1). The organic phase was filtered through a phase separator and volatiles were removed under reduced pressure. The crude product was purified by flash column chromatography (0% to 10% of MeOH/DCM) to afford the title compound (85 mg, 0.120 mmol, 73.09% yield) as a white solid. MS (ESP) m/z=725.5 [M+H]⁺.

### Step 2: 6-[8-[[4,8-difluoro-2-[(2S,4R)-4-hydroxy-1-methyl-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

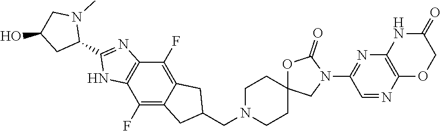 [SMILES: CN1C[C@H](O)C[C@H]1c1nc2c(F)c3c(c(F)c2[nH]1)CC(CN1CCC2(CC1)CN(c1cnc4c(n1)NC(=O)CO4)C(=O)O2)C3]

To a solution of 6-[8-[[2-[(2S,4R)-4-[tert-butyl(dimethyl)silyl]oxy-1-methyl-pyrrolidin-2-yl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one (80.0 mg, 0.110 mmol, 1 eq) in THF (1.1 mL) was added tetrabutylammonium fluoride (0.5 mL, 0.500 mmol, 4.5 eq) and the mixture was stirred at r.t. for 0.75 h. Volatiles were removed under reduced pressure and the residue evaporated with MeCN several times. The residue was suspended with DCM and pentane and stirred, the supernatant was removed and the operation was replicated 3 times. The remaining solid was dried under reduced pressure to give the crude product which was purified by preparative HPLC to give the title compound (14.5 mg, 0.020 mmol, 21.09% yield) as an off-white solid. MS (ESP) m/z=611.4 [M+H]⁺.

### Example 59

### 6-[8-[[8-Fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer A

### and

### Example 60

### 6-[8-[[8-Fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer B

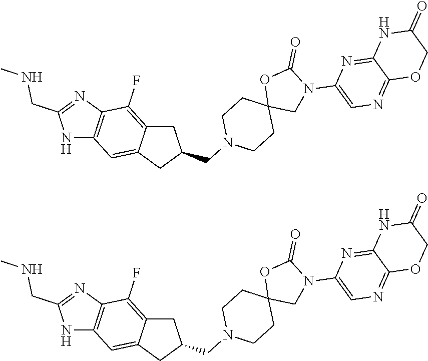 [SMILES: CNCc1nc2c(F)c3c(cc2[nH]1)C[C@H](CN1CCC2(CC1)CN(c1cnc4c(n1)NC(=O)CO4)C(=O)O2)C3 ; CNCc1nc2c(F)c3c(cc2[nH]1)C[C@@H](CN1CCC2(CC1)CN(c1cnc4c(n1)NC(=O)CO4)C(=O)O2)C3]

The enantiomers of 6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid (Example 12, 29.7 mg, 0.030 mmol, 1 eq) were separated by chiral SFC to give peak 1: 6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer A (6.4 mg, 0.010 mmol, 37.87% yield) as a white solid; MS (ESP) m/z=537.3 [M+H]f; and peak 2: 6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer B (6.8 mg, 0.010 mmol, 40.24% yield) as an off-white solid. MS (ESP) m/z=537.3 [M+H]⁺.

### Example 61

### N-[2-[4,8-Difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(methylamino)acetamide

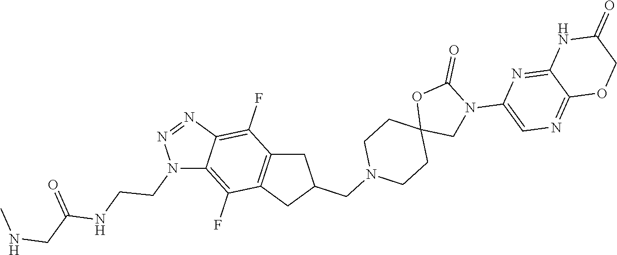 [SMILES: CNCC(=O)NCCn1nnc2c(F)c3c(c(F)c21)CC(CN1CCC2(CC1)CN(c1cnc4c(n1)NC(=O)CO4)C(=O)O2)C3]

### Step 1: tert-butyl N-[2-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethylamino]-2-oxo-ethyl]-N-methyl-carbamate

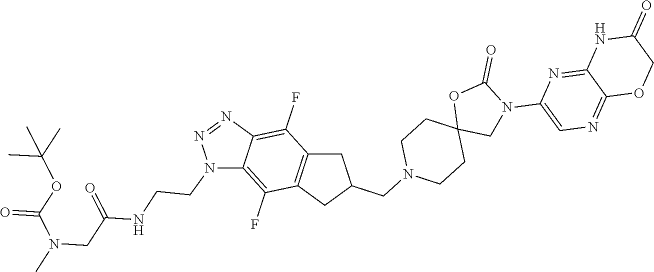 [SMILES: CN(CC(=O)NCCn1nnc2c(F)c3c(c(F)c21)CC(CN1CCC2(CC1)CN(c1cnc4c(n1)NC(=O)CO4)C(=O)O2)C3)C(=O)OC(C)(C)C]

To a stirred solution of N-BOC sarcosine [CAS #13734-36-6] (20.44 mg, 0.110 mmol, 1 eq) in DMF (1.03 mL) N,N-diisopropylethylamine (0.03 mL, 0.160 mmol, 1.5 eq), HATU (49.28 mg, 0.130 mmol, 1.2 eq) and 6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 42, 60.0 mg, 0.110 mmol, 1 eq) were added and the resulting mixture was stirred at 25° C. for 1.5 h. Then the mixture was diluted with water (3 mL) and the resulting mixture was extracted with EtOAc (3×10 mL). The organic layers were collected, dried over Na₂SO₄, filtered and concentrated under vacuum to give the crude product that was purified by silica cartridge chromatography (NH-silica, 28 g, DCM/MeOH 100:0 to 90:10) to afford the title compound (57 mg, 0.080 mmol, 72.62% yield) as a light yellow solid. MS (ESP) m/z=727.4 [M+H]⁺.

### Step 2: N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(methylamino)acetamide

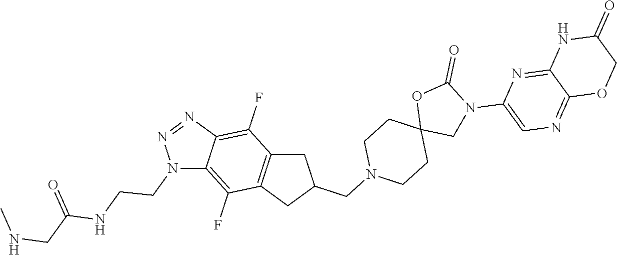 [SMILES: CNCC(=O)NCCn1nnc2c(F)c3c(c(F)c21)CC(CN1CCC2(CC1)CN(c1cnc4c(n1)NC(=O)CO4)C(=O)O2)C3]

To a stirred solution of tert-butyl N-[2-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethylamino]-2-oxo-ethyl]-N-methyl-carbamate (54.0 mg, 0.070 mmol, 1 eq) in DCM (0.784 mL) trifluoroacetic acid (0.11 mL, 1.49 mmol, 20 eq) was added and the resulting mixture was stirred at 25° C. for 1.5 h. Then the mixture was concentrated under vacuum to give the crude product that was purified by silica cartridge chromatography (NH-silica, 28 g, DCM/MeOH 100:0 to 90:10) to afford the title compound (33 mg, 0.050 mmol, 70.17% yield) as a white solid. MS (ESP) m/z=627.3 [M+H]⁺.

### Example 62

### 6-[8-[[2-[2-(Dimethylamino)ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one

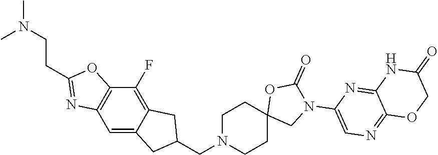 [SMILES: CN(C)CCc1nc2cc3c(c(F)c2o1)CC(CN1CCC2(CC1)CN(c1cnc4c(n1)NC(=O)CO4)C(=O)O2)C3]

The title compound was prepared from Intermediate 2 and Intermediate 58 by analogy with Example 1 and was obtained as white solid. MS (ESP) m/z=566.2 [M+H]⁺.

### Example 63

### 6-[8-[[2-[(2S)-2-(Dimethylamino)-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

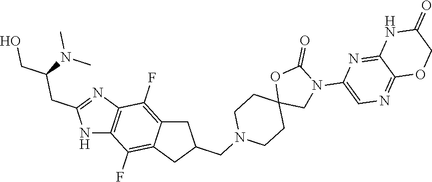 [SMILES: CN(C)[C@H](CO)Cc1nc2c(F)c3c(c(F)c2[nH]1)CC(CN1CCC2(CC1)CN(c1cnc4c(n1)NC(=O)CO4)C(=O)O2)C3]

### Step 1: tert-butyl N-[(1S)-1-[[tert-butyl(dimethyl)silyl]oxymethyl]-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]ethyl]carbamate

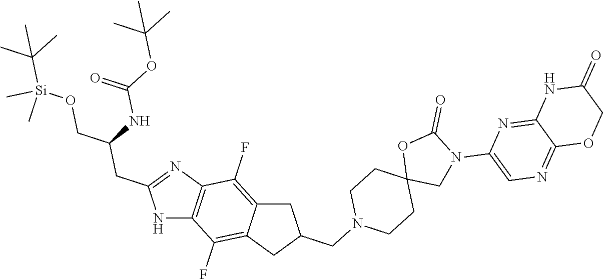 [SMILES: CC(C)(C)OC(=O)N[C@H](CO[Si](C)(C)C(C)(C)C)Cc1nc2c(F)c3c(c(F)c2[nH]1)CC(CN1CCC2(CC1)CN(c1cnc4c(n1)NC(=O)CO4)C(=O)O2)C3]

The title compound was prepared from Intermediate 2 and Intermediate 59 by analogy with Example 1 and was obtained as a yellow solid. MS (ESP) m/z=799.5 [M+H]⁺.

### Step 2: tert-butyl N-[(1S)-1-[[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]methyl]-2-hydroxy-ethyl]carbamate

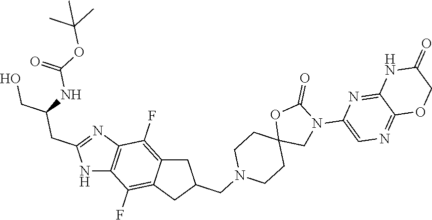 [SMILES: CC(C)(C)OC(=O)N[C@H](CO)Cc1nc2c(F)c3c(c(F)c2[nH]1)CC(CN1CCC2(CC1)CN(c1cnc4c(n1)NC(=O)CO4)C(=O)O2)C3]

A mixture of tert-butyl N-[(1S)-1-[[tert-butyl(dimethyl)silyl]oxymethyl]-2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]ethyl]carbamate (88.0 mg, 0.11 mmol, 1.0 eq) and tetrabutylammonium fluoride (1M in THF, 0.22 mL, 0.22 mmol, 2.0 eq) in THF (2 mL) was stirred at 20° C. for 16 h. The mixture was concentrated and purified by flash column chromatography (Silica, 0-10% MeOH in DCM) to give the title compound (46.0 mg, 0.07 mmol, 61.0% yield) as a light yellow oil. MS (ESP) m/z=685.5 [M+H]⁺.

### Step 3: 6-[8-[[2-[(2S)-2-amino-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

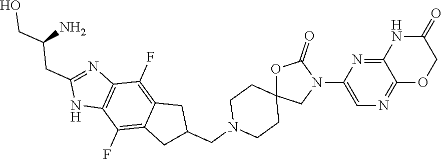 [SMILES: N[C@H](CO)Cc1nc2c(F)c3c(c(F)c2[nH]1)CC(CN1CCC2(CC1)CN(c1cnc4c(n1)NC(=O)CO4)C(=O)O2)C3]

A mixture of tert-butyl N-[(1S)-1-[[4,8-difluoro-6-[[2-oxo-3-(3-oxo-4H-pyrazino[2,3-b][1,4]oxazin-6-yl)-1-oxa-3,8-diazaspiro [4.5]decan-8-yl]methyl]-3,5,6,7-tetrahydrocyclopenta[f]benzimidazol-2-yl]methyl]-2-hydroxy-ethyl]carbamate (46.0 mg, 0.07 mmol, 1.0 eq) and trifluoroacetic acid (0.05 mL, 0.67 mmol, 10.0 eq) in DCM (1.5 mL) was stirred at RT for 4 h. The mixture was concentrated and purified by flash column chromatography (Silica-NH, 0-20% MeOH in DCM) to give the title compound (27.7 mg, 0.05 mmol, 70.5% yield) as a white solid. MS (ESP) m/z=585.3 [M+H]⁺.

### Step 4: 6-[8-[[2-[(2S)-2-(dimethylamino)-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one

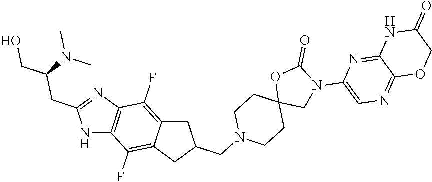 [SMILES: CN(C)[C@H](CO)Cc1nc2c(F)c3c(c(F)c2[nH]1)CC(CN1CCC2(CC1)CN(c1cnc4c(n1)NC(=O)CO4)C(=O)O2)C3]

A mixture of 6-[8-[[2-[(2S)-2-amino-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one (27.7 mg, 0.05 mmol, 1.0 eq) and formaldehyde (9.61 mg, 0.12 mmol, 2.5 eq) in tetrahydrofuran (1 mL) and DMA (0.5 mL) was stirred at RT for 15 min. then sodium triacetoxyborohydride (25.1 mg, 0.12 mmol, 2.5 eq) was added. The mixture was stirred at RT overnight. Further formaldehyde (9.61 mg, 0.12 mmol, 2.5 eq), acetic acid (2.85 mg, 0.05 mmol, 1.0 eq) and sodium cyanoborohydride (4.47 mg, 0.07 mmol, 1.5 eq) were added and the mixture was stirred for a further 18 h. The mixture was concentrated and purified by flash column chromatography (Silica-NH, 0-20% MeOH in DCM) to give the title compound (8.8 mg, 0.01 mmol, 28.8% yield) as a white solid. MS (ESP) m/z=613.3 [M+H]⁺.

### Example 64

### 6-[8-[[1-[2-(Dimethylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one

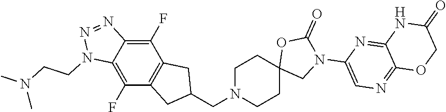 [SMILES: CN(C)CCn1nnc2c(F)c3c(c(F)c21)CC(CN1CCC2(CC1)CN(c1cnc4c(n1)NC(=O)CO4)C(=O)O2)C3]

The title compound was prepared from 6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 42) by analogy with Example 63, Step 4 and was obtained as a white solid. MS (ESP) m/z=584.3 [M+H]⁺.

### Example 65

### 6-[8-[[3-[2-(Dimethylamino)ethyl]-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one

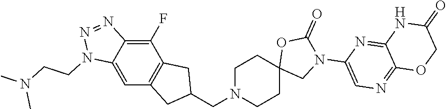 [SMILES: CN(C)CCn1nnc2c(F)c3c(cc21)CC(CN1CCC2(CC1)CN(c1cnc4c(n1)NC(=O)CO4)C(=O)O2)C3]

The title compound was prepared from 6-[8-[[3-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 75) by analogy with Example 63, Step 4 and was obtained as a white solid. MS (ESP) m/z=566.3 [M+H]⁺.

### Example 66

### 6-[8-[[4,8-Difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer

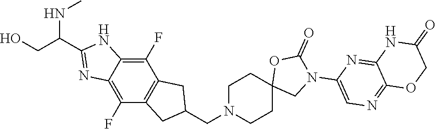 [SMILES: CNC(CO)c1nc2c(F)c3c(c(F)c2[nH]1)CC(CN1CCC2(CC1)CN(c1cnc4c(n1)NC(=O)CO4)C(=O)O2)C3]

### and

### Example 67

### 6-[8-[[4,8-Difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer B

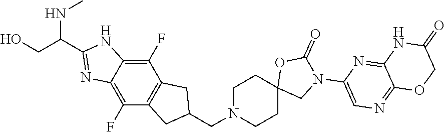 [SMILES: CNC(CO)c1nc2c(F)c3c(c(F)c2[nH]1)CC(CN1CCC2(CC1)CN(c1cnc4c(n1)NC(=O)CO4)C(=O)O2)C3]

### Step 1: tert-butyl N-[2-tert-butoxy-1-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]ethyl]-N-methyl-carbamate

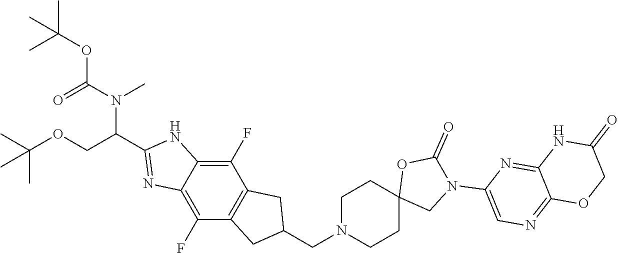 [SMILES: CN(C(=O)OC(C)(C)C)C(COC(C)(C)C)c1nc2c(F)c3c(c(F)c2[nH]1)CC(CN1CCC2(CC1)CN(c1cnc4c(n1)NC(=O)CO4)C(=O)O2)C3]

To a suspension of tert-butyl N-[2-tert-butoxy-1-(4,8-difluoro-6-formyl-3,5,6,7-tetrahydrocyclopenta[f]benzimidazol-2-yl)ethyl]-N-methyl-carbamate (Intermediate 60, 142.0 mg, 0.31 mmol, 1.0 eq) and 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2, 131.9 mg, 0.31 mmol, 1.0 eq) in tetrahydrofuran (2.6 mL)/DMA (0.5 mL) at RT. N-Ethyldiisopropylamine (82.17 uL, 0.47 mmol, 1.5 eq) was added and the reaction mixture was stirred at RT for 15 min. Acetic acid (45.0 uL, 0.79 mmol, 2.5 eq) and sodium triacetoxyborohydride (100.0 mg, 0.47 mmol, 1.5 eq) were added and the reaction mixture was stirred at RT for 15 h. The reaction mixture was diluted with EtOAc and quenched with NaHCO₃ (sat. aq.). The phases were separated and the organic layer was washed with water (twice). Combined organic phases were filtered through a phase separator and volatiles were removed under reduced pressure to afford the crude compound (63 mg, light yellow foam) which was purified by silica cartridge chromatography (10 g silica, from DCM 100% to DCM/MeOH 97:3) and further purified by silica cartridge chromatography (11 g silica-NH, from DCM 100% to DCM/[EtOAc/MeOH 9:1] 85:15) to give the title compound (85.0 mg, 0.11 mmol, 36.5% yield) as a white amorphous solid. MS (ESN) m/z=739.6 [M−H]⁻.

### Step 2: 6-[8-[[4,8-difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one; 2,2,2-trifluoroacetic acid

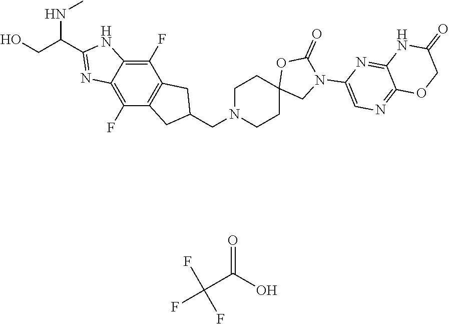 [SMILES: CNC(CO)c1nc2c(F)c3c(c(F)c2[nH]1)CC(CN1CCC2(CC1)CN(c1cnc4c(n1)NC(=O)CO4)C(=O)O2)C3 ; O=C(O)C(F)(F)F]

A solution of tert-butyl N-[2-tert-butoxy-1-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]ethyl]-N-methyl-carbamate (85.0 mg, 0.11 mmol, 1.0 eq) in trifluoroacetic acid (1.5 mL, 19.5 mmol, 170 eq) was stirred at RT for 21 h. Volatiles were removed under reduced pressure, the residue was redissolved in MeCN and added to Et₂O. The resulting white precipitate was collected by centrifugation to give the crude title compound (84.0 mg, 0.1 mmol, 90.1% yield) as a white solid.

### Step 3: 6-[8-[[4,8-Difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer A and 6-[8-[[4,8-difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer B

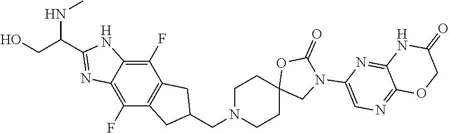 [SMILES: CNC(CO)c1nc2c(F)c3c(c(F)c2[nH]1)CC(CN1CCC2(CC1)CN(c1cnc4c(n1)NC(=O)CO4)C(=O)O2)C3]

The enantiomers of 6-[8-[[4,8-difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one; 2,2,2-trifluoroacetic acid (90.0 mg, 0.11 mmol, 1.0 eq) were separated by chiral HPLC to give Peak 1: 6-[8-[[4,8-difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer A (30.0 mg, 0.05 mmol, 44.0% yield) as a white solid and Peak 2: 6-[8-[[4,8-difluoro-2-[2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one Enantiomer B (11.0 mg, 0.02 mmol, 17.0% yield) as a white solid. MS (ESP) m/z=585.3 [M+H]⁺.

The following examples were prepared by analogy to Example 67. Single stereoisomers were prepared by separation of the corresponding stereoisomer mixture obtained in Step 1 or Step 2 by chiral HPLC or chiral SFC. Stereoisomer mixtures that are not salts were obtained from the corresponding 2,2,2-trifluoroacetic acid salt by flash column chromatography (NH-silica).

| Ex. | Name, Structure, MS | Starting Materials |
|---|---|---|
| 68 | 6-[8-[[2-[(1R)-1-aminoethyl]-4,8-difluoro-1-methyl-6,7-dihydro-5H- | Intermediate 2 and |
| | cyclopenta[f]benzimidazol-6-yl]methyl]-2-oxo-1-oxa-3,8- | Intermediate 61 |
| | diazaspiro[4.5]decan-3-yl]-4H-pyrazino[2,3-b][1,4]oxazin-3-one | |
| | 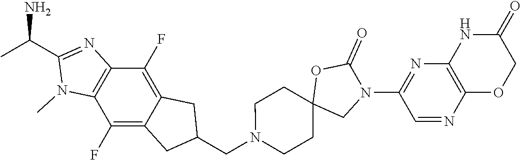 [SMILES: C[C@@H](N)c1nc2c(F)c3c(c(F)c2n1C)CC(CN1CCC2(CC1)CN(c1cnc4c(n1)NC(=O)CO4)C(=O)O2)C3] | |
| | ESP [M + H]⁺: 569.3 | |
| 69 | 6-[8-[[8-fluoro-2-[2-(methylamino)ethyl]-1,5,6,7- | Intermediate 2 and |
| | tetrahydrocyclopenta[f]benzimidazol-6-yl]methyl]-2-oxo-1-oxa-3,8- | Intermediate 62 |
| | diazaspiro[4.5]decan-3-yl]-4H-pyrazinio[2,3-b][1,4]oxazin-3-one | |
| | 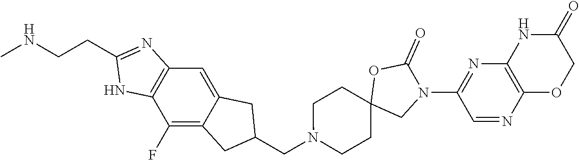 [SMILES: CNCCc1nc2cc3c(c(F)c2[nH]1)CC(CN1CCC2(CC1)CN(c1cnc4c(n1)NC(=O)CO4)C(=O)O2)C3] | |
| | ESP [M + H]⁺: 551.3 | |
| 70 | 6-[8-[[8-fluoro-2-[(2-methoxyethylamino)methyl]-3,5,6,7- | Intermediate 2 and |
| | tetrahydrocyclopenta[f]benzimidazol-6-yl]methyl]-2-oxo-1-oxa-3,8- | Intermediate 63 |
| | diazaspiro[4.5]decan-3-yl]-4H-pyrazino[2,3-b][1,4]oxazin-3-one | |
| | 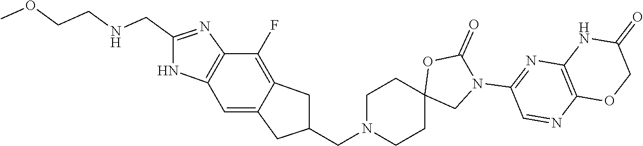 [SMILES: COCCNCc1nc2c(F)c3c(cc2[nH]1)CC(CN1CCC2(CC1)CN(c1cnc4c(n1)NC(=O)CO4)C(=O)O2)C3] | |
| | ESP [M + H]⁺: 581.3 | |
| 71 | 2-[[8-fluoro-6-[[2-oxo-3-(3-oxo-4H-pyrazino[2,3-b][1,4]oxazin-6- | Intermediate 2 and |
| | yl)-1-oxa-3,8-diazaspiro[4.5]decan-8-yl]methyl]-3,5,6,7- | Intermediate 64 |
| | tetrahydrocyclopenta[f]benzimidazol-2-yl]methylamino]-N-methyl- | |
| | acetamide | |
| | 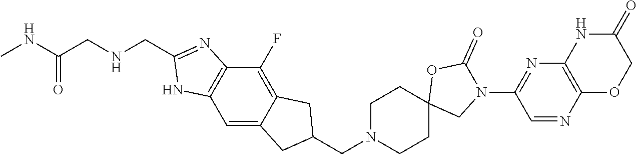 [SMILES: CNC(=O)CNCc1nc2c(F)c3c(cc2[nH]1)CC(CN1CCC2(CC1)CN(c1cnc4c(n1)NC(=O)CO4)C(=O)O2)C3] | |
| | ESP [M + H]⁺: 594.3 | |
| 72 | 6-[8-[[3-(2-aminoethyl)-8-fluoro-2-methyl-6,7-dihydro-5H- | Intermediate 2 and |
| | cyclopenta[f]benzimidazol-6-yl]methyl]-2-oxo-1-oxa-3,8- | Intermediate 65 |
| | diazaspiro[4.5]decan-3-yl]-4H-pyrazino[2,3-b][1,4]oxazin-3-one | |
| | 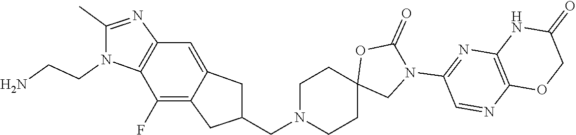 [SMILES: Cc1nc2cc3c(c(F)c2n1CCN)CC(CN1CCC2(CC1)CN(c1cnc4c(n1)NC(=O)CO4)C(=O)O2)C3] | |
| | ESP [M + H]⁺: 551.3 | |
| 73 | 6-[8-[[3-(2-Aminoethyl)-8-fluoro-2-methyl-6,7-dihydro-5H- | Intermediate 2 and |
| | cyclopenta[f]benzimidazol-6-yl]methyl]-2-oxo-1-oxa-3,8- | Intermediate 65 |
| | diazaspiro[4.5]decan-3-yl]-4H-pyrazino[2,3-b][1,4]oxain-3-one | |
| | 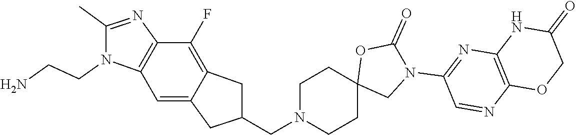 [SMILES: Cc1nc2c(F)c3c(cc2n1CCN)CC(CN1CCC2(CC1)CN(c1cnc4c(n1)NC(=O)CO4)C(=O)O2)C3] | |
| | ESP [M + H]⁺: 551.3 | |
| 74 | 6-[8-[[2-(2-aminoethyl)-8-fluoro-6,7-dihydro-5H- | Intermediate 2 and |
| | cyclopenta[f]benzotriazol-6-yl]methyl]-2-oxo-1-oxa-3,8- | Intermediate 66 |
| | diazaspiro[4.5]decan-3-yl]-4H-pyrazino[2,3-b][1,4]oxazin-3-one | |
| | 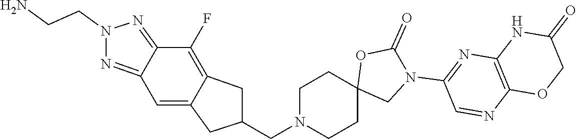 [SMILES: NCCn1nc2cc3c(c(F)c2n1)CC(CN1CCC2(CC1)CN(c1cnc4c(n1)NC(=O)CO4)C(=O)O2)C3] | |
| | ESP [M + H ]⁺: 538.3 | |
| 75 | 6-[8-[[3-(2-aminoethyl)-8-fluoro-6,7-dihydro-5H- | Intermediate 2 and |
| | cyclopenta[f]benzotriazol-6-yl]methyl]-2-oxo-1-oxa-3,8- | Intermediate 67 |
| | diazaspiro[4.5]decan-3-yl]-4H-pyrazino[2,3-b][1,4]oxazin-3-one | |
| | 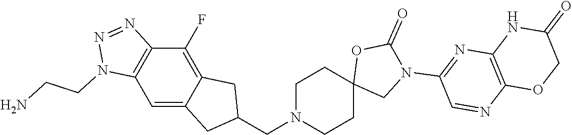 [SMILES: NCCn1nnc2c(F)c3c(cc21)CC(CN1CCC2(CC1)CN(c1cnc4c(n1)NC(=O)CO4)C(=O)O2)C3] | |
| | ESP [M + H]⁺: 538.3 | |
| 76 | 6-[8-[[1-(2-aminoethyl)-8-fluoro-6,7-dihydro-5H- | Intermediate 2 and |
| | cyclopenta[f]benzotriazol-6-yl]methyl]-2-oxo-1-oxa-3,8- | Intermediate 68 |
| | diazaspiro[4.5]decan-3-yl]-4H-pyrazino[2,3-b][1,4]oxazin-3-one | |
| | 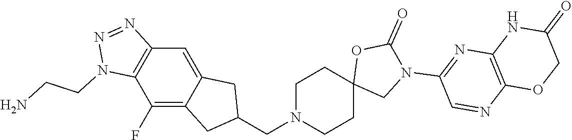 [SMILES: NCCn1nnc2cc3c(c(F)c21)CC(CN1CCC2(CC1)CN(c1cnc4c(n1)NC(=O)CO4)C(=O)O2)C3] | |
| | ESP [M + H]⁺: 538.3 | |
| 77 | 6-[8-[[2-[(1-aminocyclopropyl)methyl]-4,8-difluoro-6,7-dihydro-5H- | Intermediate 2 and |
| | cyclopenta[f]benzotriazol-6-yl]methyl]-2-oxo-1-oxa-3,8- | Intermediate 69 |
| | diazaspiro[4.5]decan-3-yl]-4H-pyrazino[2,3-b][1,4]oxazin-3- | |
| | one; 2,2,2-trifluoroacetic acid | |
| | 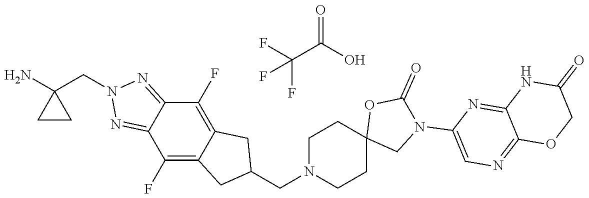 [SMILES: NC1(Cn2nc3c(F)c4c(c(F)c3n2)CC(CN2CCC3(CC2)CN(c2cnc5c(n2)NC(=O)CO5)C(=O)O3)C4)CC1 ; O=C(O)C(F)(F)F] | |
| | ESP [M + H]⁺: 582.3 | |
| 78 | 6-[8-[[1-[(1-aminocyclopropyl)methyl]-4,8-difluoro-6,7-dihydro-5H- | Intermediate 2 and |
| | cyclopenta[f]benzotriazol-6-yl]methyl]-2-oxo-1-oxa-3,8- | Intermediate 70 |
| | diazaspiro[4.5]decan-3-yl]-4H-pyrazino[2,3-b][1,4]oxazin-3-one | |
| | 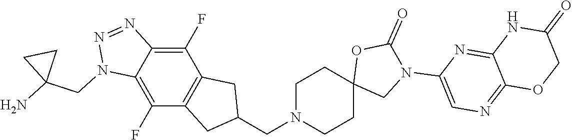 [SMILES: NC1(Cn2nnc3c(F)c4c(c(F)c32)CC(CN2CCC3(CC2)CN(c2cnc5c(n2)NC(=O)CO5)C(=O)O3)C4)CC1] | |
| | ESP [M + H]⁺: 582.3 | |
| 79 | 6-[8-[[2-(azetidin-3-yl)-4,8-difluoro-6,7-dihydro-5H- | Intermediate 2 and |
| | cyclopenta[f]benzotriazol-6-yl]methyl]-2-oxo-1-oxa-3,8- | Intermediate 71 |
| | diazaspiro[4.5]decan-3-yl]-4H-pyrazino[2,3-b][1,4]oxazin-3- | |
| | one; formic acid | |
| | 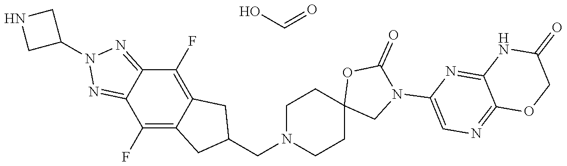 [SMILES: O=C1COc2ncc(N3CC4(CCN(CC5Cc6c(c(F)c7nn(C8CNC8)nc7c6F)C5)CC4)OC3=O)nc2N1 ; O=CO] | |
| | ESP [M + H]⁺: 568.1 | |
| 80 | 6-[8-[[1-(azetidin-3-yl)-4,8-difluoro-6,7-dihydro-5H- | Intermediate 2 and |
| | cyclopenta[f]benzotriazol-6-yl]methyl]-2-oxo-1-oxa-3,8- | Intermediate 72 |
| | diazaspiro[4.5]decan-3-yl]-4H-pyrazino[2,3-b][1,4]oxazin-3-one | |
| | 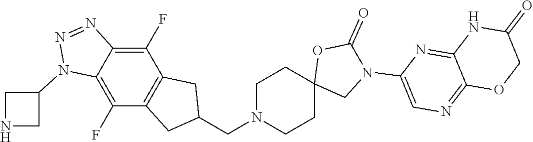 [SMILES: O=C1COc2ncc(N3CC4(CCN(CC5Cc6c(c(F)c7c(nnn7C7CNC7)c6F)C5)CC4)OC3=O)nc2N1] | |
| | ESP [M + H]⁺: 568.2 | |
| 81 | 6-[2-oxo-8-[[1-(2-aminoethyl)-8-fluoro-6,7-dihydro-5H- | Intermediate 2 and |
| | cyclopenta[f]benzotriazol-6-yl]methyl]-1-oxa-3,8- | Intermediate 68 |
| | diazaspiro[4.5]decan-3-yl]-4H-pyrazino[2,3-b][1,4]oxazin-3-one | |
| | Enantiomer A | |
| | 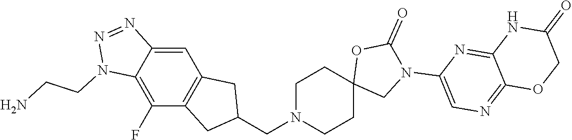 [SMILES: NCCn1nnc2cc3c(c(F)c21)CC(CN1CCC2(CC1)CN(c1cnc4c(n1)NC(=O)CO4)C(=O)O2)C3] | |
| | ESP [M + H]⁺: 538.3 | |
| 82 | 6-[2-oxo-8-[[1-(2-aminoethyl)-8-fluoro-6,7-dihydro-5H- | Intermediate 2 and |
| | cyclopenta[f]benzotriazol-6-yl]methyl]-1-oxa-3,8- | Intermediate 68 |
| | diazaspiro[4.5]decan-3-yl]-4H-pyrazino[2,3-b][1,4]oxazin-3-one | |
| | Enantiomer B | |
| | 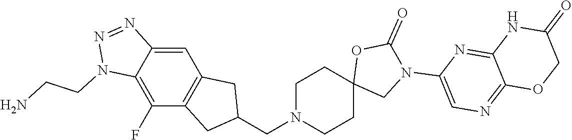 [SMILES: NCCn1nnc2cc3c(c(F)c21)CC(CN1CCC2(CC1)CN(c1cnc4c(n1)NC(=O)CO4)C(=O)O2)C3] | |
| | ESP [M + H]⁺: 538.3 | |
| 83 | 6-[8-[[2-(2-aminoethyl)-5-fluoro-7,8-dihydro-6H- | Intermediate 2 and |
| | cyclopenta[e]benzotriazol-7-yl]methyl]-2-oxo-1-oxa-3,8- | Intermediate 73 |
| | diazaspiro[4.5]decan-3-yl]-4H-pyrazino[2,3-b][1,4]oxazin-3- | |
| | one; 2,2,2-trifluoroacetic acid | |
| | 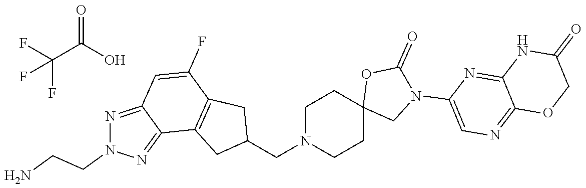 [SMILES: O=C(O)C(F)(F)F ; NCCn1nc2cc(F)c3c(c2n1)CC(CN1CCC2(CC1)CN(c1cnc4c(n1)NC(=O)CO4)C(=O)O2)C3] | |
| | ESP [M + H]⁺: 538.3 | |
| 84 | 6-[8-[[1-(2-aminoethyl)-5-fluoro-7,8-dihydro-6H- | Intermediate 2 and |
| | cyclopenta[e]benzotriazol-7-yl]methyl]-2-keto-1-oxa-3,8- | Intermediate 74 |
| | diazaspiro[4.5]decan-3-yl]-4H-pyrazino[2,3-b][1,4]oxazin-3-one | |
| | 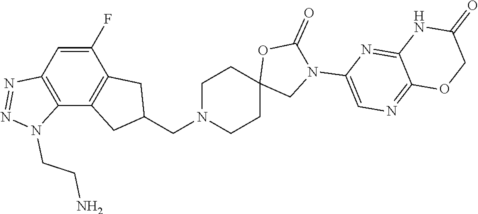 [SMILES: NCCn1nnc2cc(F)c3c(c21)CC(CN1CCC2(CC1)CN(c1cnc4c(n1)NC(=O)CO4)C(=O)O2)C3] | |
| | ESP [M + H]⁺: 538.3 | |
| 85 | 6-[8-[[3-(2-aminoethyl)-5-fluoro-7,8-dihydro-6H- | Intermediate 2 and |
| | cyclopenta[e]benzotriazol-7-yl]methyl]-2-keto-1-oxa-3,8- | Intermediate 74 |
| | diazaspiro[4.5]decan-3-yl]-4H-pyrazino[2,3-b][1,4]oxazin-3-one | |
| | 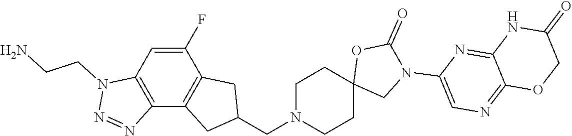 [SMILES: NCCn1nnc2c3c(c(F)cc21)CC(CN1CCC2(CC1)CN(c1cnc4c(n1)NC(=O)CO4)C(=O)O2)C3] | |
| | ESP [M + H]⁺: 538.2 | |
| 86 | 6-[8-[[5-fluoro-2-[2-(methylamino)ethyl]-7.8-dihydro-6H- | Intermediate 2 and |
| | cyclopenta[e]benzotriazol-7-yl]methyl]-2-oxo-1-oxa-3,8- | Intermediate 75 |
| | diazaspiro[4.5]decan-3-yl]-4H-pyrazino[2,3-b][1,4]oxazin-3-one | |
| | Enantiomer A | |
| | 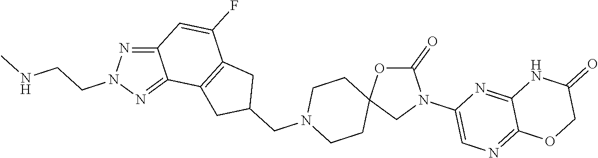 [SMILES: CNCCn1nc2cc(F)c3c(c2n1)CC(CN1CCC2(CC1)CN(c1cnc4c(n1)NC(=O)CO4)C(=O)O2)C3] | |
| | ESP [M + H]⁺: 552.2 | |
| 87 | 6-[8-[[5-fluoro-2-[2-(methylamino)ethyl]-7,8-dihydro-6H- | Intermediate 2 and |
| | cyclopenta[e]benzotriazol-7-yl]methyl]-2-oxo-1-oxa-3,8- | Intermediate 75 |
| | diazaspiro[4.5]decan-3-yl]-4H-pyrazino[2,3-b][1,4]oxazin-3-one | |
| | Enantiomer B | |
| | 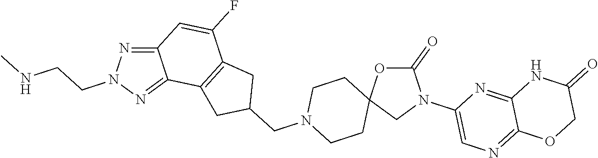 [SMILES: CNCCn1nc2cc(F)c3c(c2n1)CC(CN1CCC2(CC1)CN(c1cnc4c(n1)NC(=O)CO4)C(=O)O2)C3] | |
| | ESP [M + H]⁺: 552.2 | |
| 88 | 6-[8-[[2-[(2S)-2-aminopropyl]-5-fluoro-7,8-dihydro-6H- | Intermediate 2 and |
| | cyclopenta[e]benzotriazol-7-yl]methyl]-2-oxo-1-oxa-3,8- | Intermediate 76 |
| | diazaspiro[4.5]decan-3-yl]-4H-pyrazino[2,3-b][1,4]oxazin-3-one | |
| | Epimer A | |
| | 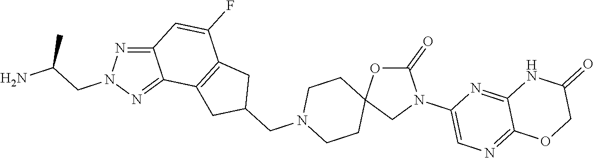 [SMILES: C[C@H](N)Cn1nc2cc(F)c3c(c2n1)CC(CN1CCC2(CC1)CN(c1cnc4c(n1)NC(=O)CO4)C(=O)O2)C3] | |
| | ESP [M + H]⁺: 522.1 | |
| 89 | 6-[8-[[2-[(2S)-2-aminopropyl]-5-fluoro-7,8-dihydro-6H- | Intermediate 2 and |
| | cyclopenta[e]benzotriazol-7-yl]methyl]-2-oxo-1-oxa-3,8- | Intermediate 76 |
| | diazaspiro[4.5]decan-3-yl]-4H-pyrazino[2,3-b][1,4]oxazin-3-one | |
| | Epimer B | |
| | 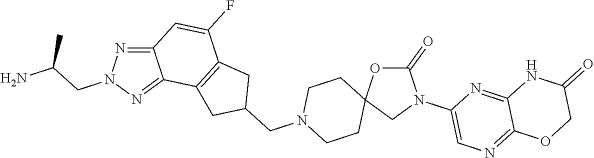 [SMILES: C[C@H](N)Cn1nc2cc(F)c3c(c2n1)CC(CN1CCC2(CC1)CN(c1cnc4c(n1)NC(=O)CO4)C(=O)O2)C3] | |
| | ESP [M + H]⁺: 522.1 | |
| 90 | 6-[8-[[2-[(1R)-1-aminoethyl]-8-fluoro-6,7-dihydro-5H- | Intermediate 2 and |
| | cyclopenta[f][1,3]benzoxazol-6-yl]methyl]-2-oxo-1-oxa-,38- | Intermediate 77 |
| | diazaspiro[4.5]decan-3-yl]-4H-pyrazino[2,3-b][1,4]oxazin-3-one | |
| | 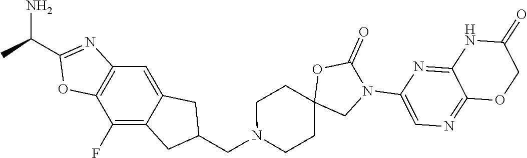 [SMILES: C[C@@H](N)c1nc2cc3c(c(F)c2o1)CC(CN1CCC2(CC1)CN(c1cnc4c(n1)NC(=O)CO4)C(=O)O2)C3] | |
| | ESP [M + H]⁺: 538.4 | |
| 91 | 6-[8-[[8-fluoro-2-[(1R)-2-hydroxy-1-(methylamino)ethyl]-6,7- | Intermediate 2 and |
| | dihydro-5H-cyclopenta[f][1,3]benzoxazol-6-yl]methyl]-2-oxo-1-oxa- | Intermediate 78 |
| | 3,8-diazaspiro[4.5]decan-3-yl]-4H-pyrazino[2,3-b][1,4]oxazin-3-one | |
| | 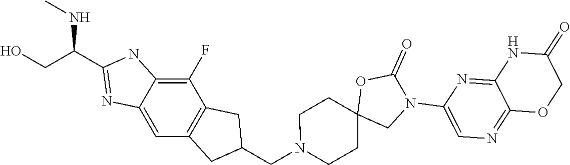 [SMILES: CN[C@H](CO)c1nc2cc3c(c(F)c2[nH]1)CC(CN1CCC2(CC1)CN(c1cnc4c(n1)NC(=O)CO4)C(=O)O2)C3] | |
| | ESP [M + H]⁺: 537.3 | |
| 92 | 6-[8-[[5-fluoro-2-(methylaminomethyl)-1,6,7,8- | Intermediate 2 and |
| | tetrahydrocyclopenta[e]benzimidazol-7-yl]methyl]-2-oxo-1-oxa-3,8- | Intermediate 79 |
| | diazaspiro[4.5]decan-3-yl]-4H-pyrazino[2,3-b][1,4]oxazin-3-one | |
| | 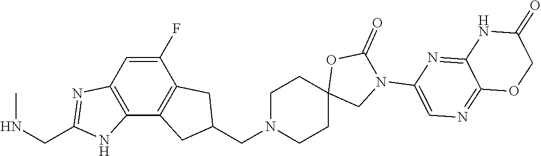 [SMILES: CNCc1nc2cc(F)c3c(c2[nH]1)CC(CN1CCC2(CC1)CN(c1cnc4c(n1)NC(=O)CO4)C(=O)O2)C3] | |
| | ESP [M + H]⁺: 537.3 | |
| 93 | 6-[8-[[4-fluoro-2-(methylaminomethyl)-6,7-dihydro-5H- | Intermediate 2 and |
| | cyclopenta[f][1,3]benzoxazol-6-yl]methyl]-2-oxo-1-oxa-3,8- | Intermediate 80 |
| | diazaspiro[4.5]decan-3-yl]-4H-pyrazino[2,3-b][1,4]oxazin-3-one | |
| | 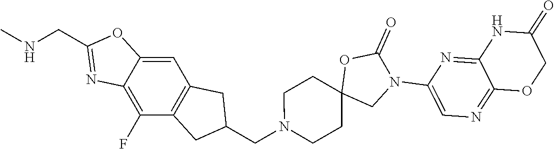 [SMILES: CNCc1nc2c(F)c3c(cc2o1)CC(CN1CCC2(CC1)CN(c1cnc4c(n1)NC(=O)CO4)C(=O)O2)C3] | |
| | ESP [M + H]⁺: 538.3 | |
| 94 | 6-[8-[[8-fluoro-2-(methylaminomethyl)-6,7-dihydro-5H- | Intermediate 2 and |
| | cyclopenta[f][1,3]benzoxazol-6-yl]methyl]-2-keto-1-oxa-3,8- | Intermediate 29 |
| | diazaspiro[4.5]decan-3-yl]-4H-pyrazino[2,3-b][1,4]oxazin-3-one | |
| | Enantiomer A | |
| | 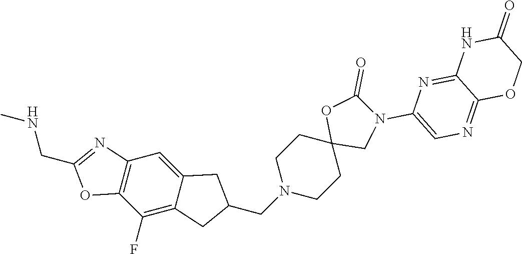 [SMILES: CNCc1nc2cc3c(c(F)c2o1)CC(CN1CCC2(CC1)CN(c1cnc4c(n1)NC(=O)CO4)C(=O)O2)C3] | |
| | ESP [M + H]⁺: 538.2 | |
| 95 | 6-[8-[[8-fluoro-2-(methylaminomethyl)-6,7-dihydro-5H- | Intermediate 2 and |
| | cyclopenta[f][1,3]benzoxazol-6-yl]methyl]-2-keto-1-oxa-3,8- | Intermediate 29 |
| | diazaspiro[4.5]decan-3-yl]-4H-pyrazino[2,3-b][1,4]oxazin-3-one | |
| | Enantiomer B | |
| | 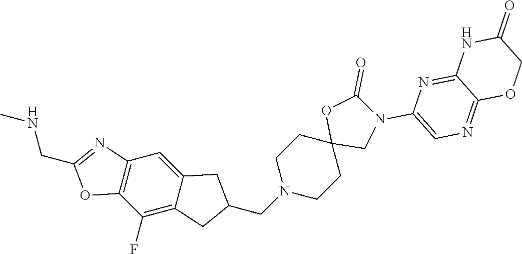 [SMILES: CNCc1nc2cc3c(c(F)c2o1)CC(CN1CCC2(CC1)CN(c1cnc4c(n1)NC(=O)CO4)C(=O)O2)C3] | |
| | ESP [M + H]⁺: 538.2 | |
| 96 | 6-[8-[[5-fluoro-2-(methylaminomethyl)-7,8-dihydro-6H- | Intermediate 2 and |
| | cyclopenta[e][1,3]benzoxazol-7-yl]methyl]-2-oxo-1-oxa-3,8- | Intermediate 81 |
| | diazaspiro[4.5]decan-3-yl]-4H-pyrazino[2,3-b][1,4]oxazin-3-one | |
| | 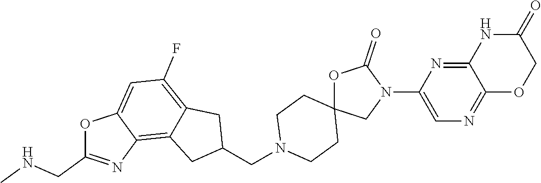 [SMILES: CNCc1nc2c3c(c(F)cc2o1)CC(CN1CCC2(CC1)CN(c1cnc4c(n1)NC(=O)CO4)C(=O)O2)C3] | |
| | ESP [M + H]⁺: 538.2 | |
| 97 | 6-[8-[[2-[(1S)-1-aminoethyl]-8-fluoro-6,7-dihydro-5H- | Intermediate 2 and |
| | cyclopenta[f][1,3]benzoxazol-6-yl]methyl]-2-oxo-1-oxa-3,8- | Intermediate 82 |
| | diazaspiro[4.5]decan-3-yl]-4H-pyrazino[2,3-b][1,4]oxazin-3-one | |
| | 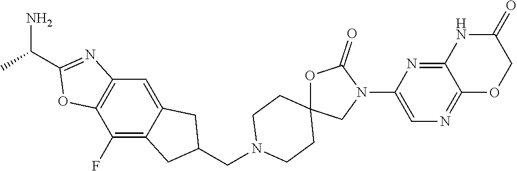 [SMILES: C[C@H](N)c1nc2cc3c(c(F)c2o1)CC(CN1CCC2(CC1)CN(c1cnc4c(n1)NC(=O)CO4)C(=O)O2)C3] | |
| | ESP [M + H]⁺: 538.3 | |
| 98 | 6-[8-[[8-fluoro-2-[(3-hydroxyazetidin-1-yl)methyl]-6,7-dihydro-5H- | Intermediate 2 and |
| | cyclopenta[f][1,3]benzoxazol-6-yl]methyl]-2-oxo-1-oxa-3,8- | Intermediate 83 |
| | diazaspiro[4.5]decan-3-yl]-4H-pyrazino[2,3-b][1,4]oxazin-3-one | |
| | Enantiomer A | |
| | 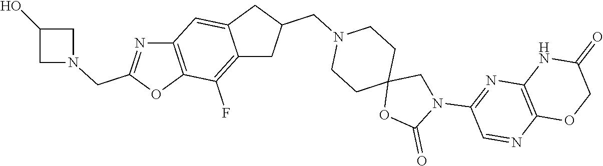 [SMILES: O=C1COc2ncc(N3CC4(CCN(CC5Cc6cc7nc(CN8CC(O)C8)oc7c(F)c6C5)CC4)OC3=O)nc2N1] | |
| | ESP [M + H]⁺: 580.2 | |
| 99 | 6-[8-[[8-fluoro-2-[(3-hydroxyazetidin-1-yl)methyl]-6,7-dihydro-5H- | Intermediate 2 and |
| | cyclopenta[f][1,3]benzoxazol-6-yl]methyl]-2-oxo-1-oxa-3,8- | Intermediate 83 |
| | diazaspiro[4.5]decan-3-yl]-4H-pyrazino[2,3-b][1,4]oxazin-3-one | |
| | Enantiomer B | |
| | 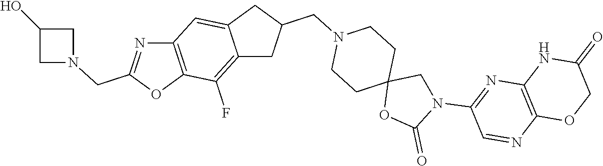 [SMILES: O=C1COc2ncc(N3CC4(CCN(CC5Cc6cc7nc(CN8CC(O)C8)oc7c(F)c6C5)CC4)OC3=O)nc2N1] | |
| | ESP [M + H]⁺: 580.2 | |
| 100 | 6-[2-oxo-8-[[2-(azetidin-3-yl)-8-fluoro-6,7-dihydro-5H- | Intermediate 2 and |
| | cyclopenta[f][1,3]benzoxazol-6-yl]methyl]-1-oxa-3,8- | Intermediate 84 |
| | diazaspiro[4.5]decan-3-yl]-4H-pyrazino[2,3-b][1,4]oxazin-3-one | |
| | Enantiomer A | |
| | 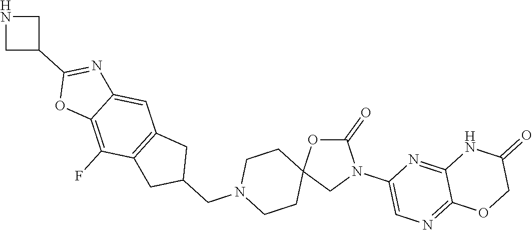 [SMILES: O=C1COc2ncc(N3CC4(CCN(CC5Cc6cc7nc(C8CNC8)oc7c(F)c6C5)CC4)OC3=O)nc2N1] | |
| | ESP [M + H]⁺: 550.2 | |
| 101 | 6-[2-oxo-8-[[2-(azetidin-3-yl)-8-fluoro-6,7-dihydro-5H- | Intermediate 2 and |
| | cyclopenta[f][1,3]benzoxazol-6-yl]methyl]-1-oxa-3,8- | Intermediate 84 |
| | diazaspiro[4.5]decan-3-yl]-4H-pyrazino[2,3-b][1,4]oxazin-3-one | |
| | Enantiomer B | |
| | 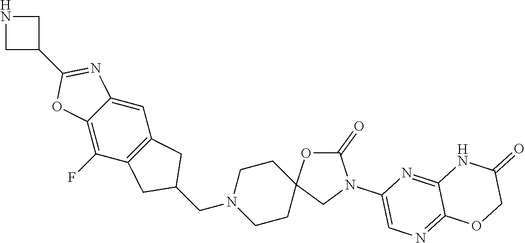 [SMILES: O=C1COc2ncc(N3CC4(CCN(CC5Cc6cc7nc(C8CNC8)oc7c(F)c6C5)CC4)OC3=O)nc2N1] | |
| | ESP [M + H]⁺: 550.2 | |
| 102 | 6-[8-[[2-(2-aminoethyl)-8-fluoro-6,7-dihydro-5H- | Intermediate 2 and |
| | cyclopenta[f][1,3]benzoxazol-6-yl]methyl]-2-oxo-1-oxa-3,8- | Intermediate 85 |
| | diazaspiro[4.5]decan-3-yl]-4H-pyrazino[2,3-b][1,4]oxazin-3-one | |
| | Enantiomer A | |
| | 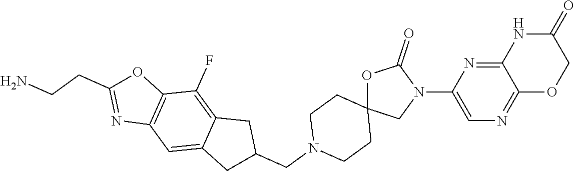 [SMILES: NCCc1nc2cc3c(c(F)c2o1)CC(CN1CCC2(CC1)CN(c1cnc4c(n1)NC(=O)CO4)C(=O)O2)C3] | |
| | ESP [M + H]⁺: 538.1 | |
| 103 | 6-[8-[[2-(2-aminoethyl)-8-fluoro-6,7-dihydro-5H- | Intermediate 2 and |
| | cyclopenta[f][1,3]benzoxazol-6-yl]methyl]-2-oxo-1-oxa-3,8- | Intermediate 85 |
| | diazaspiro[4.5]decan-3-yl]-4H-pyrazino[2,3-b][1,4]oxazin-3-one | |
| | Enantiomer B | |
| | 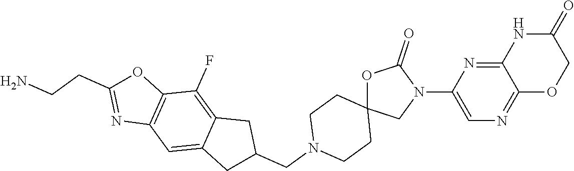 [SMILES: NCCc1nc2cc3c(c(F)c2o1)CC(CN1CCC2(CC1)CN(c1cnc4c(n1)NC(=O)CO4)C(=O)O2)C3] | |
| | ESP [M + H]⁺: 538.2 | |
| 104 | 6-[2-oxo-8-[[2-(aminomethyl)-8-fluoro-6,7-dihydro-5H- | Intermediate 2 and |
| | cyclopenta[f][1,3]benzoxazol-6-yl]methyl]-1-oxa-3,8- | Intermediate 86 |
| | diazaspiro[4.5]decan-3-yl]-4H-pyrazino[2,3-b][1,4]oxazin-3-one | |
| | Enantiomer A | |
| | 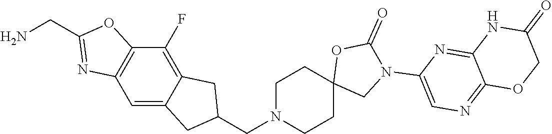 [SMILES: NCc1nc2cc3c(c(F)c2o1)CC(CN1CCC2(CC1)CN(c1cnc4c(n1)NC(=O)CO4)C(=O)O2)C3] | |
| | ESP [M + H]⁺: 524.1 | |
| 105 | 6-[2-oxo-8-[[2-(aminomethyl)-8-fluoro-6,7-dihydro-5H- | Intermediate 2 and |
| | cyclopenta[f][1,3]benzoxazol-6-yl]methyl]-1-oxa-3,8- | Intermediate 86 |
| | diazaspiro[4.5]decan-3-yl]-4H-pyrazino[2,3-b][1,4]oxazin-3-one | |
| | Enantiomer B | |
| | 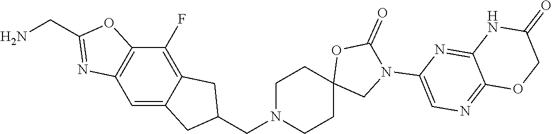 [SMILES: NCc1nc2cc3c(c(F)c2o1)CC(CN1CCC2(CC1)CN(c1cnc4c(n1)NC(=O)CO4)C(=O)O2)C3] | |
| | ESP [M + H]⁺: 524.1 | |
| 106 | 6-[8-[[2-[(2S)-azetidin-2-yl]-8-fluoro-6,7-dihydro-5H- | Intermediate 2 and |
| | cyclopenta[f][1,3]benzoxazol-6-yl]methyl]-2-oxo-1-oxa-3,8- | Intermediate 87 |
| | diazaspiro[4.5]decan-3-yl]-4H-pyrazino[2,3-b][1,4]oxazin-3-one | |
| | Epimer A | |
| | 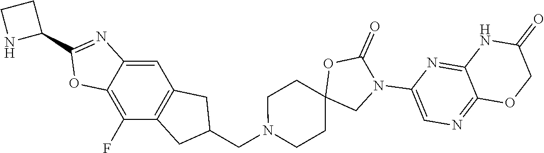 [SMILES: O=C1COc2ncc(N3CC4(CCN(CC5Cc6cc7nc([C@@H]8CCN8)oc7c(F)c6C5)CC4)OC3=O)nc2N1] | |
| | ESP [M + H]⁺: 550.1 | |
| 107 | 6-[8-[[2-[(2S)-azetidin-2-yl]-8-fluoro-6,7-dihydro-5H- | Intermediate 2 and |
| | cyclopenta[f][1,3]benzoxazol-6-yl]methyl]-2-oxo-1-oxa-3,8- | Intermediate 87 |
| | diazaspiro[4.5]decan-3-yl]-4H-pyrazino[2,3-b][1,4]oxazin-3-one | |
| | Epimer B | |
| | 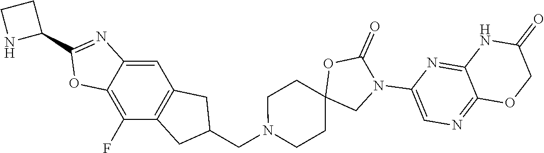 [SMILES: O=C1COc2ncc(N3CC4(CCN(CC5Cc6cc7nc([C@@H]8CCN8)oc7c(F)c6C5)CC4)OC3=O)nc2N1] | |
| | ESP [M + H]⁺: 550.1 | |
| 108 | 6-[8-[[2-[(2R)-azetidin-2-yl]-8-fluoro-6,7-dihydro-5H- | Intermediate 2 and |
| | cyclopenta[f][1,3]benzoxazol-6-yl]methyl]-2-oxo-1-oxa-3,8- | Intermediate 88 |
| | diazaspiro[4.5]decan-3-yl]-4H-pyrazino[2,3-b][1,4]oxazin-3-one | |
| | Epimer A | |
| | 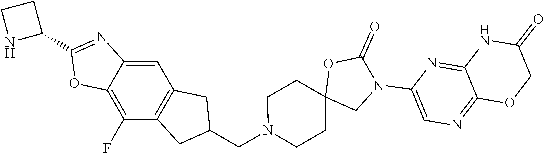 [SMILES: O=C1COc2ncc(N3CC4(CCN(CC5Cc6cc7nc([C@H]8CCN8)oc7c(F)c6C5)CC4)OC3=O)nc2N1] | |
| | ESP [M + H]⁺: 550.1 | |
| 109 | 6-[2-oxo-8-[[2-(azetidin-3-yl)-8-fluoro-6,7-dihydro-5H- | Intermediate 2 and |
| | cyclopenta[f]benzotriazol-6-yl]methyl]-1-oxa-3,8- | Intermediate 89 |
| | diazaspiro[4.5]decan-3-yl]-4H-pyrazino[2,3-b][1,4]oxazin-3-one | |
| | Enantiomer A | |
| | 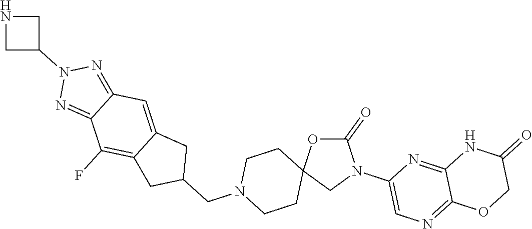 [SMILES: O=C1COc2ncc(N3CC4(CCN(CC5Cc6cc7nn(C8CNC8)nc7c(F)c6C5)CC4)OC3=O)nc2N1] | |
| | ESP [M + H]⁺: 550.3 | |
| 110 | 6-[2-oxo-8-[[2-(azetidin-3-yl)-8-fluoro-6,7-dihydro-5H- | Intermediate 2 and |
| | cyclopenta[f]benzotriazol-6-yl]methyl]-1-oxa-3,8- | Intermediate 89 |
| | diazaspiro[4.5]decan-3-yl]-4H-pyrazino[2,3-b][1,4]oxazin-3-one | |
| | Enantiomer B | |
| | 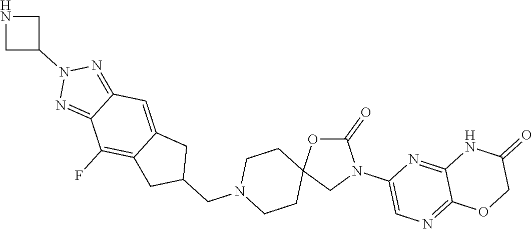 [SMILES: O=C1COc2ncc(N3CC4(CCN(CC5Cc6cc7nn(C8CNC8)nc7c(F)c6C5)CC4)OC3=O)nc2N1] | |
| | ESP [M + H]⁺: 550.1 | |
| 111 | 6-[8-[[2-(azetidin-3-yl)-8-fluoro-6,7-dihydro-5H- | Intermediate 3 and |
| | cyclopenta[f][1,3]benzoxazol-6-yl]methyl]-2-oxo-1-oxa-3,8- | Intermediate 84 |
| | diazaspiro[4.5]decan-3-yl]-4H-pyrido[3,2-b][1,4]oxazin-3-one | |
| | Enantiomer A | |
| | 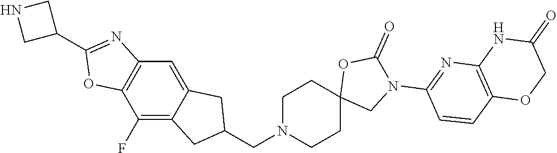 [SMILES: O=C1COc2ccc(N3CC4(CCN(CC5Cc6cc7nc(C8CNC8)oc7c(F)c6C5)CC4)OC3=O)nc2N1] | |
| | ESP [M + H]⁺: 549.1 | |
| 112 | 6-[8-[[2-(azetidin-3-yl)-8-fluoro-6,7-dihydro-5H- | Intermediate 3 and |
| | cyclopenta[f][1,3]benzoxazol-6-yl]methyl]-2-oxo-1-oxa-3,8- | Intermediate 84 |
| | diazaspiro[4.5]decan-3-yl]-4H-pyrido[3,2-b][1,4]oxazin-3-one | |
| | Enantiomer B | |
| | 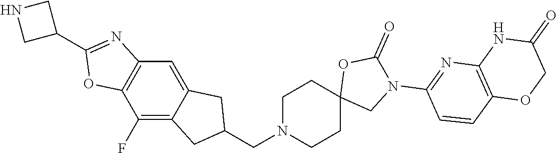 [SMILES: O=C1COc2ccc(N3CC4(CCN(CC5Cc6cc7nc(C8CNC8)oc7c(F)c6C5)CC4)OC3=O)nc2N1] | |
| | ESP [M + H]⁺: 549.2 | |

Examples 72 and 73: the two regioisomers were separated by preparative LCMS and were obtained as an off-white solid and a white solid respectively. The structures of the compounds were determined by 2D NMR.

Example 79: the compound was further purified by preparative HPLC (formic acid/MeCN).

### Example 113

6-[2-Oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrido[2,3-b][1,4]oxazin-3-one; formic acid Enantiomer A

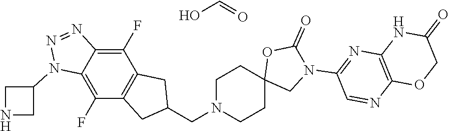 [SMILES: O=C1COc2ncc(N3CC4(CCN(CC5Cc6c(c(F)c7c(nnn7C7CNC7)c6F)C5)CC4)OC3=O)nc2N1 ; O=CO]

### Step 1: tert-Butyl 3-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]azetidine-1-carboxylate Enantiomer A

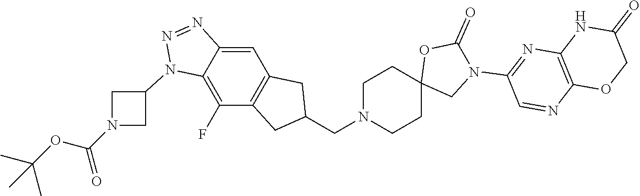 [SMILES: CC(C)(C)OC(=O)N1CC(n2nnc3cc4c(c(F)c32)CC(CN2CCC3(CC2)CN(c2cnc5c(n2)NC(=O)CO5)C(=O)O3)C4)C1]

To a solution of 6-(2-oxo-1-oxa-3,8-diazaspiro[4.5]decan-3-yl)-4H-pyrazino[2,3-b][1,4]oxazin-3-one (Intermediate 2, 200.0 mg, 0.66 mmol, 1.0 eq) and tert-butyl 3-(8-fluoro-6-formyl-6,7-dihydro-5H-cyclopenta[f]benzotriazol-1-yl)azetidine-1-carboxylate (Intermediate 90, 283.3 mg, 0.79 mmol, 1.2 eq) in methanol (5 mL) and THF (2 mL) was added N,N-diisopropylethylamine (0.17 mL, 0.98 mmol, 1.5 eq), then the mixture was stirred at 20° C. for 20 min. Acetic acid (0.09 mL, 1.64 mmol, 2.5 eq) was added, followed by sodium cyanoborohydride (0.1 mL, 1.64 mmol, 2.5 eq). The mixture was stirred at 20° C. for 2 h. To the mixture was added NaHCO₃ to adjust pH to >7 and then the mixture was filtered. The filter cake was collected and the filtrate was extracted with EtOAc (20 mL×3). The combined organic phase was concentrated in vacuo to give the crude racemic product (0.09 g). The enantiomers were separated by chiral SFC to give the title compound (41.0 mg, 0.06 mmol, 9.5% yield) and tert-butyl 3-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]azetidine-1-carboxylate Enantiomer B (33.0 mg, 0.05 mmol, 7.8% yield). MS (ESP) m/z=650.2 [M+H]⁺ and 650.3 [M+H]⁺ respectively.

### Step 2: 6-[2-Oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one; formic acid Enantiomer A

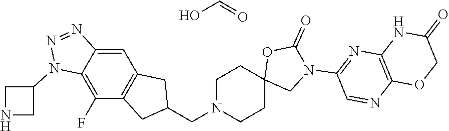 [SMILES: O=C1COc2ncc(N3CC4(CCN(CC5Cc6cc7nnn(C8CNC8)c7c(F)c6C5)CC4)OC3=O)nc2N1 ; O=CO]

tert-Butyl 3-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]azetidine-1-carboxylate Enantiomer A (41.0 mg, 0.06 mmol, 1.0 eq) was dissolved in formic acid (2.0 mL, 50.4 mmol, 800 eq) and the mixture was stirred at 20° C. for 3 h. The mixture was washed with water (20 mL) then dried by lyophilization to give the title compound (25.5 mg, 0.04 mmol, 57.5% yield) as a white solid. MS (ESP) m/z=550.0 [M+H]⁺.

The following examples were prepared by analogy to Example 113.

| Ex. | Name, Structure, MS | Starting Materials |
|---|---|---|
| 114 | 6-[2-oxo-8-[[1-(azetidin-3-yl)-8-fluoro-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one; formic acid Enantiomer B 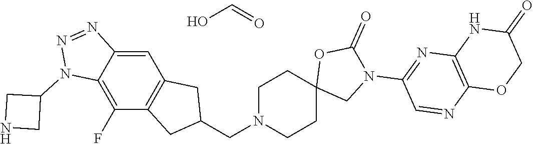 [SMILES: O=C1COc2ncc(N3CC4(CCN(CC5Cc6cc7nnn(C8CNC8)c7c(F)c6C5)CC4)OC3=O)nc2N1 ; O=CO] ESP [M + H]⁺: 550.0 | Intermediate 2 and Intermediate 90 |
| 115 | 6-[2-oxo-8-[[3-(azetidin-3-yl)-8-fluoro-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one; formic acid Enantiomer A 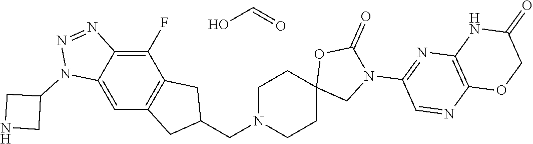 ESP [M + H]⁺: 549.9 | Intermediate 2 and Intermediate 91 |
| 116 | 6-[2-oxo-8-[[3-(azetidin-3-yl)-8-fluoro-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one; formic acid Enantiomer B 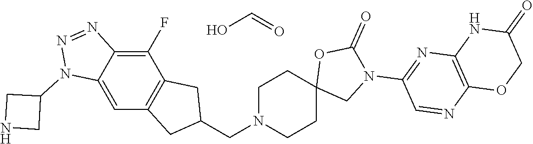 ESP [M + H]⁺: 550.0 | Intermediate 2 and Intermediate 91 |
| 117 | 6-[8-[[4,8-difluoro-2-[2-(methylamino)ethyl]-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3-one; formic acid 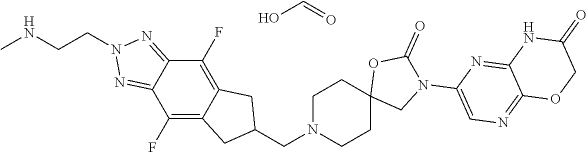 ESP [M + H]⁺: 538.3 | Intermediate 2 and Intermediate 92 |
| 118 | 6-[2-oxo-8-[[4,8-difluoro-1-[2-(methylamino)ethyl]-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one Enantiomer A 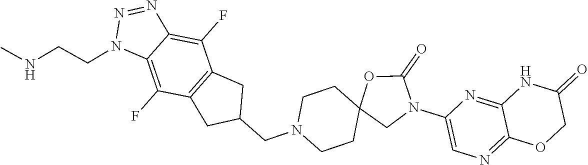 [SMILES: CNCCn1nnc2c(F)c3c(c(F)c21)CC(CN1CCC2(CC1)CN(c1cnc4c(n1)NC(=O)CO4)C(=O)O2)C3] ESP [M + H]⁺: 570.1 | Intermediate 2 and Intermediate 93 |
| 119 | 6-[2-oxo-8-[[4,8-difluoro-1-[2-(methylamino)ethyl]-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one Enantiomer B 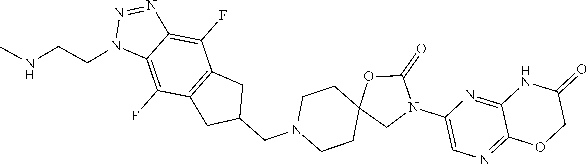 [SMILES: CNCCn1nnc2c(F)c3c(c(F)c21)CC(CN1CCC2(CC1)CN(c1cnc4c(n1)NC(=O)CO4)C(=O)O2)C3] ESP [M + H]⁺: 570.1 | Intermediate 2 and Intermediate 93 |
| 120 | 6-[8-[[1-(azetidin-3-yl)-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3- one; formic acid Enantiomer A 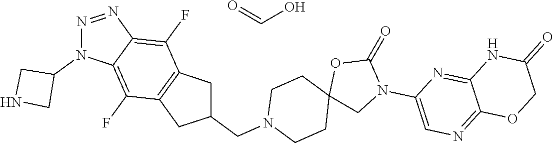 [SMILES: O=C1COc2ncc(N3CC4(CCN(CC5Cc6c(c(F)c7c(nnn7C7CNC7)c6F)C5)CC4)OC3=O)nc2N1 ; O=CO] ESP [M + H]⁺: 568.1 | Intermediate 2 and Intermediate 72 |
| 121 | 6-[8-[[1-(azetidin-3-yl)-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3- one; formic acid Enantiomer B 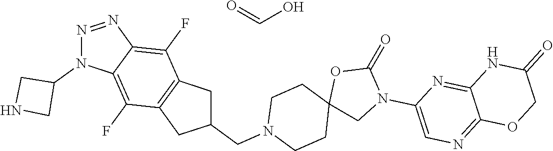 [SMILES: O=C1COc2ncc(N3CC4(CCN(CC5Cc6c(c(F)c7c(nnn7C7CNC7)c6F)C5)CC4)OC3=O)nc2N1 ; O=CO] ESP [M + H]⁺: 568.1 | Intermediate 2 and Intermediate 72 |
| 122 | 6-[8-[2-[2-(cyclopropylamino)ethyl]-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3-one 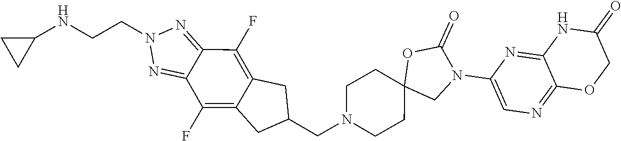 [SMILES: O=C1COc2ncc(N3CC4(CCN(CC5Cc6c(c(F)c7nn(CCNC8CC8)nc7c6F)C5)CC4)OC3=O)nc2N1] ESP [M + H]+: 596.5 | Intermediate 2 and Intermediate 94 |
| 123 | 6-[8-[[2-[(2S)-2-aminopropyl]-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3- one; formic acid 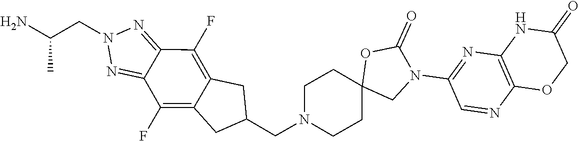 [SMILES: C[C@H](N)Cn1nc2c(F)c3c(c(F)c2n1)CC(CN1CCC2(CC1)CN(c1cnc4c(n1)NC(=O)CO4)C(=O)O2)C3] ESP [M + H]⁺: 570.3 | Intermediate 2 and Intermediate 96 |

### Example 124

### 6-[8-[[1-[2-(Cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid Enantiomer A

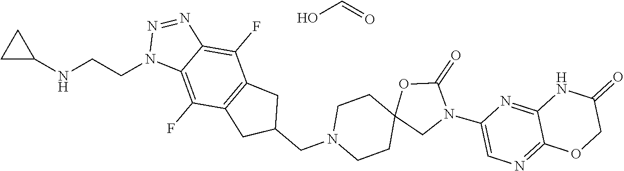 [SMILES: O=C1COc2ncc(N3CC4(CCN(CC5Cc6c(c(F)c7c(nnn7CCNC7CC7)c6F)C5)CC4)OC3=O)nc2N1 ; O=CO]

### and

### Example 125

### 6-[8-[[1-[2-(Cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid Enantiomer B

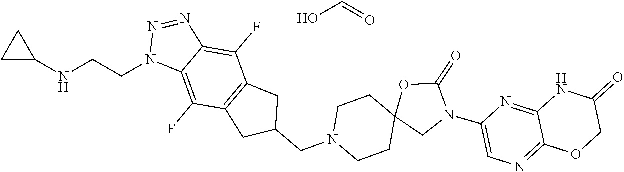 [SMILES: O=C1COc2ncc(N3CC4(CCN(CC5Cc6c(c(F)c7c(nnn7CCNC7CC7)c6F)C5)CC4)OC3=O)nc2N1 ; O=CO]

### Step 1: tert-butyl N-cyclopropyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]carbamate

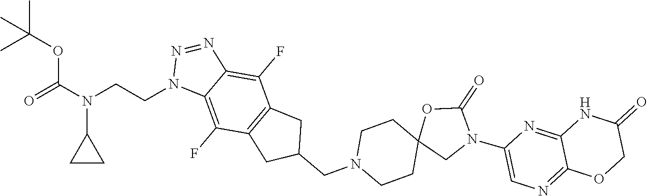 [SMILES: CC(C)(C)OC(=O)N(CCn1nnc2c(F)c3c(c(F)c21)CC(CN1CCC2(CC1)CN(c1cnc4c(n1)NC(=O)CO4)C(=O)O2)C3)C1CC1]

To a solution of 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2, 309.5 mg, 0.74 mmol, 1.0 eq) in methanol (3 mL) and THF (1.5 mL) was added DIPEA (143.09 mg, 1.11 mmol, 1.5 eq) at 25° C. The solution was stirred at 25° C. for 5 min. To the solution was added tert-butyl N-cyclopropyl-N-[2-(4,8-difluoro-6-formyl-6,7-dihydro-5H-cyclopenta[f]benzotriazol-1-yl)ethyl]carbamate (Intermediate 95, 300.0 mg, 0.74 mmol, 1.0 eq) and acetic acid (110.8 mg, 1.85 mmol, 2.5 eq) at 25° C. The resulting solution was stirred at 25° C. for 20 min. To the mixture was added sodium cyanoborohydride (116.3 mg, 1.85 mmol, 2.5 eq). The mixture was stirred at 25° C. for 12 h. The reaction mixture was poured into NH₄Cl (sat. aq. 50 mL) and extracted with ethyl acetate (20 mL×3). The combined organic layers were washed with brine (20 mL×2), dried over Na₂SO₄, filtered and concentrated to give a residue which was triturated with MTBE (5 mL) and stirred for 5 min. The mixture was filtered and the residue was concentrated under reduced pressure to give the title compound (150.0 mg, 0.22 mmol, 28.6% yield) as an off-white solid. MS (ESP) m/z=696.1 [M+H]⁺.

### Step 2: 6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one hydrochloride

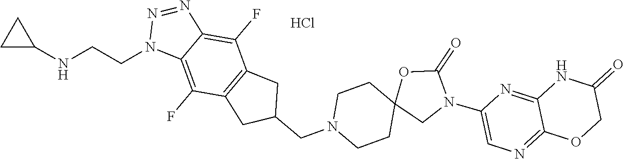 [SMILES: O=C1COc2ncc(N3CC4(CCN(CC5Cc6c(c(F)c7c(nnn7CCNC7CC7)c6F)C5)CC4)OC3=O)nc2N1 ; Cl]

To a solution of tert-butyl N-cyclopropyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]carbamate (150.0 mg, 0.22 mmol, 1.0 eq) in DCM (2 mL) was added HCl in dioxane (2.0 mL, 8.0 mmol, 37.1 eq). The mixture was stirred at 25° C. for 1 h. The reaction mixture was triturated in MTBE (20 ml×2). The top solution was poured off and the residue was concentrated under reduced pressure to give the title compound (145.0 mg, 0.23 mmol, quant. yield) as an off-white solid. MS (ESP) m/z=596.1 [M+H]⁺.

### Step 3: 6-[8-[[1-[2-(Cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid Enantiomer A and Enantiomer B

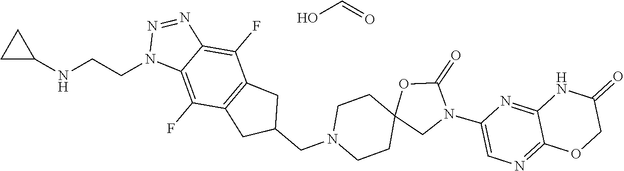 [SMILES: O=C1COc2ncc(N3CC4(CCN(CC5Cc6c(c(F)c7c(nnn7CCNC7CC7)c6F)C5)CC4)OC3=O)nc2N1 ; O=CO]

The enantiomers of 6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one hydrochloride (90.0 mg, 0.14 mmol, 1.0 eq) were separated by preparative SFC. Peak 1 was concentrated under reduced pressure to give a residue which was purified by preparative HPLC (formic acid conditions) and lyophilized to give the title compound Enantiomer A (18.65 mg, 0.03 mmol, 18.2% yield) as a white solid. Peak 2 was concentrated under reduced pressure to give a residue which was purified by preparative HPLC (formic acid conditions) and lyophilized to give the title compound Enantiomer B (8.25 mg, 0.01 mmol, 8.1% yield) as a white solid. MS (ESP) m/z=596.1 [M+H]⁺.

The following examples were prepared by analogy to Examples 124 and 125.

| Ex. | Name, Structure, MS | Starting Materials |
|---|---|---|
| 126 | 6-[2-oxo-8-[[4,8-difluoro-1-[2-(methylamino)ethyl]-6,7-dihydro-5H- cyclopenta[f]benzimidazol-6-yl]methyl]-1-oxa-3,8- diazaspiro[4.5]decan-3-yl]-4H-pyrazino[2,3-b][1,4]oxazin-3-one Enantiomer A 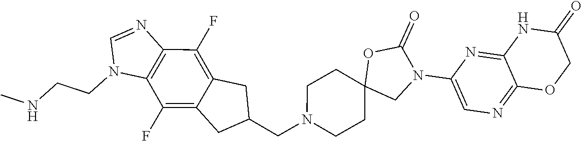 [SMILES: CNCCn1cnc2c(F)c3c(c(F)c21)CC(CN1CCC2(CC1)CN(c1cnc4c(n1)NC(=O)CO4)C(=O)O2)C3] ESP [M + H]⁺: 569.3 | Intermediate 2 and Intermediate 98 |
| 127 | 6-[2-oxo-8-[4,8-difluoro-1-[2-(methylamino)ethyl]-6,7-dihydro-5H- cyclopenta[f]benzimidazol-6-yl]methyl]-1-oxa-3,8- diazaspiro[4.5]decan-3-yl]-4H-pyrazino[2,3-b] [1,4]oxazin-3-one Enantiomer B 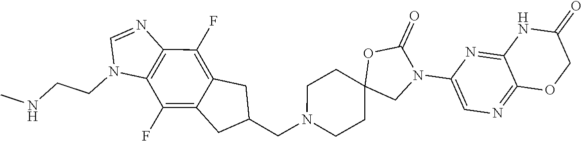 [SMILES: CNCCn1cnc2c(F)c3c(c(F)c21)CC(CN1CCC2(CC1)CN(c1cnc4c(n1)NC(=O)CO4)C(=O)O2)C3] ESP [M + H]⁺: 569.4 | Intermediate 2 and Intermediate 98 |
| 128 | 6-[8-[[1-[(2S)-2-aminopropyl]-4,8-difluoro-6,7-dihydro-5H- cyclopenta[f]benzimidazol-6-yl]methyl]-2-oxo-1-oxa-3,8- diazaspiro[4.5]decan-3-yl]-4H-pyrazino[2,3-b] [1,4]oxazin-3-one Epimer A 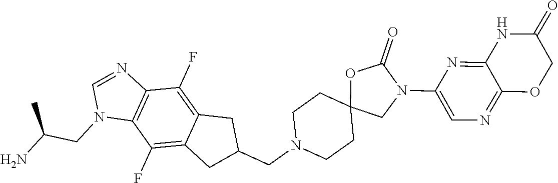 [SMILES: C[C@H](N)Cn1cnc2c(F)c3c(c(F)c21)CC(CN1CCC2(CC1)CN(c1cnc4c(n1)NC(=O)CO4)C(=O)O2)C3] ESP [M + H]⁺: 569.3 | Intermediate 2 and Intermediate 99 |
| 129 | 6-[8-[[1-[(2S)-2-aminopropyl]-4,8-difluoro-6,7-dihydro-5H- cyclopenta[f]benzimidazol-6-yl]methyl]-2-oxo-1-oxa-3,8- diazaspiro[4.5]decan-3-yl]-4H-pyrazino[2,3-b][1,4]oxazin-3- one; formic acid Epimer B 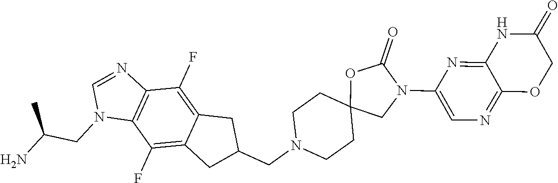 [SMILES: C[C@H](N)Cn1cnc2c(F)c3c(c(F)c21)CC(CN1CCC2(CC1)CN(c1cnc4c(n1)NC(=O)CO4)C(=O)O2)C3] ESP [M + H]⁺: 569.3 | Intermediate 2 and Intermediate 99 |
| 130 | 6-[8-[[1-[(2S)-2-aminopropyl]-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b][1,4]oxazin-3-one hydrochloride Epimer A 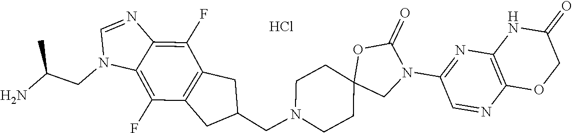 [SMILES: C[C@H](N)Cn1cnc2c(F)c3c(c(F)c21)CC(CN1CCC2(CC1)CN(c1cnc4c(n1)NC(=O)CO4)C(=O)O2)C3 ; Cl] ESP [M + H]⁺: 570.2 | Intermediate 2 and Intermediate 97 |
| 131 | 6-[8-[[1-[(2S)-2-aminopropyl]-4,8-difluoro-6,7-dihydro-5H- cyclopenta[f]benzotriazol-6-yl]methyl]-2-oxo-1-oxa-3,8- diazaspiro[4.5]decan-3-yl]-4H-pyrazino[2,3-b] [1,4]oxazin-3-one hydrochloride Epimer B 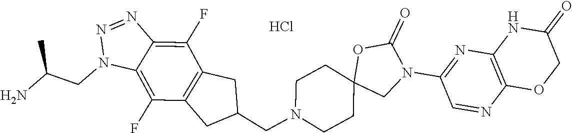 [SMILES: C[C@H](N)Cn1nnc2c(F)c3c(c(F)c21)CC(CN1CCC2(CC1)CN(c1cnc4c(n1)NC(=O)CO4)C(=O)O2)C3 ; Cl] ESP [M + H]⁺: 570.3 | Intermediate 2 and Intermediate 97 |
| 132 | 6-[2-oxo-8-[[2-(2-aminoethyl)-8-fluoro-6,7-dihydro-5H- cyclopenta[f]benzotriazol-6-yl]methyl]-1-oxa-3,8- diazaspiro[4.5]decan-3-yl]-4H-pyrido[3,2-b][1,4]oxazin-3-one dihydrochloride Enantiomer A 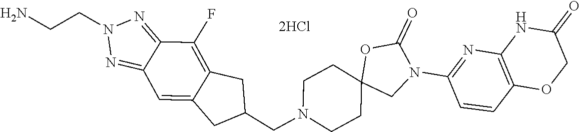 [SMILES: NCCn1nc2cc3c(c(F)c2n1)CC(CN1CCC2(CC1)CN(c1ccc4c(n1)NC(=O)CO4)C(=O)O2)C3 ; "2HCl"] ESP [M + H]⁺: 537.2 | Intermediate 3 and Intermediate 66 |
| 133 | 6-[2-oxo-8-[[2-(2-aminoethyl)-8-fluoro-6,7-dihydro-5H- cyclopenta[f]benzotriazol-6-yl]methyl]-1-oxa-3,8- diazaspiro[4.5]decan-3-yl]-4H-pyrido[3,2-b][1,4]oxazin-3-one dihydrochloride Enantiomer B 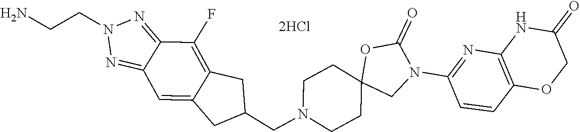 [SMILES: NCCn1nc2cc3c(c(F)c2n1)CC(CN1CCC2(CC1)CN(c1ccc4c(n1)NC(=O)CO4)C(=O)O2)C3 ; "2HCl"] ESP [M + H]⁺: 537.2 | Intermediate 3 and Intermediate 66 |
| 134 | 6-[2-oxo-8-[[2-(2-aminoethyl)-8-fluoro-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one dihydrochloride Enantiomer A 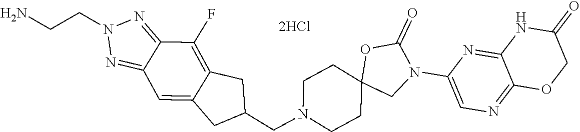 [SMILES: NCCn1nc2cc3c(c(F)c2n1)CC(CN1CCC2(CC1)CN(c1cnc4c(n1)NC(=O)CO4)C(=O)O2)C3 ; "2HCl"] ESP [M + H]⁺: 538.1 | Intermediate 2 and Intermediate 66 |
| 135 | 6-[2-oxo-8-[[2-(2-aminoethyl)-8-fluoro-6,7-dihydro-5H- cyclopenta[f]benzotriazol-6-yl]methyl]-1-oxa-3,8- diazaspiro[4.5]decan-3-yl]-4H-pyrazino[2,3-b][1,4]oxazin-3-one dihydrochloride Enantiomer B 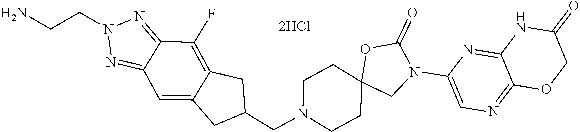 [SMILES: NCCn1nc2cc3c(c(F)c2n1)CC(CN1CCC2(CC1)CN(c1cnc4c(n1)NC(=O)CO4)C(=O)O2)C3 ; "2HCl"] ESP [M + H]⁺: 538.1 | Intermediate 2 and Intermediate 66 |

Examples 130 and 131: the epimers were separated by chiral SFC after Step 1.

Examples 132 to 135: the enantiomers were separated by chiral SFC after Step 1.

### Example 136

### 6-[8-[[8-Fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one Enantiomer A

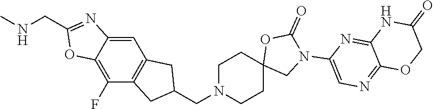 [SMILES: CNCc1nc2cc3c(c(F)c2o1)CC(CN1CCC2(CC1)CN(c1cnc4c(n1)NC(=O)CO4)C(=O)O2)C3]

### Step 1: tert-butyl N-[[8-fluoro-6-[[2-oxo-3-(3-oxo-4H-pyrido[3,2-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-N-methyl-carbamate Enantiomer A

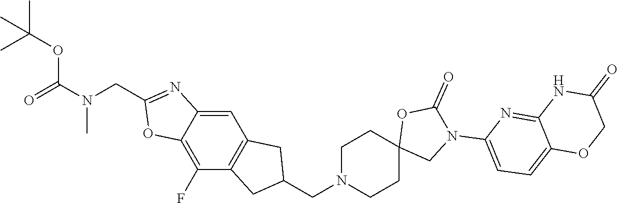 [SMILES: CN(Cc1nc2cc3c(c(F)c2o1)CC(CN1CCC2(CC1)CN(c1ccc4c(n1)NC(=O)CO4)C(=O)O2)C3)C(=O)OC(C)(C)C]

The racemic title compound was prepared from Intermediate 3 and Intermediate 29 by analogy with Example 125, Step 1 and was obtained as a white solid. MS (ESP) m/z=637.2 [M+H]⁺. The enantiomers were separated by chiral SFC to give Peak 1: tert-butyl N-[[8-fluoro-6-[[2-oxo-3-(3-oxo-4H-pyrido[3,2-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-N-methyl-carbamate Enantiomer A and Peak 2: tert-butyl N-[[8-fluoro-6-[[2-oxo-3-(3-oxo-4H-pyrido[3,2-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-N-methyl-carbamate Enantiomer B.

### Step 2: 6-[8-[[8-Fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one Enantiomer A

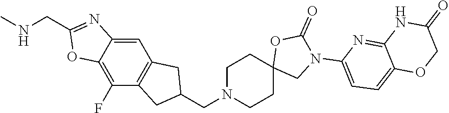 [SMILES: CNCc1nc2cc3c(c(F)c2o1)CC(CN1CCC2(CC1)CN(c1ccc4c(n1)NC(=O)CO4)C(=O)O2)C3]

To a solution of tert-butyl N-[[8-fluoro-6-[[2-oxo-3-(3-oxo-4H-pyrido[3,2-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-N-methyl-carbamate Enantiomer A (100.0 mg, 0.16 mmol, 1.0 eq) in DCM (2.5 mL) was added iodo(trimethyl)silane (0.65 mL, 0.16 mmol, 1.0 eq). The resulting mixture was stirred at −20° C. for 0.75 h. The reaction mixture was diluted with EtOAc (6 mL). The solid was collected by filtration under the protection of nitrogen. Then the filter cake was quickly dissolved in methanol (12 mL), Amberlyst® A21 basic resin was added to the solution until the pH was >7. The suspension was filtered and the filter cake was washed with methanol (10 mL×2). The combined filtrate was concentrated and triturated with MTBE (4 mL). The resulting solid was collected by filtration and the filter cake was dried under vacuum to give the title compound (45.2 mg, 0.08 mmol, 51.8% yield) as a light grey solid. MS (ESP) m/z=537.3 [M+H]⁺.

### Example 137

### 6-[8-[[8-Fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one Enantiomer B

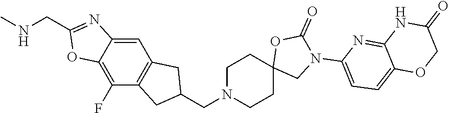 [SMILES: CNCc1nc2cc3c(c(F)c2o1)CC(CN1CCC2(CC1)CN(c1ccc4c(n1)NC(=O)CO4)C(=O)O2)C3]

The title compound was prepared from tert-butyl N-[[8-fluoro-6-[[2-oxo-3-(3-oxo-4H-pyrido[3,2-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-N-methyl-carbamate Enantiomer B (Example 136, Step 1) by analogy with Example 136, Step 2 and was obtained as a light grey solid. MS (ESP) m/z=537.3 [M+H]⁺.

### Example 138

### 6-[8-[[2-[(3R,4R)-4-Aminotetrahydrofuran-3-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

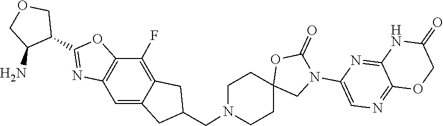 [SMILES: N[C@H]1COC[C@@H]1c1nc2cc3c(c(F)c2o1)CC(CN1CCC2(CC1)CN(c1cnc4c(n1)NC(=O)CO4)C(=O)O2)C3]

The title compound was prepared from Intermediate 2 and Intermediate 100 by analogy with Example 136 and was obtained as a light grey solid. MS (ESP) m/z=580.4 [M+H]⁺.

### Example 139

### 6-[8-[[2-[(3R,4R)-4-Aminotetrahydrofuran-3-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

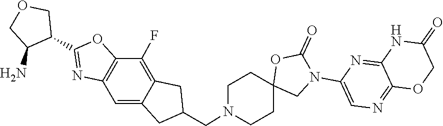 [SMILES: N[C@H]1COC[C@@H]1c1nc2cc3c(c(F)c2o1)CC(CN1CCC2(CC1)CN(c1cnc4c(n1)NC(=O)CO4)C(=O)O2)C3]

The title compound was prepared from Intermediate 2 and Intermediate 100 by analogy with Example 136 and was obtained as a light grey solid. MS (ESP) m/z=580.3 [M+H]⁺.

### Example 140

### 6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

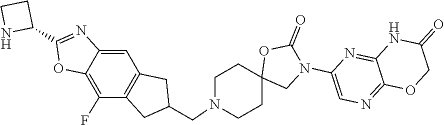 [SMILES: O=C1COc2ncc(N3CC4(CCN(CC5Cc6cc7nc([C@H]8CCN8)oc7c(F)c6C5)CC4)OC3=O)nc2N1]

### Step 1: tert-butyl (2R)-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]azetidine-1-carboxylate Epimer B

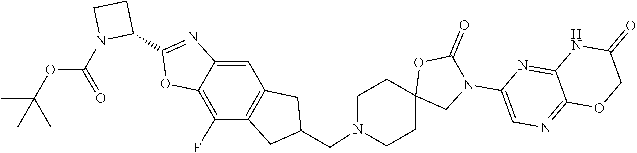 [SMILES: CC(C)(C)OC(=O)N1CC[C@@H]1c1nc2cc3c(c(F)c2o1)CC(CN1CCC2(CC1)CN(c1cnc4c(n1)NC(=O)CO4)C(=O)O2)C3]

The enantiomers of tert-butyl (2R)-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]azetidine-1-carboxylate (Example 108, Step 1) were separated on chiral SFC to give the title compound as a white solid. MS (ESP) m/z=650.1 [M+H]⁺.

### Step 2: 6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

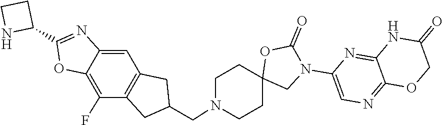 [SMILES: O=C1COc2ncc(N3CC4(CCN(CC5Cc6cc7nc([C@H]8CCN8)oc7c(F)c6C5)CC4)OC3=O)nc2N1]

To a solution of tert-butyl (2R)-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]azetidine-1-carboxylate Epimer B (100.0 mg, 0.15 mmol, 1.0 eq) in DCE (1 mL) was added ZnBr₂ (341.4 mg, 1.54 mmol, 10.0 eq) and the resulting mixture was stirred at 60° C. for 2 h. The mixture was concentrated under reduced pressure to give a residue. The residue was purified by preparative HPLC to give the title compound (31.9 mg, 0.06 mmol, 37.7% yield) as a white solid. MS (ESP) m/z=550.2 [M+H]⁺.

### Example 141

### 6-[8-[[8-Fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

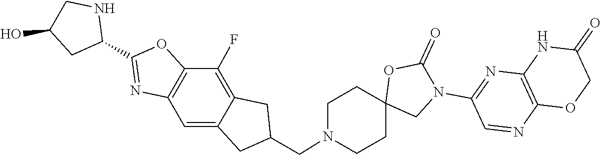 [SMILES: O=C1COc2ncc(N3CC4(CCN(CC5Cc6cc7nc([C@@H]8C[C@@H](O)CN8)oc7c(F)c6C5)CC4)OC3=O)nc2N1]

### Step 1: tert-butyl (2S,4R)-4-[tert-butyl(diphenyl)silyl]oxy-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]pyrrolidine-1-carboxylate Epimer A

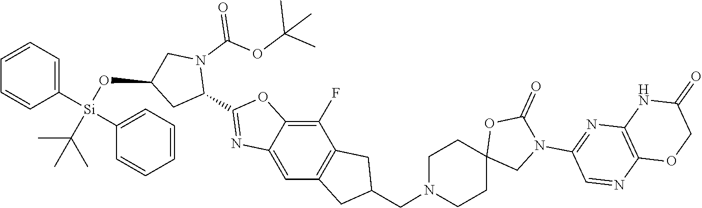 [SMILES: CC(C)(C)OC(=O)N1C[C@H](O[Si](c2ccccc2)(c2ccccc2)C(C)(C)C)C[C@H]1c1nc2cc3c(c(F)c2o1)CC(CN1CCC2(CC1)CN(c1cnc4c(n1)NC(=O)CO4)C(=O)O2)C3]

The racemic title compound was prepared from Intermediate 2 and Intermediate 101 by analogy with Example 125, Step 1 and was obtained as a white solid. MS (ESP) m/z=918.1 [M+H]⁺. The enantiomers were separated by chiral SFC to give Peak 1: tert-butyl (2S,4R)-4-[tert-butyl(diphenyl)silyl]oxy-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]pyrrolidine-1-carboxylate Epimer A and Peak 2: tert-butyl (2S,4R)-4-[tert-butyl(diphenyl)silyl]oxy-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]pyrrolidine-1-carboxylate Epimer B.

### Step 2: tert-butyl (2S,4R)-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]-4-hydroxy-pyrrolidine-1-carboxylate Epimer A

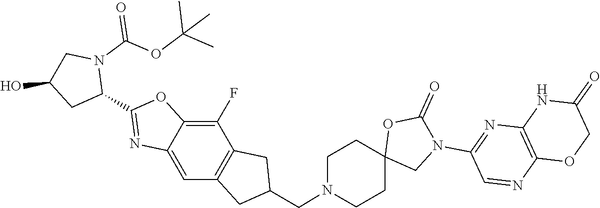 [SMILES: CC(C)(C)OC(=O)N1C[C@H](O)C[C@H]1c1nc2cc3c(c(F)c2o1)CC(CN1CCC2(CC1)CN(c1cnc4c(n1)NC(=O)CO4)C(=O)O2)C3]

To a solution of tert-butyl (2S,4R)-4-[tert-butyl(diphenyl)silyl]oxy-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]pyrrolidine-1-carboxylate Epimer A (598.0 mg, 0.65 mmol, 1.0 eq) in methanol (3 mL) was added potassium fluoride (1200.0 mg, 20.66 mmol, 31.71 eq). The resulting mixture was stirred at 70° C. for 17 h. The mixture was filtered and the filter cake was washed by a mixed solution of 10% MeOH/DCM (100 mL). The filtrate was collected and concentrated in vacuo to give a crude product which was washed with MTBE (20 mL×2) to give the title compound (368.0 mg, 0.54 mmol, 83.1% yield). MS (ESP) m/z=680.2 [M+H]⁺.

### Step 3: 6-[8-[[8-Fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

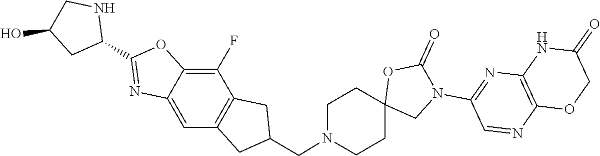 [SMILES: O=C1COc2ncc(N3CC4(CCN(CC5Cc6cc7nc([C@@H]8C[C@@H](O)CN8)oc7c(F)c6C5)CC4)OC3=O)nc2N1]

The title compound was prepared by analogy with Example 136, Step 2 and was obtained as a white solid. MS (ESP) m/z=580.2 [M+H]⁺.

### Example 142

### 6-[8-[[8-Fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

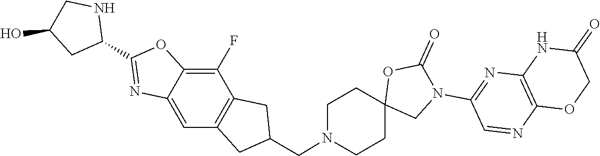 [SMILES: O=C1COc2ncc(N3CC4(CCN(CC5Cc6cc7nc([C@@H]8C[C@@H](O)CN8)oc7c(F)c6C5)CC4)OC3=O)nc2N1]

The title compound was prepared from tert-butyl (2S,4R)-4-[tert-butyl(diphenyl)silyl]oxy-2-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]pyrrolidine-1-carboxylate Epimer B (Example 141, Step 1) by analogy with Example 141, Steps 2 and 3, and was obtained as a white solid. MS (ESP) m/z=580.2 [M+H]⁺.

### Example 143

### 6-[8-[[2-[(1S)-1-Amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

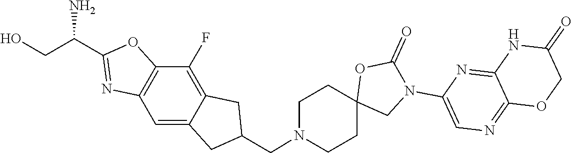 [SMILES: N[C@@H](CO)c1nc2cc3c(c(F)c2o1)CC(CN1CCC2(CC1)CN(c1cnc4c(n1)NC(=O)CO4)C(=O)O2)C3]

### Steps 1 and 2: tert-butyl ((1S)-1-(8-fluoro-6-((2-oxo-3-(3-oxo-3,4-dihydro-2H-pyrazino[2,3-b][1,4]oxazin-6-yl)-1-oxa-3,8-diazaspiro[4.5]decan-8-yl)methyl)-6,7-dihydro-5H-indeno[5,6-d]oxazol-2-yl)-2-hydroxyethyl)carbamate Epimer A

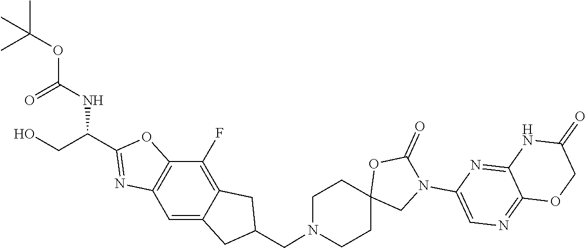 [SMILES: CC(C)(C)OC(=O)N[C@@H](CO)c1nc2cc3c(c(F)c2o1)CC(CN1CCC2(CC1)CN(c1cnc4c(n1)NC(=O)CO4)C(=O)O2)C3]

The title compound was prepared from Intermediate 2 and Intermediate 102 by analogy with Example 141, Steps 1 and 2, and was obtained as a yellow solid. MS (ESP) m/z=654.2 [M+H]⁺.

### Step 3: 6-[8-[[2-[(1S)-1-Amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

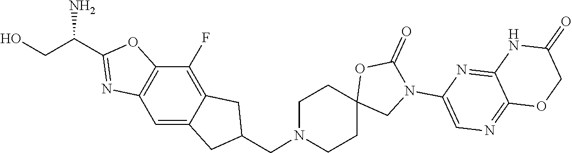 [SMILES: N[C@@H](CO)c1nc2cc3c(c(F)c2o1)CC(CN1CCC2(CC1)CN(c1cnc4c(n1)NC(=O)CO4)C(=O)O2)C3]

The title compound was prepared by analogy with Example 63, Step 3 and was obtained as a white solid. MS (ESP) m/z=554.1 [M+H]⁺.

### Example 144

### 6-[8-[[2-[(1S)-1-Amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

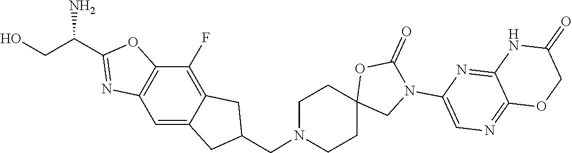 [SMILES: N[C@@H](CO)c1nc2cc3c(c(F)c2o1)CC(CN1CCC2(CC1)CN(c1cnc4c(n1)NC(=O)CO4)C(=O)O2)C3]

The title compound was prepared from Intermediate 3 and Intermediate 102 by analogy with Example 143 and was obtained as a white solid. MS (ESP) m/z=554.1 [M+H]⁺.

### Example 145

### 6-[8-[[8-Fluoro-2-[(1S)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

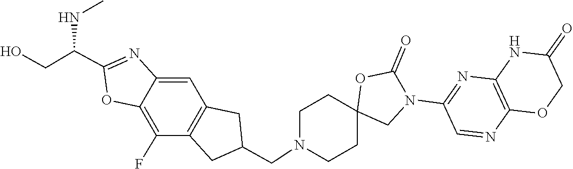 [SMILES: CN[C@@H](CO)c1nc2cc3c(c(F)c2o1)CC(CN1CCC2(CC1)CN(c1cnc4c(n1)NC(=O)CO4)C(=O)O2)C3]

### Step 1: tert-butyl N-[(1S)-2-[tert-butyl(diphenyl)silyl]oxy-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]ethyl]-N-methyl-carbamate

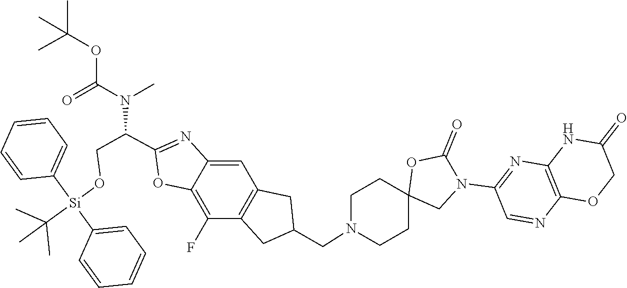 [SMILES: CN(C(=O)OC(C)(C)C)[C@@H](CO[Si](c1ccccc1)(c1ccccc1)C(C)(C)C)c1nc2cc3c(c(F)c2o1)CC(CN1CCC2(CC1)CN(c1cnc4c(n1)NC(=O)CO4)C(=O)O2)C3]

The title compound was prepared from Intermediate 2 and Intermediate 103 by analogy with Example 141, Step 1, and was obtained as a brown solid. MS (ESP) m/z=906.4 [M+H]⁺.

### Step 2: tert-butyl N-[(1S)-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]-2-hydroxy-ethyl]-N-methyl-carbamate Epimer A

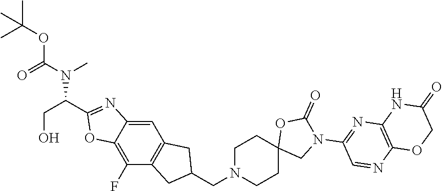 [SMILES: CN(C(=O)OC(C)(C)C)[C@@H](CO)c1nc2cc3c(c(F)c2o1)CC(CN1CCC2(CC1)CN(c1cnc4c(n1)NC(=O)CO4)C(=O)O2)C3]

To a solution of tert-butyl N-[(1S)-2-[tert-butyl(diphenyl)silyl]oxy-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]ethyl]-N-methyl-carbamate (1.0 g, 1.1 mmol, 1.0 eq) in THF (16 mL) was added tetrabutylammonium fluoride (1M in THF, 1.32 mL, 1.32 mmol, 1.2 eq). The resulting mixture was stirred at 25° C. for 1 h. The mixture was concentrated and the enantiomers were separated by chiral SFC to give Peak 1: tert-butyl N-[(1S)-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]-2-hydroxy-ethyl]-N-methyl-carbamate Epimer A and Peak 2: tert-butyl N-[(1S)-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]-2-hydroxy-ethyl]-N-methyl-carbamate Epimer B.

### Step 3: 6-[8-[[8-fluoro-2-[(1S)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer A

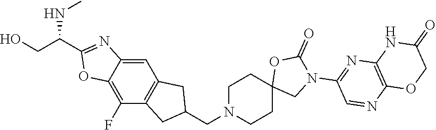 [SMILES: CN[C@@H](CO)c1nc2cc3c(c(F)c2o1)CC(CN1CCC2(CC1)CN(c1cnc4c(n1)NC(=O)CO4)C(=O)O2)C3]

The title compound was prepared by analogy with Example 136, Step 2 and was obtained as a white solid. MS (ESP) m/z=568.2 [M+H]⁺.

### Example 146

### 6-[8-[[8-Fluoro-2-[(1S)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one Epimer B

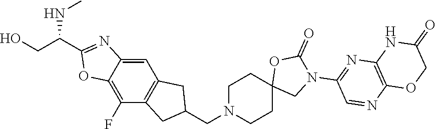 [SMILES: CN[C@@H](CO)c1nc2cc3c(c(F)c2o1)CC(CN1CCC2(CC1)CN(c1cnc4c(n1)NC(=O)CO4)C(=O)O2)C3]

The title compound was prepared from tert-butyl N-[(1S)-1-[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]-2-hydroxy-ethyl]-N-methyl-carbamate Epimer B (Example 145, Step 2) by analogy with Example 145, Step 3 and was obtained as a grey solid. MS (ESP) m/z=568.2 [M+H]⁺.

### Example 147

### 6-[8-[[8-Fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one Epimer A

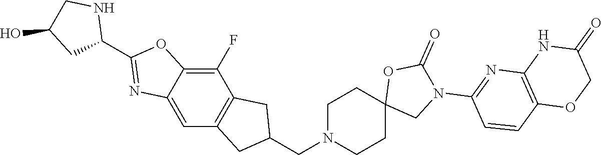 [SMILES: O=C1COc2ccc(N3CC4(CCN(CC5Cc6cc7nc([C@@H]8C[C@@H](O)CN8)oc7c(F)c6C5)CC4)OC3=O)nc2N1]

The title compound was prepared from Intermediate 3 and Intermediate 101 by analogy with Example 141 and was obtained as an off-white solid. MS (ESP) m/z=579.3 [M+H]⁺.

### Example 148

### 6-[8-[[8-Fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one Epimer B

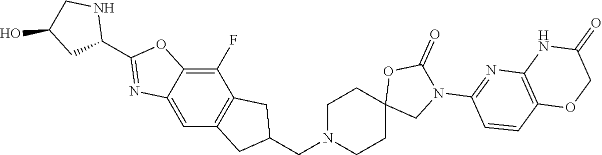 [SMILES: O=C1COc2ccc(N3CC4(CCN(CC5Cc6cc7nc([C@@H]8C[C@@H](O)CN8)oc7c(F)c6C5)CC4)OC3=O)nc2N1]

The title compound was prepared from Intermediate 3 and Intermediate 101 by analogy with Example 142 and was obtained as an off-white solid. MS (ESP) m/z=579.3 [M+H]⁺.

### Example 149

### (2R)—N-[2-[4,8-Difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)propanamide

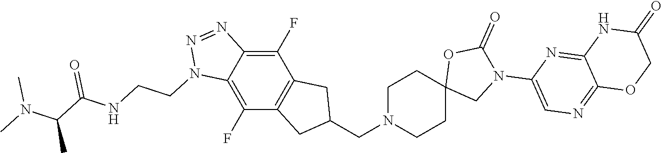 [SMILES: C[C@H](C(=O)NCCn1nnc2c(F)c3c(c(F)c21)CC(CN1CCC2(CC1)CN(c1cnc4c(n1)NC(=O)CO4)C(=O)O2)C3)N(C)C]

To a stirred solution of (2R)-2-(dimethylamino)propanoic acid [CAS #157431-09-9] (31.6 mg, 0.27 mmol, 1.0 eq) in DMF (2.7 mL) N,N-diisopropylethylamine (0.07 mL, 0.41 mmol, 1.5 eq) and O-(7-azabenzotriazol-1-yl)-N,N,N′,N′-tetramethyluronium hexafluorophosphate (123.2 mg, 0.32 mmol, 1.2 eq) were added followed by 6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 42, 150.0 mg, 0.27 mmol, 1.0 eq). The resulting mixture was stirred at 25° C. for 1.5 h. Then water was added and the mixture was extracted with EtOAc (3×10 mL). The organic layers were collected, dried over Na₂SO₄, filtered and concentrated under vacuum to give a crude compound which was purified by silica cartridge chromatography (NH-silica 28 g, DCM/MeOH from 100:0 to 90:10) to give the title compound (141.2 mg, 0.22 mmol, 75.9% yield) as an off-white solid. MS (ESP) m/z=655.3 [M+H]⁺.

The following examples were prepared by analogy to Example 149.

| Ex. | Name, Structure, MS | Starting Materials |
|---|---|---|
| 150 | (2R)-N[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-2-yl]ethyl]-2- (dimethylamino)propanamide 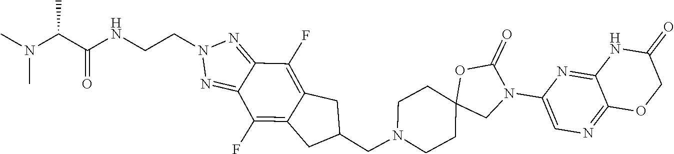 [SMILES: C[C@H](C(=O)NCCn1nc2c(F)c3c(c(F)c2n1)CC(CN1CCC2(CC1)CN(c1cnc4c(n1)NC(=O)CO4)C(=O)O2)C3)N(C)C] ESP [M + H]⁺: 655.3 | Example 41 and CAS# 157431-09-9 |
| 151 | (2R)-2-(dimethylamino)-N-[[8-fluoro-6-[[2-oxo-3-(3-oxo-4H- pyrazino[2,3-b][1,4]oxazin-6-yl)-1-oxa-3,8-diazaspiro[4.5]decan-8- yl]methyl]-1,5,6,7-tetrahydrocyclopenta[f]benzimidazol-2- yl]methyl propanamide 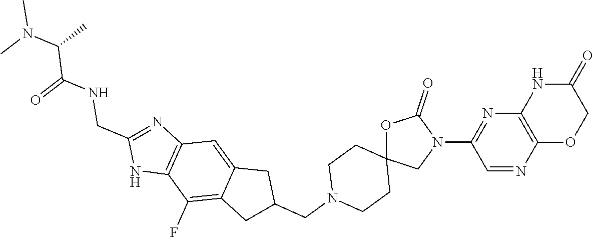 [SMILES: C[C@H](C(=O)NCc1nc2cc3c(c(F)c2[nH]1)CC(CN1CCC2(CC1)CN(c1cnc4c(n1)NC(=O)CO4)C(=O)O2)C3)N(C)C] ESP [M + H]⁺: 622.4 | Intermediate 104 and CAS# 157431-09-9 |
| 152 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]-2- (dimethylamino)-N-methyl-acetamide Enantiomer A 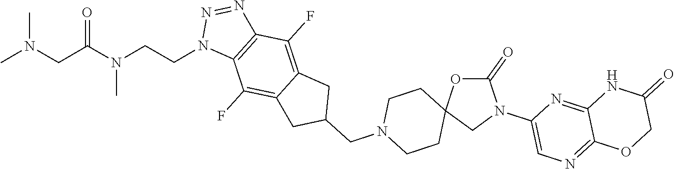 [SMILES: CN(C)CC(=O)N(C)CCn1nnc2c(F)c3c(c(F)c21)CC(CN1CCC2(CC1)CN(c1cnc4c(n1)NC(=O)CO4)C(=O)O2)C3] ESP [M + H]⁺: 655.1 | Example 118 and N,N-dimethylglycine |
| 153 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]-2- (dimethylamino)-N-methyl-acetamide Enantiomer B 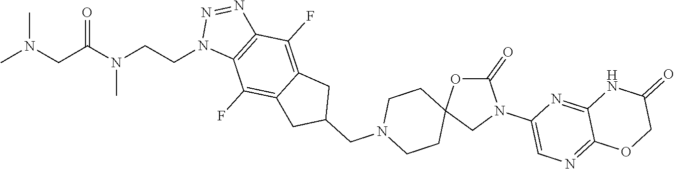 [SMILES: CN(C)CC(=O)N(C)CCn1nnc2c(F)c3c(c(F)c21)CC(CN1CCC2(CC1)CN(c1cnc4c(n1)NC(=O)CO4)C(=O)O2)C3] ESP [M + H]⁺: 655.1 | Example 119 and N,N-dimethylglycine |
| 154 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-2-yl]ethyl]-N-methyl- acetamide 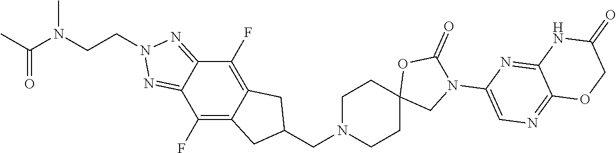 [SMILES: CC(=O)N(C)CCn1nc2c(F)c3c(c(F)c2n1)CC(CN1CCC2(CC1)CN(c1cnc4c(n1)NC(=O)CO4)C(=O)O2)C3] ESP [M + H]⁺: 612.2 | Example 117 and acetic acid |
| 155 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-2-yl]ethyl]-2- (dimethylamino)-N-methyl-acetamide 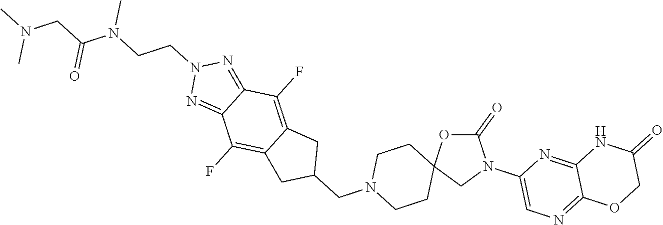 ESP [M + H]⁺: 612.2 | Example 117 and N,N-dimethylglycine |
| 156 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]-N-methyl- acetamide Enantiomer A 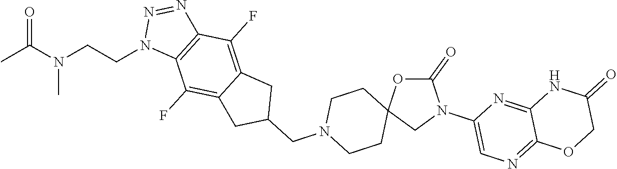 [SMILES: CC(=O)N(C)CCn1nnc2c(F)c3c(c(F)c21)CC(CN1CCC2(CC1)CN(c1cnc4c(n1)NC(=O)CO4)C(=O)O2)C3] ESP [M + H]⁺: 612.1 | Example 118 and acetic acid |
| 157 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]-N-methyl- acetamide Enantiomer B 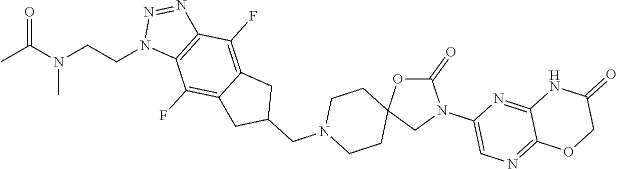 [SMILES: CC(=O)N(C)CCn1nnc2c(F)c3c(c(F)c21)CC(CN1CCC2(CC1)CN(c1cnc4c(n1)NC(=O)CO4)C(=O)O2)C3] ESP [M + H]⁺: 612.1 | Example 119 and acetic acid |

### Example 158

### (2R)—N-[2-[4,8-Difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]pyrrolidine-2-carboxamide

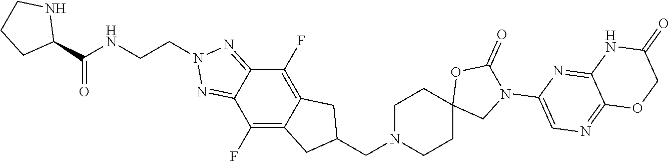 [SMILES: O=C1COc2ncc(N3CC4(CCN(CC5Cc6c(c(F)c7nn(CCNC(=O)[C@H]8CCCN8)nc7c6F)C5)CC4)OC3=O)nc2N1]

### Step 1: tert-butyl (2R)-2-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethylcarbamoyl]pyrrolidine-1-carboxylate

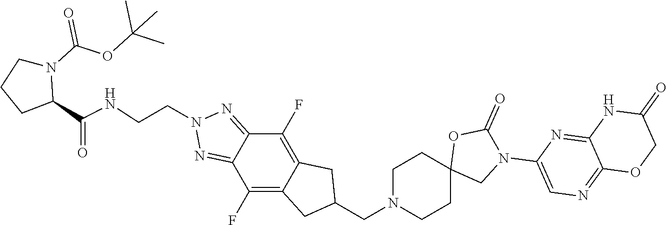 [SMILES: CC(C)(C)OC(=O)N1CCC[C@@H]1C(=O)NCCn1nc2c(F)c3c(c(F)c2n1)CC(CN1CCC2(CC1)CN(c1cnc4c(n1)NC(=O)CO4)C(=O)O2)C3]

The title compound was prepared from 6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 41) and BOC-D-PRO-OH [CAS #37784-17-1] and was obtained as a light yellow solid. MS (ESP) m/z=753.5 [M+H]⁺.

### Step 2: (2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]pyrrolidine-2-carboxamide

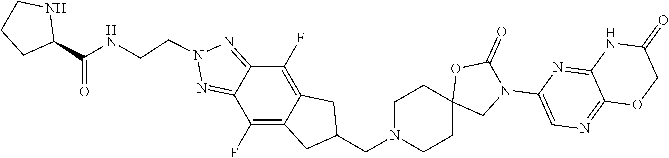 [SMILES: O=C1COc2ncc(N3CC4(CCN(CC5Cc6c(c(F)c7nn(CCNC(=O)[C@H]8CCCN8)nc7c6F)C5)CC4)OC3=O)nc2N1]

A mixture of tert-butyl (2R)-2-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethylcarbamoyl]pyrrolidine-1-carboxylate (143.0 mg, 0.19 mmol, 1.0 eq) and trifluoroacetic acid (0.15 mL, 1.9 mmol, 10.0 eq) in DCM (3 mL) was stirred at RT for 4 h. The mixture was concentrated and purified by flash column chromatography (Silica-NH, 0-20% MeOH/DCM) to give the product which was triturated in MeCN/Et₂O (1:1, 2 mL) to give the title compound (61.0 mg, 0.09 mmol, 49.2% yield) as a white sold. MS (ESP) m/z=653.2 [M+H]⁺.

The following examples were prepared by analogy to Example 158.

| | | Starting |
|---|---|---|
| Ex. | Name, Structure, MS | Materials |
| 159 | N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-2-yl]ethyl]-2- (methylamino)acetamide 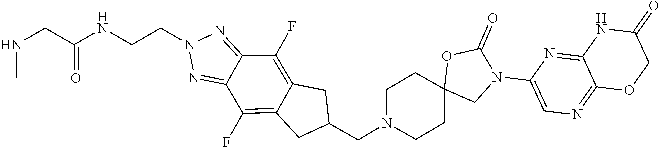 [SMILES: CNCC(=O)NCCn1nc2c(F)c3c(c(F)c2n1)CC(CN1CCC2(CC1)CN(c1cnc4c(n1)NC(=O)CO4)C(=O)O2)C3] ESP [M + H]⁺: 627.3 | Example 41 and CAS# 13734-36-6 |
| 160 | N-[[8-fluoro-6-[[2-oxo-3-(3-oxo-4H-pyrazino[2,3-b][1,4]oxazin-6- yl)-1-oxa-3,8-diazaspiro[4.5]decan-8-yl]methyl]-1,5,6,7- tetrahydrocyclopenta[f]benzimidazol-2-yl]methyl]-2- (methylamino)acetamide; 2,2,2-trifluoroacetic acid 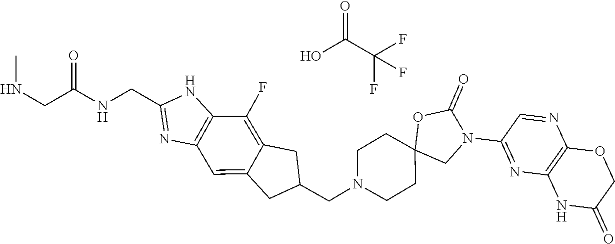 ESP [M + H]⁺: 594.3 | Intermediate 104 and CAS# 13734-36-6 |
| 161 | (2R)-N-[[8-fluoro-6-[[2-oxo-3-(3-oxo-4H-pyrazino[2,3- b][1,4]oxazin-6-yl)-1-oxa-3,8-diazaspiro[4.5]decan-8-yl]methyl]- 1,5,6,7-tetrahydrocyclopenta[f]benzimidazol-2- yl]methyl]pyrrolidine-2-carboxamide; 2,2,2-trifluoroacetic acid 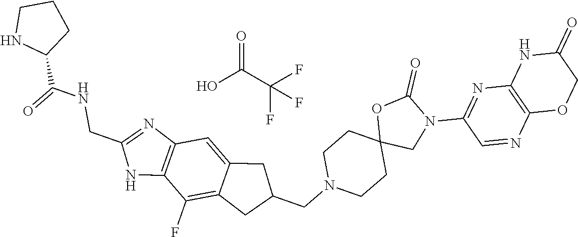 ESP [M + H]⁺: 620.3 | Intermediate 104 and CAS# 37784-17-1 |
| 162 | (2R)-N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]pyrrolidine-2- carboxamide 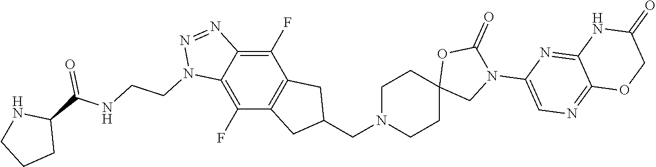 [SMILES: O=C1COc2ncc(N3CC4(CCN(CC5Cc6c(c(F)c7c(nnn7CCNC(=O)[C@H]7CCCN7)c6F)C5)CC4)OC3=O)nc2N1] ESP [M + H]⁺: 653.39 | Example 42 and CAS# 37784-17-1 |
| 163 | (2R,4S)-N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-2-yl]ethyl]-4-hydroxy- pyrrolidine-2-carboxamide 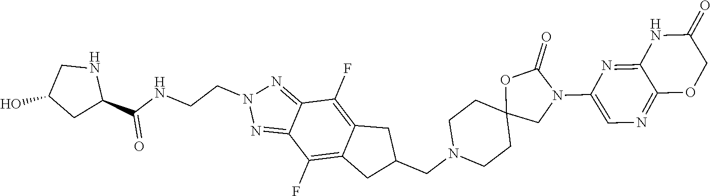 [SMILES: O=C1COc2ncc(N3CC4(CCN(CC5Cc6c(c(F)c7nn(CCNC(=O)[C@H]8C[C@H](O)CN8)nc7c6F)C5)CC4)OC3=O)nc2N1] ESP [M + H]⁺: 669.38 | Example 41 and CAS# 147266-92-0 |
| 164 | (2R,4S)-N-[2-[4,8-difluoro-6-[[2-oxo-3-(3-oxo-4H-pyrazino[2,3- b][1,4]oxazin-6-yl)-1-oxa-3,8-diazaspiro[4.5]decan-8-yl]methyl]-6,7- dihydro-5H-cyclopenta[f]benzotriazol-1-yl]ethyl]-4-hydroxy- pyrrolidine-2-carboxamide 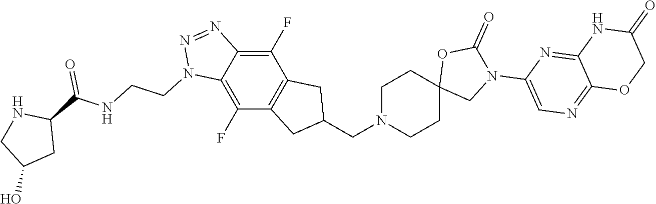 [SMILES: O=C1COc2ncc(N3CC4(CCN(CC5Cc6c(c(F)c7c(nnn7CCNC(=O)[C@H]7C[C@H](O)CN7)c6F)C5)CC4)OC3=O)nc2N1] ESP [M + H]⁺: 669.38 | Example 42 and CAS# 147266-92-0 |

### Example 165

### 2-Amino-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;formic acid

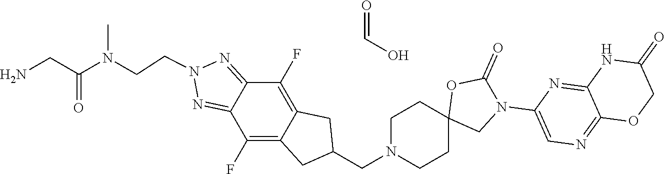 [SMILES: CN(CCn1nc2c(F)c3c(c(F)c2n1)CC(CN1CCC2(CC1)CN(c1cnc4c(n1)NC(=O)CO4)C(=O)O2)C3)C(=O)CN ; O=CO]

The title compound was prepared from 6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one (Example 41) and BOC-glycine by analogy with Example 158, except that formic acid was used in place of trifluoroacetic acid and DCM in Step 2, and was obtained as a yellow solid. MS (ESP) m/z=627.0 [M+H]⁺.

### Example 166

### 2-Amino-N-methyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]acetamide Enantiomer A

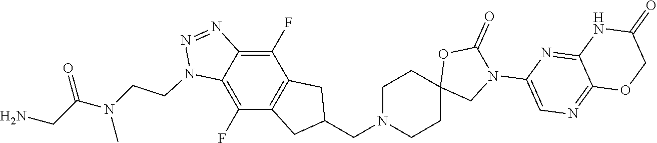 [SMILES: CN(CCn1nnc2c(F)c3c(c(F)c21)CC(CN1CCC2(CC1)CN(c1cnc4c(n1)NC(=O)CO4)C(=O)O2)C3)C(=O)CN]

### and

### Example 167

### 2-Amino-N-methyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]acetamide Enantiomer B

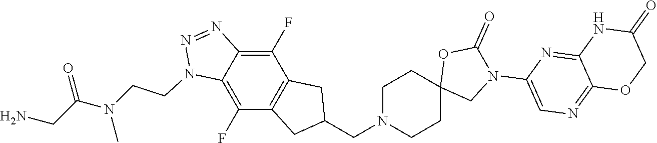 [SMILES: CN(CCn1nnc2c(F)c3c(c(F)c21)CC(CN1CCC2(CC1)CN(c1cnc4c(n1)NC(=O)CO4)C(=O)O2)C3)C(=O)CN]

The racemic title compound was prepared from racemic 6-[8-[[4,8-difluoro-1-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one hydrochloride (prepared by analogy with Example 118) and BOC-glycine by analogy with Example 158, except that hydrochloric acid was used in place of trifluoroacetic acid and EtOH was used in place of DCM in Step 2, and was obtained as a yellow solid. MS (ESP) m/z=627.0 [M+H]⁺. The enantiomers were separated by chiral SFC and were obtained as white solids.

### Example 168

### N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)acetamide

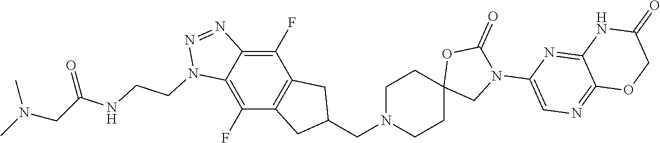 [SMILES: CN(C)CC(=O)NCCn1nnc2c(F)c3c(c(F)c21)CC(CN1CCC2(CC1)CN(c1cnc4c(n1)NC(=O)CO4)C(=O)O2)C3]

The title compound was prepared from N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(methylamino)acetamide (Example 61) by analogy with Example 63, Step 4 and was obtained as a white solid. MS (ESP) m/z=641.3 [M+H]⁺.

### Example 169

### (2R)—N-[[8-Fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-2-(methylamino)propanamide

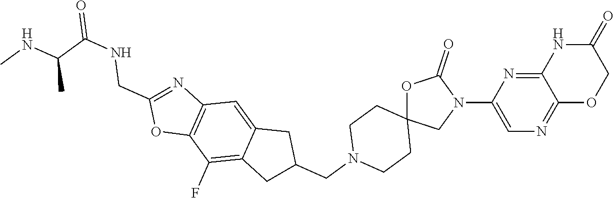 [SMILES: CN[C@H](C)C(=O)NCc1nc2cc3c(c(F)c2o1)CC(CN1CCC2(CC1)CN(c1cnc4c(n1)NC(=O)CO4)C(=O)O2)C3]

The title compound was prepared from 6-(2-oxo-1-oxa-3,8-diazaspiro[4.5]decan-3-yl)-4H-pyrazino[2,3-b][1,4]oxazin-3-one;2,2,2-trifluoroacetic acid (Intermediate 2) and tert-butyl N-[(1R)-2-[(8-fluoro-6-formyl-6,7-dihydro-5H-cyclopenta[f][1,3]benzoxazol-2-yl)methylamino]-1-methyl-2-oxo-ethyl]-N-methyl-carbamate (Intermediate 105) by analogy with Example 9 and was obtained as an off-white solid. MS (ESP) m/z=609.3 [M+H]⁺.

### Example 170

### 6-[2-Oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride Enantiomer A

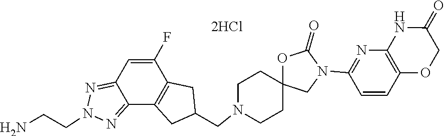 [SMILES: NCCn1nc2cc(F)c3c(c2n1)CC(CN1CCC2(CC1)CN(c1ccc4c(n1)NC(=O)CO4)C(=O)O2)C3]

### and

### Example 171

### 6-[2-Oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride Enantiomer B

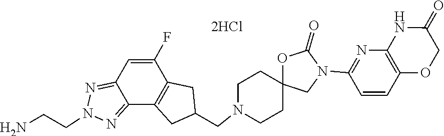 [SMILES: NCCn1nc2cc(F)c3c(c2n1)CC(CN1CCC2(CC1)CN(c1ccc4c(n1)NC(=O)CO4)C(=O)O2)C3]

The title compounds were prepared from Intermediate 3 and Intermediate 73 by analogy with Example 124 and Example 125, Steps 1 and 2, except that the enantiomers were separated at the penultimate step, and were obtained as white solids. MS (ESP) m/z=537.3 [M+H]⁺.

### INTERMEDIATES

### Intermediate 1

### 6-[5-(2-Aminoethyl)-2-oxo-oxazolidin-3-yl]-4H-pyrazino[2,3-b][1,4]oxazin-3-one; 2,2,2-trifluoroacetic acid

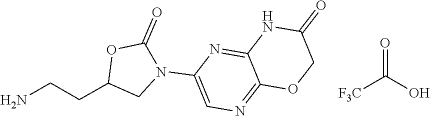 [SMILES: NCCC1CN(c2cnc3c(n2)NC(=O)CO3)C(=O)O1 ; O=C(O)C(F)(F)F]

### Step 1: 6-bromo-4H-pyrazino[2,3-b][1,4]oxazin-3-one

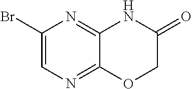 [SMILES: O=C1COc2ncc(Br)nc2N1]

To a solution of methyl glycolate (60.0 mL, 777.31 mmol, 7.86 eq) in 3,6-dibromopyrazin-2-amine [CAS #2376926-19-9] (25.0 g, 98.86 mmol, 1 eq) was added potassium tert-butoxide (34.0 g, 303 mmol, 3.07 eq) at 60° C. under N₂. Methyl glycolate (10.0 mL, 98.86 mmol, 1 eq) was added and the mixture was stirred at 60° C. for 3 h. The reaction mixture was poured into aqueous HCl (1 N, 350 mL) and filtered. The filter cake was washed with H₂O (50 mL×3) and MTBE (50 mL×3), dried under reduced pressure. The aqueous washings were extracted with EtOAc (150 mL×3) and combined with the organic filtrate (MTBE), the mixture was concentrated to give a residue. The filter cake and the residue were combined to give the title compound (23 g, 100 mmol, quant. yield).

### Step 2: 5: 6-bromo-4-(2-trimethylsilylethoxymethyl)pyrazino[2,3-b][1,4]oxazin-3-one

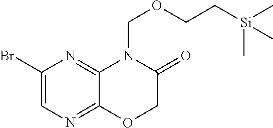 [SMILES: C[Si](C)(C)CCOCN1C(=O)COc2ncc(Br)nc21]

To a mixture of 6-bromo-4H-pyrazino[2,3-b][1,4]oxazin-3-one (15.0 g, 65.21 mmol, 1 eq) and potassium carbonate (27.0 g, 195.36 mmol, 3 eq) in DMF (100 mL) was added 2-(trimethylsilyl)ethoxymethyl chloride (18.0 mL, 101.7 mmol, 1.56 eq) at 0° C. Then the reaction was stirred at 15° C. for 0.5 h. The reaction mixture was diluted with H₂O (600 mL) and the resulting solid was collected by filtration. The solid was washed with water (50 mL×3), dried under reduced pressure to give the title compound (23 g, 63.84 mmol, 97.9% yield) as a yellow solid.

### Step 3: tert-butyl N-[2-[2-oxo-3-[3-oxo-4-(2-trimethylsilylethoxymethyl)pyrazino[2,3-b][1,4]oxazin-6-yl]oxazolidin-5-yl]ethyl]carbamate

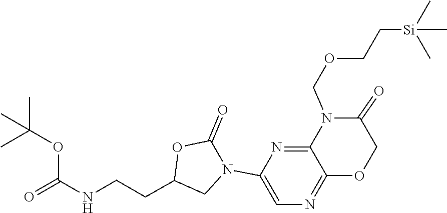 [SMILES: CC(C)(C)OC(=O)NCCC1CN(c2cnc3c(n2)N(COCC[Si](C)(C)C)C(=O)CO3)C(=O)O1]

To a solution of 6-bromo-4-(2-trimethylsilylethoxymethyl)pyrazino[2,3-b][1,4]oxazin-3-one (23.47 g, 65.14 mmol, 1 eq) and tert-butyl N-[2-(2-oxooxazolidin-5-yl)ethyl]carbamate [CAS #2353503-50-9](15.0 g, 65.14 mmol, 1 eq) in 1,4-dioxane (150 mL) was added copper(I) iodide (3722 mg, 19.54 mmol, 0.300 eq) and trans-N,N′-dimethylcyclohexane-1,2-diamine (5560.5 mg, 39.09 mmol, 0.600 eq) and potassium carbonate (27009.8 mg, 195.43 mmol, 3 eq) The mixture was stirred at 100° C. for 3 h. The mixture was concentrated to give a residue. The residue was diluted with EtOAc (200 mL) and washed with brine (100 mL×2). The organic phase was dried over Na₂SO₄, then concentrated to give the crude product. The crude was purified by flash column chromatography (PE/EtOAc=3:1-1:1) and concentrated to give the title compound (26.25 g, 51.51 mmol, 79.1% yield).

### Step 4: 6-[5-(2-aminoethyl)-2-oxo-oxazolidin-3-yl]-4H-pyrazino[2,3-b][1,4]oxazin-3-one; 2,2,2-trifluoroacetic acid

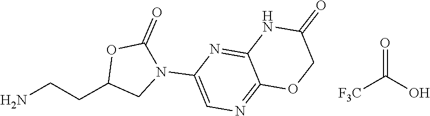 [SMILES: NCCC1CN(c2cnc3c(n2)NC(=O)CO3)C(=O)O1 ; O=C(O)C(F)(F)F]

To a mixture of tert-butyl N-[2-[2-oxo-3-[3-oxo-4-(2-trimethylsilylethoxymethyl)pyrazino[2,3-b][1,4]oxazin-6-yl]oxazolidin-5-yl]ethyl]carbamate (47.2 g, 92.62 mmol, 1 eq) in DCM (100 mL) was added trifluoroacetic acid (472.0 mL, 6126 mmol, 66.15 eq), and then the solution was stirred at 40° C. for 16 h. About 100 mL of solvent was removed by air distillation, and then another 100 mL trifluoroacetic acid was added and the temperature was stirred at 70° C. for 48 h. The solvent was removed by concentration and then the residue was taken up 100 mL water, and then solution was extracted with EtOAc (100 mL×2), the organics were discarded and a solid was formed. The solid was collected by filtration and dried under reduced pressure to give the title compound (14.0 g, 35.6 mmol, 35.36% yield) as a white solid. The filtrate was concentrated to give a crude, which was azeotroped with 100 mL toluene, and the resulting solid was triturated in 100 mL MeCN, and then the solid was collected by filtration and dried under reduced pressure to give further title compound (15.3 g, 38.9 mmol, 38.6% yield) as an off-white solid. MS (ESP) m/z=280.1 [M+H]⁺.

Intermediate 2

### 6-(2-Oxo-1-oxa-3,8-diazaspiro[4.5]decan-3-yl)-4H-pyrazino[2,3-b][1,4]oxazin-3-one; 2,2,2-trifluoroacetic acid

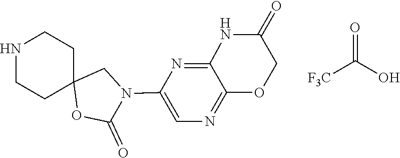 [SMILES: O=C1COc2ncc(N3CC4(CCNCC4)OC3=O)nc2N1 ; O=C(O)C(F)(F)F]

The title compound was prepared by analogy to Intermediate 1 using tert-butyl 2-oxo-1-oxa-3,8-diazaspiro[4.5]decane-8-carboxylate [CAS #169206-55-7] in place of tert-butyl N-[2-(2-oxooxazolidin-5-yl)ethyl]carbamate and was obtained as a light yellow solid. MS (ESP) m/z=306.1 [M+H]⁺.

### Intermediate 3

### 6-(2-Oxo-1-oxa-3,8-diazaspiro[4.5]decan-3-yl)-4H-pyrido[3,2-b][1,4]oxazin-3-one hydrochloride

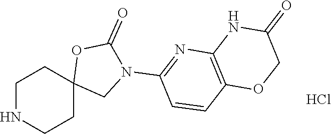 [SMILES: O=C1COc2ccc(N3CC4(CCNCC4)OC3=O)nc2N1 ; Cl]

### Step 1: tert-butyl 3-[5-(2-ethoxy-2-oxo-ethoxy)-6-nitro-2-pyridyl]-2-oxo-1-oxa-3,8-diazaspiro[4.5]decane-8-carboxylate

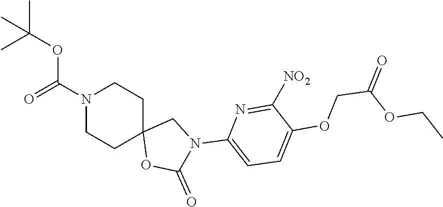 [SMILES: CCOC(=O)COc1ccc(N2CC3(CCN(C(=O)OC(C)(C)C)CC3)OC2=O)nc1[N+](=O)[O-]]

To a solution of potassium carbonate (2.43 g, 17.56 mmol, 1.5 eq) and N,N-dimethyl-ethylenediamine (412.49 mg, 4.68 mmol, 0.400 eq) in 1,4-dioxane (40 mL) was added copper (I) iodide (445.84 mg, 2.34 mmol, 0.200 eq), tert-butyl 2-oxo-1-oxa-3,8-diazaspiro[4.5]decane-8-carboxylate [CAS #169206-55-7](3.0 g, 11.71 mmol, 1 eq) and ethyl 2-[(6-bromo-2-nitro-3-pyridyl)oxy]acetate [CAS #443956-09-0] (3.57 g, 11.71 mmol, 1 eq). The mixture was degassed with nitrogen three times and stirred at 100° C. for 2 h. The reaction was filtered and the filtrate was concentrated to dryness. The residue was diluted with EtOAc (10 mL). The organic layer was washed with water (3 mL) and brine (3 mL), dried over anhydrous Na₂SO₄, concentrated under reduced pressure to afford the crude product which was purified by silica column chromatography (PE/EtOAc=1:1) to give the title compound (5.0 g, 10.41 mmol, 88.91% yield) as a yellow solid.

### Step 2: tert-butyl 2-oxo-3-(3-oxo-4H-pyrido[3,2-b][1,4]oxazin-6-yl)-1-oxa-3,8-diazaspiro[4.5]decane-8-carboxylate

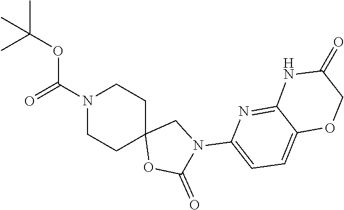 [SMILES: CC(C)(C)OC(=O)N1CCC2(CC1)CN(c1ccc3c(n1)NC(=O)CO3)C(=O)O2]

To a solution of tert-butyl 3-[5-(2-ethoxy-2-oxo-ethoxy)-6-nitro-2-pyridyl]-2-oxo-1-oxa-3,8-diazaspiro[4.5]decane-8-carboxylate (5.0 g, 10.41 mmol, 1 eq) in acetic acid (50 mL) was added iron (2.91 g, 52.03 mmol, 5 eq) and the mixture stirred for 2 h at 60° C. The reaction was filtered and concentrated to dryness. The residue was diluted with EtOAc (50 mL). The organic layer was washed with water (30 mL) and brine (30 mL), dried over anhydrous Na₂SO₄, concentrated under reduced pressure to afford crude product. The residue was purified by silica column chromatography (PE/EtOAc=1:1) to give the title compound (4 g, 9.89 mmol, 95.04% yield) as a yellow solid. MS (ESP) m/z=405.2 [M+H]⁺.

### Step 3: 6-(2-oxo-1-oxa-3,8-diazaspiro[4.5]decan-3-yl)-4H-pyrido[3,2-b][1,4]oxazin-3-one hydrochloride

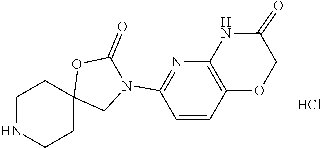 [SMILES: O=C1COc2ccc(N3CC4(CCNCC4)OC3=O)nc2N1 ; Cl]

A solution of tert-butyl 2-oxo-3-(3-oxo-4H-pyrido[3,2-b][1,4]oxazin-6-yl)-1-oxa-3,8-diazaspiro[4.5]decane-8-carboxylate (500.0 mg, 1.24 mmol, 1 eq) in 4M hydrochloric acid in methanol (20.0 mL, 80 mmol, 64.7 eq) was stirred for 4 h at 25° C. The mixture was concentrated under reduced pressure. To the residue was added EtOAc (20 mL) and the mixture filtered. The filter cake was washed with EtOAc (10 mL) and then dried to give the title compound (238.8 mg, 0.700 mmol, 55.5% yield) as a grey solid. The combined filtrate and washing were concentrated to give additional title compound (150 mg, 0.440 mmol, 35.0% yield) as a yellow solid. MS (ESP): m/z=305.0 [M+H]⁺.

### Intermediate 4

### 6-(2-Amino-6-oxo-5-oxa-7-azaspiro[3.4]octan-7-yl)-4H-pyrazino[2,3-b][1,4]oxazin-3-one;2,2,2-trifluoroacetic acid

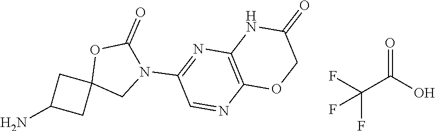

### Step 1: benzyl N-(1-oxaspiro [2.3]hexan-5-yl)carbamate

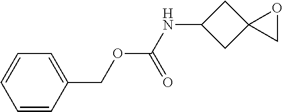 [SMILES: O=C(NC1CC2(CO2)C1)OCc1ccccc1]

3-Chloroperoxybenzoic acid (787.95 mg, 4.57 mmol, 1.24 eq) was added to a solution of benzyl N-(3-methylenecyclobutyl)carbamate [CAS #130368-98-8] (800.0 mg, 3.68 mmol, 14 eq in DCM (7.87 mL) at 0° C. The reaction mixture was stirred for 30 min. at 0° C., then allowed to warm to RT and stirred at RT for 2 h. More 3-chloroperoxybenzoic acid (127.09 mg, 0.740 mmol, 0.200 eq) was added and the mixture was stirred at RT for 2 h. The reaction mixture was diluted with DCM and washed with sat. aq. Na₂SO₃, sat. aq. NaHCO₃ (2×) and brine. The organic layer was dried, filtered and concentrated to afford the crude title compound (860 mg, 3.69 mmol, quant. yield) as a white solid (as mixture of cis:trans isomers). MS (ESP): m/z=234.4 [M+H]⁺.

### Step 2: benzyl N-[3-(azidomethyl)-3-hydroxy-cyclobutyl]carbamate

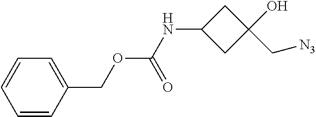 [SMILES: [N-]=[N+]=NCC1(O)CC(NC(=O)OCc2ccccc2)C1]

Sodium azide (479.37 mg, 7.37 mmol, 2 eq) and ammonium chloride (394.42 mg, 7.37 mmol, 2 eq) were added to a solution of benzyl N-(1-oxaspiro[2.3]hexan-5-yl)carbamate (860.0 mg, 3.69 mmol, 1 eq) in methanol (14.46 mL) and the mixture was stirred at RT overnight. The reaction mixture was diluted with EtOAc and and washed with water and sat. aq. NaHCO₃. The organic phase was dried, filtered and concentrated under vacuum to give the crude title compound (1020 mg, 3.69 mmol, quant. yield) as light yellow oil. MS (ESP): m/z=277 [M+H]⁺.

### Step 3: benzyl N-[3-(aminomethyl)-3-hydroxy-cyclobutyl]carbamate

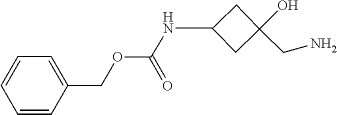 [SMILES: NCC1(O)CC(NC(=O)OCc2ccccc2)C1]

Triphenylphosphine (1934.73 mg, 7.38 mmol, 2 eq) was added to a solution of benzyl N-[3-(azidomethyl)-3-hydroxy-cyclobutyl]carbamate (1019.0 mg, 3.69 mmol, 1 eq) in THF (12 mL)/water (12 mL). The mixture was stirred at RT for 18 h, then diluted with sat. aq. NaHCO₃ solution and extracted with EtOAc (2×) and EtOAc/MeOH 9:1 (2×). The combined organic phase was concentrated under vacuum and the crude was purified by flash column chromatography (0-10% MeOH in DCM) to give the title compound (783 mg, 3.13 mmol, 85% yield) as a white gum. MS (ESP): m/z=251.3 [M+H]⁺. Step 4: benzyl N-(6-oxo-5-oxa-7-azaspiro[3.4]octan-2-yl)carbamate

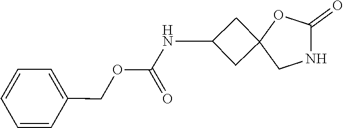 [SMILES: O=C(NC1CC2(CNC(=O)O2)C1)OCc1ccccc1]

To a stirred solution of benzyl N-[3-(aminomethyl)-3-hydroxy-cyclobutyl]carbamate (273.0 mg, 1.09 mmol, 1 eq) in THF (6.54 mL) and DMF (1.31 mL) cooled to 0° C., N,N′-carbonyldiimidazole (185.71 mg, 1.15 mmol, 1.05 eq) was added portionwise. The mixture was stirred at 0° C. for 20 min. The mixture was allowed to reach RT and stirred at RT for 20 h. The mixture was concentrated under reduced pressure then diluted with EtOAc and washed with sat. aq. NH₄Cl. The organic phase was dried over Na₂SO₄ and concentrated in vacuo. Water was added to the residue and the resulting white precipitate was filtered, washed with water and dried under vacuum to afford the title compound (268 mg, 0.968 mmol, 89% yield). MS (ESP): m/z=277.2 [M+H]⁺.

### Step 5: benzyl N-[6-oxo-7-[3-oxo-4-(2-trimethylsilylethoxymethyl)pyrazino[2,3-b][1,4]oxazin-6-yl]-5-oxa-7-azaspiro[3.4]octan-2-yl]carbamate

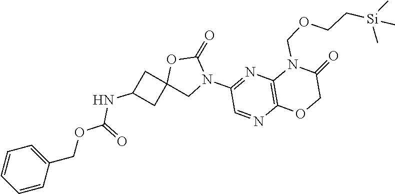 [SMILES: C[Si](C)(C)CCOCN1C(=O)COc2ncc(N3CC4(CC(NC(=O)OCc5ccccc5)C4)OC3=O)nc21]

A mixture of benzyl N-(6-oxo-5-oxa-7-azaspiro[3.4]octan-2-yl)carbamate (660.0 mg, 2.39 mmol, 1 eq), 6-bromo-4-(2-trimethylsilylethoxymethyl)pyrazino[2,3-b][1,4]oxazin-3-one (860.63 mg, 2.39 mmol, 1 eq), potassium carbonate (990.47 mg, 7.17 mmol, 3 eq), copper(I) iodide (136.48 mg, 0.720 mmol, 0.300 eq) and (R,R)-(−)-N,N′-dimethyl-1,2-cyclohexanediamine (203.87 mg, 1.43 mmol, 0.600 eq) was degassed by purging the system with vacuum/nitrogen atmosphere (three times). 2-Methyltetrahydrofuran (15.93 mL) was added, then the mixture was degassed and heated at 80° C. for 6 h. The reaction mixture was diluted water and extracted with EtOAc. The organic phase was dried, filtered and volatiles were removed under reduced pressure. The crude was purified by silica cartridge chromatography (10-60% EtOAc/cyclohexane) to afford the title compound (730 mg, 1.31 mmol, 55% yield) as a light yellow solid. MS (ESP): m/z=556.2 [M+H]⁺.

### Step 6: 6-(2-amino-6-oxo-5-oxa-7-azaspiro[3.4]octan-7-yl)-4-(2-trimethylsilylethoxymethyl)pyrazino[2,3-b][1,4]oxazin-3-one

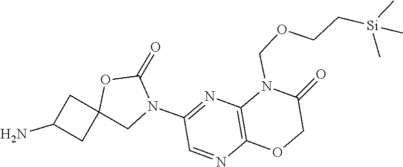 [SMILES: C[Si](C)(C)CCOCN1C(=O)COc2ncc(N3CC4(CC(N)C4)OC3=O)nc21]

A stirred solution of benzyl N-[6-oxo-7-[3-oxo-4-(2-trimethylsilylethoxymethyl)pyrazino[2,3-b][1,4]oxazin-6-yl]-5-oxa-7-azaspiro[3.4]octan-2-yl]carbamate (370.0 mg, 0.670 mmol, 1 eq) in THF (60.47 mL)/ethanol (4.32 mL) was submitted to 3 nitrogen/vacuum cycles. Palladium on carbon 10% wt (106.29 mg, 0.100 mmol, 0.150 eq) was added and the mixture was submitted to 3 nitrogen/vacuum cycles then to 3 hydrogen/vacuum cycles, then the reaction mixture was stirred under 1 atmosphere of hydrogen at RT for 16 h. The reaction mixture was filtered, washed with ethanol and then the filtrate was concentrated under reduced pressure to afford the crude title compound (303 mg, 0.720 mmol, quant. yield) as a brown solid. MS (ESP): m/z=422.2 [M+H]⁺.

### Step 7: 6-(2-amino-6-oxo-5-oxa-7-azaspiro[3.4]octan-7-yl)-4H-pyrazino[2,3-b][1,4]oxazin-3-one;2,2,2-trifluoroacetic acid

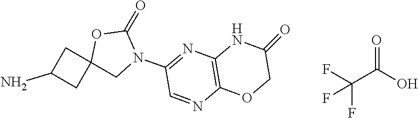 [SMILES: NC1CC2(C1)CN(c1cnc3c(n1)NC(=O)CO3)C(=O)O2 ; O=C(O)C(F)(F)F]

A solution of 6-(2-amino-6-oxo-5-oxa-7-azaspiro[3.4]octan-7-yl)-4-(2-trimethylsilylethoxymethyl)pyrazino[2,3-b][1,4]oxazin-3-one (280.0 mg, 0.660 mmol, 1 eq) in trifluoroacetic acid (6.23 mL, 80.83 mmol, 121.68 eq) was stirred at 60° C. for 15 h. Volatiles were removed under reduced pressure to give the crude, impure title compound (313 mg) as a dark brown solid which was used in the next reaction without further purification. MS (ESP): m/z=292.2 [M+H]⁺.

### Intermediate 5

### Ethyl 5,6-diamino-4,7-difluoro-indane-2-carboxylate

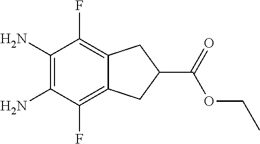 [SMILES: CCOC(=O)C1Cc2c(F)c(N)c(N)c(F)c2C1]

### Step 1: diethyl 4,7-difluoroindane-2,2-dicarboxylate

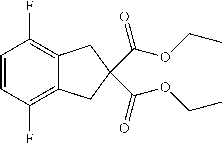 [SMILES: CCOC(=O)C1(C(=O)OCC)Cc2c(F)ccc(F)c2C1]

To a solution of diethyl malonate (3.39 mL, 22.34 mmol, 1 eq) in THF (709.14 mL), potassium tert-butoxide (5.26 g, 46.91 mmol, 2.1 eq) was added at 0° C. The mixture was stirred at the same temperature for 1 h, then 2,3-bis(bromomethyl)-1,4-difluoro-benzene [CAS #912999-77-0] (6.7 g, 22.34 mmol, 1 eq) dissolved in THF (354.57 mL) was added and the mixture was stirred at 60° C. for 24 h. The reaction was concentrated in vacuo. The mixture was partioned between water and EtOAC. The phases were separated and the aqueous phase was extracted with EtOAc (×1). The combined organic phase was dried over Na₂SO₄, filtered and concentrated under vacuum to afford the crude title compound (6.7 g, 22.46 mmol, quant. yield) which was used in the following reaction without further purification.

### Step 2: ethyl 4,7-difluoroindane-2-carboxylate

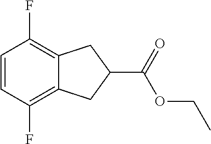 [SMILES: CCOC(=O)C1Cc2c(F)ccc(F)c2C1]

A mixture of diethyl 4,7-difluoroindane-2,2-dicarboxylate (3.969 g, 13.3 mmol, 1 eq), water (3.94 mL) and lithium chloride (1.13 g, 26.6 mmol, 2 eq) in DMSO (26.3 mL) was stirred at 170° C. for 17 h. The reaction was cooled to room temperature and diluted with EtOAc and washed with water (×1). The combined organic phase was dried over Na₂SO₄ and concentrated in vacuo to afford the crude product which was purified by flash column chromatography (70 g silica, 1% to 10% EtOAc/heptane) to afford the title compound (2.047 g, 9.06 mmol, 66% yield) as a light yellow oil. MS (ESP) m/z=227.2 [M+H]⁺.

### Step 3: ethyl 4,7-difluoro-5-nitro-indane-2-carboxylate

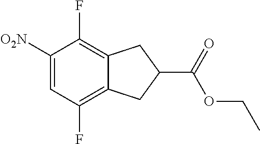 [SMILES: CCOC(=O)C1Cc2c(F)cc([N+](=O)[O-])c(F)c2C1]

To a solution of ethyl 4,7-difluoroindane-2-carboxylate (1.02 g, 4.52 mmol, 1 eq) in sulfuric acid (13.97 mL, 262.03 mmol, 58 eq) at 0° C., nitric acid (0.29 mL, 4.52 mmol, 1 eq) was added and the mixture was stirred for 30 min. at 0° C. The mixture was poured into water and ice at 0° C. then extracted with EtOAc. The organic phase was washed with brine, dried over Na₂SO₄, filtered and concentrated under vacuum. The resulting crude material was purified by by flash chromatography (10 g, cyclohexane/EtOAc, 100% cyclohexane to 70:30) to obtain the title compound (1240 mg, 4.57 mmol, quant. yield) as a light yellow liquid. MS (ESP) m/z=272.1 [M+H]⁺.

### Step 4: ethyl 5-amino-4,7-difluoro-indane-2-carboxylate

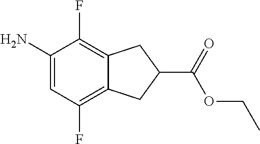 [SMILES: CCOC(=O)C1Cc2c(F)cc(N)c(F)c2C1]

Ethyl 4,7-difluoro-5-nitro-indane-2-carboxylate (1300.0 mg, 4.79 mmol, 1 eq) was stirred in ethanol (47.93 mL) under an atmosphere of hydrogen (765 mm Hg) in the presence of 10% palladium (510.09 mg, 0.480 mmol, 0.100 eq) on carbon at 25° C. for 16 h. The reaction was filtered through a Celite® pad and concentrated in vacuo to afford the title compound (1115 mg, 4.62 mmol, 96.43% yield) as a pink oil. MS (ESP) m/z=242.1 [M+H]⁺.

### Step 5: ethyl 5-acetamido-4,7-difluoro-indane-2-carboxylate

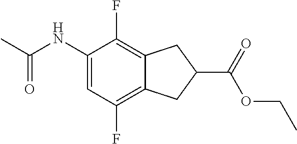 [SMILES: CCOC(=O)C1Cc2c(F)cc(NC(C)=O)c(F)c2C1]

To a stirred solution of ethyl 5-amino-4,7-difluoro-indane-2-carboxylate (375.0 mg, 1.55 mmol, 1 eq) in DCM (15.55 mL), acetic anhydride (154.01 uL, 1.63 mmol, 1.05 eq) was added and the mixture was stirred for 2.5 h at 25° C. A saturated solution of NaHCO₃ (aq.) was added to the reaction. The phases were separated and the organic phase was dried over Na₂SO₄ and concentrated in vacuo to afford the title compound (430 mg, 1.52 mmol, 97.65% yield) as a red solid. MS (ESP) m/z=284.2 [M+H]⁺.

### Step 6: ethyl 5-acetamido-4,7-difluoro-6-nitro-indane-2-carboxylate

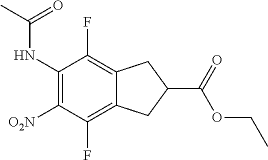 [SMILES: CCOC(=O)C1Cc2c(F)c(NC(C)=O)c([N+](=O)[O-])c(F)c2C1]

To a solution of ethyl 5-acetamido-4,7-difluoro-indane-2-carboxylate (430.0 mg, 1.52 mmol, 1 eq) in sulfuric acid (4.69 mL, 88.04 mmol, 58 eq) at 0° C., nitric acid (106.37 uL, 1.67 mmol, 1.1 eq) was added and the mixture was stirred for 80 min. at 0° C. The reaction was slowly added to a saturated aqueous solution of NaHCO₃ at 0° C. The aqueous phase was extracted with DCM (×1), organic phase was dried over Na₂SO₄ and concentrated in vacuo to afford the title compound (490.0 mg, 1.50 mmol, 98.3% yield) as a brown gum. MS (ESP) m/z=329.4 [M+H]⁺.

### Step 7: ethyl 5-amino-4,7-difluoro-6-nitro-indane-2-carboxylate

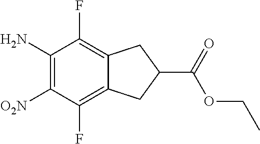 [SMILES: CCOC(=O)C1Cc2c(F)c(N)c([N+](=O)[O-])c(F)c2C1]

To a stirred solution of ethyl 5-acetamido-4,7-difluoro-6-nitro-indane-2-carboxylate (1170.0 mg, 3.56 mmol, 1 eq) in ethanol (35.64 mL), sulfuric acid (2.09 mL, 39.2 mmol, 11 eq) was added and the mixture was stirred at 78° C. for 16 h. The reaction was concentrated in vacuo and then slowly added to a stirred aqueous solution of NaHCO₃ cooled down to 0° C. The aqueous phase was extracted with DCM (×2). The combined organic phase was dried over Na₂SO₄ and concentrated in vacuo. The mixture was purified by silica cartridge chromatography (cyclohexane/EtOAc from 97:3 to 70:30) to afford the title compound (850 mg, 2.97 mmol, 83.32% yield), as a yellow gum. MS (ESP) m/z=287.2 [M+H]⁺.

### Step 8: ethyl 5,6-diamino-4,7-difluoro-indane-2-carboxylate

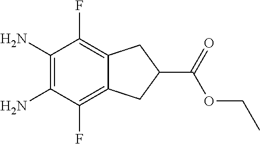 [SMILES: CCOC(=O)C1Cc2c(F)c(N)c(N)c(F)c2C1]

Ethyl 5-amino-4,7-difluoro-6-nitro-indane-2-carboxylate (850.0 mg, 2.97 mmol, 1 eq) was stirred in ethanol (40 mL) under an atmosphere of hydrogen (765 mmHg) in the presence of 10% palladium (316.03 mg, 0.300 mmol, 0.100 eq) on carbon at 25° C. for 2 h. The reaction was filtered through a Celite® pad and concentrated in vacuo to give the title compound (690 mg, 2.69 mmol, 90.67% yield) as a light yellow solid. MS (ESP) m/z=258.1 [M+H]⁺.

### Intermediate 6

### Methyl 5,6-diamino-4,7-difluoro-indane-2-carboxylate

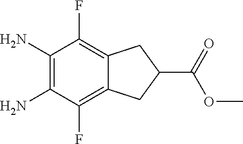 [SMILES: COC(=O)C1Cc2c(F)c(N)c(N)c(F)c2C1]

### Step 1: methyl 4,7-difluoro-5-nitro-indane-2-carboxylate

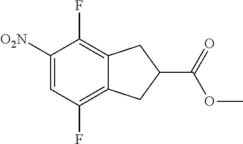 [SMILES: COC(=O)C1Cc2c(F)cc([N+](=O)[O-])c(F)c2C1]

To a solution of methyl 4,7-difluoroindane-2-carboxylate (Intermediate 6) (35.6 g, 167.77 mmol, 1 eq) in sulfuric acid (518.7 mL, 9731 mmol, 58 eq) at 0° C., nitric acid (10.69 mL, 167.77 mmol, 1 eq) was added and the mixture was stirred for 30 min. at 0° C. The mixture was poured into ice water and then extracted with EtOAc (700 mL×3). The combined organic phase was dried over Na₂SO₄, filtered and concentrated under vacuum to obtain the title compound (44.7 g, 173.8 mmol, quant. yield) as a light brown oil. MS (ESP) m/z=258.1 [M+H]⁺.

### Step 2: methyl 5-amino-4,7-difluoro-indane-2-carboxylate

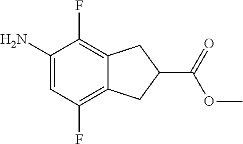 [SMILES: COC(=O)C1Cc2c(F)cc(N)c(F)c2C1]

Methyl 4,7-difluoro-5-nitro-indane-2-carboxylate (44.7 g, 173.8 mmol, 1 eq) was stirred in methanol (555.28 mL) under an atmosphere of hydrogen (100 psi) in the presence of 10% palladium on carbon (50% wet) (18.5 g, 8.69 mmol, 0.050 eq) at 25° C. for 16 h. The catalyst was filtered off and the organic phase concentrated in vacuo to afford the title compound (34.6 g, 152.28 mmol, 87.62% yield) as a purple oil. MS (ESP) m/z=228.1 [M+H]⁺.

### Step 3: methyl 5-acetamido-4,7-difluoro-indane-2-carboxylate

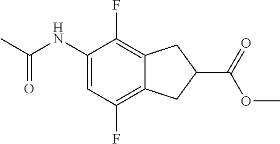 [SMILES: COC(=O)C1Cc2c(F)cc(NC(C)=O)c(F)c2C1]

To a stirred solution of methyl 5-amino-4,7-difluoro-indane-2-carboxylate (34.6 g, 152.28 mmol, 1 eq) in DCM (1437 mL), acetic anhydride (15.09 mL, 159.9 mmol, 1.05 eq) was added and the mixture was stirred for 16 h at 25° C. A saturated solution of NaHCO₃ was added to the reaction. The phases were separated and the organic phase was dried over Na₂SO₄ and concentrated in vacuo to afford the title compound (36.4 g, 135.2 mmol, 88.78% yield) as a pink solid. MS (ESP) m/z=270.2 [M+H]⁺.

### Step 4: methyl 5-acetamido-4,7-difluoro-6-nitro-indane-2-carboxylate

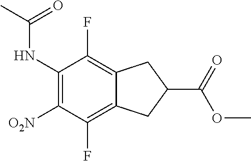 [SMILES: COC(=O)C1Cc2c(F)c(NC(C)=O)c([N+](=O)[O-])c(F)c2C1]

To a solution of methyl 5-acetamido-4,7-difluoro-indane-2-carboxylate (36.4 g, 135.2 mmol, 1 eq) in sulfuric acid (418 mL, 7841 mmol, 58 eq) at 0° C., nitric acid (9.47 mL, 148.71 mmol, 1.1 eq) was added and the mixture was stirred for 30 min. at 0° C. The reaction was slowly added to ice-water and extracted with DCM (×2). The combined organic phase was dried over Na₂SO₄ and concentrated in vacuo to afford the title compound (36.4 g, 115.8 mmol, 85.68% yield) as a light brown solid. ¹H NMR (400 MHz, DMSO) δ 10.22 (s, 1H), 3.73-3.53 (m, 4H), 3.43-3.22 (m, 4H), 2.04 (s, 3H).

### Step 5: methyl 5-amino-4,7-difluoro-6-nitro-indane-2-carboxylate

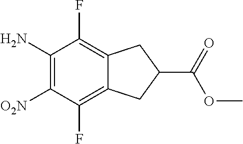 [SMILES: COC(=O)C1Cc2c(F)c(N)c([N+](=O)[O-])c(F)c2C1]

To a stirred solution of methyl 5-acetamido-4,7-difluoro-6-nitro-indane-2-carboxylate (36.4 g, 115.84 mmol, 1 eq) in methanol (1000 mL), sulfuric acid (67.9 mL, 1274 mmol, 11 eq) was added and the mixture was stirred at 65° C. for 16 h. The reaction was concentrated in vacuo and then slowly added to ice-cold sat. aq. NaHCO₃. The aqueous phase was extracted with DCM (1000 mL×3). The combined organic phase was washed with water (500 mL), dried over Na₂SO₄ and concentrated in vacuo to afford the title compound (32.2 g, 118.3 mmol, quant. yield) as a brown solid. MS (ESP) m/z=273.1 [M+H]⁺.

### Step 6: methyl 5,6-diamino-4,7-difluoro-indane-2-carboxylate

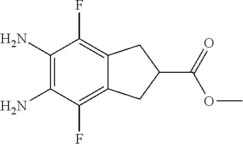 [SMILES: COC(=O)C1Cc2c(F)c(N)c(N)c(F)c2C1]

Methyl 5-amino-4,7-difluoro-6-nitro-indane-2-carboxylate (27.2 g, 99.93 mmol, 1 eq) was suspended in methanol (358.16 mL) and the mixture was stirred under an atmosphere of hydrogen (100 psi) in the presence of 10% palladium on carbon (50% wet) (6380 mg, 3 mmol, 0.030 eq) at 25° C. for 4 h. The catalyst was filtered off and the organic phase was concentrated in vacuo to afford the title compound (23.42 g, 96.69 mmol, 96.8% yield) as a brown solid. MS (ESP) m/z=243.1 [M+H]⁺.

### Intermediate 7

### Ethyl 5,6-diamino-4-fluoro-indane-2-carboxylate

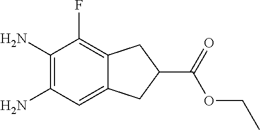 [SMILES: CCOC(=O)C1Cc2cc(N)c(N)c(F)c2C1]

### Route A:

### Step 1: ethyl 4-fluoro-1-oxo-indane-2-carboxylate

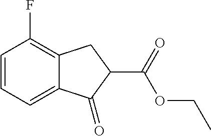 [SMILES: CCOC(=O)C1Cc2c(F)cccc2C1=O]

A mixture of sodium hydride, 60% in oil (7.99 g, 199.8 mmol, 3 eq) in toluene (120 mL) was cooled to 0° C. then diethyl carbonate (18.56 mL, 153.18 mmol, 2.3 eq) was added dropwise. The mixture was heated to 120° C. and a solution of 4-fluoroindan-1-one (10.0 g, 66.6 mmol, 1 eq) in toluene (120 mL) was added dropwise over 1.5 h. The mixture was left stirring at 120° C. for 1 h. It was allowed to reach RT then the reaction mixture was slowly poured into cooled NH₄Cl (sat. aq.) and acetic acid was added to adjust the pH to 5. The product was extracted with EtOAc, the phases were separated, the organic phase was dried over Na₂SO₄, filtered and concentrated. The obtained crude (18.1 g) was purified by silica cartridge chromatography (200 g, 0-12% EtOAc/cyclohexane). The fractions were concentrated under reduced pressure to give the title compound (13.86 g, 62.37 mmol, 91.78% yield) as a yellow oil. MS (ESP) m/z=223.1 [M+H]⁺.

### Step 2: ethyl 4-fluoroindane-2-carboxylate

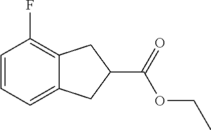

A solution of ethyl 4-fluoro-1-oxo-indane-2-carboxylate (13.2 g, 59.4 mmol, 1 eq) in ethanol (480 mL) and HCl (4.5 mL) was stirred for 4 h under hydrogen atmosphere in presence of Pd/C (12.64 g, 11.88 mmol, 0.200 eq). The mixture was filtered and washed with EtOAc then it was concentrated in vacuo. It was diluted with EtOAc, washed with water (2×) and brine (1×) and concentrated in vacuo to afford the title compound (12.7 g, 60.99 mmol, quant. yield). MS (ESP) m/z=209.1 [M+H]⁺.

### Step 3: ethyl 4-fluoro-5-(4,4,5,5-tetramethyl-1,3,2-dioxaborolan-2-yl)indane-2-carboxylate; ethyl 4-fluoro-6-(4,4,5,5-tetramethyl-1,3,2-dioxaborolan-2-yl)indane-2-carboxylate; ethyl 4-fluoro-7-(4,4,5,5-tetramethyl-1,3,2-dioxaborolan-2-yl)indane-2-carboxylate

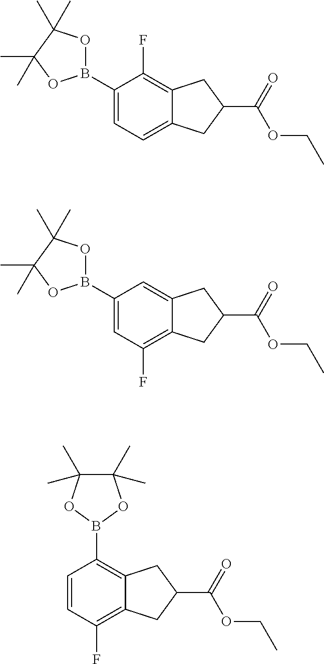

A stirred solution of 4,4,5,5-tetramethyl-1,3,2-dioxaborolane (27.09 mL, 186.72 mmol, 2 eq) and 3,4,7,8-tetramethyl-1,10-phenanthroline (882.46 mg, 3.73 mmol, 0.040 eq) was degassed 3 times and (1,5-cyclooctadiene)(methoxy)iridium(I) dimer (1237.67 mg, 1.87 mmol, 0.020 eq) was added under N₂ atmosphere. N₂ was bubbled into the reaction mixture for 15 minutes, then ethyl 4-fluoroindane-2-carboxylate (19.44 g, 93.36 mmol, 1 eq) was added over 20 minutes. The mixture was stirred at 65° C. for 4 h and then cooled to RT. EtOAc (200 mL) was added, the mixture was stirred for 30 minutes, the catalyst was filtered off and solvent was evaporated under reduced pressure to obtain the crude material containing the title compound mixture (35 g, 104.73 mmol, quant. yield) which was used directly in the next step. MS (ESP) m/z=335.3 [M+H]⁺.

### Step 4: ethyl 4-fluoro-6-hydroxy-indane-2-carboxylate;ethyl 4-fluoro-7-hydroxy-indane-2-carboxylate

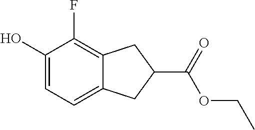 [SMILES: CCOC(=O)C1Cc2ccc(O)c(F)c2C1]

To a stirred solution of ethyl 4-fluoro-5-(4,4,5,5-tetramethyl-1,3,2-dioxaborolan-2-yl)indane-2-carboxylate; ethyl 4-fluoro-6-(4,4,5,5-tetramethyl-1,3,2-dioxaborolan-2-yl)indane-2-carboxylate; ethyl 4-fluoro-7-(4,4,5,5-tetramethyl-1,3,2-dioxaborolan-2-yl)indane-2-carboxylate (3.24 g, 9.7 mmol, 1 eq), in THF (10 mL), sodium perborate monohydrate (2.9 g, 29.1 mmol, 3 eq) in water (10 mL) was added. The mixture was stirred for 2 h. Then water was added and the product extracted with EtOAc. The organic phase was dried over Na₂SO₄, filtered and concentrated. The crude material was purified by silica cartridge chromatography (200 g, 0-20% EtOAc/cyclohexane) to give as the first eluted isomer the title compound (253.2 mg, 1.13 mmol, 11.64% yield) as a colorless oil. MS (ESP) m/z=225.1 [M+H]⁺.

### Step 5: ethyl 4-fluoro-5-hydroxy-6-nitro-indane-2-carboxylate

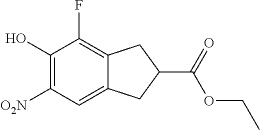 [SMILES: CCOC(=O)C1Cc2cc([N+](=O)[O-])c(O)c(F)c2C1]

To a solution of ethyl 4-fluoro-5-hydroxy-indane-2-carboxylate (1210.0 mg, 5.4 mmol, 1 eq) in DCM (25 mL) at 0° C., nitric acid (385.02 uL, 6.04 mmol, 1.12 eq) was added drop-wise. The reaction mixture was stirred at 0° C. for 0.5 h. The reaction mixture was diluted with water, the phases were separated and the aqueous layer was extracted with DCM (×1). The combined organic phases were filtered through a phase separator and volatiles were removed under reduced pressure to give the crude compound which was purified by silica cartridge chromatography (25 g, cyclohexane 100% to cyclohexane/EtOAc 9:1) to give the title compound (1267 mg, 4.71 mmol, 87.21% yield) as a bright orange solid. MS (ESP) m/z=270.1 [M+H]⁺. ¹H NMR (400 MHz, DMSO-d6) δ 1.20 (t, J=7.1 Hz, 3H), 3.05-3.31 (m, 4H), 3.47 (tt, J=8.8, 7.0 Hz, 1H), 4.11 (q, J=7.1 Hz, 2H), 7.64 (d, J=1.4 Hz, 1H), 10.83 (s, 1H).; correlation between aromatic CH proton and the adjacent benzylic CH₂ protons.

### Step 6: ethyl 4-fluoro-6-nitro-5-(trifluoromethylsulfonyloxy)indane-2-carboxylate

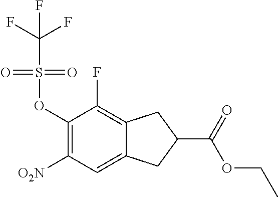 [SMILES: CCOC(=O)C1Cc2cc([N+](=O)[O-])c(OS(=O)(=O)C(F)(F)F)c(F)c2C1]

To a solution of ethyl 4-fluoro-5-hydroxy-6-nitro-indane-2-carboxylate (1.0 g, 3.71 mmol, 1 eq) in DCM (7.43 mL) at 0° C., DIPEA (0.65 mL, 3.71 mmol, 1 eq) was added, followed by drop-wise addition of a solution of trifluoromethanesulfonic anhydride (1.26 mL, 7.43 mmol, 2 eq) in DCM (7.43 mL). The reaction mixture was stirred at 0° C. for 0.5 h. The reaction mixture was diluted with DCM and brine, the phases were separated and the aqueous layer was extracted with DCM (×2). The combined organic phases were filtered through a phase separator and volatiles were removed under reduced pressure to afford the crude product which was purified by silica cartridge chromatography (50 g, cyclohexane 100% to cyclohexane/EtOAc 9:1) to give the title compound (1.26 g, 3.13 mmol, 84.2% yield) as a light yellow oil. MS (ESP) m/z=402.0 [M+H]⁺.

### Step 7: ethyl 5-acetamido-4-fluoro-6-nitro-indane-2-carboxylate

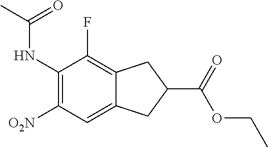 [SMILES: CCOC(=O)C1Cc2cc([N+](=O)[O-])c(NC(C)=O)c(F)c2C1]

In a two-neck round bottom flask equipped with a condenser, a suspension of ethyl 4-fluoro-6-nitro-5-(trifluoromethylsulfonyloxy)indane-2-carboxylate (1255.0 mg, 3.13 mmol, 1 eq), 9,9-dimethyl-4,5-bis(diphenylphosphino)xanthene (361.92 mg, 0.630 mmol, 0.200 eq), tris(dibenzylideneacetone)dipalladium (0) (286.38 mg, 0.310 mmol, 0.100 eq), acetamide (738.95 mg, 12.51 mmol, 4 eq) and cesium carbonate (3056.92 mg, 9.38 mmol, 3 eq) in THF (15.64 mL) was degassed by purging the system with vacuum/argon atmosphere (three cycles). The reaction mixture was stirred at 75° C. for 0.75 h. The reaction mixture was diluted with EtOAc, filtered through a phase separator and volatiles were removed under reduced pressure to give the crude product which was purified by silica cartridge chromatography (100 g silica, from cyclohexane 100% to cyclohexane/EtOAc 7:3) to give the title compound (356 mg, 1.15 mmol, 36.69% yield) as a dark brown viscous oil and ethyl 4-fluoro-5-hydroxy-6-nitro-indane-2-carboxylate (261 mg, 0.970 mmol, 31% yield) as a light yellow solid. MS (ESP) m/z=311.1 [M+H]⁺.

### Step 8: ethyl 5-amino-4-fluoro-6-nitro-indane-2-carboxylate

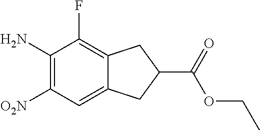 [SMILES: CCOC(=O)C1Cc2cc([N+](=O)[O-])c(N)c(F)c2C1]

To a solution of ethyl 5-acetamido-4-fluoro-6-nitro-indane-2-carboxylate (419.0 mg, 1.35 mmol, 1 eq) in ethanol (13.5 mL) at RT, sulfuric acid (1079.73 uL, 20.26 mmol, 15 eq) was added. The reaction mixture was stirred at 78° C. for 18 h. The reaction mixture was cooled to RT, volatiles were removed under reduced pressure and the residue was partitioned between NaHCO₃ (sat. aq.) and EtOAc. The phases were separated and the aqueous layer was extracted with EtOAc (×1). The combined organic phases were filtered through a phase separator and volatiles removed under reduced pressure to give the crude product which was purified by silica cartridge chromatography (25 g silica, from cyclohexane 100% to cyclohexane/EtOAc 9:1) to give the title compound (95 mg, 0.350 mmol, 26.23% yield) as a bright yellow solid. MS (ESP) m/z=269.1 [M+H]⁺.

### Step 9: ethyl 5,6-diamino-4-fluoro-indane-2-carboxylate

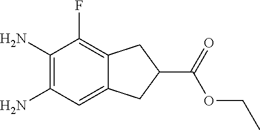 [SMILES: CCOC(=O)C1Cc2cc(N)c(N)c(F)c2C1]

A suspension of ethyl 5-amino-4-fluoro-6-nitro-indane-2-carboxylate (89.03 mg, 0.330 mmol, 1 eq) and 10% Pd/C, 55-65% wet (70.64 mg, 0.030 mmol, 0.100 eq) in ethanol (6.64 mL) was purged with vacuum/nitrogen atmosphere (three cycles) and vacuum/hydrogen atmosphere (three cycles). The reaction mixture was stirred under hydrogen atmosphere at RT for 16 h. The reaction mixture was purged with vacuum/nitrogen atmosphere (three cycles) and filtered through a Celite® pad, washing the cake with EtOH; the volatiles were removed under reduced pressure to give the title compound (75 mg, 0.310 mmol, 94.84% yield) as a light green waxy solid. MS (ESP) m/z=239.1 [M+H]⁺.

### Route B:

### Step 1: methyl 5-acetamido-4-fluoro-7-methylsulfanyl-6-nitro-indane-2-carboxylate

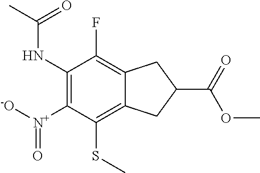

To a solution of methyl 5-acetamido-4,7-difluoro-6-nitro-indane-2-carboxylate (Intermediate 6, Step 4) (15.0 g, 47.73 mmol, 1 eq) in DMF (225 mL) at 0° C., a solution of sodium methanethiolate (6.69 g, 95.47 mmol, 2 eq) in water (127 mL) was added dropwise. The reaction mixture was stirred at 0° C. for 45 min. then warmed to r.t. and stirred for an additional 1 h. The reaction mixture was quenched with 1 M HCl (aq.) and extracted with DCM (×2). The combined organic phases were filtered through a phase separator and the volatiles were removed under reduced pressure to give a residue which was diluted with water, sonicated and the precipitate was collected by filtration on a phase separator to give the crude title compound (29.1 g) as a black viscous oil. MS (ESP) m/z=343.1 [M+H]⁺.

### Step 2: ethyl 5-amino-4-fluoro-7-methylsulfanyl-6-nitro-indane-2-carboxylate

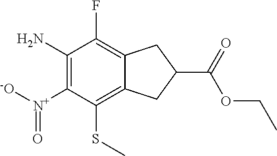

To a solution of methyl 5-acetamido-4-fluoro-7-methylsulfanyl-6-nitro-indane-2-carboxylate (17.98 g, 52.52 mmol, 1 eq) in ethanol (262.6 mL) at r.t., sulfuric acid (30.8 mL, 577.73 mmol, 11 eq) was added. The reaction mixture was stirred at 78° C. for 17 h. The reaction mixture was cooled to r.t., the volatiles were removed under reduced pressure and the residue was partitioned between water and EtOAc. The aqueous phase was extracted with EtOAc (×3). The combined organic phases were filtered through a phase separator and volatiles removed under reduced pressure to give the crude title compound (16.18 g, 51.47 mmol, 98.01% yield) as an orange solid. MS (ESP) m/z=315.2 [M+H]⁺.

### Step 3: ethyl 5,6-diamino-4-fluoro-7-methylsulfanyl-indane-2-carboxylate

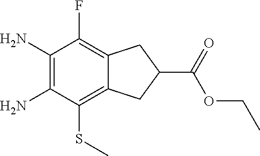 [SMILES: CCOC(=O)C1Cc2c(F)c(N)c(N)c(SC)c2C1]

A suspension of ethyl 5-amino-4-fluoro-7-methylsulfanyl-6-nitro-indane-2-carboxylate (15.18 g, 48.29 mmol, 1 eq) and Raney nickel (slurry in water, 50%) (37.5 g, 48.29 mmol, 1 eq) in ethanol (150 mL) was purged with a nitrogen atmosphere (five times), then with a hydrogen atmosphere (five times) and stirred at r.t. under hydrogen atmosphere (7 bar) for 23 h. The reaction mixture was filtered through a Celite® pad, washing the cake with ethanol. The volatiles were removed to give the crude title compound (10.28 g, 36.15 mmol, 74.86% yield) as a light brown gum. MS (ESP) m/z=285.2 [M+H]⁺.

### Step 4: ethyl 5,6-diamino-4-fluoro-indane-2-carboxylate

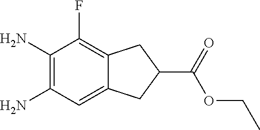 [SMILES: CCOC(=O)C1Cc2cc(N)c(N)c(F)c2C1]

To a solution of ethyl 5,6-diamino-4-fluoro-7-methylsulfanyl-indane-2-carboxylate (10.0 g, 35.17 mmol, 1 eq) in THF (325.63 mL) palladium(II) chloride (623.63 mg, 3.52 mmol, 0.100 eq) was added followed by triethylsilane (11.8 mL, 73.9 mmol, 2.1 eq) and trimethylchlorosilane (4.46 mL, 35.17 mmol, 1 eq) and the mixture was stirred at 35° C. for 15 h. The reaction mixture was diluted with water and then EtOAc (200 mL) was added. The phases were separated and the aqueous phase was basified with NaHCO₃ (sat. aq.) to pH 8 and extracted with EtOAc (200 mL×2). The combined organic extracts were evaporated to give the title compound (2650 mg, 11.12 mmol, 31.63% yield) as an orange solid. MS (ESP) m/z=239.1 [M+H]⁺.

### Intermediate 8

### 4,8-Difluoro-2-methyl-3,5,6,7-tetrahydrocyclopenta[f]benzimidazole-6-carbaldehyde

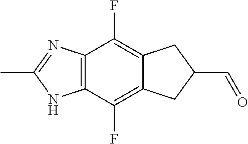 [SMILES: Cc1nc2c(F)c3c(c(F)c2[nH]1)CC(C=O)C3]

### Step 1: ethyl 5-acetamido-6-amino-4,7-difluoro-indane-2-carboxylate

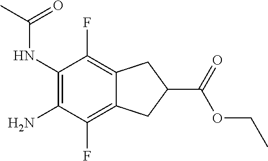 [SMILES: CCOC(=O)C1Cc2c(F)c(N)c(NC(C)=O)c(F)c2C1]

Ethyl 5-acetamido-4,7-difluoro-6-nitro-indane-2-carboxylate (Intermediate 5, Step 6) (185.0 mg, 0.560 mmol, 1 eq) was stirred in ethanol (20 mL) under an atmosphere of hydrogen (765 mm Hg) in the presence of 10% palladium on carbon (59.97 mg, 0.060 mmol, 0.100 eq) at 25° C. for 16 h. The reaction was filtered through a Celite® pad and concentrated in vacuo to afford the title compound (159 mg, 0.530 mmol, 94.58% yield) as a light grey solid. MS (ESP) m/z=299.2 [M+H]⁺.

### Step 2: ethyl 4,8-difluoro-2-methyl-3,5,6,7-tetrahydrocyclopenta[f]benzimidazole-6-carboxylate

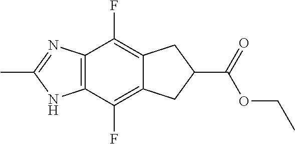 [SMILES: CCOC(=O)C1Cc2c(c(F)c3[nH]c(C)nc3c2F)C1]

Ethyl 5-acetamido-6-amino-4,7-difluoro-indane-2-carboxylate (148.0 mg, 0.500 mmol, 1 eq) was stirred in acetic acid (4 mL) at 60° C. for 16 h. The reaction was concentrated in vacuo then azeotroped with toluene to afford the title compound (132 mg, 0.472 mmol, 94.3% yield), as a light grey solid. MS (ESP) m/z=281.2 [M+H]⁺.

### Step 3: (4,8-difluoro-2-methyl-3,5,6,7-tetrahydrocyclopenta[f]benzimidazol-6-yl)methanol

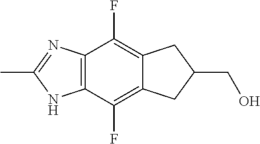 [SMILES: Cc1nc2c(F)c3c(c(F)c2[nH]1)CC(CO)C3]

To a solution of ethyl 4,8-difluoro-2-methyl-3,5,6,7-tetrahydrocyclopenta[f]benzimidazole-6-carboxylate (118.0 mg, 0.420 mmol, 1 eq) in THF (4.21 mL) at 0° C., lithium aluminum hydride 2.3 M in Me-THF (411.87 uL, 0.950 mmol, 2.25 eq) was carefully added. The reaction mixture was stirred at r.t. for 0.5 h. The reaction mixture was quenched with sodium sulfate decahydrate at 0° C. The reaction was concentrated in vacuo and purified by silica cartridge chromatography (DCM/MeOH from 99.5:0.5 to 93:7) to afford the title compound (82.7 mg, 0.347 mmol, 82.69% yield). MS (ESP) m/z=239.3 [M+H]⁺.

### Step 4: 4,8-difluoro-2-methyl-3,5,6,7-tetrahydrocyclopenta[f]benzimidazole-6-carbaldehyde

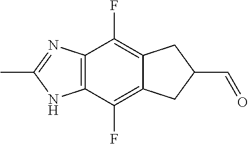 [SMILES: Cc1nc2c(F)c3c(c(F)c2[nH]1)CC(C=O)C3]

A suspension of (4,8-difluoro-2-methyl-3,5,6,7-tetrahydrocyclopenta[f]benzimidazol-6-yl)methanol (85.0 mg, 0.360 mmol, 1 eq) and Dess-Martin periodinane (317.94 mg, 0.750 mmol, 2.1 eq) in DCM (4 mL) and DMF (4 mL) was stirred at r.t. for 1 h. DMSO (2 mL) was added and stirring prolonged for 16 h. The reaction was diluted with EtOAc, Na₂S₂O₃ sat. aq. and NaHCO₃ sat. aq. and stirred for 15 min. Brine was added and phases were separated and organic phase was dried over Na₂SO₄ and concentrated in vacuo to afford the crude title compound (217 mg) as a brown oil which was used in the following reaction without further purification. MS (ESP) m/z=237.1 [M+H]⁺.

### Intermediate 9

### 2-[(Dimethylamino)methyl]-4,8-difluoro-3,5,6,7-tetrahydrocyclopenta[f]benzimidazole-6-carbaldehyde

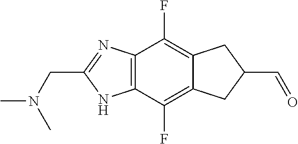 [SMILES: CN(C)Cc1nc2c(F)c3c(c(F)c2[nH]1)CC(C=O)C3]

### Step 1: ethyl 5-amino-6-[[2-(dimethylamino)acetyl]amino]-4,7-difluoro-indane-2-carboxylate

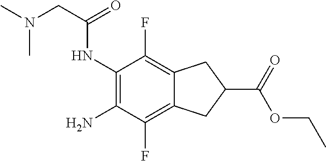 [SMILES: CCOC(=O)C1Cc2c(F)c(N)c(NC(=O)CN(C)C)c(F)c2C1]

To a stirred solution of ethyl 5,6-diamino-4,7-difluoro-indane-2-carboxylate (Intermediate 5) (73 mg, 0.280 mmol, 1 eq) and N,N-dimethylglycine (29.38 mg, 0.280 mmol, 1 eq) in dry MeCN (2.85 mL), 1-methylimidazole (68.12 uL, 0.850 mmol, 3 eq) was added, followed by TCFH (127.89 mg, 0.460 mmol, 1.6 eq). The mixture was stirred for 15 min. at RT. It was concentrated in vacuo and purified by silica cartridge chromatography (cyclohexane/EtOAc from 97:3 to 0:100) to afford the title compound (90 mg, 0.260 mmol, 92.55% yield) as a light yellow oil. MS (ESP) m/z=342.4 [M+H]⁺.

### Steps 2 to 4: ethyl 2-[(dimethylamino)methyl]-4,8-difluoro-3,5,6,7-tetrahydrocyclopenta[f]benzimidazole-6-carboxylate

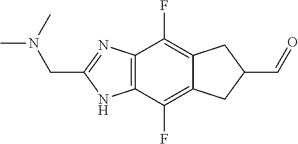 [SMILES: CN(C)Cc1nc2c(F)c3c(c(F)c2[nH]1)CC(C=O)C3]

The title compound was prepared by analogy to Intermediate 8, Steps 2 to 4 and was obtained as a yellow oil which was used in the following reaction without further purification. MS (ESP): m/z=280.2 [M+H]⁺.

The following intermediates were prepared by analogy to Intermediate 9:

| Int. | Name, Structure, MS | Starting Materials |
|---|---|---|
| 10 | 2-[(1,1-dioxo-1,4-thiazinan-4-yl)methyl]-4,8-difluoro-3,5,6,7- tetrahydrocyclopenta[f]benzimidazole-6-carbaldehyde 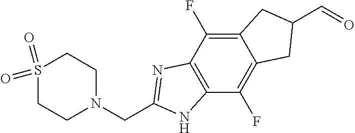 [SMILES: O=CC1Cc2c(c(F)c3[nH]c(CN4CCS(=O)(=O)CC4)nc3c2F)C1] ESP [M + H]⁺: 370.0 | Intermediate 6 and CAS# 155480-08-3 |
| 11 | tert-butyl N-[(4,8-difluoro-6-formyl-3,5,6,7- tetrahydrocyclopenta[f]benzimidazol-2-yl)methyl]-N-methyl- carbamate 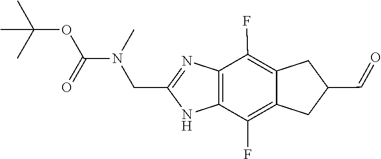 [SMILES: CN(Cc1nc2c(F)c3c(c(F)c2[nH]1)CC(C=O)C3)C(=O)OC(C)(C)C] ESP [M + H]⁺: 366.2 | Intermediate 6 and CAS# 13734-36-6 |
| 12 | tert-butyl N-[(4,8-difluoro-6-formyl-3,5,6,7- tetrahydrocyclopenta[f]benzimidazol-2-yl)methyl]carbamate 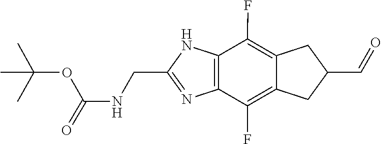 [SMILES: CC(C)(C)OC(=O)NCc1nc2c(F)c3c(c(F)c2[nH]1)CC(C=O)C3] ESP [M + H]⁺: 352.1 | Intermediate 6 and CAS# 4530-20-5 |
| 13 | tert-butyl (2S)-2-(4,8-difluoro-6-formyl-3,5,6,7-tetrahydro- cyclopenta[f]benzimidazol-2-yl)pyrrolidine-1-carboxylate 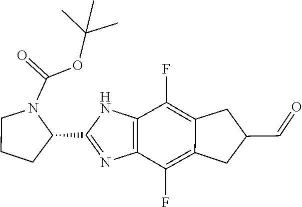 [SMILES: CC(C)(C)OC(=O)N1CCC[C@H]1c1nc2c(F)c3c(c(F)c2[nH]1)CC(C=O)C3] ESP [M + H]⁺: 392.2 | Intermediate 6 and CAS# 15761-39-4 |
| 14 | tert-butyl N-[(8-fluoro-6-formyl-3,5,6,7- tetrahydrocyclopenta[f]benzimidazol-2-yl)methyl]-N-methyl- carbamate 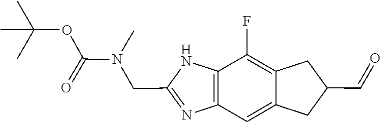 [SMILES: CN(Cc1nc2cc3c(c(F)c2[nH]1)CC(C=O)C3)C(=O)OC(C)(C)C] ESP [M + H]⁺: 348.2 | Intermediate 7 and CAS# 13734-36-6 |
| 15 | tert-butyl N-[2-(4,8-difluoro-6-formyl-3,5,6,7-tetrahydro- cyclopenta[f]benzimidazol-2-yl)ethyl]-N-methyl-carbamate 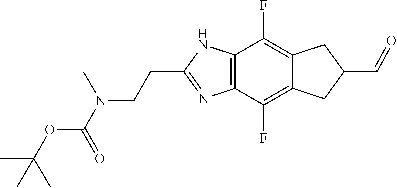 [SMILES: CN(CCc1nc2c(F)c3c(c(F)c2[nH]1)CC(C=O)C3)C(=O)OC(C)(C)C] ESP [M + H]⁺: 380.1 | Intermediate 6 and CAS# 124072-61-3 |
| 16 | tert-butyl (3S)-3-(4,8-difluoro-6-formyl-3,5,6,7-tetrahydro- cyclopenta[f]benzimidazol-2-yl)pyrrolidine-1-carboxylate 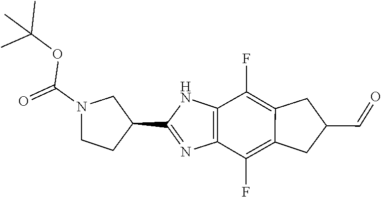 [SMILES: CC(C)(C)OC(=O)N1CC[C@H](c2nc3c(F)c4c(c(F)c3[nH]2)CC(C=O)C4)C1] ESP [M + H]⁺: 392.2 | Intermediate 6 and CAS# 140148-70-5 |
| 17 | tert-butyl 2-(4,8-difluoro-6-formyl-3,5,6,7-tetrahydro- cyclopenta[f]benzimidazol-2-yl)morpholine-4-carboxylate 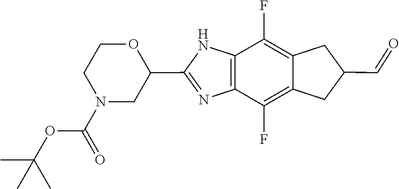 [SMILES: CC(C)(C)OC(=O)N1CCOC(c2nc3c(F)c4c(c(F)c3[nH]2)CC(C=O)C4)C1] ESP [M + H]⁺: 408.1 | Intermediate 6 and CAS# 189321-66-2 |
| 18 | tert-butyl N-[(1S)-4-amino-1-(4,8-difluoro-6-formyl-3,5,6,7-tetra- hydrocyclopenta[f]benzimidazol-2-yl)-4-oxo-butyl]carbamate 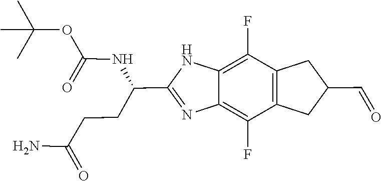 [SMILES: CC(C)(C)OC(=O)N[C@@H](CCC(N)=O)c1nc2c(F)c3c(c(F)c2[nH]1)CC(C=O)C3] | Intermediate 6 and CAS# 13726-85-7 |
| 19 | tert-butyl (2R)-2-(4,8-difluoro-6-formyl-3,5,6,7-tetrahydro- cyclopenta[f]benzimidazol-2-yl)pyrrolidine-1-carboxylate 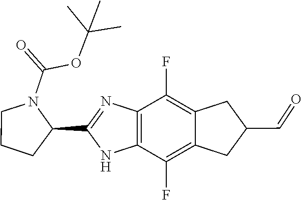 [SMILES: CC(C)(C)OC(=O)N1CCC[C@@H]1c1nc2c(F)c3c(c(F)c2[nH]1)CC(C=O)C3] ESP [M + H]⁺: 392.3 | Intermediate 6 and CAS# 37784-17-1 |
| 20 | tert-butyl N-[(1R)-1-(4,8-difluoro-6-formyl-3,5,6,7-tetra- hydrocyclopenta[f]benzimidazol-2-yl)ethyl]carbamate 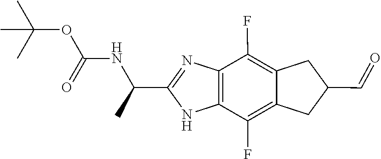 [SMILES: C[C@@H](NC(=O)OC(C)(C)C)c1nc2c(F)c3c(c(F)c2[nH]1)CC(C=O)C3] ESP [M + H]⁺: 366.2 | Intermediate 6 and CAS# 7764-95-6 |
| 21 | tert-butyl (2S)-2-(4,8-difluoro-6-formyl-3,5,6,7-tetrahydro- cyclopenta[f]benzimidazol-2-yl)-4-hydroxy-piperidine-1- carboxylate 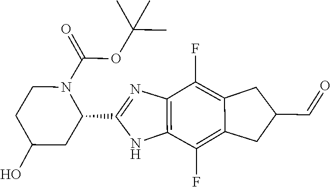 [SMILES: CC(C)(C)OC(=O)N1CCC(O)C[C@H]1c1nc2c(F)c3c(c(F)c2[nH]1)CC(C=O)C3] ESP [M + H]⁺: 422.3 | Intermediate 6 and CAS# 198646-60-5 |
| 22 | 4,8-difluoro-2-(morpholinomethyl)-3,5,6,7- tetrahydrocyclopenta[f]benzimidazole-6-carbaldehyde 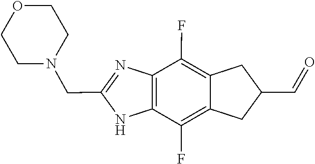 [SMILES: O=CC1Cc2c(c(F)c3[nH]c(CN4CCOCC4)nc3c2F)C1] ESP [M + H]⁺: 320.2 | Intermediate 6 and CAS# 89531-58-8 |

### Intermediate 23

### 4,8-Difluoro-2-(1-hydroxy-1l-methyl-ethyl)-3,5,6,7-tetrahydrocyclopenta[f]benzimidazole-6-carbaldehyde

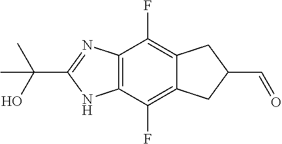 [SMILES: CC(C)(O)c1nc2c(F)c3c(c(F)c2[nH]1)CC(C=O)C3]

### Step 1: ethyl 5-amino-4,7-difluoro-6-[(2-hydroxy-2-methyl-propanoyl)amino]indane-2-carboxylate

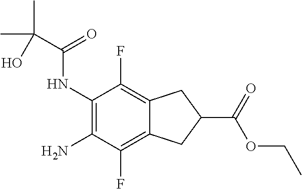 [SMILES: CCOC(=O)C1Cc2c(F)c(N)c(NC(=O)C(C)(C)O)c(F)c2C1]

To a stirred solution of 2-hydroxy-2-methyl-propanoic acid (72.9 mg, 0.700 mmol, 1 eq) in dry DMF (5.27 mL), DIPEA (305.87 uL, 1.76 mmol, 2.5 eq) was added, followed by HATU (534.18 mg, 1.4 mmol, 2 eq) and the mixture was stirred for 10 minutes at RT. The mixture was slowly added to a stirred solution of ethyl 5,6-diamino-4,7-difluoro-indane-2-carboxylate (Intermediate 5, 180.0 mg, 0.700 mmol, 1 eq) in dry DMF (1.76 mL) at RT and the mixture was stirred at this temperature for 16 h. It was diluted with EtOAc and washed with a saturated solution of NaHCO₃ (×2) and then with brine (×1). The organic phase was dried over Na₂SO₄ and concentrated in vacuo. The mixture was purified by silica cartridge chromatography (cyclohexane/EtOAc from 80:20 to 30:70) to afford the title compound (160 mg, 0.470 mmol, 66.54% yield) as a yellow gum. MS (ESP): m/z=343.4 [M+H]⁺.

Steps 2 to 4: 4,8-difluoro-2-(1-hydroxy-1-methyl-ethyl)-3,5,6,7-tetrahydrocyclopenta[f]benzimidazole-6-carbaldehyde

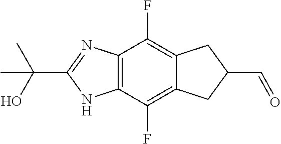 [SMILES: CC(C)(O)c1nc2c(F)c3c(c(F)c2[nH]1)CC(C=O)C3]

The title compound was prepared by analogy to Intermediate 9, Steps 2 to 4 and was obtained as a pink gum which was used in the following reaction without further purification. MS (ESP): m/z=281.0 [M+H]⁺.

### Intermediate 24

### 4,8-Difluoro-2-(4-methylmorpholin-2-yl)-3,5,6,7-tetrahydrocyclopenta[f]benzimidazole-6-carbaldehyde

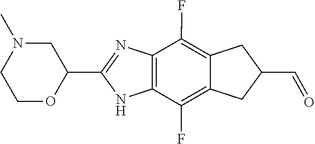 [SMILES: CN1CCOC(c2nc3c(F)c4c(c(F)c3[nH]2)CC(C=O)C4)C1]

### Step 1: [4,8-difluoro-2-(4-methylmorpholin-2-yl)-3,5,6,7-tetrahydrocyclopenta[f]benzimidazol-6-yl]methanol

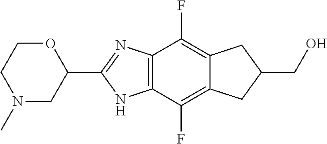 [SMILES: CN1CCOC(c2nc3c(F)c4c(c(F)c3[nH]2)CC(CO)C4)C1]

To a solution of tert-butyl 2-[4,8-difluoro-6-(hydroxymethyl)-3,5,6,7-tetrahydrocyclopenta[f]benzimidazol-2-yl]morpholine-4-carboxylate (Intermediate 17, Step 3) (58.0 mg, 0.140 mmol, 1 eq) in dry THF (1.42 mL) was added lithium aluminum hydride solution (0.18 mL, 0.420 mmol, 3 eq) (2M solution in THF) at 0° C. The reaction mixture was heated gradually to 50° C. and stirred at this temperature for 3 h. After cooling, Na₂SO₄.10H₂O was carefully added at 0° C. and the mixture was stirred at RT for 30 min. The white solid was filtered-off under vacuum, washing with EtOAc and the filtrate was evaporated under reduced pressure to afford the title compound (26 mg, 0.080 mmol, 56.76% yield) as a colorless oil which was used as such in the next step.

### Step 2: 4,8-difluoro-2-(4-methylmorpholin-2-yl)-3,5,6,7-tetrahydrocyclopenta[f]benzimidazole-6-carbaldehyde

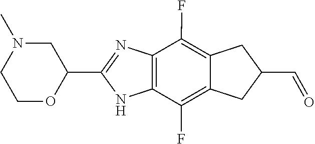 [SMILES: CN1CCOC(c2nc3c(F)c4c(c(F)c3[nH]2)CC(C=O)C4)C1]

The title compound was prepared by analogy to Intermediate 8, Step 4 and was obtained as a light yellow foam which was used in the following reaction without further purification. MS (ESP): m/z=322.1 [M+H]⁺.

### Intermediate 25

### 2-[2-(Dimethylamino)ethyl]-4,8-difluoro-3,5,6,7-tetrahydrocyclopenta[f]benzimidazole-6-carbaldehyde

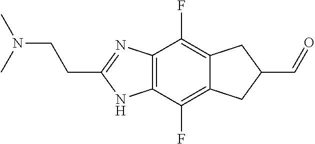 [SMILES: CN(C)CCc1nc2c(F)c3c(c(F)c2[nH]1)CC(C=O)C3]

### Step 1: methyl 2-[2-(dimethylamino)ethyl]-4,8-difluoro-3,5,6,7-tetrahydrocyclopenta[f]benzimidazole-6-carboxylate

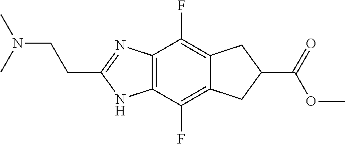

To a solution of methyl 4,8-difluoro-2-[2-(methylamino)ethyl]-3,5,6,7-tetrahydrocyclopenta[f]benzimidazole-6-carboxylate (Intermediate 15, Step 2) (55.0 mg, 0.180 mmol, 1 eq) and formaldehyde (26.48 uL, 0.360 mmol, 2 eq) in THF (1.78 mL) at RT, sodium triacetoxyborohydride (113.06 mg, 0.530 mmol, 3 eq) was added in one portion. The reaction mixture was stirred at RT for 16 h. The reaction mixture was diluted with EtOAc and NaHCO₃ sat. aq., the phases were separated and the aqueous layer was extracted with EtOAc (×2). The combined organic phases were filtered through a phase separator and volatiles were removed under reduced pressure to afford the crude product which was purified by silica cartridge chromatography (from DCM 100% to DCM/MeOH 9:1) to give the title compound (30 mg, 0.090 mmol, 52.18% yield) as a white foam. MS (ESN): m/z=322.3 [M−H]⁻.

### Steps 2 and 3: 2-[2-(dimethylamino)ethyl]-4,8-difluoro-3,5,6,7-tetrahydrocyclopenta[f]benzimidazole-6-carbaldehyde

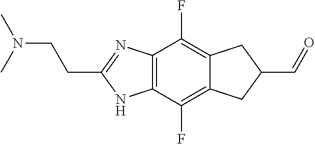 [SMILES: CN(C)CCc1nc2c(F)c3c(c(F)c2[nH]1)CC(C=O)C3]

The title compound was prepared by analogy to Intermediate 9, Steps 3 and 4 and was obtained as a purple oil which was used in the following reaction without further purification.

### Intermediate 26

### tert-Butyl N-[(1R)-1-(4,8-difluoro-6-formyl-3,5,6,7-tetrahydrocyclopenta[f]benzimidazol-2-yl)ethyl]-N-methyl-carbamate

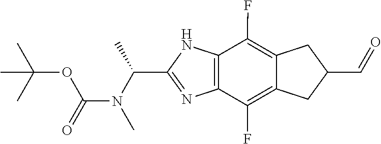 [SMILES: C[C@H](c1nc2c(F)c3c(c(F)c2[nH]1)CC(C=O)C3)N(C)C(=O)OC(C)(C)C]

### Step 1: methyl 5-amino-6-[[(2R)-2-[tert-butoxycarbonyl(methyl)amino]propanoyl]amino]-4,7-difluoro-indane-2-carboxylate

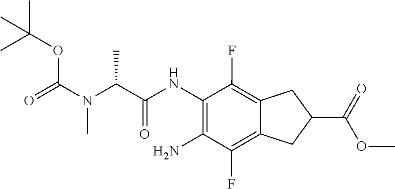 [SMILES: COC(=O)C1Cc2c(F)c(N)c(NC(=O)[C@@H](C)N(C)C(=O)OC(C)(C)C)c(F)c2C1]

A mixture of BOC-N-methyl-D-alanine [CAS #19914-38-6] (169.32 mg, 0.830 mmol, 1.01 eq) in DCM (11.47 mL) was cooled to −15° C.; 4-dimethylaminopyridine (302.62 mg, 2.48 mmol, 3 eq) was added followed by T3P (630.53 mg 50% solution, 0.990 mmol, 1.2 eq) dropwise. The mixture was stirred at -15° C. for 1 h then methyl 5,6-diamino-4,7-difluoro-indane-2-carboxylate (Intermediate 6) (200.0 mg, 0.830 mmol, 1 eq) was added. The mixture was stirred for 1 h maintaining the temperature, then it was left to reach room temperature gradually and stirred at RT for 16 h. DCM and NaHCO₃ were added, the phases were separated, the aqueous phase was extracted with DCM (×3). The combined organic phases were dried over Na₂SO₄, filtered and concentrated under reduced pressure. The crude product was purified by flash column chromatography (silica 10 g, 0-3% MeOH/DCM) to give the title compound (208 mg, 0.490 mmol, 58.93% yield) as a yellow oil. MS (ESN) m/z=426.5 [M−H]⁻.

### Steps 2 to 4: tert-butyl N-[(1R)-1-(4,8-difluoro-6-formyl-3,5,6,7-tetrahydrocyclopenta[f]benzimidazol-2-yl)ethyl]-N-methyl-carbamate

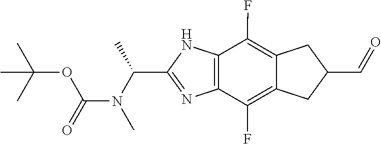 [SMILES: C[C@H](c1nc2c(F)c3c(c(F)c2[nH]1)CC(C=O)C3)N(C)C(=O)OC(C)(C)C]

The title compound was prepared by analogy to Intermediate 8, Steps 2 to 4 and was obtained as a light yellow oil which was used in the following reaction without purification. MS (ESP) m/z=380.1 [M+H]⁺.

### Intermediate 27

### tert-Butyl (3R)-3-(4,8-difluoro-6-formyl-3,5,6,7-tetrahydrocyclopenta[f]benzimidazol-2-yl)morpholine-4-carboxylate

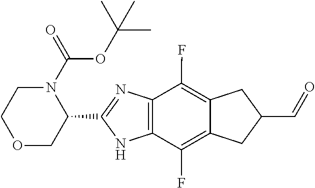 [SMILES: CC(C)(C)OC(=O)N1CCOC[C@H]1c1nc2c(F)c3c(c(F)c2[nH]1)CC(C=O)C3]

The title compound was prepared from methyl 5,6-diamino-4,7-difluoro-indane-2-carboxylate (Intermediate 6) and 4-BOC-3(R)-morpholinecarboxylic acid [CAS #869681-70-9] by analogy to Intermediate 23. MS (ESP) m/z=408.2 [M+H]⁺.

### Intermediate 28

### tert-Butyl N-[2-(4,8-difluoro-6-formyl-1-methyl-6,7-dihydro-5H-cyclopenta[f]benzimidazol-2-yl)ethyl]-N-methyl-carbamate

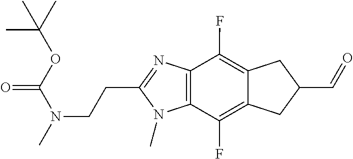 [SMILES: CN(CCc1nc2c(F)c3c(c(F)c2n1C)CC(C=O)C3)C(=O)OC(C)(C)C]

### Step 1: methyl 2-[2-[tert-butoxycarbonyl(methyl)amino]ethyl]-4,8-difluoro-1-methyl-6,7-dihydro-5H-cyclopenta[f]benzimidazole-6-carboxylate

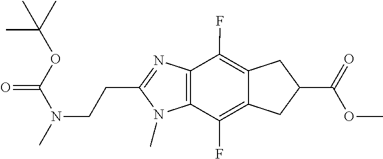 [SMILES: COC(=O)C1Cc2c(c(F)c3c(nc(CCN(C)C(=O)OC(C)(C)C)n3C)c2F)C1]

A vial was charged with methyl 2-[2-[tert-butoxycarbonyl(methyl)amino]ethyl]-4,8-difluoro-3,5,6,7-tetrahydrocyclopenta[f]benzimidazole-6-carboxylate (Intermediate 15, Step 2) (171.0 mg, 0.420 mmol, 1 eq), 1,8-diazabicyclo[5.4.0]undec-7-ene (0.06 mL, 0.420 mmol, 1 eq), and dimethyl carbonate (4.61 mL, 54.69 mmol, 130.94 eq). The mixture was heated to 90° C. overnight. The mixture was diluted with EtOAc, washed twice with sat. aq. NaHCO₃, filtered through a hydrophobic phase separator and dried in vacuo. The crude was purified by silica cartridge chromatography (10 g, 0% to 50% EtOAc/cyclohexane) to afford the title compound (168 mg, 0.400 mmol, 94.99% yield) as a light yellow oil. MS (ESP) m/z=424.3 [M+H]⁺.

### Step 2: tert-butyl N-[2-[4,8-difluoro-6-(hydroxymethyl)-1-methyl-6,7-dihydro-5H-cyclopenta[f]benzimidazol-2-yl]ethyl]-N-methyl-carbamate

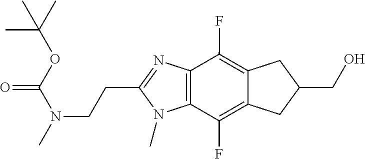 [SMILES: CN(CCc1nc2c(F)c3c(c(F)c2n1C)CC(CO)C3)C(=O)OC(C)(C)C]

To a stirred solution of methyl 2-[2-[tert-butoxycarbonyl(methyl)amino]ethyl]-4,8-difluoro-1-methyl-6,7-dihydro-5H-cyclopenta[f]benzimidazole-6-carboxylate (223.0 mg, 0.530 mmol, 1 eq) in dry THF (2.64 mL), ethanol (1.32 mL), calcium chloride (116.89 mg, 1.05 mmol, 2 eq) and sodium borohydride (59.77 mg, 1.58 mmol, 3 eq) were added and the mixture was stirred at 25° C. for 18 h. The mixture was cooled to 0° C., carefully quenched with sat. aq. NH₄Cl and extracted with EtOAc (×3). The organic phase was dried over Na₂SO₄ and concentrated in vacuo. The crude was purified by flash column chromatography (0-50% EtOAc/cyclohexane) to give the title compound (167 mg, 0.420 mmol, 80.19% yield) as a light yellow foam. MS (ESP) m/z=408.2 [M+H]⁺.

### Step 3: tert-butyl N-[2-(4,8-difluoro-6-formyl-1-methyl-6,7-dihydro-5H-cyclopenta[f]benzimidazol-2-yl)ethyl]-N-methyl-carbamate

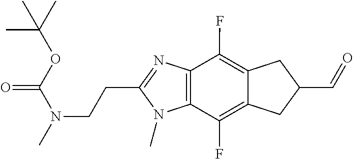 [SMILES: CN(CCc1nc2c(F)c3c(c(F)c2n1C)CC(C=O)C3)C(=O)OC(C)(C)C]

The title compound was prepared by analogy to Intermediate 8, Step 4 and was obtained as a light yellow oil. MS (ESP) m/z=396.4 [M+H]⁺.

### Intermediate 29

### tert-Butyl N-[(8-fluoro-6-formyl-6,7-dihydro-5H-cyclopenta[f][1,3]benzoxazol-2-yl)methyl]-N-methyl-carbamate

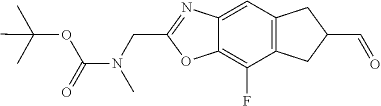 [SMILES: CN(Cc1nc2cc3c(c(F)c2o1)CC(C=O)C3)C(=O)OC(C)(C)C]

### Step 1: ethyl 6-amino-4-fluoro-5-hydroxy-indane-2-carboxylate

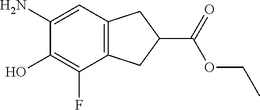 [SMILES: CCOC(=O)C1Cc2cc(N)c(O)c(F)c2C1]

A suspension of ethyl 4-fluoro-5-hydroxy-6-nitro-indane-2-carboxylate (Intermediate 7, Step 5) (500.0 mg, 1.86 mmol, 1 eq) and Pd/C 55-65% wet (79.06 mg, 0.040 mmol, 0.020 eq) in ethanol (37.9 mL) was purged with vacuum/nitrogen atmosphere (three cycles) and with vacuum/hydrogen atmosphere (four cycles). The reaction mixture was stirred at RT under hydrogen atmosphere for 16 h. The reaction mixture was purged with vacuum/nitrogen atmosphere (three cycles) and filtered through a Celite® pad, washing the cake with ethanol. The filtrate was concentrated under reduced pressure to give the crude title compound (411 mg, 1.72 mmol, 87.88% yield) as a colorless viscous oil. MS (ESP) m/z=240.1 [M+H]⁺.

### Step 2: ethyl 6-[[2-[tert-butoxycarbonyl(methyl)amino]acetyl]amino]-4-fluoro-5-hydroxy-indane-2-carboxylate

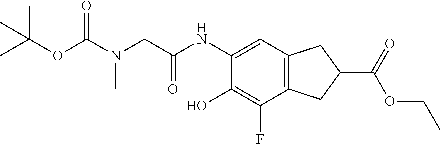 [SMILES: CCOC(=O)C1Cc2cc(NC(=O)CN(C)C(=O)OC(C)(C)C)c(O)c(F)c2C1]

To a stirred solution of BOC-SAR-OH (235.68 mg, 1.25 mmol, 1 eq) and HATU (506.77 mg, 1.33 mmol, 1.07 eq) in DMF (6.84 mL) was added DIPEA (1.08 mL, 6.23 mmol, 5 eq). After stirring for 5 minutes the resulting suspension was charged with ethyl 6-amino-4-fluoro-5-hydroxy-indane-2-carboxylate (298.0 mg, 1.25 mmol, 1 eq) and the mixture was stirred at RT for 24 h. The reaction mixture was partitioned between EtOAc and NaHCO₃. The organic phase was washed with brine, dried over Na₂SO₄ and evaporated under vacuum to obtain a residue which was purified by by silica cartridge chromatography (25 g, cyclohexane 100% to cyclohexane/EtOAc 50:50) to give the title compound (228 mg, 0.560 mmol, 40.14% yield) as a light yellow oil. MS (ESP) m/z=411.2 [M+H]⁺.

### Step 3: ethyl 2-[[tert-butoxycarbonyl(methyl)amino]methyl]-8-fluoro-6,7-dihydro-5H-cyclopenta[f][1,3]benzoxazole-6-carboxylate

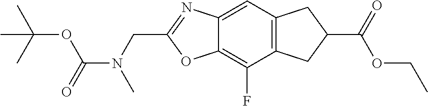 [SMILES: CCOC(=O)C1Cc2cc3nc(CN(C)C(=O)OC(C)(C)C)oc3c(F)c2C1]

Ethyl 6-[[2-[tert-butoxycarbonyl(methyl)amino]acetyl]amino]-4-fluoro-5-hydroxy-indane-2-carboxylate (220.0 mg, 0.540 mmol, 1 eq) was dissolved in dry THF (2.74 mL), then triphenylphosphine (309.29 mg, 1.18 mmol, 2.2 eq) and freshly activated 4 Å molecular sieves were added. The mixture was stirred at 0° C. for 1 h under nitrogen. A solution of diisopropyl azodicarboxylate (0.23 mL, 1.18 mmol, 2.2 eq) was added dropwise. The resulting mixture was then allowed to warm to RT. After stirring for 1 h, the solvent was removed under vacuum. Diethyl ether was added and the resulting mixture was stirred for 1 h. The filtrate was concentrated and the residue was purified by silica cartridge chromatography (from 100% cyclohexane to 100% EtOAc) to afford the impure title compound (388 mg). This material was used in the next step without any further purification. MS (ESP) m/z=393.2 [M+H]⁺.

### Step 4: tert-butyl N-[[8-fluoro-6-(hydroxymethyl)-6,7-dihydro-5H-cyclopenta[f][1,3]benzoxazol-2-yl]methyl]-N-methyl-carbamate

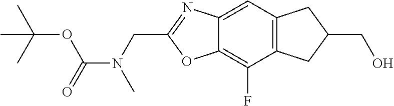 [SMILES: CN(Cc1nc2cc3c(c(F)c2o1)CC(CO)C3)C(=O)OC(C)(C)C]

To a stirred solution of ethyl 2-[[tert-butoxycarbonyl(methyl)amino]methyl]-8-fluoro-6,7-dihydro-5H-cyclopenta[f][1,3]benzoxazole-6-carboxylate (210.0 mg, 0.550 mmol, 1 eq) in dry methanol (3.7 mL) and THF (7.4 mL) cooled to 0° C., calcium chloride (123.18 mg, 1.11 mmol, 2 eq) was added, followed by sodium borohydride (62.99 mg, 1.66 mmol, 3 eq) and the mixture was stirred at this temperature for 5 minutes. The reaction was poured into a saturated solution of NH₄Cl, cooled to 0° C., and extracted with EtOAc (×1). The organic phase was dried over Na₂SO₄ and concentrated in vacuo. The residue was purified by silica cartridge chromatography (from 100% DCM to 95:5 DCM/MeOH) to afford the title compound (110 mg, 0.310 mmol, 44.52% yield) as a light yellow gum. MS (ESP) m/z=351.5 [M+H]⁺.

### Step 5: tert-butyl N-[(8-fluoro-6-formyl-6,7-dihydro-5H-cyclopenta[f][1,3]benzoxazol-2-yl)methyl]-N-methyl-carbamate

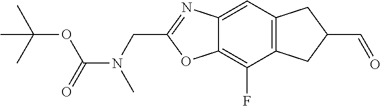 [SMILES: CN(Cc1nc2cc3c(c(F)c2o1)CC(C=O)C3)C(=O)OC(C)(C)C]

To a stirred solution of tert-butyl N-[[8-fluoro-6-(hydroxymethyl)-6,7-dihydro-5H-cyclopenta[f][1,3]benzoxazol-2-yl]methyl]-N-methyl-carbamate (85.0 mg, 0.240 mmol, 1 eq) in DCM (2.67 mL), Dess-Martin periodinane (133.76 mg, 0.320 mmol, 1.3 eq) was added and the mixture was stirred for 50 minutes at RT. More DMP was added and stirring prolonged for 30 min. The reaction was diluted with DCM, Na₂S₂O₃ (sat. aq.) and NaHCO₃ (sat. aq.) and stirred for 20 min. The organic phase was dried over Na₂SO₄ and concentrated in vacuo to afford the title compound (75 mg, 0.220 mmol, 88.74% yield) as a light yellow oil. MS (ESP) m/z=349.2 [M+H]⁺.

### Intermediate 30

### tert-Butyl 10,16-difluoro-13-formyl-2,5,8-triazatetracyclo[7.7.0.02,7.011,15]hexadeca-1(16),7,9,11(15)-tetraene-5-carboxylate

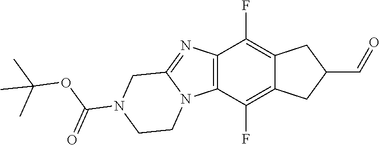 [SMILES: CC(C)(C)OC(=O)N1CCn2c(nc3c(F)c4c(c(F)c32)CC(C=O)C4)C1]

### Step 1: O5-tert-butyl O13-methyl 10,16-difluoro-2,5,8-triazatetracyclo[7.7.0.02,7.011,15]hexadeca-1(16),7,9,11(15)-tetraene-5,13-dicarboxylate

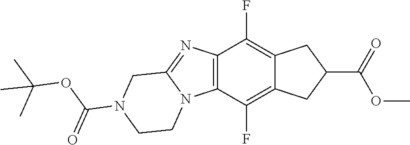 [SMILES: COC(=O)C1Cc2c(c(F)c3c(nc4n3CCN(C(=O)OC(C)(C)C)C4)c2F)C1]

To a stirred solution of methyl 2-[(tert-butoxycarbonylamino)methyl]-4,8-difluoro-3,5,6,7-tetrahydrocyclopenta[f]benzimidazole-6-carboxylate (Intermediate 12, Step 2) (127.0 mg, 0.330 mmol, 1 eq) in DMF (1.4 mL) was added 1,2-dibromoethane (86.09 uL, 1 mmol, 3 eq) followed by cesium carbonate (217.0 mg, 0.670 mmol, 2 eq). The resulting mixture was stirred at 50° C. for 2 h. Then further cesium carbonate (217.0 mg, 0.670 mmol, 2 eq) was added and the reaction mixture was stirred at 50° C. for 3 h. Then a saturated aqueous solution of NH₄Cl was added and the mixture was extracted with EtOAc (3×5 mL). The organic layers were collected, dried over Na₂SO₄, filtered and concentrated under vacuum to give a crude that was purified by silica cartridge chromatography (25 g, cyclohexane/EtOAc from 100:0 to 80:20) to afford the title compound (94 mg, 0.230 mmol, 69.28% yield) as a white foam. MS (ESP) m/z=408.2 [M+H]⁺.

Steps 2 and 3: tert-butyl 10,16-difluoro-13-formyl-2,5,8-triazatetracyclo[7.7.0.02,7.011,15]hexadeca-1(16),7,9,11(15)-tetraene-5-carboxylate

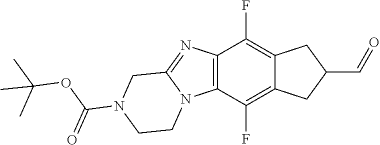 [SMILES: CC(C)(C)OC(=O)N1CCn2c(nc3c(F)c4c(c(F)c32)CC(C=O)C4)C1]

The title compound was prepared by analogy to Intermediate 8, Steps 3 and 4 and was obtained as a white foam. MS (ESP) m/z=378.2 [M+H]⁺.

### Intermediate 31

### tert-Butyl (2S,4R)-4-[tert-butoxycarbonyl(methyl)amino]-2-(4,8-difluoro-6-formyl-3,5,6,7-tetrahydrocyclopenta[f]benzimidazol-2-yl)pyrrolidine-1-carboxylate

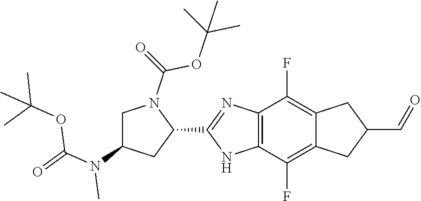 [SMILES: CN(C(=O)OC(C)(C)C)[C@@H]1C[C@@H](c2nc3c(F)c4c(c(F)c3[nH]2)CC(C=O)C4)N(C(=O)OC(C)(C)C)C1]

### Step 1: (2S,4R)-1-tert-butoxycarbonyl-4-[tert-butoxycarbonyl(methyl)amino]pyrrolidine-2-carboxylic acid

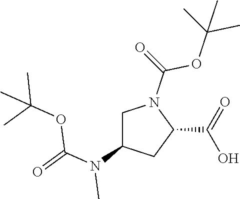 [SMILES: CN(C(=O)OC(C)(C)C)[C@@H]1C[C@@H](C(=O)O)N(C(=O)OC(C)(C)C)C1]

A solution of (2S,4R)-1-tert-butoxycarbonyl-4-(tert-butoxycarbonylamino)pyrrolidine-2-carboxylic acid [CAS #254881-66-8] (200.0 mg, 0.610 mmol, 1 eq) in dry THF (5.82 mL) was treated with NaH, dispersion in mineral oil (72.64 mg, 1.82 mmol, 3 eq) and stirred at RT for 10 minutes. Iodomethane (0.08 mL, 1.21 mmol, 2 eq) was added and the resulting mixture was stirred 3 h at RT. Additional NaH, dispersion in mineral oil (48.43 mg, 1.21 mmol, 2 eq) and iodomethane (0.04 mL, 0.610 mmol, 1 eq) were added and stirring continued for 2 h. Additional iodomethane (0.08 mL, 1.21 mmol, 2 eq) was added and stirring continued for 2 h then the reaction was carefully quenched with water. The aqueous phase was washed with Et₂O (×2) then acidified to pH=3 by addition of HCl (1M aqueous), then extracted with CH₂Cl₂ (3×15 mL) and with EtOAc (×1). The combined organic extracts were filtered through a phase separator, concentrated under reduced pressure to afford the title compound (207 mg, 0.600 mmol, 99.29% yield) as a light yellow foam.

### Steps 2 and 3: methyl 2-[(2S,4R)-1-tert-butoxycarbonyl-4-[tert-butoxycarbonyl(methyl)amino]pyrrolidin-2-yl]-4,8-difluoro-3,5,6,7-tetrahydrocyclopenta[f]benzimidazole-6-carboxylate

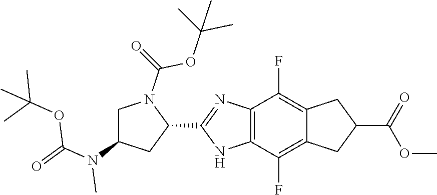 [SMILES: COC(=O)C1Cc2c(c(F)c3[nH]c([C@@H]4C[C@@H](N(C)C(=O)OC(C)(C)C)CN4C(=O)OC(C)(C)C)nc3c2F)C1]

The title compound was prepared by analogy to Intermediate 9, Steps 1 and 2 and was obtained as a pale yellow foam.

### Step 4: tert-butyl (2S,4R)-4-[tert-butoxycarbonyl(methyl)amino]-2-[4,8-difluoro-6-(hydroxymethyl)-3,5,6,7-tetrahydrocyclopenta[f]benzimidazol-2-yl]pyrrolidine-1-carboxylate

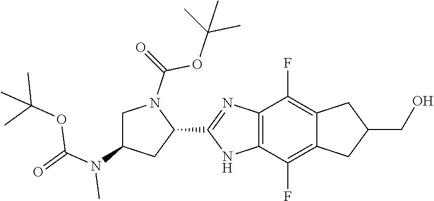 [SMILES: CN(C(=O)OC(C)(C)C)[C@@H]1C[C@@H](c2nc3c(F)c4c(c(F)c3[nH]2)CC(CO)C4)N(C(=O)OC(C)(C)C)C1]

To a stirred solution of methyl 2-[(2S,4R)-1-tert-butoxycarbonyl-4-[tert-butoxycarbonyl(methyl)amino]pyrrolidin-2-yl]-4,8-difluoro-3,5,6,7-tetrahydrocyclopenta[f]benzimidazole-6-carboxylate (195.28 mg, 0.350 mmol, 1 eq) in THF (3 mL) at 0° C., DIBAL-H (1 M in THF) (1.06 mL, 1.06 mmol, 3 eq) was added dropwise. The reaction mixture was stirred at 25° C. for 3 h, then cooled to 0° C., carefully diluted with saturated aq. NH₄Cl and extracted with EtOAc (100 mL×3). The organic phases were collected, dried over Na₂SO₄, filtered and concentrated under vacuum to give a crude product that was purified by silica cartridge chromatography (25 g, DCM/MeOH from 100:0 to 90:10) to give the title compound (52 mg, 0.170 mmol, 43.3% yield) as a colorless oil. MS (ESP) m/z=523.5 [M+H]⁺.

### Step 5: tert-butyl (2S,4R)-4-[tert-butoxycarbonyl(methyl)amino]-2-(4,8-difluoro-6-formyl-3,5,6,7-tetrahydrocyclopenta[f]benzimidazol-2-yl)pyrrolidine-1-carboxylate

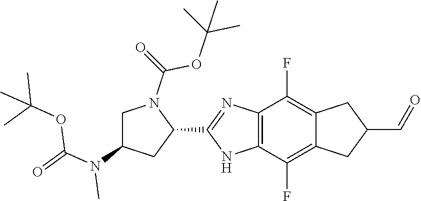 [SMILES: CN(C(=O)OC(C)(C)C)[C@@H]1C[C@@H](c2nc3c(F)c4c(c(F)c3[nH]2)CC(C=O)C4)N(C(=O)OC(C)(C)C)C1]

The title compound was prepared by analogy to Intermediate 8, Step 4 and was obtained as a yellow oil. MS (ESP) m/z=521.3 [M+H]⁺.

### Intermediate 32

### tert-Butyl (2S,4R)-4-[tert-butyl(dimethyl)silyl]oxy-2-(4,8-difluoro-6-formyl-3,5,6,7-tetrahydrocyclopenta[f]benzimidazol-2-yl)pyrrolidine-1-carboxylate

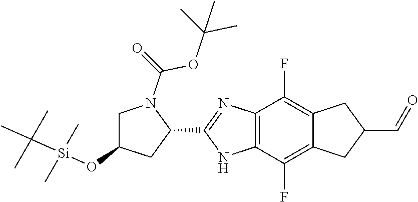 [SMILES: CC(C)(C)OC(=O)N1C[C@H](O[Si](C)(C)C(C)(C)C)C[C@H]1c1nc2c(F)c3c(c(F)c2[nH]1)CC(C=O)C3]

### Step 1: (2S,4R)-1-tert-butoxycarbonyl-4-[tert-butyl(dimethyl)silyl]oxy-pyrrolidine-2-carboxylic acid

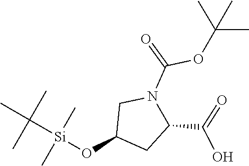 [SMILES: CC(C)(C)OC(=O)N1C[C@H](O[Si](C)(C)C(C)(C)C)C[C@H]1C(=O)O]

To a solution of (2S,4R)-1-[(tert-butoxy)carbonyl]-4-hydroxypyrrolidine-2-carboxylic acid [CAS #13726-69-7] (1000.0 mg, 4.32 mmol, 1 eq) in DCM (10 mL) and DMF (5 mL) at 0° C., imidazole (1471.94 mg, 21.62 mmol, 5 eq) was added in one portion. The mixture was stirred at 0° C. for 5 min. then a solution of tert-butyldimethylchlorosilane (1433.88 mg, 9.51 mmol, 2.2 eq) in DCM (10 mL) was added drop-wise at 0° C. The reaction mixture was stirred at 0° C. for 5 min. then at RT for 17 h. The reaction mixture was quenched with NH₄Cl (sat. aq.), the phases were separated and the aqueous layer was extracted with EtOAc (×2). The combined organic phases were filtered through a phase separator and volatiles were removed under reduced pressure to afford the crude product (2.75 g, colorless viscous oil) which was purified by silica gel chromatography on (25 g silica cartridge, from DCM 100% to DCM/MeOH 95:5) to give the title compound (770 mg, 2.23 mmol, 51.54% yield) as a colorless viscous oil. MS (ESN) m/z=344.6 [M−H]⁻.

### Steps 2 to 5: tert-butyl (2S,4R)-4-[tert-butyl(dimethyl)silyl]oxy-2-(4,8-difluoro-6-formyl-3,5,6,7-tetrahydrocyclopenta[f]benzimidazol-2-yl)pyrrolidine-1-carboxylate

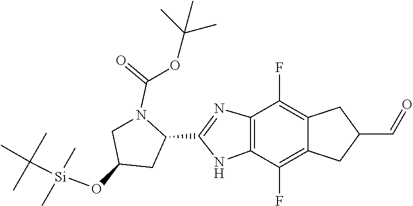 [SMILES: CC(C)(C)OC(=O)N1C[C@H](O[Si](C)(C)C(C)(C)C)C[C@H]1c1nc2c(F)c3c(c(F)c2[nH]1)CC(C=O)C3]

The title compound was prepared by analogy to Intermediate 9 using (2S,4R)-1-tert-butoxycarbonyl-4-[tert-butyl(dimethyl)silyl]oxy-pyrrolidine-2-carboxylic acid in place of N,N-dimethylglycine in the first step and was obtained as a white solid. MS (ESP) m/z=540.5 [M+H+H₂O]⁺.

The following intermediates were prepared by analogy to Intermediate 32:

| Int. | Name, Structure, MS | Starting Materials |
|---|---|---|
| 33 | tert-butyl(2S,4R)-4-[tert-butyl(dimethyl)silyl]oxy-2- (8-fluoro-6-formyl-3,5,6,7-tetrahydrocyclopenta[f] benzimidazol-2-yl)pyrrolidine-1-carboxylate 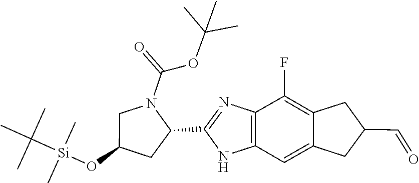 [SMILES: CC(C)(C)OC(=O)N1C[C@H](O[Si](C)(C)C(C)(C)C)C[C@H]1c1nc2c(F)c3c(cc2[nH]1)CC(C=O)C3] ESP [M + H]⁺: 504.4 | Intermediate 7 and CAS# 13726-69-7 |
| 34 | tert-butyl (2R,4S)-4-[tert-butyl(dimethyl)silyl]oxy-2-(4,8- difluoro-6-formyl-3,5,6,7-tetrahydrocyclopenta[f] benzimidazol-2-yl)pyrrolidine-1-carboxylate 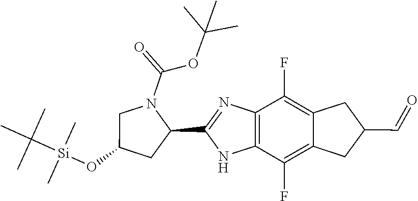 [SMILES: CC(C)(C)OC(=O)N1C[C@@H](O[Si](C)(C)C(C)(C)C)C[C@@H]1c1nc2c(F)c3c(c(F)c2[nH]1)CC(C=O)C3] ESP [M + H]⁺: 522.3 | Intermediate 6 and CAS# 147266-92-0 |
| 35 | tert-butyl (2R,4R)-4-[tert-butyl(dimethyl)silyl]oxy-2-(4,8- difluoro-6-formyl-3,5,6,7-tetrahydro cyclopenta[f] benzimidazol-2-yl)pyrrolidine-1-carboxylate 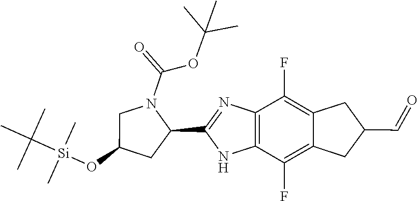 [SMILES: CC(C)(C)OC(=O)N1C[C@H](O[Si](C)(C)C(C)(C)C)C[C@@H]1c1nc2c(F)c3c(c(F)c2[nH]1)CC(C=O)C3] ESP [M + H]⁺: 522.2 | Intermediate 6 and CAS# 135042-12-5 |
| 36 | tert-butyl (2S,4S)-4-[tert-butyl(dimethyl)silyl]oxy-2-(4,8- difluoro-6-formyl-3,5,6,7-tetrahydro cyclopenta[f]benz- imidazol-2-yl)pyrrolidine-1-carboxylate 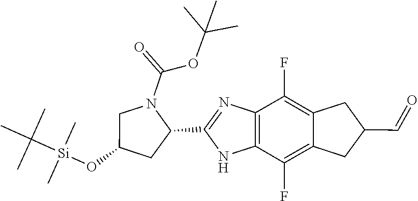 [SMILES: CC(C)(C)OC(=O)N1C[C@@H](O[Si](C)(C)C(C)(C)C)C[C@H]1c1nc2c(F)c3c(c(F)c2[nH]1)CC(C=O)C3] ESP [M + H]⁺: 522.3 | Intermediate 6 and CAS# 87691-27-8 |
| 37 | tert-butyl N-[(1S,2S)-1-(4,8-difluoro-6-formyl-3,5,6,7- tetrahydrocyclopenta[f]benzimidazol-2-yl)-2-hydroxy- propyl]carbamate 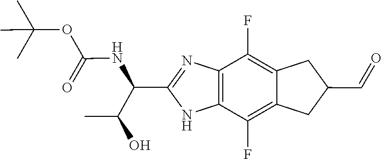 [SMILES: C[C@H](O)[C@@H](NC(=O)OC(C)(C)C)c1nc2c(F)c3c(c(F)c2[nH]1)CC(C=O)C3] ESP [M + H]⁺: 396.3 | Intermediate 6 and CAS# 55674-67-4 |

### Intermediate 38

### tert-butyl (2R,3S)-3-[tert-butyl(dimethyl)silyl]oxy-2-(4,8-difluoro-6-formyl-3,5,6,7-tetrahydro cyclopenta[f]benzimidazol-2-yl)pyrrolidine-1-carboxylate

### Step 1 to 3

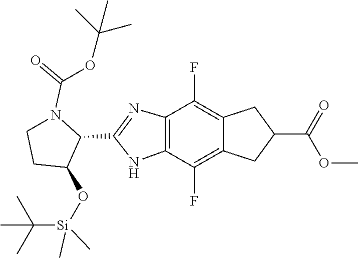 [SMILES: COC(=O)C1Cc2c(c(F)c3[nH]c([C@@H]4[C@@H](O[Si](C)(C)C(C)(C)C)CCN4C(=O)OC(C)(C)C)nc3c2F)C1]

The title compound was prepared from (2S,3S)-1-tert-butoxycarbonyl-3-hydroxy-pyrrolidine-2-carboxylic acid [CAS #187039-57-2] by analogy to Intermediate 32, Steps 1 to 3 and was obtained as a yellow oil. MS (ESP) m/z=552.2 [M+H]⁺.

### Steps 4 to 5

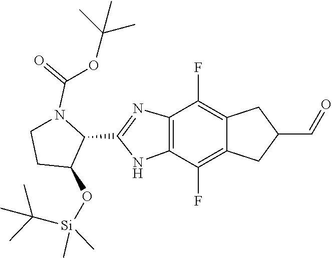 [SMILES: CC(C)(C)OC(=O)N1CC[C@H](O[Si](C)(C)C(C)(C)C)[C@H]1c1nc2c(F)c3c(c(F)c2[nH]1)CC(C=O)C3]

The title compound was prepared by analogy to Intermediate 28, Steps 2 to 3 and was obtained as a light yellow oil. MS (ESP) m/z=522.2 [M+H]⁺.

### Intermediate 39

### tert-Butyl (2S,4R)-4-[tert-butyl(dimethyl)silyl]oxy-2-(4,8-difluoro-6-formyl-1-methyl-6,7-dihydro-5H-cyclopenta[f]benzimidazol-2-yl)pyrrolidine-1-carboxylate

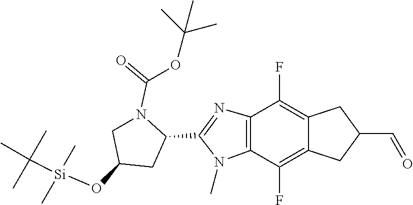 [SMILES: Cn1c([C@@H]2C[C@@H](O[Si](C)(C)C(C)(C)C)CN2C(=O)OC(C)(C)C)nc2c(F)c3c(c(F)c21)CC(C=O)C3]

The title compound was prepared from methyl 2-[(2S,4R)-1-tert-butoxycarbonyl-4-[tert-butyl(dimethyl)silyl]oxy-pyrrolidin-2-yl]-4,8-difluoro-3,5,6,7-tetrahydrocyclopenta[f]benzimidazole-6-carboxylate (Intermediate 32, Step 3) by analogy to Intermediate 28 and was obtained as a colorless oil. MS (ESP) m/z=536.4 [M+H]⁺.

### Intermediate 40

### tert-Butyl N-[2-[tert-butyl(dimethyl)silyl]oxy-1-[4,8-difluoro-6-(hydroxymethyl)-3,5,6,7-tetrahydrocyclopenta[f]benzimidazol-2-yl]ethyl]-N-methyl-carbamate

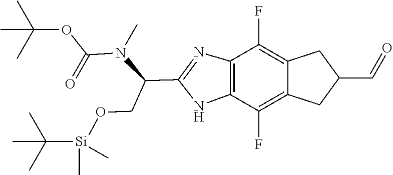 [SMILES: CN(C(=O)OC(C)(C)C)[C@H](CO[Si](C)(C)C(C)(C)C)c1nc2c(F)c3c(c(F)c2[nH]1)CC(C=O)C3]

### Step 1: (2S)-2-[tert-butoxycarbonyl(methyl)amino]-3-[tert-butyl(dimethyl)silyl]oxy-propanoic acid

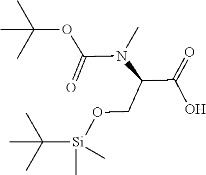 [SMILES: CN(C(=O)OC(C)(C)C)[C@H](CO[Si](C)(C)C(C)(C)C)C(=O)O]

The title compound was prepared by analogy to Intermediate 32, Step 1 using (S)-2-((tert-butoxycarbonyl)(methyl)amino)-3-hydroxypropanoic acid [CAS #101772-29-6] in place of (2S,4R)-1-[(tert-butoxy)carbonyl]-4-hydroxypyrrolidine-2-carboxylic acid and was obtained as a white solid. MS (ESP) m/z=334.3 [M+H]⁺.

### Steps 2 to 5: tert-butyl N-[(2R)-2-[tert-butyl(dimethyl)silyl]oxy-1-[4,8-difluoro-6-(hydroxymethyl)-3,5,6,7-tetrahydrocyclopenta[f]benzimidazol-2-yl]ethyl]-N-methyl-carbamate

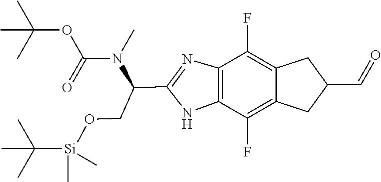 [SMILES: CN(C(=O)OC(C)(C)C)[C@H](CO[Si](C)(C)C(C)(C)C)c1nc2c(F)c3c(c(F)c2[nH]1)CC(C=O)C3]

The title compound was prepared by analogy to Intermediate 23 using (2S)-2-[tert-butoxycarbonyl(methyl)amino]-3-[tert-butyl(dimethyl)silyl]oxy-propanoic acid in place of 2-hydroxy-2-methyl-propanoic acid and was obtained as a colourless oil. MS (ESP) m/z=510.2 [M+H]⁺.

### Intermediate 41

### tert-Butyl N-[(3R,4S)-4-(4,8-difluoro-6-formyl-3,5,6,7-tetrahydrocyclopenta[f]benzimidazol-2-yl)tetrahydrofuran-3-yl]carbamate

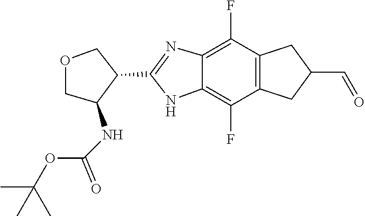 [SMILES: CC(C)(C)OC(=O)N[C@H]1COC[C@@H]1c1nc2c(F)c3c(c(F)c2[nH]1)CC(C=O)C3]

The title compound was prepared by analogy to Intermediate 31, Steps 2 to 5 using (3R,4R)-4-(tert-butoxycarbonylamino)tetrahydrofuran-3-carboxylic acid [CAS #1821806-18-1] in place of (2S,4R)-1-tert-butoxycarbonyl-4-[tert-butoxycarbonyl(methyl)amino]pyrrolidine-2-carboxylic acid and was obtained as a light brown oil. MS (ESP) m/z=408.1 [M+H]⁺.

### Intermediate 42

### tert-Butyl N-(4,8-difluoro-6-formyl-3,5,6,7-tetrahydrocyclopenta[f]benzimidazol-2-yl)carbamate

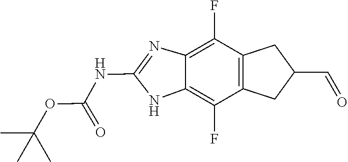 [SMILES: CC(C)(C)OC(=O)Nc1nc2c(F)c3c(c(F)c2[nH]1)CC(C=O)C3]

### Step 1: methyl 2-(tert-butoxycarbonylamino)-4,8-difluoro-3,5,6,7-tetrahydrocyclopenta[f]benzimidazole-6-carboxylate

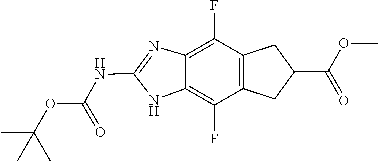 [SMILES: COC(=O)C1Cc2c(c(F)c3[nH]c(NC(=O)OC(C)(C)C)nc3c2F)C1]

To a stirred solution of methyl 5,6-diamino-4,7-difluoro-indane-2-carboxylate (Intermediate 6, 350.0 mg, 1.44 mmol, 1 eq) in acetic acid (8.54 mL) N,N′-bis(tert-butoxycarbonyl)-S-methylisothiourea [CAS #107819-90-9] (503.51 mg, 1.73 mmol, 1.2 eq) was added and the resulting mixture was stirred at 50° C. for 1.5 h. Then the mixture was concentrated under vacuum to give a crude compound that was purified by flash column chromatography (silica 50 g, cyclohexane/ethyl acetate from 100:0 to 50:50) to afford the title compound (625 mg, 1.7 mmol, quant. yield) as a light yellow solid. MS (ESP) m/z=368.2 [M+H]⁺.

### Steps 2 and 3: tert-butyl N-(4,8-difluoro-6-formyl-3,5,6,7-tetrahydrocyclopenta[f]benzimidazol-2-yl)carbamate

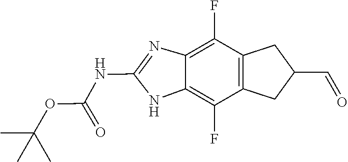 [SMILES: CC(C)(C)OC(=O)Nc1nc2c(F)c3c(c(F)c2[nH]1)CC(C=O)C3]

The title compound was prepared by analogy to Intermediate 8, Steps 3 and 4 and was obtained as a light yellow foam. MS (ESP) m/z=338.2 [M+H]⁺.

### Intermediate 43

### tert-Butyl N-[(4,8-difluoro-6-formyl-1-methyl-6,7-dihydro-5H-cyclopenta[f]benzimidazol-2-yl)methyl]-N-methyl-carbamate

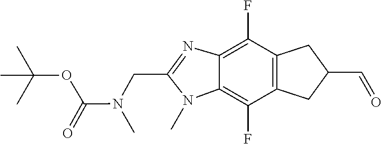 [SMILES: CN(Cc1nc2c(F)c3c(c(F)c2n1C)CC(C=O)C3)C(=O)OC(C)(C)C]

The title compound was prepared by analogy to Intermediate 28 using methyl 2-[[tert-butoxycarbonyl(methyl)amino]methyl]-4,8-difluoro-3,5,6,7-tetrahydrocyclopenta[f]benzimidazole-6-carboxylate (Intermediate 11, Step 2) in place of methyl 2-[2-[tert-butoxycarbonyl(methyl)amino]ethyl]-4,8-difluoro-3,5,6,7-tetrahydrocyclopenta[f]benzimidazole-6-carboxylate and was obtained as a colorless oil. MS (ESP) m/z=380.1 [M+H]⁺.

### Intermediate 44

### tert-Butyl 1-(4,8-difluoro-6-formyl-3,5,6,7-tetrahydrocyclopenta[f]benzimidazol-2-yl)-2-azabicyclo[2.1.1]hexane-2-carboxylate

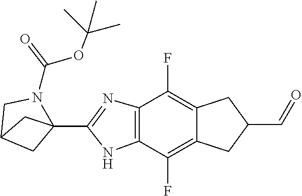 [SMILES: CC(C)(C)OC(=O)N1CC2CC1(c1nc3c(F)c4c(c(F)c3[nH]1)CC(C=O)C4)C2]

### Step 1: tert-butyl 1-[(6-amino-4,7-difluoro-2-methoxycarbonyl-indan-5-yl)carbamoyl]-2-azabicyclo[2.1.1]hexane-2-carboxylate

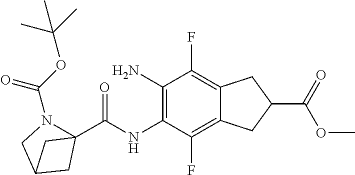 [SMILES: COC(=O)C1Cc2c(F)c(N)c(NC(=O)C34CC(CN3C(=O)OC(C)(C)C)C4)c(F)c2C1]

The title compound was prepared by analogy to Intermediate 9, Step 1 using 2-tert-butoxycarbonyl-2-azabicyclo[2.1.1]hexane-1-carboxylic acid [CAS #127926-24-3] in place of N,N-dimethylglycine and was obtained as a yellow foam. MS (ESP) m/z=452.3 [M+H]⁺.

### Step 2: methyl 2-(2-tert-butoxycarbonyl-2-azabicyclo[2.1.1]hexan-1-yl)-4,8-difluoro-3,5,6,7-tetrahydrocyclopenta[f]benzimidazole-6-carboxylate

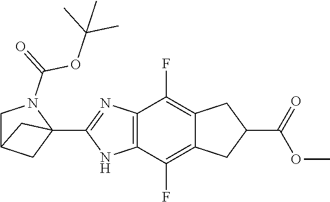 [SMILES: COC(=O)C1Cc2c(c(F)c3[nH]c(C45CC(CN4C(=O)OC(C)(C)C)C5)nc3c2F)C1]

A mixture of tert-butyl 1-[(6-amino-4,7-difluoro-2-methoxycarbonyl-indan-5-yl)carbamoyl]-2-azabicyclo[2.1.1]hexane-2-carboxylate (140.0 mg, 0.310 mmol, 1 eq) and Burgess reagent [CAS #29684-56-8] (295.59 mg, 1.24 mmol, 4 eq) in 1,2-dichloroethane (1.4 mL) was stirred under microwave irradiation for 30 min. at 120° C. The mixture was diluted with DCM and washed with water. The phases were separated, the aqueous phase was extracted with DCM, then the combined organic phases were dried over Na₂SO₄, filtered and concentrated. The obtained crude was purified by flash column chromatography (10-80% EtOAc/cyclohexane) to give the title compound (79 mg, 0.180 mmol, 58.77% yield) as a white solid. MS (ESP) m/z=434.3 [M+H]⁺.

### Steps 3 and 4: tert-butyl 1-(4,8-difluoro-6-formyl-3,5,6,7-tetrahydrocyclopenta[f]benzimidazol-2-yl)-2-azabicyclo[2.1.1]hexane-2-carboxylate

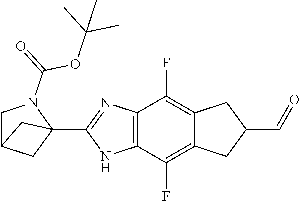 [SMILES: CC(C)(C)OC(=O)N1CC2CC1(c1nc3c(F)c4c(c(F)c3[nH]1)CC(C=O)C4)C2]

The title compound was prepared by analogy to Intermediate 8, Steps 3 and 4 and was obtained as a yellow oil. MS (ESP) m/z=404.4 [M+H]⁺.

### Intermediate 45

### tert-Butyl N-[2-[(4,8-difluoro-6-formyl-3,5,6,7-tetrahydrocyclopenta[f]benzimidazol-2-yl)amino]-2-oxo-ethyl]-N-methyl-carbamate

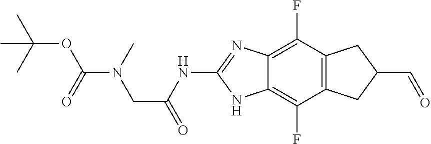 [SMILES: CN(CC(=O)Nc1nc2c(F)c3c(c(F)c2[nH]1)CC(C=O)C3)C(=O)OC(C)(C)C]

### Step 1: methyl 2-amino-4,8-difluoro-3,5,6,7-tetrahydrocyclopenta[f]benzimidazole-6-carboxylate

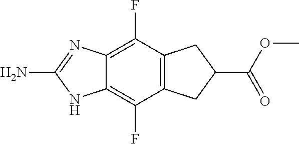 [SMILES: COC(=O)C1Cc2c(c(F)c3[nH]c(N)nc3c2F)C1]

To a stirred solution of methyl 2-(tert-butoxycarbonylamino)-4,8-difluoro-3,5,6,7-tetrahydrocyclopenta[f]benzimidazole-6-carboxylate (Intermediate 42, Step 1, 100.0 mg, 0.270 mmol, 1 eq) in DCM (2.72 mL), trifluoroacetic acid (0.4 mL, 5.25 mmol, 19.29 eq) was added and the resulting mixture was stirred at 25° C. for 1.5 h. Then the mixture was concentrated under vacuum to give a crude that was purified by silica cartridge chromatography (silica-NH 28 g, DCM/MeOH from 100:0 to 90:10) to give the title compound (55 mg, 0.210 mmol, 75.61% yield) as a light yellow solid. MS (ESP) m/z=268.2 [M+H]⁺.

### Step 2: methyl 2-[[2-[tert-butoxycarbonyl(methyl)amino]acetyl]amino]-4,8-difluoro-3,5,6,7-tetrahydrocyclopenta[f]benzimidazole-6-carboxylate

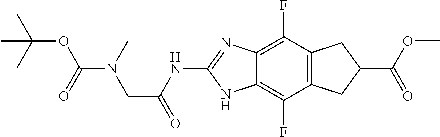 [SMILES: COC(=O)C1Cc2c(c(F)c3[nH]c(NC(=O)CN(C)C(=O)OC(C)(C)C)nc3c2F)C1]

The title compound was prepared from methyl 2-amino-4,8-difluoro-3,5,6,7-tetrahydrocyclopenta[f]benzimidazole-6-carboxylate and N-BOC sarcosine [CAS #13734-36-6] by analogy to Intermediate 9, Step 1 and was obtained as a white solid. MS (ESP) m/z=439.5 [M+H]⁺.

### Steps 3 and 4: tert-butyl N-[2-[(4,8-difluoro-6-formyl-3,5,6,7-tetrahydrocyclopenta[f]benzimidazol-2-yl)amino]-2-oxo-ethyl]-N-methyl-carbamate

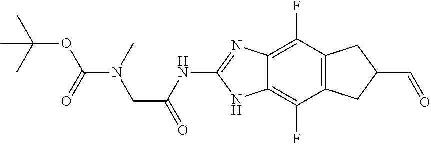 [SMILES: CN(CC(=O)Nc1nc2c(F)c3c(c(F)c2[nH]1)CC(C=O)C3)C(=O)OC(C)(C)C]

The title compound was prepared by analogy to Intermediate 31, Steps 4 and 5 and was obtained as a light yellow foam. MS (ESP) m/z=409.2 [M+H]⁺.

### Intermediate 46

### tert-Butyl N-[1-(4,8-difluoro-6-formyl-3,5,6,7-tetrahydrocyclopenta[f]benzimidazol-2-yl)-1-methyl-ethyl]carbamate

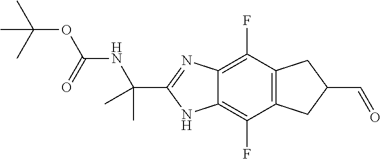 [SMILES: CC(C)(C)OC(=O)NC(C)(C)c1nc2c(F)c3c(c(F)c2[nH]1)CC(C=O)C3]

The title compound was prepared by analogy to Intermediate 23 using 2-(tert-butoxycarbonylamino)isobutyric acid [CAS #30992-29-1] in place of 2-hydroxy-2-methyl-propanoic acid. MS (ESP) m/z=380.2 [M+H]⁺.

### Intermediate 47

### tert-Butyl N-[(1S)-1-(4,8-difluoro-6-formyl-3,5,6,7-tetrahydrocyclopenta[f]benzimidazol-2-yl)ethyl]-N-methyl-carbamate

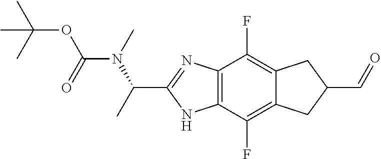 [SMILES: C[C@@H](c1nc2c(F)c3c(c(F)c2[nH]1)CC(C=O)C3)N(C)C(=O)OC(C)(C)C]

The title compound was prepared by analogy to Intermediate 26 using BOC-N-methyl-L-alanine [CAS #16948-16-6] in place of BOC-N-methyl-D-alanine and was obtained as a light yellow foam. MS (ESP) m/z=380.2 [M+H]⁺.

### Intermediate 48

### tert-Butyl N-[(1S)-1-[[tert-butyl(dimethyl)silyl]oxymethyl]-2-(4,8-difluoro-6-formyl-3,5,6,7-tetrahydrocyclopenta[f]benzimidazol-2-yl)ethyl]-N-methyl-carbamate

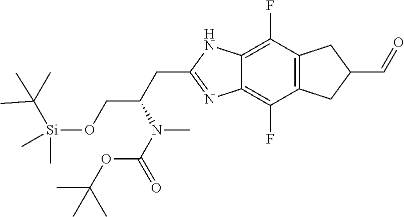 [SMILES: CN(C(=O)OC(C)(C)C)[C@H](CO[Si](C)(C)C(C)(C)C)Cc1nc2c(F)c3c(c(F)c2[nH]1)CC(C=O)C3]

### Step 1: (3S)-3-(tert-butoxycarbonylamino)-4-[tert-butyl(dimethyl)silyl]oxy-butanoic acid

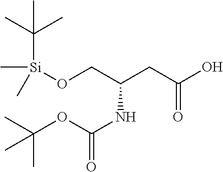 [SMILES: CC(C)(C)OC(=O)N[C@H](CO[Si](C)(C)C(C)(C)C)CC(=O)O]

The title compound was prepared by analogy to Intermediate 32, Step 1 using (3S)-3-[(tert-butoxycarbonyl)amino]-4-hydroxybutanoic acid [CAS #83345-44-2] in place of (2S,4R)-1-[(tert-butoxy)carbonyl]-4-hydroxypyrrolidine-2-carboxylic acid and was obtained as a colorless viscous oil. MS (ESP) m/z=334.3 [M+H]⁺.

### Step 2: (3S)-3-[tert-butoxy carbonyl(methyl)amino]-4-[tert-butyl(dimethyl)silyl]oxy-butanoic acid

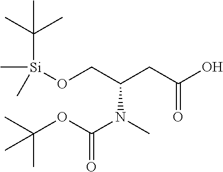 [SMILES: CN(C(=O)OC(C)(C)C)[C@H](CO[Si](C)(C)C(C)(C)C)CC(=O)O]

To a solution of (3S)-3-(tert-butoxycarbonylamino)-4-[tert-butyl(dimethyl)silyl]oxy-butanoic acid [CAS #(340.0 mg, 1.02 mmol, 14 eq in THF (4.85 mL) at 0° C., sodium hydride, 60% in oil (89.71 mg, 2.24 mmol, 2.2 eq was added portion-wise; the reaction mixture was stirred at 0° C. for 10 min. then iodomethane (130.11 uL, 2.09 mmol, 2.05 eq was added. The reaction mixture was stirred at 0° C. for 2.5 h. Then sodium hydride. 60% in oil (53.82 mg, 2.24 mmol. 2.2 ea was added at 0° C. and the reaction mixture was further stirred for 15 min., then iodomethane (130.11 uL, 2.09 mmol, 2.05 eq) was added at 0° C. and stirring was prolonged at 0° C. for 1 h, then at 5° C. for an additional 23 h. The reaction mixture was quenched with water/ice. The aqueous layer was cooled with an ice bath, the pH was set to about 2-3 by adding solid KHSO₄ and the mixture extracted with EtOAc (×4). The combined organic phases were filtered through a phase separator and volatiles were removed under reduced pressure to give the crude compound which was purified by silica cartridge chromatography (25 g, 100% DCM to 97:3 DCM/MeOH) to give the title compound (250 mg, 0.720 mmol, 70.56% yield) as a colorless viscous oil. MS (ESP) m/z=348.3 [M+H]⁺.

### Steps 3 to 6: tert-butyl N-[(1S)-1-[[tert-butyl(dimethyl)silyl]oxymethyl]-2-(4,8-difluoro-6-formyl-3,5,6,7-tetrahydrocyclopenta[f]benzimidazol-2-yl)ethyl]-N-methyl-carbamate

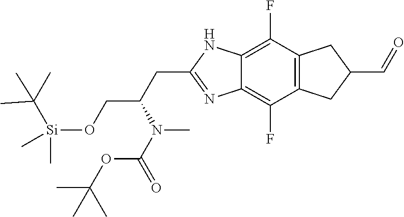 [SMILES: CN(C(=O)OC(C)(C)C)[C@H](CO[Si](C)(C)C(C)(C)C)Cc1nc2c(F)c3c(c(F)c2[nH]1)CC(C=O)C3]

The title compound was prepared from (3S)-3-[tert-butoxycarbonyl(methyl)amino]-4-[tert-butyl(dimethyl)silyl]oxy-butanoic acid and methyl 5,6-diamino-4,7-difluoro-indane-2-carboxylate (Intermediate 6) by analogy to Intermediate 9 and was obtained as a brown oil. MS (ESP) m/z=524.3 [M+H]⁺.

### Intermediate 49

### 3-[(2,4-Dimethoxyphenyl)methylamino]-5,9-difluoro-7,8-dihydro-6H-cyclopenta[g]quinoxaline-7-carbaldehyde

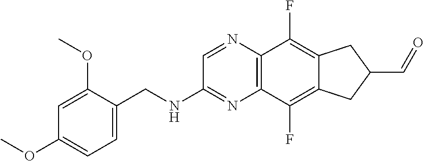 [SMILES: COc1ccc(CNc2cnc3c(F)c4c(c(F)c3n2)CC(C=O)C4)c(OC)c1]

### Step 1: methyl 5,9-difluoro-3-hydroxy-7,8-dihydro-6H-cyclopenta[g]quinoxaline-7-carboxylate

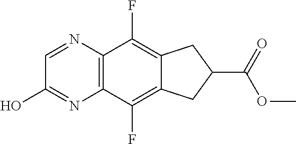 [SMILES: COC(=O)C1Cc2c(c(F)c3nc(O)cnc3c2F)C1]

To a stirred solution of methyl 5,6-diamino-4,7-difluoro-indane-2-carboxylate (Intermediate 6, 800.0 mg, 3.3 mmol, 1 eq) in dry MeCN (22.02 mL), ethyl glyoxalate (1.31 mL, 6.61 mmol, 2 eq) was added. The mixture was stirred for 24 h. The reaction mixture was concentrated in vacuo to afford the title compound (1250 mg, 4.46 mmol, 99.94% yield) as a cream solid. The material was used without further purification for the next step. MS (ESP) m/z=281.2 [M+H]⁺.

### Step 2: methyl 3-chloro-5,9-difluoro-7,8-dihydro-6H-cyclopenta[g]quinoxaline-7-carboxylate

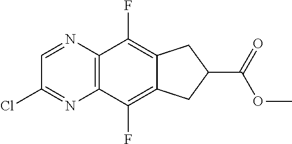 [SMILES: COC(=O)C1Cc2c(c(F)c3nc(Cl)cnc3c2F)C1]

To a flask containing methyl 5,9-difluoro-3-hydroxy-7,8-dihydro-6H-cyclopenta[g]quinoxaline-7-carboxylate (1250.0 mg, 3.3 mmol, 1 eq), phosphorus oxychloride (7.69 mL, 82.52 mmol, 25 eq) was added at RT then the mixture was stirred and heated to 100° C. for 3.5 h. Then the reaction mixture was cooled to RT and concentrated under reduced pressure. The crude product was diluted with EtOAc and washed with water (×1). The phases were separated and the aqueous phase was extracted with EtOAc (×2). Then the combined organic layers were filtered through a phase separator and concentrated in vacuo to give a residue which was purified by silica cartridge chromatography (50 g, cyclohexane/EtOAc 100:0 to 70:30) to give the title compound (267 mg, 0.940 mmol, 28.42% yield) as an off-white solid. MS (ESP) m/z=299.1 [M+H]⁺.

### Step 3: methyl 3-[(2,4-dimethoxyphenyl)methylamino]-5,9-difluoro-7,8-dihydro-6H-cyclopenta[g]quinoxaline-7-carboxylate

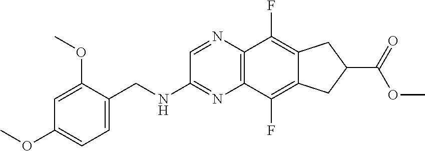

In a sealed vial, to a solution of methyl 3-chloro-5,9-difluoro-7,8-dihydro-6H-cyclopenta[g]quinoxaline-7-carboxylate (600.0 mg, 2.01 mmol, 1 eq) in dry THF (13.39 mL), 2,4-dimethoxybenzylamine (452.71 uL, 3.01 mmol, 1.5 eq) and DIPEA (600.0 uL, 3.44 mmol, 1.71 eq) were added and the reaction mixture was heated to 110° C. in the microwave for 2 h, then to 120° C. for 30 min. Then the reaction mixture was cooled to RT and diluted with EtOAc. The organic phase was washed with NaHCO₃ (sat. aq.) and then the water phase was extracted with EtOAc (×1). The combined organic layers were filtered through a phase separator and concentrated in vacuo and the residue purified by silica cartridge chromatography (50 g, EtOAc/cyclohexane 10% to 60%) to afford the title compound (689 mg, 1.6 mmol, 79.87% yield) as an off-white foam. MS (ESP) m/z=430.5 [M+H]⁺.

### Steps 4 and 5: 3-[(2,4-dimethoxyphenyl)methylamino]-5,9-difluoro-7,8-dihydro-6H-cyclopenta[g]quinoxaline-7-carbaldehyde

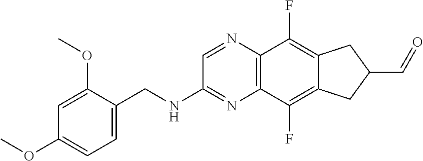 [SMILES: COc1ccc(CNc2cnc3c(F)c4c(c(F)c3n2)CC(C=O)C4)c(OC)c1]

The title compound was prepared by analogy to Intermediate 31, Steps 4 and 5 and was obtained as a yellow oil. MS (ESP) m/z=400.2 [M+H]⁺.

### Intermediate 50

### tert-Butyl N-[2-[(5,9-difluoro-7-formyl-7,8-dihydro-6H-cyclopenta[g]quinoxalin-3-yl)amino]ethyl]-N-methyl-carbamate

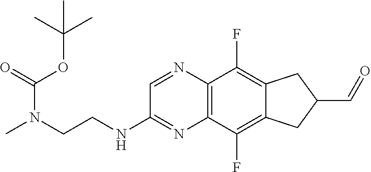 [SMILES: CN(CCNc1cnc2c(F)c3c(c(F)c2n1)CC(C=O)C3)C(=O)OC(C)(C)C]

The title compound was prepared by analogy to Intermediate 49, Steps 3 to 5 using N-(2-aminoethyl)-N-methyl carbamic acid tert-butyl ester [CAS #121492-06-6] in place of 2,4-dimethoxybenzylamine in the first step. MS (ESP) m/z=407.2 [M+H]⁺.

### Intermediate 51

### tert-Butyl N-[2-(4,8-difluoro-6-formyl-6,7-dihydro-5H-cyclopenta[f]benzotriazol-2-yl)ethyl]carbamate

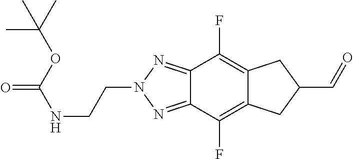 [SMILES: CC(C)(C)OC(=O)NCCn1nc2c(F)c3c(c(F)c2n1)CC(C=O)C3]

### Step 1: methyl 4,8-difluoro-3,5,6,7-tetrahydrocyclopenta[f]benzotriazole-6-carboxylate

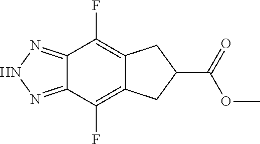 [SMILES: COC(=O)C1Cc2c(c(F)c3n[nH]nc3c2F)C1]

To a stirred solution of methyl 5,6-diamino-4,7-difluoro-indane-2-carboxylate (Intermediate 6, 500.0 mg, 2.06 mmol, 1 eq) in HCl (3.0 mL, 36 mmol, 17.44 eq), a solution of sodium nitrite (156.68 mg, 2.27 mmol, 1.1 eq) in water (1.03 mL) was added dropwise at 0° C. The resulting mixture was stirred at 0° C. for 15 min. Then the mixture was extracted with EtOAc (3×10 mL). The organic layers were collected, dried over Na₂SO₄, filtered and concentrated under vacuum to give a residue that was purified by silica cartridge chromatography (50 g, DCM/MeOH from 100:0 to 90:10) to afford the title compound (417 mg, 1.65 mmol, 79.78% yield) as a colorless oil. MS (ESP) m/z=254.1 [M+H]⁺.

### Step 2: methyl 2-[2-(tert-butoxycarbonylamino)ethyl]-4,8-difluoro-6,7-dihydro-5H-cyclopenta[f]benzotriazole-6-carboxylate and methyl 1-[2-(tert-butoxycarbonylamino)ethyl]-4,8-difluoro-6,7-dihydro-5H-cyclopenta[f]benzotriazole-6-carboxylate

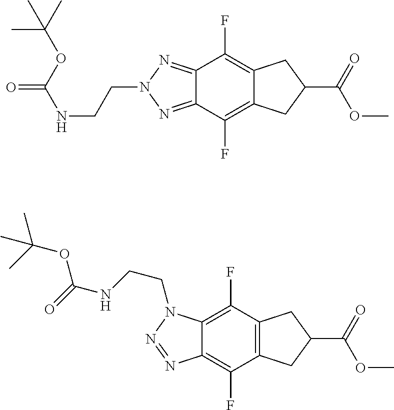

To a stirred solution of methyl 4,8-difluoro-3,5,6,7-tetrahydrocyclopenta[f]benzotriazole-6-carboxylate (417.0 mg, 1.65 mmol, 1 eq) in MeCN (13.18 mL)N-BOC-2-chloroethylamine (443.78 mg, 2.47 mmol, 1.5 eq) and potassium carbonate (1365.68 mg, 9.88 mmol, 6 eq) were added. The resulting mixture was stirred at 80° C. for 6 h. Then the reaction mixture was diluted with EtOAc (25 mL) and water (25 mL) was added. The mixture was extracted with EtOAc (3×20 mL). The combined organic layers were dried over Na₂SO₄, filtered and concentrated under vacuum to give a residue that was purified by silica cartridge chromatography (50 g, cyclohexane/ethyl acetate from 100:0 to 80:20) to give methyl 2-[2-(tert-butoxycarbonylamino)ethyl]-4,8-difluoro-6,7-dihydro-5H-cyclopenta[f]benzotriazole-6-carboxylate (279 mg, 0.700 mmol, 42.74% yield) as a colorless oil and methyl 1-[2-(tert-butoxycarbonylamino)ethyl]-4,8-difluoro-6,7-dihydro-5H-cyclopenta[f]benzotriazole-6-carboxylate (242 mg, 0.610 mmol, 37.07% yield) as a colorless oil. MS (ESP) m/z=397.5 [M+H]⁺.

### Steps 3 and 4: tert-butyl N-[2-(4,8-difluoro-6-formyl-6,7-dihydro-5H-cyclopenta[f]benzotriazol-2-yl)ethyl]carbamate

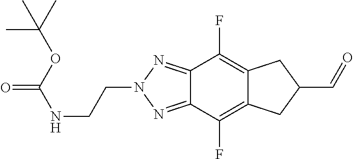 [SMILES: CC(C)(C)OC(=O)NCCn1nc2c(F)c3c(c(F)c2n1)CC(C=O)C3]

The title compound was prepared from methyl 2-[2-(tert-butoxycarbonylamino)ethyl]-4,8-difluoro-6,7-dihydro-5H-cyclopenta[f]benzotriazole-6-carboxylate by analogy to Intermediate 8, Steps 3 and 4 and was obtained as a white foam. (ESP) m/z=367.1 [M+H]⁺.

### Intermediate 52

### tert-Butyl N-[2-(4,8-difluoro-6-formyl-6,7-dihydro-5H-cyclopenta[f]benzotriazol-1-yl)ethyl]carbamate

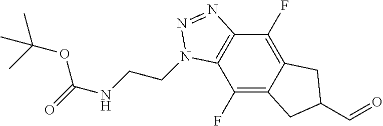 [SMILES: CC(C)(C)OC(=O)NCCn1nnc2c(F)c3c(c(F)c21)CC(C=O)C3]

The title compound was prepared from methyl 1-[2-(tert-butoxycarbonylamino)ethyl]-4,8-difluoro-6,7-dihydro-5H-cyclopenta[f]benzotriazole-6-carboxylate (Intermediate 51, Step 2) by analogy to Intermediate 8, Steps 3 and 4. MS (ESP) m/z=367.1 [M+H]⁺.

### Intermediate 53

### tert-Butyl N-[2-[1-[2-(tert-butoxycarbonylamino)ethyl]-4,8-difluoro-6-formyl-2-oxo-6,7-dihydro-5H-cyclopenta[f]benzimidazol-3-yl]ethyl]carbamate

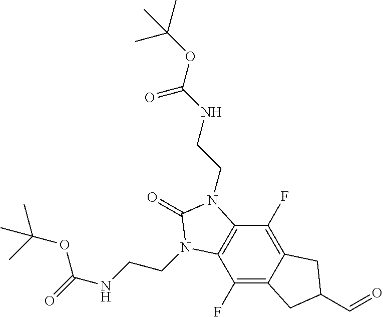 [SMILES: CC(C)(C)OC(=O)NCCn1c(=O)n(CCNC(=O)OC(C)(C)C)c2c(F)c3c(c(F)c21)CC(C=O)C3]

### Step 1: methyl 4,8-difluoro-2-oxo-3,5,6,7-tetrahydro-1H-cyclopenta[f]benzimidazole-6-carboxylate

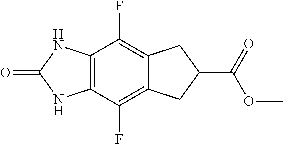 [SMILES: COC(=O)C1Cc2c(c(F)c3[nH]c(=O)[nH]c3c2F)C1]

To a solution of methyl 5,6-diamino-4,7-difluoro-indane-2-carboxylate (Intermediate 6, 100.0 mg, 0.410 mmol, 1 eq) in DCM (1.38 mL) at r.t., N,N-carbonyldiimidazole (67.61 mg, 0.420 mmol, 1.01 eq) was added in one portion; the reaction mixture was stirred at r.t. for 18 h. N,N′-carbonyldiimidazole (20.08 mg, 0.120 mmol, 0.300 eq) was added and stirring at r.t. was prolonged for an additional 1 h. The reaction mixture was filtered through a phase separator and the precipitate was washed with DCM. The solid was collected to give the crude title compound (95 mg, 0.350 mmol, 85.79% yield) as an off-white solid. MS (ESP) m/z=269.1 [M+H]⁺.

### Step 2: methyl 1,3-bis[2-(tert-butoxycarbonylamino)ethyl]-4,8-difluoro-2-oxo-6,7-dihydro-5H-cyclopenta[f]benzimidazole-6-carboxylate

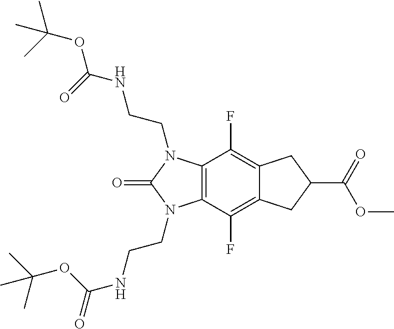 [SMILES: COC(=O)C1Cc2c(c(F)c3c(c2F)n(CCNC(=O)OC(C)(C)C)c(=O)n3CCNC(=O)OC(C)(C)C)C1]

To a solution of methyl 4,8-difluoro-2-oxo-3,5,6,7-tetrahydro-1H-cyclopenta[f]benzimidazole-6-carboxylate (30.0 mg, 0.110 mmol, 1 eq), N-BOC-ethanolamine (36.06 mg, 0.220 mmol, 2 eq) and triphenylphosphine (44.0 mg, 0.170 mmol, 1.5 eq) in THF (2.24 mL) at 0° C., diisopropyl azodicarboxylate (33.03 uL, 0.170 mmol, 1.5 eq) was added drop-wise. The reaction mixture was stirred at 0° C. for 1 h. Triphenylphosphine (44.0 mg, 0.170 mmol, 1.5 eq) and diisopropyl azodicarboxylate (33.03 uL, 0.170 mmol, 1.5 eq) were added and the reaction mixture was further stirred at 0° C. for 1 h, then at r.t. for an additional 2 h. The reaction mixture was partitioned between water and EtOAc. The phases were separated and the organic phase was washed with water (×1). The combined organic phase was filtered through a phase separator and volatiles were removed under reduced pressure to give a crude orange viscous oil which was purified by silica cartridge chromatography (25 g silica, from DCM 100% to DCM/MeOH 97.5:2.5) to give an orange gum which was further purified by reverse phase chromatography (30 g, water+0.1% formic acid/acetonitrile 9:1 to water+0.1% formic acid/acetonitrile 4:6) to give the title compound (43.9 mg, 0.079 mmol, 72% yield) as a colorless oil. MS (ESP) m/z=555.4 [M+H]⁺.

### Steps 3 and 4: tert-butyl N-[2-[1-[2-(tert-butoxycarbonylamino)ethyl]-4,8-difluoro-6-formyl-2-oxo-6,7-dihydro-5H-cyclopenta[f]benzimidazol-3-yl]ethyl]carbamate

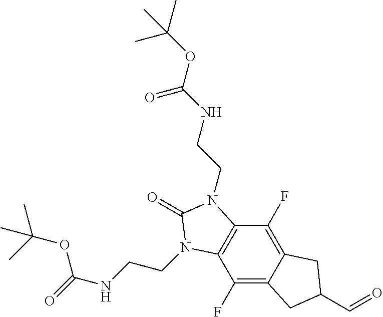 [SMILES: CC(C)(C)OC(=O)NCCn1c(=O)n(CCNC(=O)OC(C)(C)C)c2c(F)c3c(c(F)c21)CC(C=O)C3]

The title compound was prepared by analogy to Intermediate 8, Steps 3 and 4 and was obtained as a colorless viscous oil. MS (ESP) m/z=425.2 [M+H-BOC]⁺.

### Intermediate 54

### tert-Butyl N-[2-(4,8-difluoro-6-formyl-2-oxo-3,5,6,7-tetrahydrocyclopenta[f]benzimidazol-1-yl)ethyl]carbamate

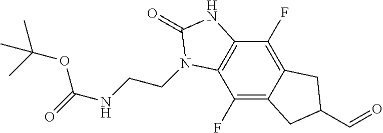 [SMILES: CC(C)(C)OC(=O)NCCn1c(=O)[nH]c2c(F)c3c(c(F)c21)CC(C=O)C3]

### Step 1: methyl 5-amino-6-[[2-(tert-butoxycarbonylamino)acetyl]amino]-4,7-difluoro-indane-2-carboxylate

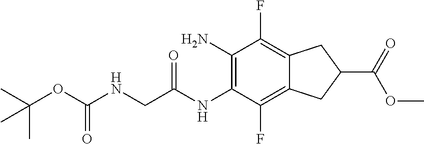 [SMILES: COC(=O)C1Cc2c(F)c(N)c(NC(=O)CNC(=O)OC(C)(C)C)c(F)c2C1]

The title compound was prepared from methyl 5,6-diamino-4,7-difluoro-indane-2-carboxylate (Intermediate 6) and N-BOC-glycine [CAS #4530-20-5] by analogy to Intermediate 9, Step 1 and was obtained as an orange viscous oil. MS (ESP) m/z=400.3 [M+H]⁺.

### Step 2: methyl 5-amino-6-[2-(tert-butoxycarbonylamino)ethylamino]-4,7-difluoro-indane-2-carboxylate

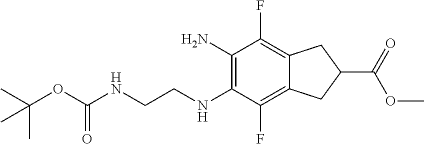 [SMILES: COC(=O)C1Cc2c(F)c(N)c(NCCNC(=O)OC(C)(C)C)c(F)c2C1]

To a solution of methyl 5-amino-6-[[2-(tert-butoxycarbonylamino)acetyl]amino]-4,7-difluoro-indane-2-carboxylate (250.0 mg, 0.630 mmol, 1 eq) in THF (3.13 mL) at 0° C., borane-THF complex (2.19 mL, 2.19 mmol, 3.5 eq) was added dropwise. The reaction mixture was stirred at 0° C. for 30 min. then at r.t. for 3.5 h. Then borane-THF complex (1.0 mL, 1 mmol, 1.6 eq) was added at 0° C. and the reaction mixture stirred at r.t. for an additional 2 h. The reaction mixture was added dropwise to NH₄Cl (sat. aq.) and extracted with EtOAc (×1). The organic phase was filtered through a phase separator and volatiles were removed under reduced pressure to give a residue which was purified by silica cartridge chromatography (5 g silica, cyclohexane 100% to cyclohexane/EtOAc 7:3) to give the title compound (105 mg, 0.270 mmol, 43.52% yield) as a colorless oil. MS (ESP) m/z=386.2 [M+H]⁺.

### Step 3: methyl 1-[2-(tert-butoxycarbonylamino)ethyl]-4,8-difluoro-2-oxo-3,5,6,7-tetrahydrocyclopenta[f]benzimidazole-6-carboxylate

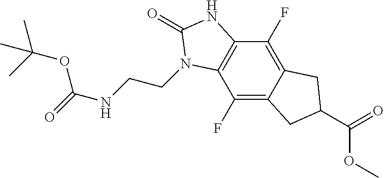 [SMILES: COC(=O)C1Cc2c(c(F)c3c([nH]c(=O)n3CCNC(=O)OC(C)(C)C)c2F)C1]

The title compound was prepared from methyl 5-amino-6-[2-(tert-butoxycarbonylamino)ethylamino]-4,7-difluoro-indane-2-carboxylate by analogy to Intermediate 53, Step 1 and was obtained as a white solid. MS (ESP) m/z=412.2 [M+H]⁺.

### Steps 4 and 5: tert-butyl N-[2-(4,8-difluoro-6-formyl-2-oxo-3,5,6,7-tetrahydrocyclopenta[f]benzimidazol-1-yl)ethyl]carbamate

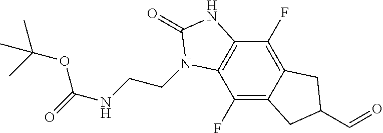 [SMILES: CC(C)(C)OC(=O)NCCn1c(=O)[nH]c2c(F)c3c(c(F)c21)CC(C=O)C3]

The title compound was prepared by analogy to Intermediate 8, Steps 3 and 4 and was obtained as a yellow viscous oil. MS (ESN) m/z=380.3 [M−H]⁻.

### Intermediate 55

### tert-Butyl N-[(1R)-1-(8-fluoro-6-formyl-1,5,6,7-tetrahydrocyclopenta[f]benzimidazol-2-yl)ethyl]carbamate

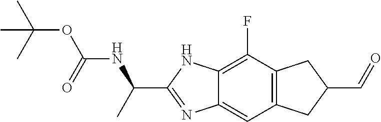 [SMILES: C[C@@H](NC(=O)OC(C)(C)C)c1nc2cc3c(c(F)c2[nH]1)CC(C=O)C3]

### Step 1: ethyl 5-amino-6-[[(2R)-2-(tert-butoxycarbonylamino)propanoyl]amino]-4-fluoro-indane-2-carboxylate and ethyl 6-amino-5-[[(2R)-2-(tert-butoxycarbonylamino)propanoyl]amino]-4-fluoro-indane-2-carboxylate

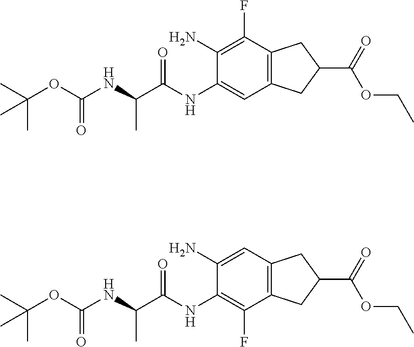

To a solution of ethyl 5,6-diamino-4-fluoro-indane-2-carboxylate (Intermediate 7, 150.0 mg, 0.630 mmol, 1 eq) and N-BOC-D-alanine [CAS #7764-95-6] (119.12 mg, 0.630 mmol, 1 eq) in DMF (3.15 mL) at r.t., PyBOP (327.62 mg, 0.630 mmol, 1 eq) was added in one portion, followed by DIPEA (0.22 mL, 1.26 mmol, 2 eq). The reaction mixture was stirred at r.t. for 2 h. The reaction mixture was quenched with brine and diluted with EtOAc. The phases were separated and the organic phase was washed with brine (×1). The combined organic phase was filtered through a phase separator and volatiles were removed under reduced pressure to give a residue which was purified by silica cartridge chromatography (10 g silica, DCM 100% to DCM/[EtOAc/MeOH 9:1] 9:1) to give ethyl 5-amino-6-[[(2R)-2-(tert-butoxycarbonylamino)propanoyl]amino]-4-fluoro-indane-2-carboxylate (155 mg, 0.380 mmol, 60.13% yield) as a light yellow solid and ethyl 6-amino-5-[[(2R)-2-(tert-butoxycarbonylamino)propanoyl]amino]-4-fluoro-indane-2-carboxylate (21 mg, 0.050 mmol, 8.15% yield) as a light yellow solid. MS (ESP) m/z=410.3 [M+H]⁺.

### Step 2: ethyl 2-[(1R)-1-(tert-butoxycarbonylamino)ethyl]-8-fluoro-3,5,6,7-tetrahydrocyclopenta[f]benzimidazole-6-carboxylate

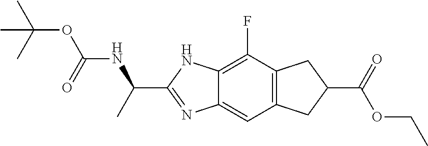 [SMILES: CCOC(=O)C1Cc2cc3nc([C@@H](C)NC(=O)OC(C)(C)C)[nH]c3c(F)c2C1]

The title compound was prepared by analogy to Intermediate 8, Step 2 and was obtained as a light yellow viscous oil. MS (ESP) m/z=392.3 [M+H]⁺.

### Steps 3 and 4: tert-butyl N-[(1R)-1-(8-fluoro-6-formyl-1,5,6,7-tetrahydrocyclopenta[f]benzimidazol-2-yl)ethyl]carbamate

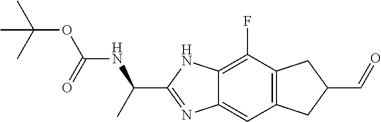 [SMILES: C[C@@H](NC(=O)OC(C)(C)C)c1nc2cc3c(c(F)c2[nH]1)CC(C=O)C3]

The title compound was prepared by analogy to Intermediate 28, Steps 2 and 3 and was obtained as a yellow viscous oil. MS (ESP) m/z=348.3 [M+H]⁺.

### Intermediate 56

### 2-[(2S,4R)-4-[tert-Butyl(dimethyl)silyl]oxy-1-methyl-pyrrolidin-2-yl]-4,8-difluoro-3,5,6,7-tetrahydrocyclopenta[f]benzimidazole-6-carbaldehyde

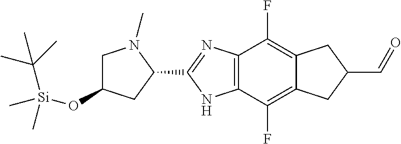 [SMILES: CN1C[C@H](O[Si](C)(C)C(C)(C)C)C[C@H]1c1nc2c(F)c3c(c(F)c2[nH]1)CC(C=O)C3]

### Step 1: methyl 2-[(2S,4R)-4-[tert-butyl(dimethyl)silyl]oxypyrrolidin-2-yl]-4,8-difluoro-3,5,6,7-tetrahydrocyclopenta[f]benzimidazole-6-carboxylate

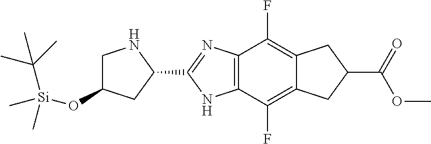 [SMILES: COC(=O)C1Cc2c(c(F)c3[nH]c([C@@H]4C[C@@H](O[Si](C)(C)C(C)(C)C)CN4)nc3c2F)C1]

A solution of methyl 2-[(2S,4R)-1-tert-butoxycarbonyl-4-[tert-butyl(dimethyl)silyl]oxy-pyrrolidin-2-yl]-4,8-difluoro-3,5,6,7-tetrahydrocyclopenta[f]benzimidazole-6-carboxylate (Intermediate 32, Step 3, 218.0 mg, 0.400 mmol, 1 eq) in 1,1,1,3,3,3-hexafluoro-2-propanol (4.36 mL, 41.51 mmol, 105.06 eq) was stirred under micro-wave irradiation at 145° C. for 1 h. Volatiles were removed under reduced pressure to give the title compound (141 mg, 0.310 mmol, 79.02% yield) as a colorless oil. MS (ESP) m/z=452.4 [M+H]⁺.

### Step 2: methyl 2-[(2S,4R)-4-[tert-butyl(dimethyl)silyl]oxy-1-methyl-pyrrolidin-2-yl]-4,8-difluoro-3,5,6,7-tetrahydrocyclopenta[f]benzimidazole-6-carboxylate

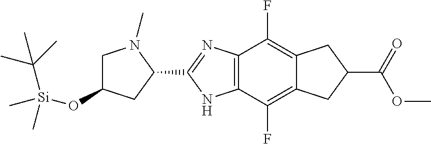 [SMILES: COC(=O)C1Cc2c(c(F)c3[nH]c([C@@H]4C[C@@H](O[Si](C)(C)C(C)(C)C)CN4C)nc3c2F)C1]

The title compound was prepared by analogy to Intermediate 25, Step 1 and was obtained as a light yellow oil. MS (ESP) m/z=466.3 [M+H]⁺.

### Steps 3 and 4: 2-[(2S,4R)-4-[tert-butyl(dimethyl)silyl]oxy-1-methyl-pyrrolidin-2-yl]-4,8-difluoro-3,5,6,7-tetrahydrocyclopenta[f]benzimidazole-6-carbaldehyde

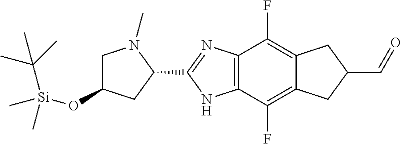 [SMILES: CN1C[C@H](O[Si](C)(C)C(C)(C)C)C[C@H]1c1nc2c(F)c3c(c(F)c2[nH]1)CC(C=O)C3]

The title compound was prepared by analogy to Intermediate 28, Steps 2 and 3 and was obtained as a light yellow oil. MS (ESP) m/z=436.4 [M+H]⁺.

### Intermediate 57

### tert-Butyl (2R,4S)-2-(4,8-difluoro-6-formyl-3,5,6,7-tetrahydrocyclopenta[f]benzimidazol-2-yl)-4-methoxy-pyrrolidine-1-carboxylate

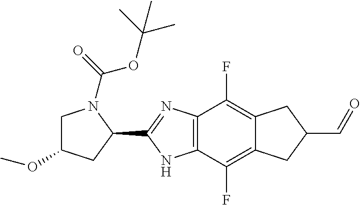 [SMILES: CO[C@H]1C[C@H](c2nc3c(F)c4c(c(F)c3[nH]2)CC(C=O)C4)N(C(=O)OC(C)(C)C)C1]

The title compound was prepared from methyl 5,6-diamino-4,7-difluoro-indane-2-carboxylate (Intermediate 6) and (2R,4S)-1-tert-butoxycarbonyl-4-methoxy-pyrrolidine-2-carboxylic acid [CAS #147266-70-4] by analogy to Intermediate 38, Steps 2 to 5 and was obtained as a colourless oil. MS (ESP) m/z=422.2 [M+H]⁺.

### Intermediate 58

### 2-[2-(Dimethylamino)ethyl]-8-fluoro-6,7-dihydro-5H-cyclopenta[f][1,3]benzoxazole-6-carbaldehyde

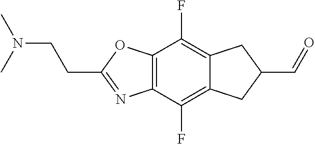 [SMILES: CN(C)CCc1nc2c(F)c3c(c(F)c2o1)CC(C=O)C3]

The title compound was prepared by analogy to Intermediate 29, Steps 2 to 5 using 3-(dimethylamino)propanoic acid in place of BOC-SAR-OH and was obtained as a light brown gum. MS (ESP) m/z=277.2 [M+H]⁺.

### Intermediate 59

### tert-Butyl N-[(1S)-1-[[tert-butyl(dimethyl)silyl]oxymethyl]-2-(4,8-difluoro-6-formyl-3,5,6,7-tetrahydrocyclopenta[f]benzimidazol-2-yl)ethyl]carbamate

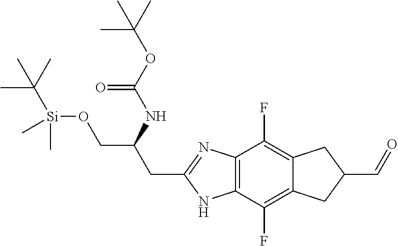 [SMILES: CC(C)(C)OC(=O)N[C@H](CO[Si](C)(C)C(C)(C)C)Cc1nc2c(F)c3c(c(F)c2[nH]1)CC(C=O)C3]

The title compound was prepared from (3S)-3-(tert-butoxycarbonylamino)-4-[tert-butyl(dimethyl)silyl]oxy-butanoic acid (Intermediate 48, Step 1) and methyl 5,6-diamino-4,7-difluoro-indane-2-carboxylate (Intermediate 6) by analogy to Intermediate 48, Steps 3 to 6 and was obtained as a yellow oil. MS (ESP) m/z=510.38 [M+H]⁺.

### Intermediate 60

### tert-Butyl N-[2-tert-butoxy-1-(4,8-difluoro-6-formyl-3,5,6,7-tetrahydrocyclopenta[f]benzimidazol-2-yl)ethyl]-N-methyl-carbamate

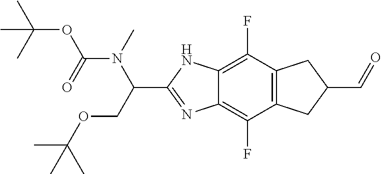 [SMILES: CN(C(=O)OC(C)(C)C)C(COC(C)(C)C)c1nc2c(F)c3c(c(F)c2[nH]1)CC(C=O)C3]

### Step 1: (2R)-3-tert-butoxy-2-[tert-butoxycarbonyl(methyl)amino]propanoic acid

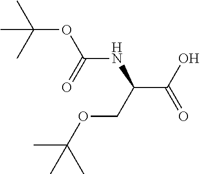 [SMILES: CC(C)(C)OC[C@@H](NC(=O)OC(C)(C)C)C(=O)O]

To a solution of (2R)-3-tert-butoxy-2-(tert-butoxycarbonylamino)propanoic acid [CAS #248921-66-6](1000.0 mg, 3.83 mmol, 1.0 eq) in THF (12.5 mL) at −15° C. (ice/NaCl bath) sodium hydride (60% in oil, 336.73 mg, 8.42 mmol, 2.2 eq) was added portion-wise. The reaction mixture was stirred at −15° C. for 10 min. then iodomethane (0.49 mL, 7.85 mmol, 2.05 eq) and DMF (0.65 mL) were added. The reaction mixture was stirred at −15° C. for 2.5 h. Further iodomethane (0.12 mL, 1.91 mmol, 0.5 eq) was added at −15° C. and the reaction mixture was further stirred at −15° C. After 1.5 h, sodium hydride, 60% in oil (82.65 mg, 3.44 mmol, 0.9 eq) was added at −15° C. and stirring was prolonged at −15° C. for 1 h, then at 5° C. (ice bath) for an additional 23 h. The reaction mixture was quenched with water/ice and washed with EtOAc (once). The aqueous layer was collected and cooled with an ice bath, then the pH was set to about 3 with 1 M HCl aq., diluted with Na₂S₂O₃ (sat. aq.) and extracted with EtOAc (four times). The combined organic phases were filtered through a phase separator and volatiles were removed under reduced pressure to give the crude title compound (1260.0 mg) as a light yellow viscous oil. MS (ESN) m/z=374.3 [M−H]⁻.

### Steps 2 and 3: methyl 2-[2-tert-butoxy-1-[tert-butoxycarbonyl(methyl)amino]ethyl]-4,8-difluoro-3,5,6,7-tetrahydrocyclopenta[f]benzimidazole-6-carboxylate

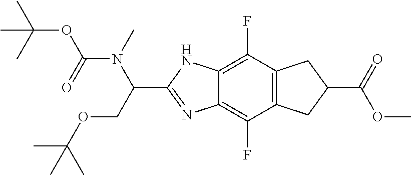 [SMILES: COC(=O)C1Cc2c(c(F)c3[nH]c(C(COC(C)(C)C)N(C)C(=O)OC(C)(C)C)nc3c2F)C1]

The title compound was prepared from methyl 5,6-diamino-4,7-difluoro-indane-2-carboxylate (Intermediate 6) and (2R)-3-tert-butoxy-2-[tert-butoxycarbonyl(methyl)amino]propanoic acid by analogy with Intermediate 23, Steps 1 and 2 and was obtained as a white foam. MS (ESP) m/z=482.3 [M+H]⁺.

### Steps 4 and 5: tert-butyl N-[2-tert-butoxy-1-(4,8-difluoro-6-formyl-3,5,6,7-tetrahydrocyclopenta[f]benzimidazol-2-yl)ethyl]-N-methyl-carbamate

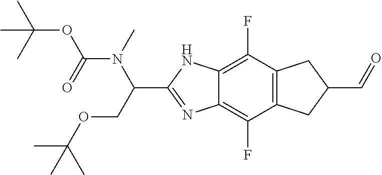 [SMILES: CN(C(=O)OC(C)(C)C)C(COC(C)(C)C)c1nc2c(F)c3c(c(F)c2[nH]1)CC(C=O)C3]

The title compound was prepared by analogy with Intermediate 28, Steps 2 and 3, and was obtained as a colorless amorphous solid. MS (ESP) m/z=452.3 [M+H]⁺.

### Intermediate 61

### tert-Butyl N-[(1R)-1-(4,8-difluoro-6-formyl-1-methyl-6,7-dihydro-5H-cyclopenta[f]benzimidazol-2-yl)ethyl]carbamate

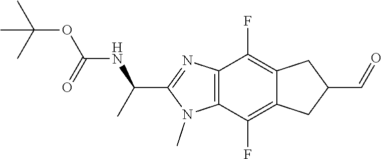 [SMILES: C[C@@H](NC(=O)OC(C)(C)C)c1nc2c(F)c3c(c(F)c2n1C)CC(C=O)C3]

The title compound was prepared from methyl 2-[(1R)-1-(tert-butoxycarbonylamino)ethyl]-4,8-difluoro-3,5,6,7-tetrahydrocyclopenta[f]benzimidazole-6-carboxylate (Intermediate 20, Step 2) by analogy with Intermediate 28 and was obtained as a light yellow oil.

### Intermediate 62

### tert-Butyl N-[2-(8-fluoro-6-formyl-1,5,6,7-tetrahydrocyclopenta[f]benzimidazol-2-yl)ethyl]-N-methyl-carbamate

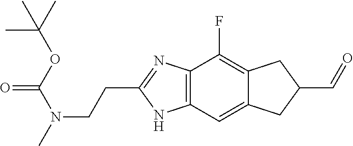 [SMILES: CN(CCc1nc2c(F)c3c(cc2[nH]1)CC(C=O)C3)C(=O)OC(C)(C)C]

The title compound was prepared from 3-[tert-butoxycarbonyl(methyl)amino]propanoic acid [CAS #124072-61-3] and Intermediate 7 by analogy with Intermediate 9 and was obtained as a light brown foam. MS (ESP) m/z=362.2 [M+H]⁺.

### Intermediate 63

### tert-Butyl N-[(8-fluoro-6-formyl-3,5,6,7-tetrahydrocyclopenta[f]benzimidazol-2-yl)methyl]-N-(2-methoxyethyl)carbamate

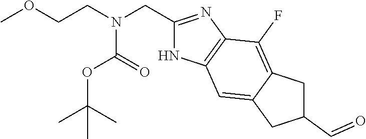 [SMILES: COCCN(Cc1nc2c(F)c3c(cc2[nH]1)CC(C=O)C3)C(=O)OC(C)(C)C]

### Step 1: ethyl 2-(chloromethyl)-8-fluoro-3,5,6,7-tetrahydrocyclopenta[f]benzimidazole-6-carboxylate

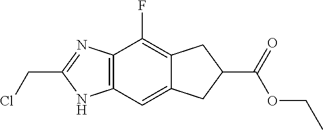 [SMILES: CCOC(=O)C1Cc2cc3[nH]c(CCl)nc3c(F)c2C1]

A flask was charged with ethyl 5,6-diamino-4-fluoro-indane-2-carboxylate (Intermediate 7, 150.0 mg, 0.63 mmol, 1.0 eq) and ethanimidic acid, 2-chloro-, ethyl ester, hydrochloride [CAS #36743-66-5] (1:1) (229.6 mg, 1.89 mmol, 3.0 eq) in methanol (6.3 mL) under a nitrogen atmosphere. The reaction mixture was heated to 65° C. for 0.5 h. The reaction was quenched with water and extracted with EtOAc (×3). The organic phases were combined and dried in vacuo to give the title compound (152.0 mg, 0.51 mmol, 81.4% yield) as a light yellow solid. MS (ESP) m/z=297.2, 299.2 [M+H]⁺.

### Step 2: ethyl 8-fluoro-2-[(2-methoxyethylamino)methyl]-3,5,6,7-tetrahydrocyclopenta[f]benzimidazole-6-carboxylate

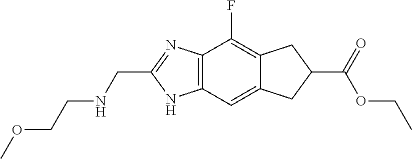 [SMILES: CCOC(=O)C1Cc2cc3[nH]c(CNCCOC)nc3c(F)c2C1]

To a flask under nitrogen atmosphere charged with ethyl 2-(chloromethyl)-8-fluoro-3,5,6,7-tetrahydrocyclopenta[f]benzimidazole-6-carboxylate (152.0 mg, 0.51 mmol, 1.0 eq) and potassium iodide (85.0 mg, 0.51 mmol, 1.0 eq) in acetonitrile (5.12 mL) was added 2-methoxyethylamine (0.11 mL, 1.28 mmol, 2.5 eq). The reaction was stirred at 25° C. for 4 h. The reaction was quenched with water and extracted with EtOAc (2×). The crude was purified by flash column chromatography (silica-NH, 11 g, DCM to 80:20 DCM/MeOH) to give the title compound (124.0 mg, 0.37 mmol, 72.2% yield) as a yellow oil. MS (ESP) m/z=336.2 [M+H]⁺.

### Step 3: ethyl 2-[[tert-butoxycarbonyl(2-methoxyethyl)amino]methyl]-8-fluoro-1,5,6,7-tetrahydrocyclopenta[f]benzimidazole-6-carboxylate

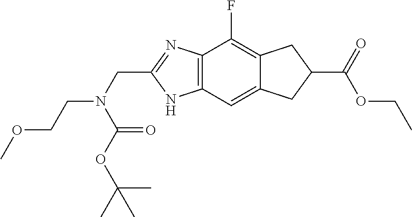 [SMILES: CCOC(=O)C1Cc2cc3[nH]c(CN(CCOC)C(=O)OC(C)(C)C)nc3c(F)c2C1]

To a stirred solution of methyl ethyl 8-fluoro-2-[(2-methoxyethylamino)methyl]-1,5,6,7-tetrahydrocyclopenta[f]benzimidazole-6-carboxylate (124.0 mg, 0.37 mmol, 1.0 eq) in dry THF (4.6 mL) was added di-tert-butyldicarbonate (177.5 mg, 0.81 mmol, 2.2 eq) and the mixture was stirred at 25° C. for 20 h. The mixture was cooled to 0° C., carefully quenched with NH₄Cl (sat. aq.) and extracted with EtOAc (×3). The combined organic phase was dried over Na₂SO₄ and concentrated in vacuo to afford the crude title compound (176.0 mg, 0.4 mmol, quant. yield) as a colorless viscous oil which was used in the next step without any purification. MS (ESP) m/z=436.4 [M+H]⁺.

### Steps 4 and 5: tert-butyl N-[(8-fluoro-6-formyl-3,5,6,7-tetrahydrocyclopenta[f]benzimidazol-2-yl)methyl]-N-(2-methoxyethyl)carbamate

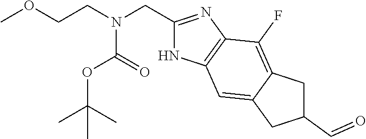 [SMILES: COCCN(Cc1nc2c(F)c3c(cc2[nH]1)CC(C=O)C3)C(=O)OC(C)(C)C]

The title compound was prepared by analogy with Intermediate 28, Steps 2 and 3, and was obtained as a colorless oil. MS (ESP) m/z=392.2 [M+H]⁺.

### Intermediate 64

### tert-Butyl N-[(8-fluoro-6-formyl-3,5,6,7-tetrahydrocyclopenta[f]benzimidazol-2-yl)methyl]-N-[2-(methylamino)-2-oxo-ethyl]carbamate

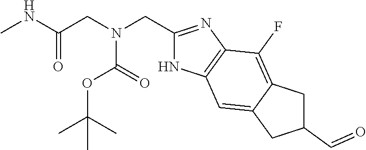 [SMILES: CNC(=O)CN(Cc1nc2c(F)c3c(cc2[nH]1)CC(C=O)C3)C(=O)OC(C)(C)C]

The title compound was prepared by analogy with Intermediate 63, Steps 2 to 5 using acetamide, 2-amino-N-methyl-, hydrochloride (1:1) [CAS #49755-94-4] in place of 2-methoxyethylamine in Step 2 and was obtained as a colorless oil. MS (ESP) m/z=405.4 [M+H]⁺.

### Intermediate 65

### tert-Butyl N-[2-(4-fluoro-6-formyl-2-methyl-6,7-dihydro-5H-cyclopenta[f]benzimidazol-3-yl)ethyl]carbamate; tert-butyl N-[2-(8-fluoro-6-formyl-2-methyl-6,7-dihydro-5H-cyclopenta[f]benzimidazol-3-yl)ethyl]carbamate

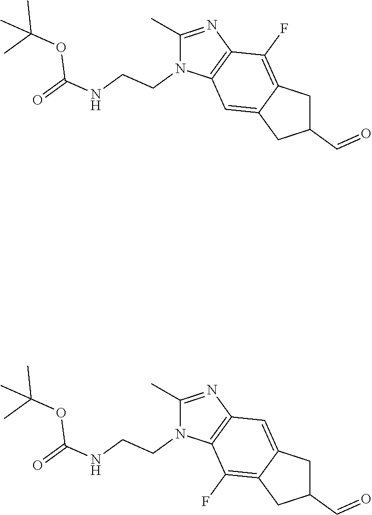 [SMILES: Cc1nc2c(F)c3c(cc2n1CCNC(=O)OC(C)(C)C)CC(C=O)C3 ; Cc1nc2cc3c(c(F)c2n1CCNC(=O)OC(C)(C)C)CC(C=O)C3]

### Step 1: ethyl 5,6-diacetamido-4-fluoro-indane-2-carboxylate

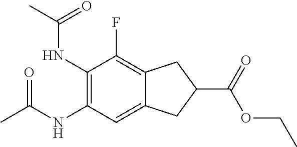 [SMILES: CCOC(=O)C1Cc2cc(NC(C)=O)c(NC(C)=O)c(F)c2C1]

To a solution of ethyl 5,6-diamino-4-fluoro-indane-2-carboxylate (Intermediate 7, 370.0 mg, 1.09 mmol, 1.0 eq) in DCM (14.1 mL) and N,N-diisopropylethylamine (0.38 mL, 2.17 mmol, 2.0 eq) was added acetyl chloride (0.09 mL, 1.3 mmol, 1.2 eq) dropwise at 0° C. in an ice bath. The reaction was then allowed to warm up at room temperature and stirred for 1 h. The mixture was quenched with NaHCO₃ (sat. aq.), extracted with DCM and then filtered through a hydrophobic phase separator, concentrated in vacuo and the reside purified by silica cartridge chromatography (25 g, 0% to 40% EtOAc/cyclohexane) to afford the title compound (350.0 mg, 1.09 mmol, 99.9% yield) as a light yellow oil. MS (ESP) m/z=323.2 [M+H]⁺.

### Step 2: N-[6-acetamido-7-fluoro-2-(hydroxymethyl)indan-5-yl]acetamide

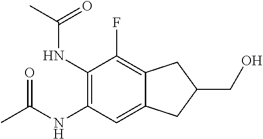 [SMILES: CC(=O)Nc1cc2c(c(F)c1NC(C)=O)CC(CO)C2]

To a stirred solution of methyl ethyl 5,6-diacetamido-4-fluoro-indane-2-carboxylate (350.0 mg, 1.09 mmol, 1.0 eq) in dry THF (7.2 mL) and ethanol (3.6 mL), calcium chloride (241.0 mg, 2.17 mmol, 2.0 eq) and sodium borohydride (123.2 mg, 3.26 mmol, 3.0 eq) were added and the mixture was stirred at RT for 48 h. The mixture was cooled to 0° C., quenched with NH₄Cl (sat. aq.) and extracted with EtOAc (×3). The combined organic phase was dried over Na₂SO₄ and concentrated in vacuo. The crude product was purified by silica cartridge chromatography (0% to 20% MeOH/DCM) to give the title compound (170.0 mg, 0.61 mmol, 55.9% yield) as a light yellow oil. MS (ESP) m/z=281.2 [M+H]⁺.

### Step 3: (8-fluoro-2-methyl-3,5,6,7-tetrahydrocyclopenta[f]benzimidazol-6-yl)methanol

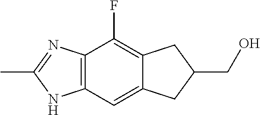 [SMILES: Cc1nc2c(F)c3c(cc2[nH]1)CC(CO)C3]

N-[6-Acetamido-7-fluoro-2-(hydroxymethyl)indan-5-yl]acetamide (170.0 mg, 0.61 mmol, 1.0 eq) was suspended in toluene (6.1 mL) and p-toluenesulfonic acid monohydrate (230.74 mg, 1.21 mmol, 2.0 eq) was added and the mixture refluxed for 16 h. The reaction was quenched with water and extracted with EtOAc (×3), the combined organic phase was concentrated and the crude product was purified by silica column chromatography (0% to 15% MeOH/DCM) to give the title compound (77.0 mg, 0.35 mmol, 57.6% yield) as a light yellow oil. MS (ESP) m/z=221.0 [M+H]⁺.

### Step 4: tert-butyl-[(8-fluoro-2-methyl-3,5,6,7-tetrahydrocyclopenta[f]benzimidazol-6-yl)methoxy]-dimethyl-silane

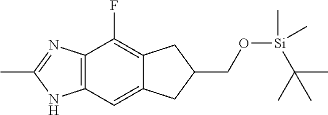 [SMILES: Cc1nc2c(F)c3c(cc2[nH]1)CC(CO[Si](C)(C)C(C)(C)C)C3]

A mixture of (8-fluoro-2-methyl-3,5,6,7-tetrahydrocyclopenta[f]benzimidazol-6-yl)methanol (77.0 mg, 0.35 mmol, 1.0 eq) and imidazole (119.0 mg, 1.75 mmol, 5.0 eq) in DCM (2.8 mL) and DMF (0.7 mL) was cooled to 0° C. then tert-butyldimethylchlorosilane (115.93 mg, 0.77 mmol, 2.2 eq) was added portionwise. The mixture was left to reach room temperature and stirred for 6 h. EtOAc was added, the mixture was washed with water (×3), then dried over Na₂SO₄ and concentrated under reduced pressure to give the title compound (125.0 mg, 0.37 mmol, quant. yield) as a light yellow viscous oil. MS (ESP) m/z=335.3 [M+H]⁺.

### Step 5: tert-butyl N-[2-[6-[[tert-butyl(dimethyl)silyl]oxymethyl]-4-fluoro-2-methyl-6,7-dihydro-5H-cyclopenta[f]benzimidazol-3-yl]ethyl]carbamate; tert-butyl N-[2-[6-[[tert-butyl(dimethyl)silyl]oxymethyl]-8-fluoro-2-methyl-6,7-dihydro-5H-cyclopenta[f]benzimidazol-3-yl]ethyl]carbamate

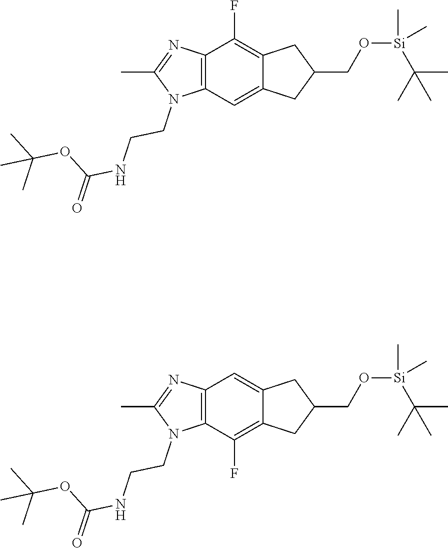

To a stirred solution of tert-butyl-[(8-fluoro-2-methyl-3,5,6,7-tetrahydrocyclopenta[f]benzimidazol-6-yl)methoxy]-dimethyl-silane (125.0 mg, 0.37 mmol, 1.0 eq) in MeCN (4.5 mL)N-BOC-2-chloroethylamine (100.7 mg, 0.56 mmol, 1.5 eq) and potassium carbonate (309.88 mg, 2.24 mmol, 6.0 eq) were added. The resulting mixture was stirred at 80° C. for 18 h. Then the reaction mixture was quenched by water addition, extracted with EtOAc (×3) and concentrated in vacuo. The crude mixture was purified by flash chromatography (silica, 10 g, 0% to 40% EtOAc/cyclohexane) to afford the title compound mixture (214.1 mg, 0.45 mmol, quant. yield) as a light yellow oil. MS (ESP) m/z=478.6, 478.4 [M+H]⁺.

### Step 6: tert-butyl N-[2-[4-fluoro-6-(hydroxymethyl)-2-methyl-6,7-dihydro-5H-cyclopenta[f]benzimidazol-3-yl]ethyl]carbamate; tert-butyl N-[2-[8-fluoro-6-(hydroxymethyl)-2-methyl-6,7-dihydro-5H-cyclopenta[f]benzimidazol-3-yl]ethyl]carbamate

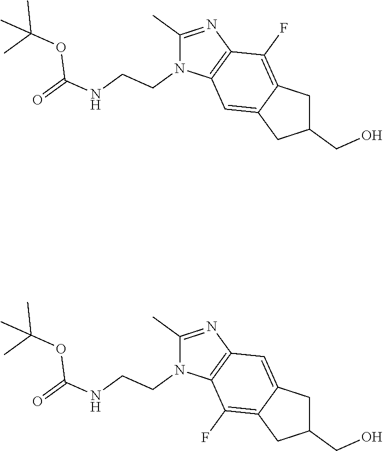 [SMILES: Cc1nc2c(F)c3c(cc2n1CCNC(=O)OC(C)(C)C)CC(CO)C3 ; Cc1nc2cc3c(c(F)c2n1CCNC(=O)OC(C)(C)C)CC(CO)C3]

To a solution of tert-butyl N-[2-[6-[[tert-butyl(dimethyl)silyl]oxymethyl]-4-fluoro-2-methyl-6,7-dihydro-5H-cyclopenta[f]benzimidazol-3-yl]ethyl]carbamate; tert-butyl N-[2-[6-[[tert-butyl(dimethyl)silyl]oxymethyl]-8-fluoro-2-methyl-6,7-dihydro-5H-cyclopenta[f]benzimidazol-3-yl]ethyl]carbamate (214.0 mg, 0.45 mmol, 1.0 eq) in tetrahydrofuran (5.2 mL) in an ice-cold bath, tetrabutylammonium fluoride (1M in THF, 1.12 mL, 1.12 mmol, 2.5 eq) was added; the reaction mixture was stirred at 0° C. for 5 min, then at RT for 6 h. The reaction mixture was diluted with EtOAc and NH₄Cl (sat. aq.). The phases were separated and the organic phase was washed with NH₄Cl (sat. aq.) (twice). The crude product was purified by silica gel chromatography (5 g silica, from 0% to 100% EtOAc/cyclohexane) to give the title compound mixture (41 mg, 0.060 mmol, 67.3% yield) as a light yellow oil. MS (ESP) m/z=364.2 [M+H]⁺.

### Step 7: tert-butyl N-[2-(4-fluoro-6-formyl-2-methyl-6,7-dihydro-5H-cyclopenta[f]benzimidazol-3-yl)ethyl]carbamate; tert-butyl N-[2-(8-fluoro-6-formyl-2-methyl-6,7-dihydro-5H-cyclopenta[f]benzimidazol-3-yl)ethyl]carbamate

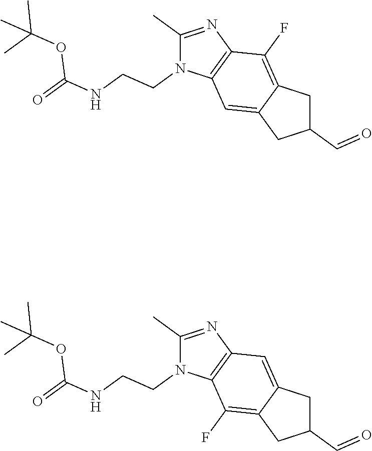 [SMILES: Cc1nc2c(F)c3c(cc2n1CCNC(=O)OC(C)(C)C)CC(C=O)C3 ; Cc1nc2cc3c(c(F)c2n1CCNC(=O)OC(C)(C)C)CC(C=O)C3]

The title compound mixture was prepared by analogy with Intermediate 63, Step 5, and was obtained as a light yellow oil. MS (ESP) m/z=362.2 [M+H]⁺.

### Intermediate 66

### tert-Butyl N-[2-(8-fluoro-6-formyl-6,7-dihydro-5H-cyclopenta[f]benzotriazol-2-yl)ethyl]carbamate

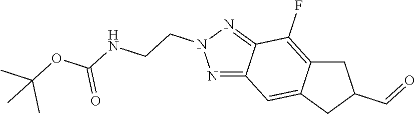 [SMILES: CC(C)(C)OC(=O)NCCn1nc2cc3c(c(F)c2n1)CC(C=O)C3]

### and

### Intermediate 67

### tert-Butyl N-[2-(8-fluoro-6-formyl-6,7-dihydro-5H-cyclopenta[f]benzotriazol-3-yl)ethyl]carbamate

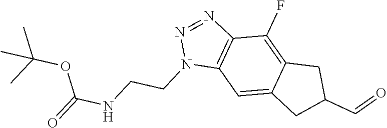 [SMILES: CC(C)(C)OC(=O)NCCn1nnc2c(F)c3c(cc21)CC(C=O)C3]

### and

### Intermediate 68

### tert-Butyl N-[2-(8-fluoro-6-formyl-6,7-dihydro-5H-cyclopenta[f]benzotriazol-1-yl)ethyl]carbamate

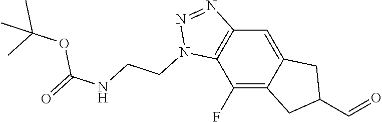 [SMILES: CC(C)(C)OC(=O)NCCn1nnc2cc3c(c(F)c21)CC(C=O)C3]

The title compounds were prepared from Intermediate 7 by analogy with Intermediate 51 and were obtained as an orange oil, a light brown oil and a light brown oil respectively. MS (ESP) m/z=293.1 [M+H-tBu]⁺.

### Intermediate 69

### tert-Butyl N-[1-[(4,8-difluoro-6-formyl-6,7-dihydro-5H-cyclopenta[f]benzotriazol-2-yl)methyl]cyclopropyl]carbamate

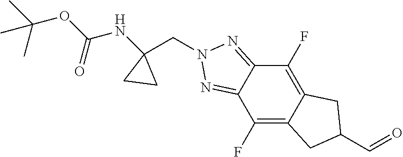 [SMILES: CC(C)(C)OC(=O)NC1(Cn2nc3c(F)c4c(c(F)c3n2)CC(C=O)C4)CC1]

### and

### Intermediate 70

### tert-Butyl N-[1-[(4,8-difluoro-6-formyl-6,7-dihydro-5H-cyclopenta[f]benzotriazol-1-yl)methyl]cyclopropyl]carbamate

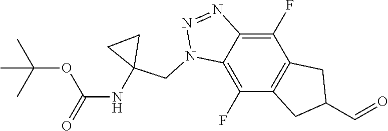 [SMILES: CC(C)(C)OC(=O)NC1(Cn2nnc3c(F)c4c(c(F)c32)CC(C=O)C4)CC1]

The title compounds were prepared from Intermediate 6 by analogy with Intermediate 51 using tert-butyl N-[1-(bromomethyl)cyclopropyl]carbamate [CAS #387845-49-0] in place of N-BOC-2-chloroethylamine in Step 2, and were obtained as white foams. MS (ESP) m/z=337.09 [M+H-tBu]⁺ and 393.04 [M+H]⁺ respectively.

### Intermediate 71

### tert-Butyl 3-(4,8-difluoro-6-formyl-6,7-dihydro-5H-cyclopenta[f]benzotriazol-2-yl)azetidine-1-carboxylate

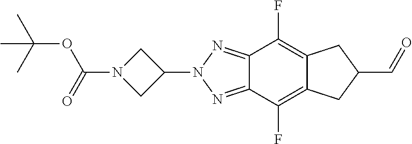 [SMILES: CC(C)(C)OC(=O)N1CC(n2nc3c(F)c4c(c(F)c3n2)CC(C=O)C4)C1]

### Steps 1 and 2: methyl 2-(1-tert-butoxycarbonylazetidin-3-yl)-4,8-difluoro-6,7-dihydro-5H-cyclopenta[f]benzotriazole-6-carboxylate

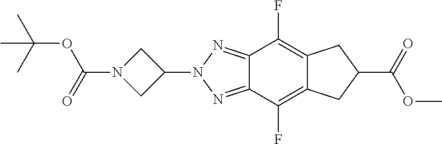 [SMILES: COC(=O)C1Cc2c(c(F)c3nn(C4CN(C(=O)OC(C)(C)C)C4)nc3c2F)C1]

### and methyl 1-(1-tert-butoxycarbonylazetidin-3-yl)-4,8-difluoro-6,7-dihydro-5H-cyclopenta[f]benzotriazole-6-carboxylate

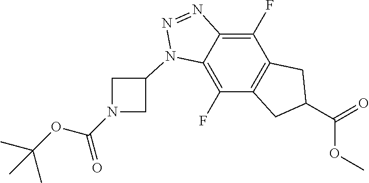 [SMILES: COC(=O)C1Cc2c(c(F)c3c(nnn3C3CN(C(=O)OC(C)(C)C)C3)c2F)C1]

The title compounds were prepared from Intermediate 6 by analogy with Intermediate 51, Steps 1 and 2, using 1-BOC-3-iodoazetidine [CAS #254454-54-1] in place of N-BOC-2-chloroethylamine in Step 2, and were obtained as colourless oils. MS (ESP) m/z=353.3 [M+H-tBu]⁺.

### Step 3: tert-butyl 3-[4,8-difluoro-6-(hydroxymethyl)-6,7-dihydro-5H-cyclopenta[f]benzotriazol-2-yl]azetidine-1-carboxylate

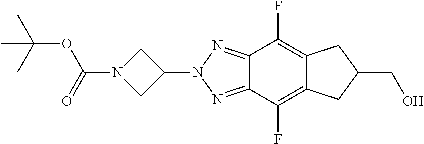 [SMILES: CC(C)(C)OC(=O)N1CC(n2nc3c(F)c4c(c(F)c3n2)CC(CO)C4)C1]

To a solution of methyl 2-(1-tert-butoxycarbonylazetidin-3-yl)-4,8-difluoro-6,7-dihydro-5H-cyclopenta[f]benzotriazole-6-carboxylate (200.0 mg, 0.49 mmol, 1.0 eq) in THF (5 mL) and ethanol (2.5 mL) was added calcium chloride (108.6 mg, 0.98 mmol, 2.0 eq) and sodium borohydride (1000.0 mg, 26.5 mmol, 54.0 eq) at 20° C. The mixture was stirred at 20° C. for 12 h. The reaction mixture was poured into water (50 mL), then extracted by EtOAc (50 mL×3). The combined organic layers were washed by brine (50 mL), dried by Na₂SO₄, filtered and concentrated to afford the title compound (180.0 mg, 0.47 mmol, 96.6% yield) as a yellow solid.

### Step 4: tert-butyl 3-(4,8-difluoro-6-formyl-6,7-dihydro-5H-cyclopenta[f]benzotriazol-2-yl)azetidine-1-carboxylate

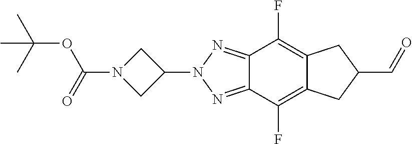 [SMILES: CC(C)(C)OC(=O)N1CC(n2nc3c(F)c4c(c(F)c3n2)CC(C=O)C4)C1]

The title compound was prepared by analogy with Intermediate 8, Step 4 and was obtained as a yellow oil. MS (ESP) m/z=379.1 [M+H]⁺.

### Intermediate 72

### tert-Butyl 3-(4,8-difluoro-6-formyl-6,7-dihydro-5H-cyclopenta[f]benzotriazol-1-yl)azetidine-1-carboxylate

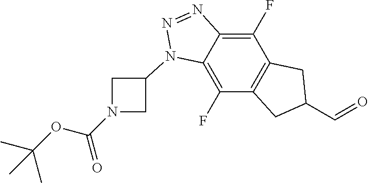 [SMILES: CC(C)(C)OC(=O)N1CC(n2nnc3c(F)c4c(c(F)c32)CC(C=O)C4)C1]

The title compound was prepared from methyl 1-(1-tert-butoxycarbonylazetidin-3-yl)-4,8-difluoro-6,7-dihydro-5H-cyclopenta[f]benzotriazole-6-carboxylate (Intermediate 71, Step 2) by analogy with Intermediate 71, Steps 3 and 4 and was obtained as a light yellow foam. MS (ESP) m/z=379.13 [M+H]⁺.

### Intermediate 73

### tert-Butyl N-[2-(5-fluoro-7-formyl-7,8-dihydro-6H-cyclopenta[e]benzotriazol-2-yl)ethyl]carbamate

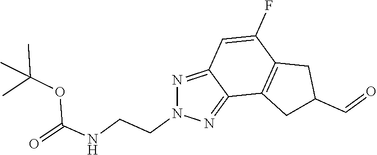 [SMILES: CC(C)(C)OC(=O)NCCn1nc2cc(F)c3c(c2n1)CC(C=O)C3]

### Step 1: ethyl 7-fluoro-4-[(4-methoxyphenyl)methylamino]-5-nitro-indane-2-carboxylate

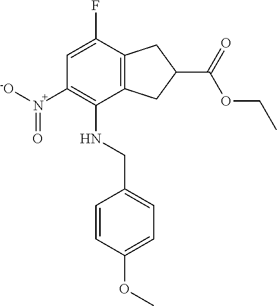

To a solution of ethyl 4,7-difluoro-5-nitro-indane-2-carboxylate (Intermediate 5, Step 3, 6000.0 mg, 22.12 mmol, 1.0 eq) in DMF (60 mL) was added 4-aminomethyl-anisole (3.18 mL, 24.33 mmol, 1.1 eq) and potassium carbonate (9172 mg, 66.37 mmol, 3.0 eq); the resulting mixture was stirred at 20° C. for 12 h. Water (100 mL) was added and the mixture was extracted with ethyl acetate (100 mL×2). The organic layer was washed with brine, dried over anhydrous sodium sulfate and concentrated under reduced pressure to give a residue. The residue was purified by silica column chromatography (petroleum ether/ethyl acetate 50:1 to 10:1) to give the title compound (6.5 g, 16.74 mmol, 75.6% yield) as an orange oil. ¹H NMR (400 MHz, CDCl₃) δ=7.77 (d, 1H), 7.20 (d, 2H), 6.88 (d, 2H), 4.65-4.52 (m, 2H), 4.24-4.16 (m, 2H), 3.81 (s, 3H), 3.54-3.45 (m, 2H), 3.41-3.30 (m, 1H), 3.24 (d, 2H), 1.32-1.28 (m, 3H).

### Step 2: ethyl 4,5-diamino-7-fluoro-indane-2-carboxylate

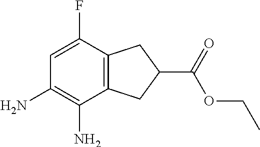 [SMILES: CCOC(=O)C1Cc2c(F)cc(N)c(N)c2C1]

To a solution of ethyl 7-fluoro-4-[(4-methoxyphenyl)methylamino]-5-nitro-indane-2-carboxylate (6.5 g, 16.74 mmol, 1.0 eq) in ethanol (200 mL) was added wet Pd/C (3.0 g), the atmosphere was exchanged with hydrogen for three times, then the mixture was stirred under hydrogen (15 Psi) at 20° C. for 12 h. The mixture was filtrated through Celite®, the filtrate was washed with EtOH (100 mL). The filtrate was collected and concentrated to give a residue which was purified by silica column chromatography (ethyl acetate/petroleum ether 0%˜55%) to give the title compound (2.8 g, 11.75 mmol, 70.2% yield) as a purple oil. MS (ESP) m/z=239.1 [M+H]⁺.

### Step 3: ethyl 5-fluoro-1,6,7,8-tetrahydrocyclopenta[e]benzotriazole-7-carboxylate

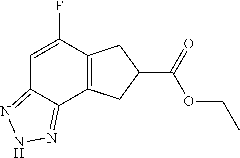 [SMILES: CCOC(=O)C1Cc2c(F)cc3n[nH]nc3c2C1]

To a stirred solution of ethyl 4,5-diamino-7-fluoro-indane-2-carboxylate (725.0 mg, 1.83 mmol, 1.0 eq) in HCl (3.04 mL, 36.51 mmol, 20.0 eq) at 0° C., a solution of sodium nitrite (163.8 mg, 2.37 mmol, 1.3 eq) in water (1.5 mL) was added drop-wise. The reaction mixture was stirred at 0° C. for 0.5 h. Then the mixture was extracted with EtOAc (twice). The combined organic phases were filtered through a phase separator and volatiles were removed under reduced pressure to give the crude compound (350 mg, brown foam) which was purified by silica cartridge chromatography (25 g, from DCM 100% to DCM/MeOH 98.5 to 1.5) to give the title compound (397.0 mg, 1.59 mmol, 87.2% yield) as a light brown solid. MS (ESP) m/z=250.1 [M+H]⁺.

### Steps 4 to 6: tert-butyl N-[2-(5-fluoro-7-formyl-7,8-dihydro-6H-cyclopenta[e]benzotriazol-2-yl)ethyl]carbamate

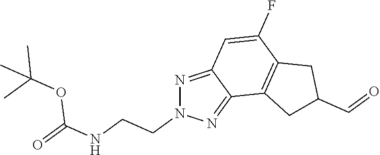 [SMILES: CC(C)(C)OC(=O)NCCn1nc2cc(F)c3c(c2n1)CC(C=O)C3]

The title compound was prepared by analogy with Intermediate 51, Steps 2 to 4, and was obtained as an off-white foam.

### Intermediate 74

### tert-Butyl N-[2-(5-fluoro-7-formyl-7,8-dihydro-6H-cyclopenta[e]benzotriazol-3-yl)ethyl]carbamate;tert-butyl N-[2-(5-fluoro-7-formyl-7,8-dihydro-6H-cyclopenta[g]benzotriazol-1-yl)ethyl]carbamate

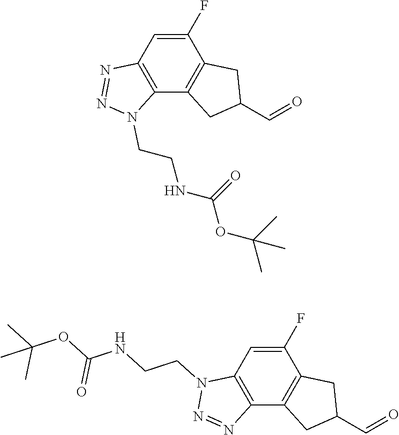 [SMILES: CC(C)(C)OC(=O)NCCn1nnc2cc(F)c3c(c21)CC(C=O)C3 ; CC(C)(C)OC(=O)NCCn1nnc2c3c(c(F)cc21)CC(C=O)C3]

The title compound mixture was prepared from ethyl 5-fluoro-1,6,7,8-tetrahydrocyclopenta[e]benzotriazole-7-carboxylate (Intermediate 73, Step 3) by analogy with Intermediate 52 and was obtained as a white foam. MS (ESP) m/z=349.2 [M+H]⁺.

### Intermediate 75

### tert-Butyl N-[2-(5-fluoro-7-formyl-7,8-dihydro-6H-cyclopenta[e]benzotriazol-2-yl)ethyl]-N-methyl-carbamate

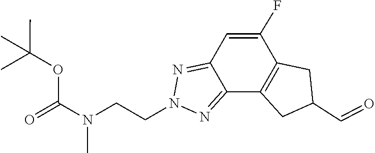 [SMILES: CN(CCn1nc2cc(F)c3c(c2n1)CC(C=O)C3)C(=O)OC(C)(C)C]

The title compound mixture was prepared from ethyl 5-fluoro-1,6,7,8-tetrahydrocyclopenta[e]benzotriazole-7-carboxylate (Intermediate 73, Step 3) by analogy with Intermediate 71, Steps 2 to 4 using tert-butyl N-(2-chloroethyl)-N-methyl-carbamate [CAS #220074-38-4] in place of 1-BOC-3-iodoazetidine and was obtained as a colourless oil. MS (ESP) m/z=263.1 [M+H-BOC]⁺.

### Intermediate 76

### tert-Butyl N-[(1S)-2-(5-fluoro-7-formyl-7,8-dihydro-6H-cyclopenta[e]benzotriazol-2-yl)-1-methyl-ethyl]carbamate

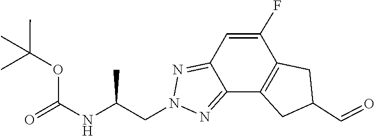 [SMILES: C[C@@H](Cn1nc2cc(F)c3c(c2n1)CC(C=O)C3)NC(=O)OC(C)(C)C]

### Step 1: ethyl 2-[(2S)-2-(tert-butoxycarbonylamino)propyl]-5-fluoro-7,8-dihydro-6H-cyclopenta[e]benzotriazole-7-carboxylate

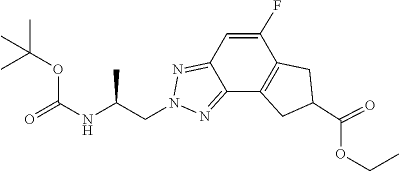 [SMILES: CCOC(=O)C1Cc2c(F)cc3nn(C[C@H](C)NC(=O)OC(C)(C)C)nc3c2C1]

A mixture of ethyl 5-fluoro-1,6,7,8-tetrahydrocyclopenta[e]benzotriazole-7-carboxylate (Intermediate 73, Step 3, 700.0 mg, 2.81 mmol, 1.0 eq), BOC-L-alaninol (2460.7 mg, 14.04 mmol, 5.0 eq), triphenylphosphine (883.3 mg, 3.37 mmol, 1.2 eq) and diisopropylazodicarboxylate (681.1 mg, 3.37 mmol, 1.2 eq) in THF (10 mL) was stirred at 20° C. under N₂ atmosphere for 12 h. Water (20 mL) was added and the mixture extracted with ethyl acetate (20 mL×2). The organic layer was washed with brine, dried over anhydrous sodium sulfate and concentrated under reduced pressure to give a residue. The residue was purified by silica column chromatography (18% ethyl acetate/petroleum ether) to give the title compound (930.0 mg, 2.29 mmol, 81.5% yield) as yellow oil. MS (ESP) m/z=351.1 [M+H-tBu]⁺.

### Step 2: tert-butyl N-[(1S)-2-[5-fluoro-7-(hydroxymethyl)-7,8-dihydro-6H-cyclopenta[e]benzotriazol-2-yl]-1-methyl-ethyl]carbamate

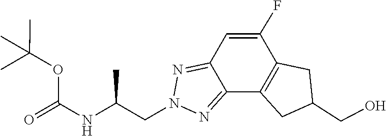 [SMILES: C[C@@H](Cn1nc2cc(F)c3c(c2n1)CC(CO)C3)NC(=O)OC(C)(C)C]

To a solution of ethyl 2-[(2S)-2-(tert-butoxycarbonylamino)propyl]-5-fluoro-7,8-dihydro-6H-cyclopenta[e]benzotriazole-7-carboxylate (700.0 mg, 1.72 mmol, 1.0 eq) in methanol (20 mL) was added lithium chloride (730.0 mg, 17.2 mmol, 10.0 eq) and sodium borohydride (1303.0 mg, 34.4 mmol, 20.0 eq) at 0° C., the mixture was stirred at 20° C. for 1 h. The reaction mixture was quenched by the addition of NH₄Cl (sat. aq., 40 mL). The mixture was extracted with ethyl acetate (50 mL×2). The combined organic layer was washed with brine, dried over anhydrous sodium sulfate and concentrated under reduced pressure to give a residue which was purified by silica column chromatography (petroleum ether/ethyl acetate 0% to 40%) to give the title compound (300.0 mg, 0.82 mmol, 47.8% yield) as colorless oil. MS (ESP) m/z=309.1 [M+H-BOC]⁺.

### Step 3: tert-butyl N-[(1S)-2-(5-fluoro-7-formyl-7,8-dihydro-6H-cyclopenta[e]benzotriazol-2-yl)-1-methyl-ethyl]carbamate

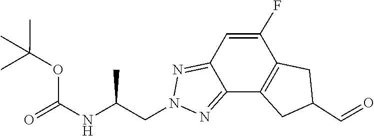 [SMILES: C[C@@H](Cn1nc2cc(F)c3c(c2n1)CC(C=O)C3)NC(=O)OC(C)(C)C]

The title compound mixture was prepared by analogy with Intermediate 8, Step 4 and was obtained as a yellow oil. MS (ESP) m/z=307.4 [M+H-tBu]⁺.

### Intermediate 77

### tert-Butyl N-[(1R)-1-(8-fluoro-6-formyl-6,7-dihydro-5H-cyclopenta[f][1,3]benzoxazol-2-yl)ethyl]carbamate

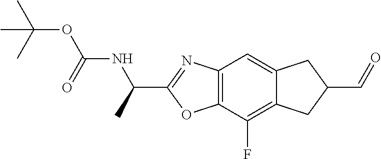 [SMILES: C[C@@H](NC(=O)OC(C)(C)C)c1nc2cc3c(c(F)c2o1)CC(C=O)C3]

The title compound was prepared by analogy to Intermediate 29, Steps 2 to 5 using BOC-D-ALA-OH in place of BOC-SAR-OH and was obtained as a brown gum. MS (ESP) m/z=349.2 [M+H]⁺.

### Intermediate 78

### tert-Butyl N-[(1R)-2-tert-butoxy-1-(8-fluoro-6-formyl-6,7-dihydro-5H-cyclopenta[f][1,3]benzoxazol-2-yl)ethyl]-N-methyl-carbamate

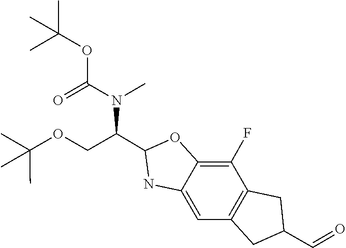

The title compound was prepared by analogy to Intermediate 29, Steps 2 to 5 using (2R)-3-tert-butoxy-2-[tert-butoxycarbonyl(methyl)amino]propanoic acid [CAS #1126450-26-7] in place of BOC-SAR-OH and was obtained as a light yellow oil. MS (ESP) m/z=435.2 [M+H]⁺.

### Intermediate 79

### tert-Butyl N-[(5-fluoro-7-formyl-1,6,7,8-tetrahydrocyclopenta[e]benzimidazol-2-yl)methyl]-N-methyl-carbamate

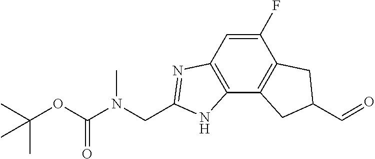 [SMILES: CN(Cc1nc2cc(F)c3c(c2[nH]1)CC(C=O)C3)C(=O)OC(C)(C)C]

The title compound was prepared from ethyl 4,5-diamino-7-fluoro-indane-2-carboxylate (Intermediate 73, Step 2) and BOC-SAR-OH by analogy to Intermediate 9 and was obtained as a brown oil. MS (ESP) m/z=348.2 [M+H]⁺.

### Intermediate 80

### tert-Butyl N-[(4-fluoro-6-formyl-6,7-dihydro-5H-cyclopenta[f][1,3]benzoxazol-2-yl)methyl]-N-methyl-carbamate

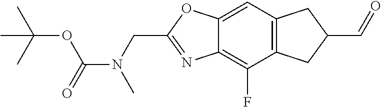 [SMILES: CN(Cc1nc2c(F)c3c(cc2o1)CC(C=O)C3)C(=O)OC(C)(C)C]

### Step 1: ethyl 4-fluoro-6-hydroxy-5-nitro-indane-2-carboxylate

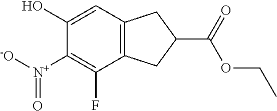

To a solution of ethyl 4-fluoro-6-hydroxy-indane-2-carboxylate (Intermediate 7, Route A, Step 4, 2545.0 mg, 11.35 mmol, 1.0 eq) in DCM (56.75 mL) at RT, a mixture of nitric acid (795.3 uL, 12.48 mmol, 1.1 eq) and sulfuric acid (1.38 mL, 25.81 mmol, 2.27 eq) was added drop-wise; the reaction mixture was then stirred at RT for 0.5 h. The reaction mixture was poured into water and extracted with DCM (three times). The combined organic phases were filtered through a phase separator and volatiles were removed under reduced pressure to give the crude product which was purified by silica cartridge chromatography (250 g silica, from cyclohexane 100% cyclohexane to cyclohexane/EtOAc 6:4) to give ethyl 7-fluoro-4-hydroxy-5-nitro-indane-2-carboxylate;ethyl 7-fluoro-5-hydroxy-4-nitro-indane-2-carboxylate (1530.0 mg, 2.84 mmol, 50.1% yield) as a yellow solid and the title compound (530.0 mg, 1.97 mmol, 17.3% yield) as a yellow solid. MS (ESP) m/z=270.2 [M+H]⁺.

### Step 2: ethyl 5-amino-4-fluoro-6-hydroxy-indane-2-carboxylate

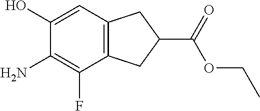 [SMILES: CCOC(=O)C1Cc2cc(O)c(N)c(F)c2C1]

A suspension of ethyl 4-fluoro-6-hydroxy-5-nitro-indane-2-carboxylate (541.0 mg, 2.01 mmol, 1.0 eq) and Pd/C 55-65% wet (85.54 mg, 0.04 mmol, 0.02 eq) in ethanol (20.09 mL) was purged with vacuum/nitrogen atmosphere (three cycles) and with vacuum/hydrogen atmosphere (four cycles); the reaction mixture was stirred at RT under a hydrogen atmosphere for 1 h. The reaction mixture was purged with vacuum/nitrogen atmosphere (three cycles) and filtered through a Celite® pad, washing the cake with ethanol. The collected filtrate was concentrated under reduced pressure to give the crude title compound (480.0 mg, 2.01 mmol, 99.8% yield) as a brown solid. MS (ESP) m/z=240.2 [M+H]⁺.

### Steps 3 to 6: tert-Butyl N-[(4-fluoro-6-formyl-6,7-dihydro-5H-cyclopenta[f][1,3]benzoxazol-2-yl)methyl]-N-methyl-carbamate

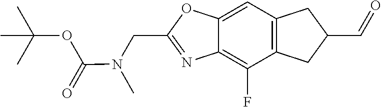 [SMILES: CN(Cc1nc2c(F)c3c(cc2o1)CC(C=O)C3)C(=O)OC(C)(C)C]

The title compound was prepared by analogy to Intermediate 29, Steps 2 to 5, and was obtained as a light brown oil. MS (ESP) m/z=293.2 [M+H-tBu]⁺.

### Intermediate 81

### tert-Butyl N-[(5-fluoro-7-formyl-7,8-dihydro-6H-cyclopenta[e][1,3]benzoxazol-2-yl)methyl]-N-methyl-carbamate

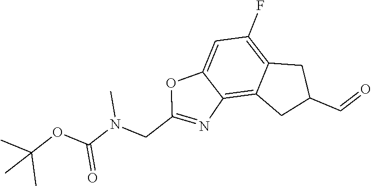 [SMILES: CN(Cc1nc2c3c(c(F)cc2o1)CC(C=O)C3)C(=O)OC(C)(C)C]

The title compound was prepared from ethyl 7-fluoro-4-hydroxy-5-nitro-indane-2-carboxylate;ethyl 7-fluoro-5-hydroxy-4-nitro-indane-2-carboxylate (Intermediate 80, Step 1) by analogy with Intermediate 80, Steps 2 to 6, and was obtained as a light brown gum. MS (ESP) m/z=349.4 [M+H]⁺.

### Intermediate 82

### tert-Butyl N-[(1S)-1-(8-fluoro-6-formyl-6,7-dihydro-5H-cyclopenta[f][1,3]benzoxazol-2-yl)ethyl]carbamate

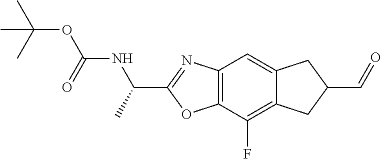 [SMILES: C[C@H](NC(=O)OC(C)(C)C)c1nc2cc3c(c(F)c2o1)CC(C=O)C3]

The title compound was prepared by analogy to Intermediate 77 using BOC-ALA-OH in place of BOC-D-ALA-OH and was obtained as a light yellow gum. MS (ESP) m/z=349.11 [M+H]⁺.

### Intermediate 83

### 2-[[3-[tert-Butyl(dimethyl)silyl]oxyazetidin-1-yl]methyl]-8-fluoro-6,7-dihydro-5H-cyclopenta[f][1,3]benzoxazole-6-carbaldehyde

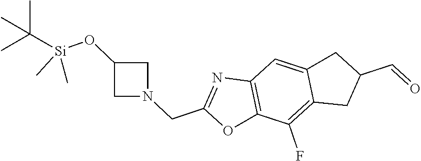 [SMILES: CC(C)(C)[Si](C)(C)OC1CN(Cc2nc3cc4c(c(F)c3o2)CC(C=O)C4)C1]

### Step 1: ethyl 2-(chloromethyl)-8-fluoro-6,7-dihydro-5H-cyclopenta[f][1,3]benzoxazole-6-carboxylate

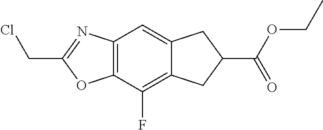 [SMILES: CCOC(=O)C1Cc2cc3nc(CCl)oc3c(F)c2C1]

Ethyl 2-chloroethanimidate hydrochloride (660.5 mg, 4.18 mmol, 1.0 eq) was added to a solution of ethyl 6-amino-4-fluoro-5-hydroxy-indane-2-carboxylate (Intermediate 29, Step 1, 1000.0 mg, 4.18 mmol, 1.0 eq) in DCM (15 mL) at 0° C. The mixture was stirred at 20° C. for 12 h. The mixture was diluted with DCM (5 mL), filtered and the filtrate was concentrated to afford the title compound (1100.0 mg, 3.69 mmol, 88.4% yield) as a yellow oil which was used in the next step directly. MS (ESP) m/z=298.3 [M+H]⁺.

### Step 2: ethyl 8-fluoro-2-[(3-hydroxyazetidin-1-yl)methyl]-6,7-dihydro-5H-cyclopenta[f][1,3]benzoxazole-6-carboxylate

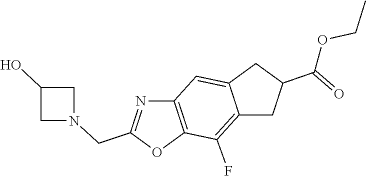 [SMILES: CCOC(=O)C1Cc2cc3nc(CN4CC(O)C4)oc3c(F)c2C1]

To a solution of 3-hydroxyazetidine hydrochloride (294.4 mg, 2.69 mmol, 2.0 eq) and DIPEA (0.59 mL, 3.36 mmol, 2.5 eq) in DMF (5 mL) was added ethyl 2-(chloromethyl)-8-fluoro-6,7-dihydro-5H-cyclopenta[f][1,3]benzoxazole-6-carboxylate (400.0 mg, 1.34 mmol, 1.0 eq) in DMF 0.2 mL at 30° C.; the mixture was stirred at 30° C. for 2 h. The mixture was quenched with water (5 mL), extracted with EtOAc (5 mL×2). The combined organic phase was dried over Na₂SO4, filtered and the filtrate was concentrated to afford the title compound (449.0 mg, 1.34 mmol, 99.9% yield) as a yellow oil which was used in the next step directly. MS (ESP) m/z=335.4 [M+H]⁺.

### Step 3: ethyl 2-[[3-[tert-butyl(dimethyl)silyl]oxyazetidin-1-yl]methyl]-8-fluoro-6,7-dihydro-5H-cyclopenta[f][1,3]benzoxazole-6-carboxylate

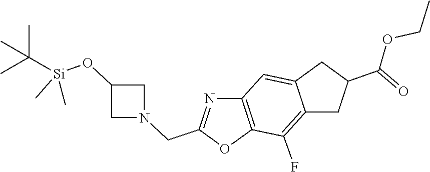 [SMILES: CCOC(=O)C1Cc2cc3nc(CN4CC(O[Si](C)(C)C(C)(C)C)C4)oc3c(F)c2C1]

To a solution of ethyl 8-fluoro-2-[(3-hydroxyazetidin-1-yl)methyl]-6,7-dihydro-5H-cyclopenta[f][1,3]benzoxazole-6-carboxylate (400.0 mg, 1.2 mmol, 1.0 eq) in DMF (5 mL) was added DIPEA (0.32 mL, 1.79 mmol, 1.5 eq) and tert-butylchlorodimethylsilane (215.4 mg, 1.44 mmol, 1.2 eq) at 20° C., then the mixture was stirred at 20° C. for 2 h. The mixture was poured into water (10 mL), extracted with EtOAc (20 mL×2). The combined organic phase was washed with brine, dried over Na₂SO4, filtered and the filtrate was concentrated to give a residue which was purified by flash column chromatography (petroleum ether/EtOAc 10:1) to afford the title compound (500.0 mg, 1.11 mmol, 93.2% yield) as a yellow oil. MS (ESP) m/z=449.3 [M+H]⁺.

### Steps 4 to 5: 2-[[3-[tert-butyl(dimethyl)silyl]oxyazetidin-1-yl]methyl]-8-fluoro-6,7-dihydro-5H-cyclopenta[f][1,3]benzoxazole-6-carbaldehyde

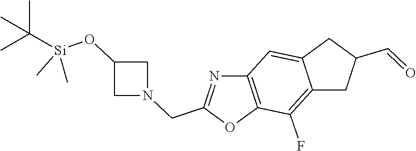 [SMILES: CC(C)(C)[Si](C)(C)OC1CN(Cc2nc3cc4c(c(F)c3o2)CC(C=O)C4)C1]

The title compound was prepared by analogy to Intermediate 71, Steps 3 and 4, and was obtained as a yellow oil. MS (ESP) m/z=405.3 [M+H]⁺.

### Intermediate 84

### tert-Butyl 3-(8-fluoro-6-formyl-6,7-dihydro-5H-cyclopenta[f][1,3]benzoxazol-2-yl)azetidine-1-carboxylate

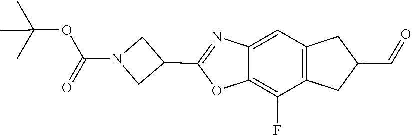 [SMILES: CC(C)(C)OC(=O)N1CC(c2nc3cc4c(c(F)c3o2)CC(C=O)C4)C1]

The title compound was prepared by analogy to Intermediate 29, Steps 2 to 5, using 1-BOC-azetidine-3-carboxylic acid in place of BOC-SAR-OH and was obtained as a brown solid. MS (ESP) m/z=383.3 [M+Na]⁺.

### Intermediate 85

### tert-Butyl N-[2-(8-fluoro-6-formyl-6,7-dihydro-5H-cyclopenta[f][1,3]benzoxazol-2-yl)ethyl]carbamate

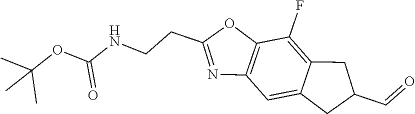 [SMILES: CC(C)(C)OC(=O)NCCc1nc2cc3c(c(F)c2o1)CC(C=O)C3]

### Steps 1 and 2: ethyl 2-[2-(tert-butoxycarbonylamino)ethyl]-8-fluoro-6,7-dihydro-5H-cyclopenta[f][1,3]benzoxazole-6-carboxylate

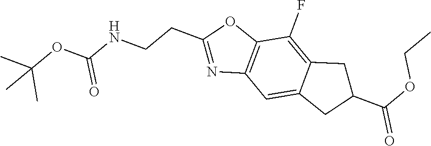 [SMILES: CCOC(=O)C1Cc2cc3nc(CCNC(=O)OC(C)(C)C)oc3c(F)c2C1]

The title compound was prepared by analogy to Intermediate 29, Steps 2 and 3, using BOC-BETA-ALA-OH in place of BOC-SAR-OH and was obtained as a yellow oil. MS (ESP) m/z=337.2 [M+H-tBu]⁺.

### Step 3: tert-butyl N-[2-[8-fluoro-6-(hydroxymethyl)-6,7-dihydro-5H-cyclopenta[f][1,3]benzoxazol-2-yl]ethyl]carbamate

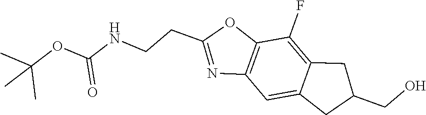 [SMILES: CC(C)(C)OC(=O)NCCc1nc2cc3c(c(F)c2o1)CC(CO)C3]

To a solution of ethyl 2-[2-(tert-butoxycarbonylamino)ethyl]-8-fluoro-6,7-dihydro-5H-cyclopenta[f][1,3]benzoxazole-6-carboxylate (3.5 g, 8.92 mmol, 1.0 eq) and lithium chloride (3781 mg, 89.19 mmol, 10.0 eq) in methanol (56 mL) was added sodium borohydride (3374 mg, 89.2 mmol, 10.0 eq) at 0° C. The reaction mixture was stirred at 0° C. for 2 h. The reaction mixture was quenched by the addition of ice-cold NH₄Cl (1M, 100 mL), then the mixture was extracted with EtOAc (100 mL×3). The combined organic layers were washed with sat. aq. NaCl (100 mL), dried over anhydrous Na₂SO₄, filtered and concentrated under reduced pressure to give a residue. The residue was purified by silica column chromatography (60% petroleum ether/ethyl acetate) to give the title compound (800.0 mg, 2.28 mmol, 24.4% yield) as a yellow oil. MS (ESP) m/z=351.2 [M+H]⁺.

### Step 4: tert-butyl N-[2-(8-fluoro-6-formyl-6,7-dihydro-5H-cyclopenta[f][1,3]benzoxazol-2-yl)ethyl]carbamate

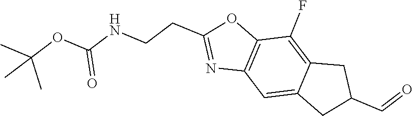 [SMILES: CC(C)(C)OC(=O)NCCc1nc2cc3c(c(F)c2o1)CC(C=O)C3]

The title compound was prepared by analogy to Intermediate 29, Step 5 and was obtained as a yellow oil. MS (ESP) m/z=293.2 [M+H-tBu]⁺.

### Intermediate 86

### tert-Butyl N-[(8-fluoro-6-formyl-6,7-dihydro-5H-cyclopenta[f][1,3]benzoxazol-2-yl)methyl]carbamate

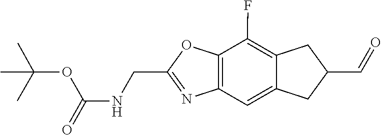 [SMILES: CC(C)(C)OC(=O)NCc1nc2cc3c(c(F)c2o1)CC(C=O)C3]

### Step 1: ethyl 2-(azidomethyl)-8-fluoro-6,7-dihydro-5H-cyclopenta[f][1,3]benzoxazole-6-carboxylate

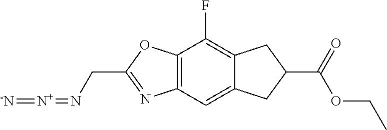

To a solution of ethyl 2-(chloromethyl)-8-fluoro-6,7-dihydro-5H-cyclopenta[f][1,3]benzoxazole-6-carboxylate (Intermediate 83, Step 1, 900.0 mg, 3.02 mmol, 1.0 eq) in DMF (12 mL) was added sodium azide (738.0 mg, 11.35 mmol, 3.76 eq) at 10° C. and then the mixture was stirred at 15° C. for 16 h. To the mixture was added water and then it was extracted with EtOAc (20 mL×3) to give the crude title compound (919.86 mg, 3.02 mmol, quant. yield) which was used in the next step directly. MS (ESP) m/z=305.1 [M+H]⁺.

### Step 2: ethyl 2-[(tert-butoxycarbonylamino)methyl]-8-fluoro-6,7-dihydro-5H-cyclopenta[f][1,3]benzoxazole-6-carboxylate

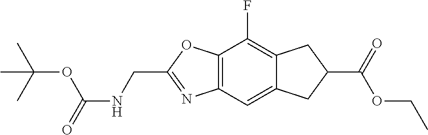 [SMILES: CCOC(=O)C1Cc2cc3nc(CNC(=O)OC(C)(C)C)oc3c(F)c2C1]

To a solution of ethyl 2-(azidomethyl)-8-fluoro-6,7-dihydro-5H-cyclopenta[f][1,3]benzoxazole-6-carboxylate (919.86 mg, 3.02 mmol, 1.0 eq) in EtOAc (12 mL) was added triphenylphosphine (1179.0 mg, 4.53 mmol, 1.5 eq) at 10° C. After stirring for 10 min., water (0.5 mL) was added and then the mixture was stirred at 60° C. for 3 h. The mixture was cooled to 10° C., then DIPEA (1.54 mL, 4.31 mmol, 1.5 eq) and di-tert-butyl dicarbonate (940.06 mg, 4.31 mmol, 1.5 eq) were added at 10° C., then the mixture was stirred at 20° C. for 2 h. To the mixture was added water (10 mL), then it was extracted with EtOAc (20 mL×2). The combined organic phase was concentrated and the crude was purified via flash column chromatography (3:1 petroleum ether/EtOAc) to give the title compound (900.0 mg, 2.38 mmol, 82.7% yield) as a yellow oil. MS (ESP) m/z=379.3 [M+H]⁺.

### Steps 3 and 4: tert-butyl N-[(8-fluoro-6-formyl-6,7-dihydro-5H-cyclopenta[f][1,3]benzoxazol-2-yl)methyl]carbamate

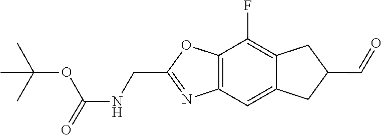 [SMILES: CC(C)(C)OC(=O)NCc1nc2cc3c(c(F)c2o1)CC(C=O)C3]

The title compound was prepared by analogy to Intermediate 29, Steps 4 and 5 and was obtained as a yellow oil.

### Intermediate 87

### tert-Butyl (2S)-2-(8-fluoro-6-formyl-6,7-dihydro-5H-cyclopenta[f][1,3]benzoxazol-2-yl)azetidine-1-carboxylate

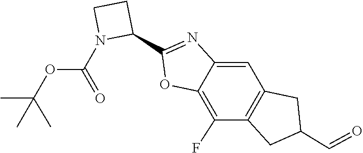 [SMILES: CC(C)(C)OC(=O)N1CC[C@H]1c1nc2cc3c(c(F)c2o1)CC(C=O)C3]

### Step 1: tert-butyl (2S)-2-[(2-ethoxycarbonyl-7-fluoro-6-hydroxy-indan-5-yl)carbamoyl]azetidine-1-carboxylate

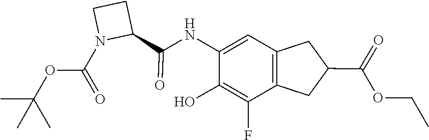 [SMILES: CCOC(=O)C1Cc2cc(NC(=O)[C@@H]3CCN3C(=O)OC(C)(C)C)c(O)c(F)c2C1]

The title compound was prepared from (2S)-1-tert-butoxycarbonylazetidine-2-carboxylic acid [CAS #51077-14-6] and ethyl 6-amino-4-fluoro-5-hydroxy-indane-2-carboxylate (Intermediate 29, Step 1) by analogy to Intermediate 9, Step1 and was obtained as a yellow oil. MS (ESP) m/z=323.2 [M+H-BOC]⁺.

### Steps 2 to 4: tert-butyl (2S)-2-(8-fluoro-6-formyl-6,7-dihydro-5H-cyclopenta[f][1,3]benzoxazol-2-yl)azetidine-1-carboxylate

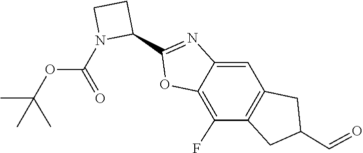 [SMILES: CC(C)(C)OC(=O)N1CC[C@H]1c1nc2cc3c(c(F)c2o1)CC(C=O)C3]

The title compound was prepared by analogy to Intermediate 29, Steps 3 to 5 and was obtained as a yellow oil. MS (ESP) m/z=305.1 [M+H-tBu]⁺.

### Intermediate 88

### tert-Butyl (2R)-2-(8-fluoro-6-formyl-6,7-dihydro-5H-cyclopenta[f][1,3]benzoxazol-2-yl)azetidine-1-carboxylate

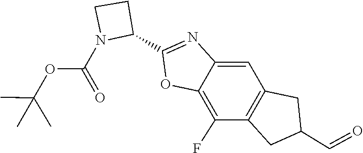 [SMILES: CC(C)(C)OC(=O)N1CC[C@@H]1c1nc2cc3c(c(F)c2o1)CC(C=O)C3]

The title compound was prepared from (2R)-1-tert-butoxycarbonylazetidine-2-carboxylic acid [CAS #228857-58-7] by analogy to Intermediate 87 and was obtained as a yellow oil. MS (ESP) m/z=305.1 [M+H-tBu]⁺.

### Intermediate 89

### tert-Butyl 3-(8-fluoro-6-formyl-6,7-dihydro-5H-cyclopenta[f]benzotriazol-2-yl)azetidine-1-carboxylate

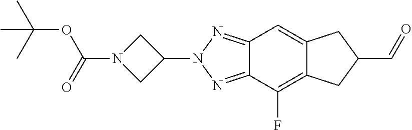 [SMILES: CC(C)(C)OC(=O)N1CC(n2nc3cc4c(c(F)c3n2)CC(C=O)C4)C1]

### and

### Intermediate 90

### tert-Butyl 3-(8-fluoro-6-formyl-6,7-dihydro-5H-cyclopenta[f]benzotriazol-1-yl)azetidine-1-carboxylate

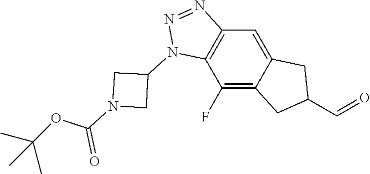 [SMILES: CC(C)(C)OC(=O)N1CC(n2nnc3cc4c(c(F)c32)CC(C=O)C4)C1]

### and

### Intermediate 91

### tert-Butyl 3-(8-fluoro-6-formyl-6,7-dihydro-5H-cyclopenta[f]benzotriazol-3-yl)azetidine-1-carboxylate

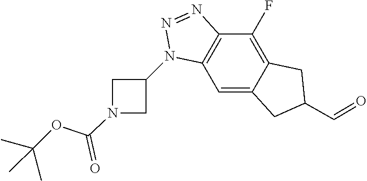 [SMILES: CC(C)(C)OC(=O)N1CC(n2nnc3c(F)c4c(cc32)CC(C=O)C4)C1]

### Step 1: 3-(1-tert-butoxycarbonylazetidin-3-yl)-8-fluoro-6,7-dihydro-5H-cyclopenta[f]benzotriazole-6-carboxylic acid; 2-(1-tert-butoxycarbonylazetidin-3-yl)-8-fluoro-6,7-dihydro-5H-cyclopenta[f]benzotriazole-6-carboxylic acid; 1-(1-tert-butoxycarbonylazetidin-3-yl)-8-fluoro-6,7-dihydro-5H-cyclopenta[f]benzotriazole-6-carboxylic acid

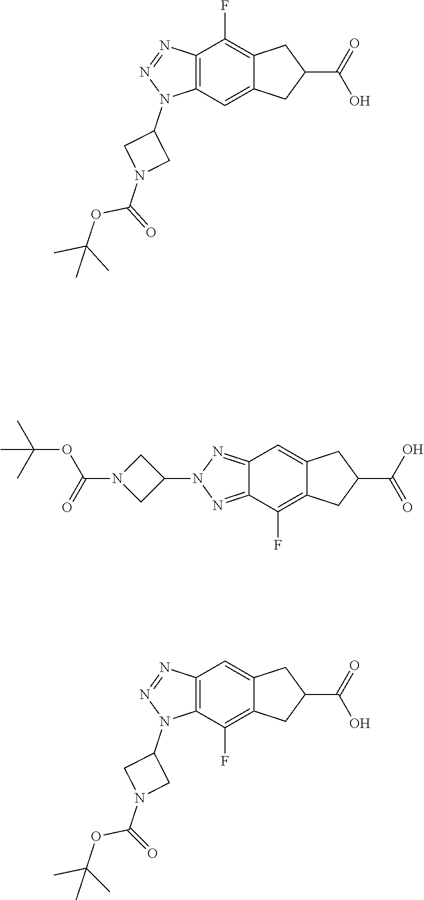 [SMILES: CC(C)(C)OC(=O)N1CC(n2nnc3c(F)c4c(cc32)CC(C(=O)O)C4)C1 ; CC(C)(C)OC(=O)N1CC(n2nc3cc4c(c(F)c3n2)CC(C(=O)O)C4)C1 ; CC(C)(C)OC(=O)N1CC(n2nnc3cc4c(c(F)c32)CC(C(=O)O)C4)C1]

To a mixture of ethyl 8-fluoro-3,5,6,7-tetrahydrocyclopenta[f]benzotriazole-6-carboxylate (Intermediate 66, Step 1, 1600.0 mg, 6.42 mmol, 1.0 eq) and cesium carbonate (6274.8 mg, 19.3 mmol, 3.0 eq) in DMF (15 mL), 1-BOC-3-iodoazetidine [CAS #254454-54-1] (2181 mg, 7.7 mmol, 1.2 eq) was added and the mixture was stirred at 100° C. for 16 h. To the mixture was added water (40 mL), the pH was adjusted to ˜6.0 and it was extracted by EtOAc (30 mL×3). The combined organic layers were washed with CaCl₂ (sat. aq., 10 mL×2) and NaCl (sat. aq, 10 mL×3) then concentrated in vacuo to give the crude title compound mixture (1600 mg, 4.26 mmol, 66.3% yield) which was used in the next step directly. MS (ESP) m/z=321.1 [M+H-tBu]⁺.

### Step 2: methyl 3-(1-tert-butoxycarbonylazetidin-3-yl)-8-fluoro-6,7-dihydro-5H-cyclopenta[f]benzotriazole-6-carboxylate and methyl 2-(1-tert-butoxycarbonylazetidin-3-yl)-8-fluoro-6,7-dihydro-5H-cyclopenta[f]benzotriazole-6-carboxylate and methyl 1-(1-tert-butoxycarbonylazetidin-3-yl)-8-fluoro-6,7-dihydro-5H-cyclopenta[f]benzotriazole-6-carboxylate

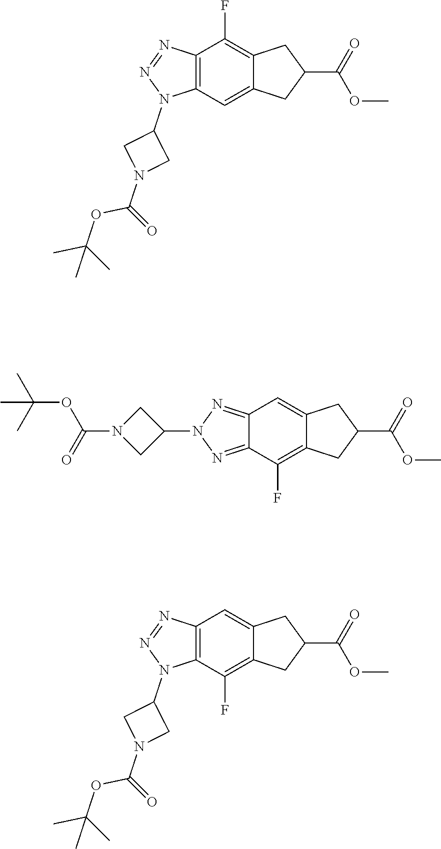

To a mixture of 3-(1-tert-butoxycarbonylazetidin-3-yl)-8-fluoro-6,7-dihydro-5H-cyclopenta[f]benzotriazole-6-carboxylic acid; 2-(1-tert-butoxycarbonylazetidin-3-yl)-8-fluoro-6,7-dihydro-5H-cyclopenta[f]benzotriazole-6-carboxylic acid; 1-(1-tert-butoxycarbonylazetidin-3-yl)-8-fluoro-6,7-dihydro-5H-cyclopenta[f]benzotriazole-6-carboxylic acid (1600 mg, 4.26 mmol, 1.0 eq) in methanol (10 mL), (trimethylsilyl)diazomethane (114.2 mg, 8.5 mmol, 2.0 eq) was added and the mixture was stirred at 100° C. for 16 h. The mixture was concentrated in vacuo and then purified by flash column chromatography (20 g silica, 1:5 EtOAc/petroleum ether) to give in order of elution methyl 2-(1-tert-butoxycarbonylazetidin-3-yl)-8-fluoro-6,7-dihydro-5H-cyclopenta[f]benzotriazole-6-carboxylate (1019.0 mg, 2.61 mmol), methyl 1-(1-tert-butoxycarbonylazetidin-3-yl)-8-fluoro-6,7-dihydro-5H-cyclopenta[f]benzotriazole-6-carboxylate (398.0 mg, 1.02 mmol) and 3-(1-tert-butoxycarbonylazetidin-3-yl)-8-fluoro-6,7-dihydro-5H-cyclopenta[f]benzotriazole-6-carboxylic acid (249 mg, 0.64 mmol, quant. overall yield). The regioisomers were determined by 2D ¹H NMR.

### Steps 3 and 4: tert-butyl 3-(8-fluoro-6-formyl-6,7-dihydro-5H-cyclopenta[f]benzotriazol-2-yl)azetidine-1-carboxylate and tert-butyl 3-(8-fluoro-6-formyl-6,7-dihydro-5H-cyclopenta[f]benzotriazol-1-yl)azetidine-1-carboxylate and and tert-butyl 3-(8-fluoro-6-formyl-6,7-dihydro-5H-cyclopenta[f]benzotriazol-3-yl)azetidine-1-carboxylate

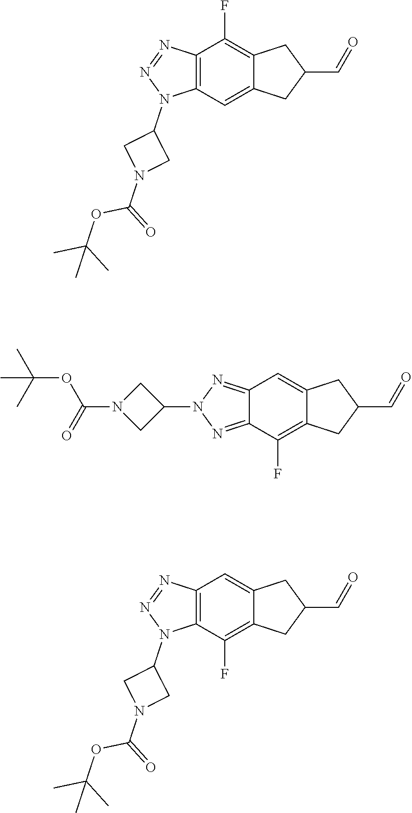 [SMILES: CC(C)(C)OC(=O)N1CC(n2nnc3c(F)c4c(cc32)CC(C=O)C4)C1 ; CC(C)(C)OC(=O)N1CC(n2nc3cc4c(c(F)c3n2)CC(C=O)C4)C1 ; CC(C)(C)OC(=O)N1CC(n2nnc3cc4c(c(F)c32)CC(C=O)C4)C1]

The title compounds were prepared by analogy with Intermediate 8, Steps 3 and 4.

### Intermediate 92

### tert-Butyl N-[2-(4,8-difluoro-6-formyl-6,7-dihydro-5H-cyclopenta[f]benzotriazol-2-yl)ethyl]-N-methyl-carbamate

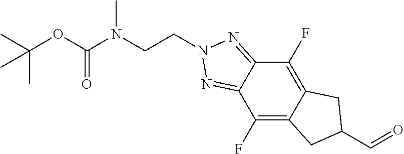 [SMILES: CN(CCn1nc2c(F)c3c(c(F)c2n1)CC(C=O)C3)C(=O)OC(C)(C)C]

### and

### Intermediate 93

### tert-Butyl N-[2-(4,8-difluoro-6-formyl-6,7-dihydro-5H-cyclopenta[f]benzotriazol-1-yl)ethyl]-N-methyl-carbamate

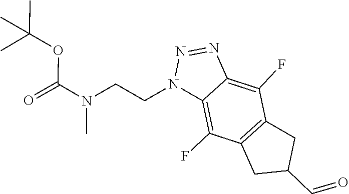 [SMILES: CN(CCn1nnc2c(F)c3c(c(F)c21)CC(C=O)C3)C(=O)OC(C)(C)C]

The title compounds were prepared from Intermediate 6 by analogy with Intermediates 66 to 68 using tert-butyl N-(2-chloroethyl)-N-methyl-carbamate [CAS #220074-38-4] in place of N-BOC-2-chloroethylamine and were obtained as yellow oils. MS (ESN) m/z=379.1 [M−H]⁻.

### Intermediate 94

### tert-Butyl N-[(8-fluoro-6-formyl-3,3a,5,6,7,8a-hexahydro-2H-cyclopenta[f][1,3]benzoxazol-2-yl)methyl]-N-methyl-carbamate

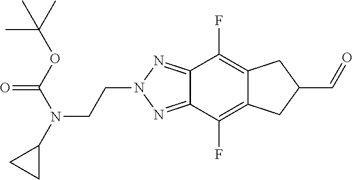 [SMILES: CC(C)(C)OC(=O)N(CCn1nc2c(F)c3c(c(F)c2n1)CC(C=O)C3)C1CC1]

### and

### Intermediate 95

### tert-Butyl N-cyclopropyl-N-[2-(4,8-difluoro-6-formyl-6,7-dihydro-5H-cyclopenta[f]benzotriazol-1-yl)ethyl]carbamate

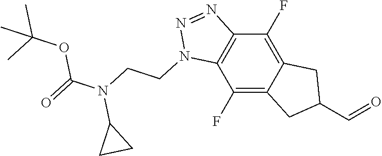 [SMILES: CC(C)(C)OC(=O)N(CCn1nnc2c(F)c3c(c(F)c21)CC(C=O)C3)C1CC1]

The title compounds were prepared from methyl 4,8-difluoro-3,5,6,7-tetrahydrocyclopenta[f]benzotriazole-6-carboxylate (Intermediate 70, Step 1) by analogy with Intermediate 76 using tert-butyl N-cyclopropyl-N-(2-hydroxyethyl)carbamate [CAS #1153134-59-8] in place of BOC-L-alaninol and were obtained as yellow oils.

### Intermediate 96

### tert-Butyl N-[(1S)-2-(4,8-difluoro-6-formyl-6,7-dihydro-5H-cyclopenta[f]benzotriazol-2-yl)-1-methyl-ethyl]carbamate

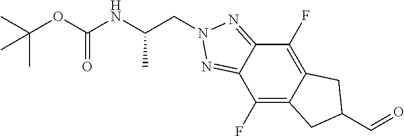 [SMILES: C[C@@H](Cn1nc2c(F)c3c(c(F)c2n1)CC(C=O)C3)NC(=O)OC(C)(C)C]

### and

### Intermediate 97

### tert-Butyl N-[(1S)-2-(4,8-difluoro-6-formyl-6,7-dihydro-5H-cyclopenta[f]benzotriazol-1-yl)-1-methyl-ethyl]carbamate

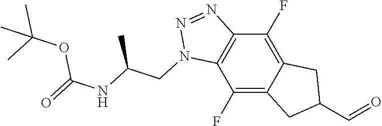 [SMILES: C[C@@H](Cn1nnc2c(F)c3c(c(F)c21)CC(C=O)C3)NC(=O)OC(C)(C)C]

The title compounds were prepared by analogy with Intermediate 71 and 72, using tert-butyl N-[(1S)-2-chloro-1-methyl-ethyl]carbamate [CAS #850139-69-4] in place of N-BOC-2-chloroethylamine, except that the regioisomers were separated at the penultimate step, and were obtained as brown oils. MS (ESP) m/z=281.3 [M+H-BOC]⁺.

### Intermediate 98

### tert-Butyl N-[2-(4,8-difluoro-6-formyl-6,7-dihydro-5H-cyclopenta[f]benzimidazol-1-yl)ethyl]-N-methyl-carbamate

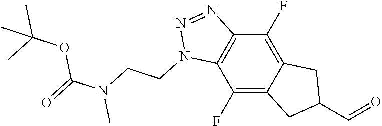 [SMILES: CN(CCn1nnc2c(F)c3c(c(F)c21)CC(C=O)C3)C(=O)OC(C)(C)C]

### Step 1: ethyl 4,8-difluoro-3,5,6,7-tetrahydrocyclopenta[f]benzimidazole-6-carboxylate

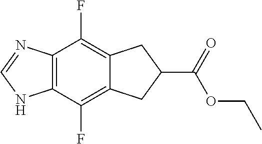 [SMILES: CCOC(=O)C1Cc2c(c(F)c3[nH]cnc3c2F)C1]

A solution of ethyl 5,6-diamino-4,7-difluoro-indane-2-carboxylate (Intermediate 5, 500.0 mg, 1.95 mmol, 1.0 eq) in formic acid (5 mL) was stirred at 100° C. for 2 h. The reaction mixture was cooled to room temperature, diluted with H₂O (20 mL) and extracted with EtOAc (15 mL×3). The combined organic layers were washed with brine (20 mL×2), dried over anhydrous Na2SO₄, filtered and concentrated under reduced pressure to give the crude title compound (460.0 mg, 1.73 mmol, 79.7% yield) as a brown solid.

### Steps 2 to 4: tert-butyl N-[2-(4,8-difluoro-6-formyl-6,7-dihydro-5H-cyclopenta[f]benzimidazol-1-yl)ethyl]-N-methyl-carbamate

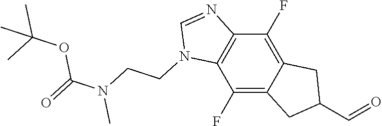 [SMILES: CN(CCn1cnc2c(F)c3c(c(F)c21)CC(C=O)C3)C(=O)OC(C)(C)C]

The title compound was prepared by analogy with Intermediate 71, Steps 2 to 4 using tert-butyl N-(2-chloroethyl)-N-methyl-carbamate [CAS #220074-38-4] in place of 1-BOC-3-iodoazetidine and was obtained as a yellow oil. MS (ESP) m/z=380.2 [M+H]⁺.

### Intermediate 99

### tert-Butyl N-[2-(4,8-difluoro-6-formyl-6,7-dihydro-5H-cyclopenta[f]benzimidazol-1-yl)ethyl]-N-methyl-carbamate

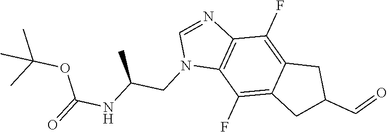 [SMILES: C[C@@H](Cn1cnc2c(F)c3c(c(F)c21)CC(C=O)C3)NC(=O)OC(C)(C)C]

The title compound was prepared from ethyl 4,8-difluoro-3,5,6,7-tetrahydrocyclopenta[f]benzimidazole-6-carboxylate (Intermediate 98, Step 1) and BOC-L-alaninol by analogy with Intermediate 76 and was obtained as a yellow oil. MS (ESP) m/z=380.2 [M+H]⁺.

### Intermediate 100

### tert-Butyl N-[(3R,4R)-4-(8-fluoro-6-formyl-6,7-dihydro-5H-cyclopenta[f][1,3]benzoxazol-2-yl)tetrahydrofuran-3-yl]carbamate

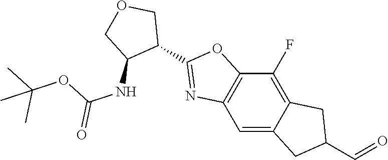 [SMILES: CC(C)(C)OC(=O)N[C@H]1COC[C@@H]1c1nc2cc3c(c(F)c2o1)CC(C=O)C3]

The title compound was prepared by analogy with Intermediate 85 using (3R,4R)-4-(tert-butoxycarbonylamino)tetrahydrofuran-3-carboxylic acid [CAS #1821806-18-1] in place of BOC-BETA-ALA-OH and was obtained as a dark brown oil. MS (ESP) m/z=335.3 [M+H-tBu]⁺.

### Intermediate 101

### tert-Butyl (2S,4R)-4-[tert-butyl(diphenyl)silyl]oxy-2-(8-fluoro-6-formyl-6,7-dihydro-5H-cyclopenta[f][1,3]benzoxazol-2-yl)pyrrolidine-1-carboxylate

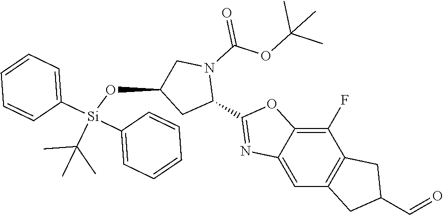 [SMILES: CC(C)(C)OC(=O)N1C[C@H](O[Si](c2ccccc2)(c2ccccc2)C(C)(C)C)C[C@H]1c1nc2cc3c(c(F)c2o1)CC(C=O)C3]

The title compound was prepared by analogy with Intermediate 85 using (2S,4R)-1-tert-butoxycarbonyl-4-[tert-butyl(diphenyl)silyl]oxy-pyrrolidine-2-carboxylic acid [CAS #1357471-22-7] in place of BOC-BETA-ALA-OH.

### Intermediate 102

### tert-Butyl N-[(1S)-2-[tert-butyl(diphenyl)silyl]oxy-1-(8-fluoro-6-formyl-6,7-dihydro-5H-cyclopenta[f][1,3]benzoxazol-2-yl)ethyl]carbamate

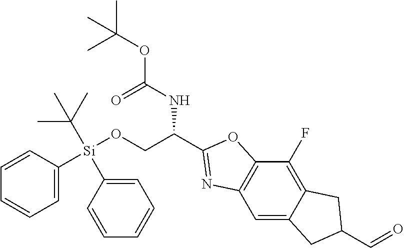 [SMILES: CC(C)(C)OC(=O)N[C@@H](CO[Si](c1ccccc1)(c1ccccc1)C(C)(C)C)c1nc2cc3c(c(F)c2o1)CC(C=O)C3]

The title compound was prepared by analogy with Intermediate 85 using (2S)-2-(tert-butoxycarbonylamino)-3-[tert-butyl(diphenyl)silyl]oxy-propanoic acid [CAS #145790-51-8] in place of BOC-BETA-ALA-OH and was obtained as a yellow oil. MS (ESP) m/z=603.2 [M+H]⁺.

### Intermediate 103

### tert-Butyl N-[2-[tert-butyl(diphenyl)silyl]oxy-1-(8-fluoro-6-formyl-6,7-dihydro-5H-cyclopenta[f][1,3]benzoxazol-2-yl)ethyl]-N-methyl-carbamate

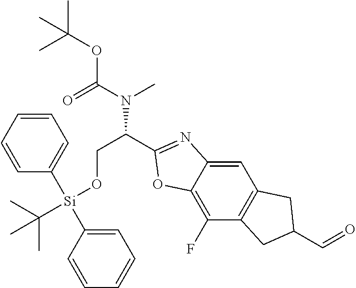 [SMILES: CN(C(=O)OC(C)(C)C)[C@@H](CO[Si](c1ccccc1)(c1ccccc1)C(C)(C)C)c1nc2cc3c(c(F)c2o1)CC(C=O)C3]

### Step 1: (2,5-dioxopyrrolidin-1-yl) (2S)-2-[tert-butoxycarbonyl(methyl)amino]-3-[tert-butyl(diphenyl)silyl]oxy-propanoate

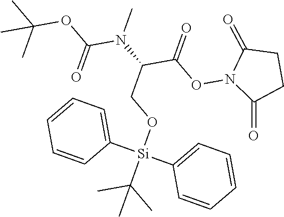 [SMILES: CN(C(=O)OC(C)(C)C)[C@@H](CO[Si](c1ccccc1)(c1ccccc1)C(C)(C)C)C(=O)ON1C(=O)CCC1=O]

To a mixture of (2S)-2-[tert-butoxycarbonyl(methyl)amino]-3-[tert-butyl(diphenyl)silyl]oxy-propanoic acid [CAS #205866-64-4] (12.5 g, 27.31 mmol, 1.0 eq), 1-hydroxypyrrolidine-2,5-dione (7.54 g, 65.56 mmol, 2.4 eq) and N,N-dimethylpyridin-2-amine (99.6 mg, 2.73 mmol, 0.1 eq) in DCM (200 mL) was added EDCI (10.47 g, 54.63 mmol, 2.0 eq) and the mixture was stirred at 10° C. for 1 h. The mixture was concentrated and the residue was purified via flash column chromatography (120 g SiO₂, 1:3 ethyl acetate/petroleum ether) to give the title compound (10.25 g, 18.47 mmol, 66.6% yield) as a colorless oil. MS (ESP) m/z=455.0 [M+H-BOC]⁺.

### Step 2: ethyl 4-fluoro-5-hydroxy-6-[[(2S)-2-[tert-butoxycarbonyl(methyl)amino]-3-[tert-butyl(diphenyl)silyl]oxy-propanoyl]amino]indane-2-carboxylate

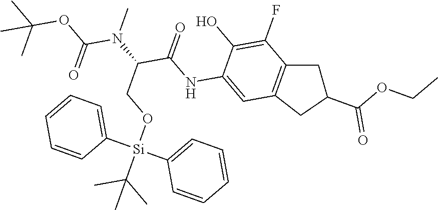 [SMILES: CCOC(=O)C1Cc2cc(NC(=O)[C@H](CO[Si](c3ccccc3)(c3ccccc3)C(C)(C)C)N(C)C(=O)OC(C)(C)C)c(O)c(F)c2C1]

To a solution of (2,5-dioxopyrrolidin-1-yl) (2S)-2-[tert-butoxycarbonyl(methyl)amino]-3-[tert-butyl(diphenyl)silyl]oxy-propanoate (7.54 g, 13.59 mmol, 1.0 eq) in DCM (60 mL) was added Et₃N (3.32 mL, 27.19 mmol, 2.0 eq) at 15° C. After 0.1 h, a solution of ethyl 6-amino-4-fluoro-5-hydroxy-indane-2-carboxylate (Intermediate 29, Step 1, 3.25 g, 13.59 mmol, 1.0 eq) in DCM (60 mL) was added at 20° C. and the resulting mixture was stirred at 15° C. for 12 h. The reaction mixture was concentrated in vacuo and was purified by silica column chromatography (1:3 ethyl acetate/petroleum ether) to give the title compound (4.88 g, 7.19 mmol, 52.1% yield) as a colorless viscous oil.

### Steps 3 to 5: tert-butyl N-[2-[tert-butyl(diphenyl)silyl]oxy-1-(8-fluoro-6-formyl-6,7-dihydro-5H-cyclopenta[f][1,3]benzoxazol-2-yl)ethyl]-N-methyl-carbamate

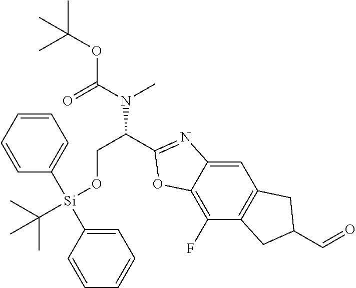 [SMILES: CN(C(=O)OC(C)(C)C)[C@@H](CO[Si](c1ccccc1)(c1ccccc1)C(C)(C)C)c1nc2cc3c(c(F)c2o1)CC(C=O)C3]

The title compound was prepared by analogy with Intermediate 85, Steps 2 to 4, and was obtained as a dark brown oil. MS (ESP) m/z=617.2 [M+H]⁺.

### Intermediate 104

### 6-[8-[[2-(Aminomethyl)-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid

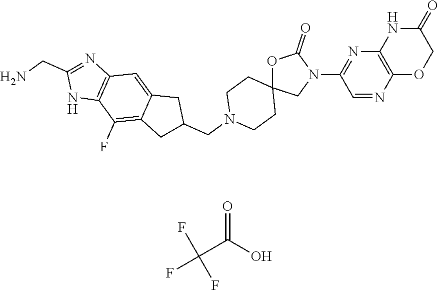 [SMILES: NCc1nc2cc3c(c(F)c2[nH]1)CC(CN1CCC2(CC1)CN(c1cnc4c(n1)NC(=O)CO4)C(=O)O2)C3 ; O=C(O)C(F)(F)F]

### Steps 1 to 4: tert-butyl N-[(8-fluoro-6-formyl-1,5,6,7-tetrahydrocyclopenta[f]benzimidazol-2-yl)methyl]carbamate

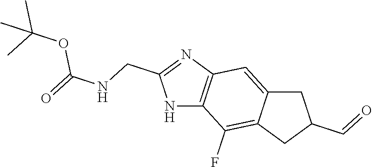 [SMILES: CC(C)(C)OC(=O)NCc1nc2cc3c(c(F)c2[nH]1)CC(C=O)C3]

The title compound was prepared from ethyl 5,6-diamino-4-fluoro-indane-2-carboxylate (Intermediate 7) and BOC-glycine by analogy with Intermediate 12 and was obtained as a brown viscous oil. MS (ESP) m/z=334.2 [M+H]⁺.

### Steps 5 and 6: 6-[8-[[2-(aminomethyl)-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid

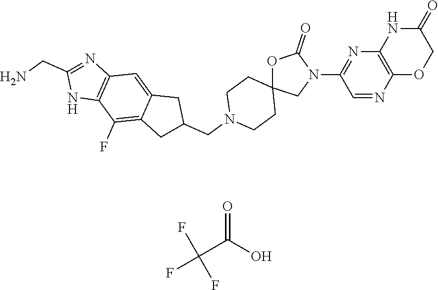 [SMILES: NCc1nc2cc3c(c(F)c2[nH]1)CC(CN1CCC2(CC1)CN(c1cnc4c(n1)NC(=O)CO4)C(=O)O2)C3 ; O=C(O)C(F)(F)F]

The title compound was prepared by analogy with Example 67 and was obtained as a light yellow solid. MS (ESP) m/z=523.3 [M+H]⁺.

### Intermediate 105

### tert-Butyl N-[(1R)-2-[(8-fluoro-6-formyl-6,7-dihydro-5H-cyclopenta[f][1,3]benzoxazol-2-yl)methylamino]-1-methyl-2-oxo-ethyl]-N-methyl-carbamate

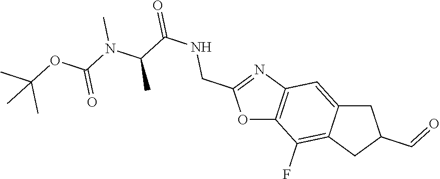 [SMILES: C[C@H](C(=O)NCc1nc2cc3c(c(F)c2o1)CC(C=O)C3)N(C)C(=O)OC(C)(C)C]

### Step 1: ethyl 2-[[[(2R)-2-[tert-butoxycarbonyl(methyl)amino]propanoyl]amino]methyl]-8-fluoro-6,7-dihydro-5H-cyclopenta[f][1,3]benzoxazole-6-carboxylate

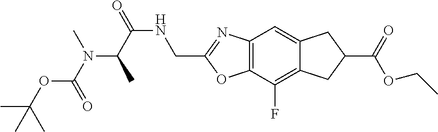 [SMILES: CCOC(=O)C1Cc2cc3nc(CNC(=O)[C@@H](C)N(C)C(=O)OC(C)(C)C)oc3c(F)c2C1]

Triphenylphosphine (1493 mg, 5.69 mmol, 1.7 eq) was added to a stirred solution of ethyl 2-(azidomethyl)-8-fluoro-6,7-dihydro-5H-cyclopenta[f][1,3]benzoxazole-6-carboxylate (Intermediate 86, Step 1, 1022 mg, 3.36 mmol, 1.0 eq) in EtOAc (16.8 mL) and the mixture was stirred for 10 min. Water (0.61 mL, 33.86 mmol, 10.08 eq) was added then the mixture was heated to 60° C. and stirred for 16 h. The mixture was concentrated in vacuo to afford the crude intermediate product (2.65 g). A solution of this product (934.0 mg, 1.0 eq), BOC-N-methyl-D-alanine (682 mg, 3.36 mmol, 1.0 eq), triethylamine (0.94 mL, 6.71 mmol, 2.0 eq) and O-(7-azabenzotriazol-1-yl)-N,N,N′,N′-tetramethyluronium hexafluorophosphate (1787 mg, 4.7 mmol, 1.4 eq) in DCM (16.8 mL) was stirred for 2 h at 25° C. The mixture was diluted with DCM and washed with a saturated solution of NaHCO₃. The organic phase was dried over Na₂SO₄ and concentrated in vacuo. The residue was purified by silica cartridge chromatography (25 g, from 100% cyclohexane to 50:50 cyclohexane/EtOAc) to afford the title compound (1299.0 mg, 2.8 mmol, 66.8% yield) as a yellow oil. MS (ESP) m/z=464.25 [M+H]⁺.

### Steps 2 and 3: tert-Butyl N-[(1R)-2-[(8-fluoro-6-formyl-6,7-dihydro-5H-cyclopenta[f][1,3]benzoxazol-2-yl)methylamino]-1-methyl-2-oxo-ethyl]-N-methyl-carbamate

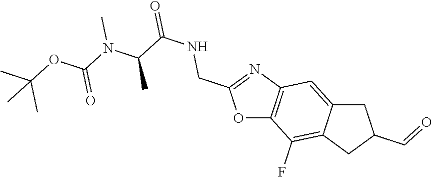 [SMILES: C[C@H](C(=O)NCc1nc2cc3c(c(F)c2o1)CC(C=O)C3)N(C)C(=O)OC(C)(C)C]

The title compound was prepared by analogy with Intermediate 28, Steps 3 and 4, and was obtained as a brown foam. MS (ESP) m/z=420.25 [M+H]⁺.

1. A compound of formula (I) 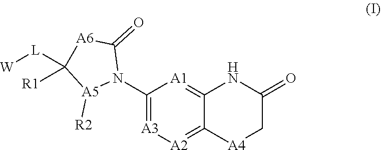
wherein
A¹ is —N—;
A² is selected from
i) —N—, and
ii) —CH—;
A³ is —CH—;
A⁴ is selected from
i) —O—, and
ii) —S—;
A⁵ is —CH—;
A⁶ is —O—;
W is selected from ring-systems: 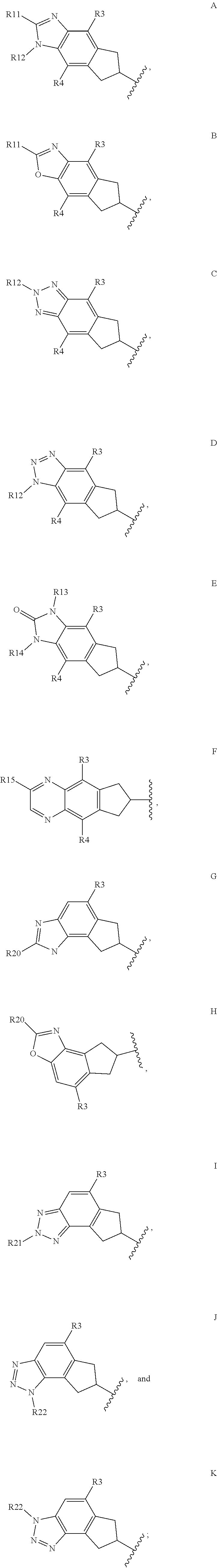
R² is H;
R³ and R⁴ are independently selected from
i) H,
ii) C₁₋₆-alkyl,
iii) halogen,
iv) OH, and
v) cyano;
L is -L₁-NR¹⁰-L₂-;
L₁ is (CH₂)ₓ, wherein x represents an integer of 1 to 6;
L₂ is (CH₂)_{y} wherein y represents an integer of 1 to 5;
R¹ is H and R¹⁰ is selected from
i) H, and
ii) C₁₋₆-alkyl;
or R¹, R¹⁰ and L₂ and the atoms to which they are bonded form a 4-6 membered heterocycle ring comprising a single N heteroatom,
or R¹ and L₂ and the atoms to which they are bonded form a 3-6 membered cycloalkyl ring;
R¹¹ is selected from
i) NH₂,
ii) C₁₋₆-alkyl,
iii) heterocycloalkyl,
iv) heterocycloalkylalkyl,
v) substituted cycloalkyl,
vi) substituted heterocycloalkyl,
vii) substituted heterocycloalkylalkyl,
viii) aminoalkyl,
ix) alkylaminoalkyl,
x) (dialkylamino)alkyl,
xi) alkylaminoacetamido,
xii) aminohydroxyalkyl,
xiii) hydroxyalkyl,
xiv) hydroxy(alkylamino)alkyl,
xv) —(CH₂)ₙNR^{a}R^{b}, and
xvi) —CH₂—O—(CH₂)ₙNR^{a}R^{b},
xvii) (dialkylamino)hydroxyalkyl, and
xviii) H
wherein n is an integer that is either 1 or 2,
wherein substituted cycloalkyl, substituted heterocycloalkyl and substituted heterocycloalkyalkyl are substituted with 1-to-2 substituents independently selected from alkyl, alkoxy, amino, alkylamino, dialkylamino, OH, oxo and dioxo,
wherein R^{a} is selected from 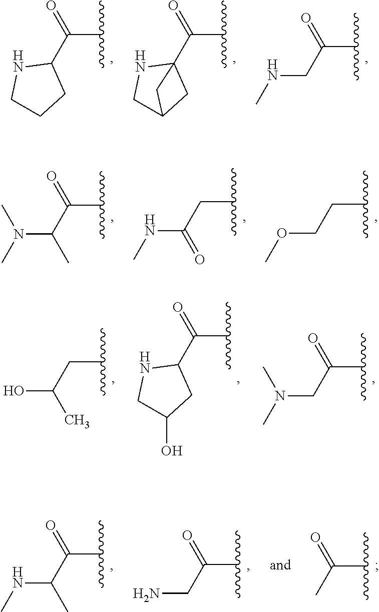
and R^{b} is selected from H or C₁₋₆-alkyl;
R¹² is selected from
i) H,
ii) C₁₋₆-alkyl,
iii) aminoalkyl,
iv) (alkylamino)acetamidoalkyl, and
v) —(CH₂)₂NR^{c}R^{d},
vi) Cycloalkylalkyl substituted by amino,
vii) Cyclopropylaminoalkyl,
viii) Heterocycloalkyl,
ix) Alkylaminoalkyl, and
x) (dialkylamino)alkyl
wherein R^{c} is selected from 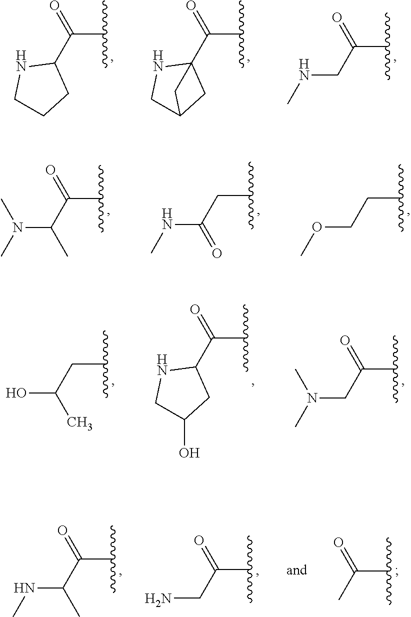
and R^{d} is selected from H or C₁₋₆-alkyl;
or R¹¹ and R¹² together form a 6-membered heterocycle ring comprising 1 or 2 N heteroatoms;
R¹³ is selected from
i) H, and
ii) aminoalkyl;
R¹⁴ is aminoalkyl;
R¹⁵ is selected from
i) NH₂, and
ii) (alkylamino)alkylamino;
R²⁰ is alkylaminoalkyl;
R²¹ is aminoalkyl, alkylaminoalkyl or (dialkylamino)alkyl;
R²² is aminoalkyl;
or pharmaceutically acceptable salts thereof.

2. The compound according to claim 1, wherein A⁴ is —O—.

3. The compound according to claim 1, wherein A² is —N—.

4. The compound according to claim 1, wherein R³ and R⁴ are independently selected from
i) H, and
ii) halogen.

5. The compound according to claim 1, wherein W is selected from ring systems A, B, C, D, G, H and I.

6. The compound according to claim 1, wherein W is selected from ring systems A, B, C, D and I.

7. The compound according to claim 1, wherein R¹¹ is selected from
i) NH₂,
ii) C₁₋₆-alkyl,
iii) a 5- to 6-membered heterocycloalkyl comprising 1 N atom, 1 O atom, or both 1 N atom and 1 O atom,
iv) A 6-membered heterocycloalkylalkyl comprising 1 N atom and 1 O atom,
v) a 5-to-6-membered substituted heterocycloalkyl comprising 1 N atom, 1 O atom, or both 1 N atom and 1 O atom,
vi) a 6-membered substituted heterocycloalkylalkyl comprising 1 N atom and 1 S atom;
vii) aminoalkyl,
viii) alkylaminoalkyl,
ix) (dialkylamino)alkyl,
x) alkylaminoacetamido,
xi) aminohydroxyalkyl,
xii) hydroxyalkyl, and
xiii) hydroxy(alkylamino)alkyl,
xiv) H,
xv) (dialkylamino)hydroxyalkyl, and
xvi) —(CH₂)ₙNR^{a}R^{b}
wherein n is an integer that is either 1 or 2,
wherein R^{a} is selected from 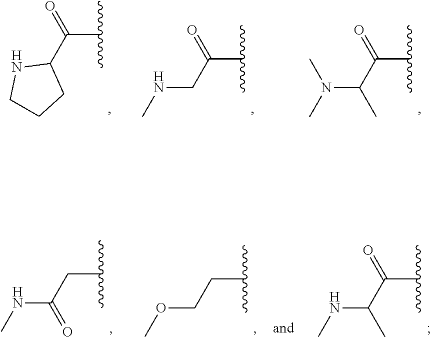
and R^{b} is H;
wherein substituted heterocycloalkyl and substituted heterocycloalkyalkyl are substituted with 1-to-2 substituents independently selected from alkyl, alkoxy, amino, alkylamino, OH, oxo and dioxo.

8. The compound according to claim 1, wherein R¹¹ is selected from
i) a 4- to 6-membered heterocycloalkyl comprising 1 N atom, 1 O atom, or both 1 N atom and 1 O atom,
ii) a 5-to-6-membered substituted heterocycloalkyl comprising 1 N atom, 1 O atom, or both 1 N atom and 1 O atom,
iii) aminoalkyl,
iv) alkylaminoalkyl,
v) (dialkylamino)alkyl,
vi) alkylaminoacetamido,
vii) aminohydroxyalkyl, and
viii) hydroxy(alkylamino)alkyl,
wherein substituted heterocycloalkyl is substituted with alkoxy, amino, or OH.

9. The compound according to claim 1, wherein R¹¹ is selected from
i) a 5-membered substituted heterocycloalkyl comprising 1 N atom,
ii) a 4-membered heterocycloalkyl cinorusubg 1 N atom,
iii) aminoalkyl,
iv) alkylaminoalkyl,
v) (dialkylamino)alkyl, and
vi) aminohydroxyalkyl
wherein substituted heterocycloalkyl is substituted with OH.

10. The compound according to claim 1, wherein R¹² is selected from
i) H,
ii) C₁₋₆-alkyl,
iii) aminoalkyl,
iv) a 4-membered heterocycloalkyl comprising 1 N atom,
v) alkylaminoalkyl, and
vi) (dialkylamino)alkyl

11. The compound according to claim 1, wherein R¹² is selected H, alkylaminoalkyl, aminoalkyl, or a 4-membered heterocycloalkyl ring comprising 1 N atom.

12. The compound according to claim 1, wherein R¹² is H.

13. The compound according to claim 1, wherein R¹¹ and R¹² together form a 6-membered heterocycle ring comprising 2 N heteroatoms.

14. The compound according to claim 1, wherein R¹ and R¹⁰ are H,
or R¹, R¹⁰ and L₂ and the atoms to which they are bonded form the piperidine ring as below 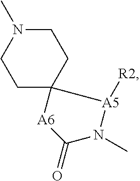
or R¹ and L₂ and the atoms to which they are bonded form the 4-membered cycloalkyl ring as below

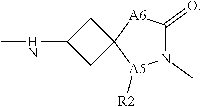

15. The compound according to claim 1, wherein R¹, R¹⁰ and L₂ and the atoms to which they are bonded form the piperidine ring as below,

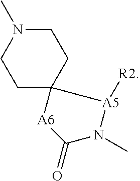

16. The compound according to claim 1, wherein
A¹ is —N—;
A² is selected from
i) —N—, and
ii) —CH—;
A³ is —CH—;
A⁴ is —O—;
A⁵ is —CH—;
A⁶ is —O—;
W is selected from ring-systems: 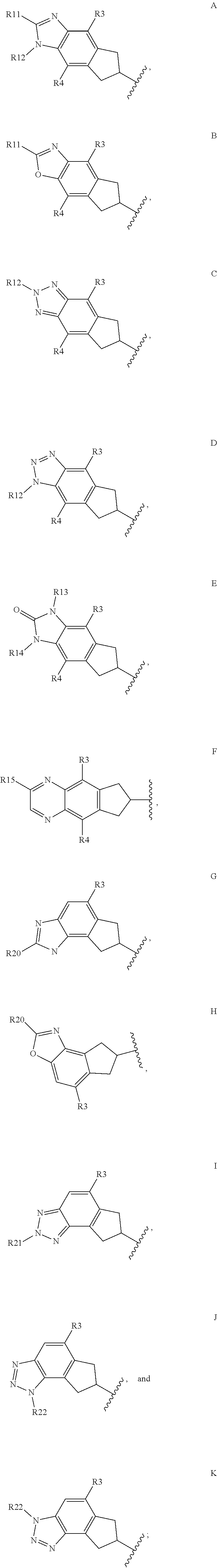
R² is H;
R³ and R⁴ are independently selected from H and halogen;
L is -L₁-NR¹⁰-L₂-;
L₁ is (CH₂)ₓ, wherein x represents an integer of 1 to 6;
L₂ is (CH₂)_{y} wherein y represents an integer of 1 to 5;
R¹ is H and R¹⁰ is selected from
i) H, and
ii) C₁₋₆-alkyl;
or R¹, R¹⁰ and L₂ and the atoms to which they are bonded form a 4-6 membered heterocycle ring comprising a single N heteroatom,
or R¹ and L₂ and the atoms to which they are bonded form a 3-6 membered cycloalkyl ring;
R¹¹ is selected from
i) NH₂,
ii) C₁₋₆-alkyl,
iii) a 5- to 6-membered heterocycloalkyl comprising 1 N atom, 1 O atom, or both 1 N atom and 1 O atom,
iv) A 6-membered heterocycloalkylalkyl comprising 1 N atom and 1 O atom,
v) a 5-to-6-membered substituted heterocycloalkyl comprising 1 N atom, 1 O atom, or both 1 N atom and 1 O atom,
vi) a 6-membered substituted heterocycloalkylalkyl comprising 1 N atom and 1 S atom;
vii) aminoalkyl,
viii) alkylaminoalkyl,
ix) (dialkylamino)alkyl,
x) alkylaminoacetamido,
xi) aminohydroxyalkyl,
xii) hydroxyalkyl, and
xiii) hydroxy(alkylamino)alkyl,
xiv) H,
xv) (dialkylamino)hydroxyalkyl, and
xvi) —(CH₂)ₙNR^{a}R^{b}
wherein n is an integer that is either 1 or 2,
wherein R^{a} is selected from 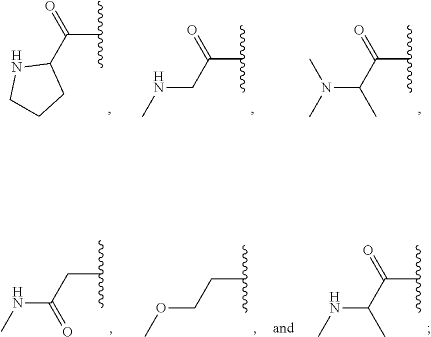
and R^{b} is H;
wherein substituted heterocycloalkyl and substituted heterocycloalkyalkyl are substituted with 1-to-2 substituents independently selected from alkyl, alkoxy, amino, alkylamino, OH, oxo and dioxo.
R¹² is selected from
i) H,
ii) C₁₋₆-alkyl,
iii) aminoalkyl,
iv) (alkylamino)acetamidoalkyl,
v) —(CH₂)₂NR^{c}R^{d},
vi) cycloalkylalkyl substituted by amino,
vii) cyclopropylaminoalkyl,
viii) heterocycloalkyl,
ix) alkylaminoalkyl, and
x) (dialkylamino)alkyl
wherein R^{c} is selected from 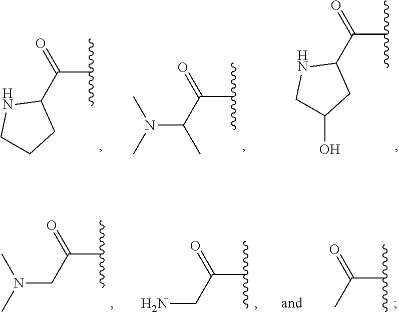
and R^{d} is selected from H or C₁₋₆-alkyl;
or R¹¹ and R¹² together form a 6-membered heterocycle ring comprising 1 or 2 N heteroatoms;
R¹³ is selected from H and aminoalkyl;
R¹⁴ is aminoalkyl;
R¹⁵ is selected from NH₂, and (alkylamino)alkylamino;
R²⁰ is alkylaminoalkyl;
R²¹ is aminoalkyl or alkylaminoalkyl;
R²² is aminoalkyl;
or pharmaceutically acceptable salts thereof.

17. The compound or a pharmaceutically acceptable salt thereof according to claim 1, wherein
A¹ is —N—;
A² is selected from
i) —N—, and
ii) —CH—;
A³ is —CH—;
A⁴ is —O—;
A⁵ is —CH—;
A⁶ is —O—;
W is selected from ring-systems A, B, C, D, G, H and I.
R² is H;
R³ and R⁴ are independently selected from
i) H, and
ii) halogen;
L is -L₁-NR¹⁰-L₂-;
L₁ is (CH₂)ₓ, wherein x is 1;
R¹, R¹⁰ and L₂ and the atoms to which they are bonded form the piperidine ring as below, 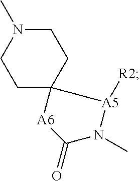
R¹¹ is selected from
i) a 4- to 6-membered heterocycloalkyl comprising 1 N atom, 1 O atom, or both 1 N atom and 1 O atom,
ii) a 5-to-6-membered substituted heterocycloalkyl comprising 1 N atom, 1 O atom, or both 1 N atom and 1 O atom,
iii) aminoalkyl,
iv) alkylaminoalkyl,
v) (dialkylamino)alkyl,
vi) alkylaminoacetamido,
vii) aminohydroxyalkyl, and
viii) hydroxy(alkylamino)alkyl,
wherein substituted heterocycloalkyl is substituted with alkoxy, amino, or OH.
R¹² is selected from
i) H,
ii) C₁₋₆-alkyl,
iii) aminoalkyl,
iv) a 4-membered heterocycloalkyl comprising 1 N atom,
v) aminoalkyl,
vi) alkylaminoalkyl, and
vii) (dialkylamino)alkyl
R²⁰ is alkylaminoalkyl;
R²¹ is alkylaminoalkyl or amimoalkyl;
or pharmaceutically acceptable salts thereof.

18. The compound or a pharmaceutically acceptable salt thereof according to claim 1, wherein
A¹ is —N—;
A² is —N—;
A³ is —CH—;
A⁴ is —O—;
A⁵ is —CH—;
A⁶ is —O—;
W is selected from ring-systems A, B, C, D and I.
R² is H;
R³ and R⁴ are independently selected from
i) H, and
ii) fluoro;
L is -L₁-NR¹⁰-L₂-;
L₁ is (CH₂)ₓ, wherein x is 1;
R¹, R¹⁰ and L₂ and the atoms to which they are bonded form the piperidine ring as below, 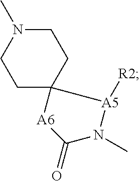
R¹¹ is selected from
i) a 5-membered substituted heterocycloalkyl comprising 1 N atom substituted with OH;
ii) a 4-membered heterocycloalkyl comprising 1 N atom,
iii) aminoalkyl,
iv) alkylaminoalkyl,
v) (dialkylamino)alkyl, and
vi) aminohydroxyalkyl
R¹² is selected from H, alkylaminoalkyl, aminoalkyl, or a 4-membered heterocycloalkyl ring comprising 1 N atom;
or pharmaceutically acceptable salts thereof.

19. A compound according to claim 1, selected from
6-[5-[2-[(4,8-difluoro-2-methyl-3,5,6,7-tetrahydrocyclopenta[f]benzimidazol-6-yl)methylamino]ethyl]-2-oxo-oxazolidin-3-yl]-4H-pyrazino[2,3-b][1,4]oxazin-3-one;
6-[5-[2-[[2-[(dimethylamino)methyl]-4,8-difluoro-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;
6-[8-[[2-[(dimethylamino)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(1-hydroxy-1-methyl-ethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1,1-dioxo-1,4-thiazinan-4-yl)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(4-methylmorpholin-2-yl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(morpholinomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3R)-morpholin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3R)-morpholin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-(methylaminomethyl)-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-5-oxopyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-5-oxopyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[(10,16-difluoro-2,5,8-triazatetracyclo[7.7.0.02,7.011,15]hexadeca-1(9),7,10,15-tetraen-13-yl)methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-[(1R)-1-(methylamino)ethyl]-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-2-[(2S,4R)-4-(methylamino)pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-4-hydroxy-2-piperidyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-4-hydroxy-2-piperidyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[2-[(dimethylamino)methyl]-4,8-difluoro-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[2-(1-amino-1-methyl-ethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S,4R)-4-aminotetrahydrofuran-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[(2-amino-4,8-difluoro-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-(methylaminomethyl)-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-azabicyclo[2.1.1]hexan-1-yl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
N-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-2-(methylamino)acetamide;
6-[8-[[2-[(1S,2S)-1-amino-2-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-methoxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[5-[2-[[4,8-difluoro-2-[(2S,4R)-4-hydroxypyrrolidin-2-yl]-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;
6-[5-[2-[[4,8-difluoro-2-[(2S,4R)-4-hydroxypyrrolidin-2-yl]-3,5,6,7-tetrahydrocyclopenta[f]benzimidazol-6-yl]methylamino]ethyl]-2-oxo-oxazolidin-3-yl]-4H-pyrazino[2,3-b][1,4]oxazin-3-one;2,2,2-trifluoroacetic acid;
6-[8-[[8-fluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(2R,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[[4,8-difluoro-2-[(2S,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-[[4,8-difluoro-2-[(2R,4R)-4-hydroxypyrrolidin-2-yl]-3,5,6,7-tetrahydrocyclopenta[f]benzimidazol-6-yl]methylamino]-6-oxo-5-oxa-7-azaspiro[3.4]octan-7-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S,4R)-4-hydroxypyrrolidin-2-yl]-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-3-hydroxy-2-(methylamino)propyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxy-1-methyl-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(methylamino)acetamide;
or pharmaceutically acceptable salts thereof.

20. A compound according to claim 1, selected from
6-[8-[[2-[2-(dimethylamino)ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-(dimethylamino)-3-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(dimethylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-[2-(dimethylamino)ethyl]-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[2-(methylamino)ethyl]-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2-methoxyethylamino)methyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
2-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]methylamino]-N-methyl-acetamide;
6-[8-[[1-(2-aminoethyl)-8-fluoro-2-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-2-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[(1-aminocyclopropyl)methyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[1-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-5-fluoro-7,8-dihydro-6H-cyclopenta[e]benzotriazol-7-yl]methyl]-2-keto-1-oxa-3,8-diazaspiro[4.5]decan-3-yl]-4H-pyrazino[2,3-b][1,4]oxazin-3-one;
6-[8-[[3-(2-aminoethyl)-5-fluoro-7,8-dihydro-6H-cyclopenta[e]benzotriazol-7-yl]methyl]-2-keto-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(1R)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-(methylaminomethyl)-1,6,7,8-tetrahydrocyclopenta[e]benzimidazol-7-yl]methyl]-2-oxo-1-oxa-3,8-diazaspiro[4.5]decan-3-yl]-4H-pyrazino[2,3-b][1,4]oxazin-3-one;
6-[8-[[4-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[8-[[5-fluoro-2-(methylaminomethyl)-7,8-dihydro-6H-cyclopenta[e][1,3]benzoxazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(3-hydroxyazetidin-1-yl)methyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(aminomethyl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(azetidin-3-yl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
formic acid;6-[2-oxo-8-[[3-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[3-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-[2-(cyclopropylamino)ethyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[2-oxo-8-[[4,8-difluoro-1-[2-(methylamino)ethyl]-6,7-dihydro-5H-cyclopenta[f]benzimidazol-6-yl]methyl]-1-oxa-3,8-diazaspiro[4.5]decan-3-yl]-4H-pyrazino[2,3-b][1,4]oxazin-3-one;
6-[8-[[1-[(2S)-2-aminopropyl]-4,8-difluoro-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[1-[(2S)-2-aminopropyl]-4,8-difluoro-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;hydrochloric acid;
6-[8-[[1-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[8-[[2-[(3R,4R)-4-aminotetrahydrofuran-3-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(1S)-2-hydroxy-1-(methylamino)ethyl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)propanamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(dimethylamino)propanamide;
(2R)-2-(dimethylamino)-N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]propanamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(dimethylamino)-N-methyl-acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-N-methyl-acetamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]pyrrolidine-2-carboxamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-2-(methylamino)acetamide;
N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]-2-(methylamino)acetamide;2,2,2-trifluoroacetic acid;
N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]-2-(methylamino)acetamide;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]pyrrolidine-2-carboxamide;2,2,2-trifluoroacetic acid;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-1,5,6,7-tetrahydrocyclopenta[f]benzimidazol-2-yl]methyl]pyrrolidine-2-carboxamide;
(2R)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]pyrrolidine-2-carboxamide;
(2R,4S)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-4-hydroxy-pyrrolidine-2-carboxamide;
(2R,4S)—N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-4-hydroxy-pyrrolidine-2-carboxamide;
2-amino-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;formic acid;
2-amino-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-2-yl]ethyl]-N-methyl-acetamide;
2-amino-N-methyl-N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]acetamide;
N-[2-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f]benzotriazol-1-yl]ethyl]-2-(dimethylamino)acetamide;
(2R)—N-[[8-fluoro-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[f][1,3]benzoxazol-2-yl]methyl]-2-(methylamino)propanamide;
6-[2-oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride;
or pharmaceutically acceptable salts thereof.

21. A compound according to claim 1 selected from
6-[8-[[2-[(dimethylamino)methyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;
6-[8-[[4,8-difluoro-2-[(3S)-pyrrolidin-3-yl]-3,5,6,7-tetrahydrocyclopenta[f]benzimidazol-6-yl]methyl]-2-oxo-1-oxa-3,8-diazaspiro[4.5]decan-3-yl]-4H-pyrido[3,2-b][1,4]oxazin-3-one;2,2,2-trifluoroacetic acid;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;
6-[8-[(4,8-difluoro-2-morpholin-2-yl-3,5,6,7-tetrahydrocyclopenta[f]benzimidazol-6-yl)methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-[2-(methylamino)ethyl]-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R)-pyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(1-amino-1-methyl-ethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(3S,4R)-4-aminotetrahydrofuran-3-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-1-methyl-2-(methylaminomethyl)-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-azabicyclo[2.1.1]hexan-1-yl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
N-[4,8-difluoro-6-[[2-oxo-3-(3-oxo-4H-pyrazino[2,3-b][1,4]oxazin-6-yl)-1-oxa-3,8-diazaspiro[4.5]decan-8-yl]methyl]-3,5,6,7-tetrahydrocyclopenta[f]benzimidazol-2-yl]-2-(methylamino)acetamide;
6-[8-[[2-[(1S,2S)-1-amino-2-hydroxy-propyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-methoxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,3S)-3-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1S)-2-hydroxy-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
or pharmaceutically acceptable salts thereof.

22. A compound according to claim 1, selected from
6-[8-[[3-[2-(dimethylamino)ethyl]-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-1-methyl-6,7-dihydro-5H-cyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[3-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[1-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-(methylaminomethyl)-1,6,7,8-tetrahydrocyclopenta[e]benzimidazol-7-yl]methyl]-2-oxo-1-oxa-3,8-diazaspiro[4.5]decan-3-yl]-4H-pyrazino[2,3-b][1,4]oxazin-3-one;
6-[8-[[4-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[8-[[5-fluoro-2-(methylaminomethyl)-7,8-dihydro-6H-cyclopenta[e][1,3]benzoxazol-7-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-aminoethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(aminomethyl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-(azetidin-3-yl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[4,8-difluoro-2-[2-(methylamino)ethyl]-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrido[3,2-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrido[3,2-b][1,4]oxazin-3-one;
6-[2-oxo-8-[[2-(2-aminoethyl)-5-fluoro-7,8-dihydro-6H-cyclopenta[e]benzotriazol-7-yl]methyl]-1-oxa-3,8-diazaspiro[4.5]decan-3-yl]-4H-pyrido[3,2-b][1,4]oxazin-3-one dihydrochloride;
or pharmaceutically acceptable salts thereof.

23. A compound according to claim 1 selected from
6-[8-[[2-[2-(dimethylamino)ethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-(methylaminomethyl)-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(aminomethyl)-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(1R)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-4,8-difluoro-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(1S)-1-(methylamino)ethyl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[1-(2-aminoethyl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1R)-1-aminoethyl]-8-fluoro-1,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2S,4R)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;
6-[8-[[4,8-difluoro-2-[(2R,4S)-4-hydroxypyrrolidin-2-yl]-3,5,6,7-tetrahydrocyclopenta[f]benzimidazol-6-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[2-oxo-8-[[(6S)-8-fluoro-2-(methylaminomethyl)-1,5,6,7-tetrahydrocyclopenta[f]benzimidazol-6-yl]methyl]-1-oxa-3,8-diazaspiro[4.5]decan-3-yl]-4H-pyrazino[2,3-b][1,4]oxazin-3-one;
or pharmaceutically acceptable salts thereof.

24. A compound according to claim 1 selected from
6-[8-[[2-(2-aminoethyl)-8-fluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;2,2,2-trifluoroacetic acid;
6-[8-[[2-(2-aminoethyl)-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[5-fluoro-2-[2-(methylamino)ethyl]-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-5-fluoro-7,8-dihydro-6H-cyclopenta[e]benzotriazol-7-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-(methylaminomethyl)-6,7-dihydro-5H-cyclopenta[f][1,3]benzoxazol-6-yl]methyl]-2-keto-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f][1,3]benzoxazol-6-yl]methyl]-1-oxa-3,8-diazaspiro[4.5]decan-3-yl]-4H-pyrazino[2,3-b][1,4]oxazin-3-one;
6-[8-[[2-(2-aminoethyl)-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
formic acid;6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[1-(azetidin-3-yl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[2-oxo-8-[[4,8-difluoro-1-[2-(methylamino)ethyl]-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[1-(azetidin-3-yl)-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;formic acid;
6-[8-[[2-[(2S)-2-aminopropyl]-4,8-difluoro-6,7-dihydro-5H-cyclopenta[f]benzotriazol-6-yl]methyl]-2-oxo-1-oxa-3,8-diazaspiro[4.5]decan-3-yl]-4H-pyrazino[2,3-b][1,4]oxazin-3-one;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one dihydrochloride;
6-[2-oxo-8-[[2-(2-aminoethyl)-8-fluoro-6,7-dihydro-5H-cyclopenta[f]benzotriazol-6-yl]methyl]-1-oxa-3,8-diazaspiro[4.5]decan-3-yl]-4H-pyrazino[2,3-b][1,4]oxazin-3-one;
6-[8-[[2-[(2R)-azetidin-2-yl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[8-fluoro-2-[(2S,4R)-4-hydroxypyrrolidin-2-yl]-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
6-[8-[[2-[(1S)-1-amino-2-hydroxy-ethyl]-8-fluoro-6,7-dihydro-5H-cyclopenta[f][1,3]benzoxazol-6-yl]methyl]-2-oxo-1-oxa-3,8-diazaspiro[4.5]decan-3-yl]-4H-pyrazino[2,3-b][1,4]oxazin-3-one;
or pharmaceutically acceptable salts thereof.

25. A pharmaceutical composition comprising a compound according to any one of claim 1 and a therapeutically inert carrier.

26. A method for the treatment or prophylaxis of infections and resulting diseases caused by Escherichia coil, which method comprises administering an effective amount of a compound according to claim 1.

27. A method for the treatment or prophylaxis of bacterial infection, which method comprises administering an effective amount of a compound according to claim 1.

28. A process to prepare a compound according to claim 1 comprising the reacting a compound of formula III with a compound of formula II in the presence of a xxx to provide a compound of formula I, in conditions that include sodium triacetoxyborohydride or sodium cyanoborohydride, optionally in the presence of additives such as N,N-diisopropylethylamine or triethlyamine or acetic acid or powdered molecular sieves in a solvent such as methanol, THF or 1,2-dichloroethane at a temperature such as room temperature. 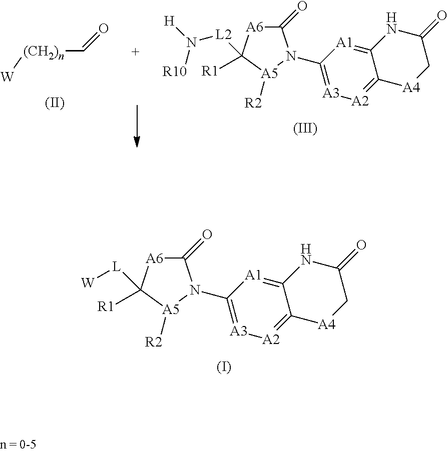
wherein R¹, R², A¹, A², A³, A⁴, A⁵, A⁶, L and W are as described above.
